(19) United States
(12) Patent Application Publication
(10) Pub. No.: US 2014/0065103 A1
(43) Pub. Date: Mar. 6, 2014

(54) COMPOUNDS AND METHODS FOR THE TREATMENT OR PREVENTION OF FLAVIVIRIDAE VIRAL INFECTIONS

(71) Applicant: VERTEX PHARMACEUTICALS INCORPORATED, (US)

(72) Inventors: Jeremy Green, Waltham, MA (US); Laval Chan Chun Kong, Kirkland (CA); Sanjoy Kumar Das, Pierrefonds (CA); Carl Poisson, Montreal (CA); Suganthini Nanthakumar, Newton, MA (US); Nathan Waal, Cambridge, MA (US); Pan Li, Lexington, MA (US); Steven Ronkin, Watertown, MA (US); David J. Lauffer, Stow, MA (US); Dean M. Wilson, Bedford, MA (US)

(73) Assignee: VERTEX PHARMACEUTICALS INCORPORATED, Cambridge, MA (US)

(21) Appl. No.: 13/767,347

(22) Filed: Feb. 14, 2013

Related U.S. Application Data
(63) Continuation of application No. PCT/US2011/048027, filed on Aug. 17, 2011.
(60) Provisional application No. 61/374,396, filed on Aug. 17, 2010.

(51) Int. Cl. C07D 417/12; A61K 31/7056; A61K 31/5377; A61K 31/4535; A61K 31/4436; A61K 31/497; A61K 31/4245; A61K 31/427; A61K 31/422; A61K 31/381; C07D 409/12; C07D 413/12; C07D 409/06; C07D 333/40; A61K 38/21

(52) U.S. Cl. CPC C07D 417/12; A61K 38/212; A61K 31/7056; A61K 31/5377; A61K 31/4535; A61K 31/4436; A61K 31/497; A61K 31/4245; A61K 31/427; A61K 31/422; A61K 31/381; C07D 409/12; C07D 413/12; C07D 409/06; C07D 333/40
USPC 424/85.7; 435/184; 514/43; 514/231.5; 514/326; 514/336; 514/339; 514/364; 514/365; 514/378; 514/444; 514/447; 544/146; 546/213; 546/281.4; 548/143; 548/204; 548/247; 549/60; 549/69

(57) ABSTRACT

A compound is selected from the structural formulae depicted in FIG. 1 or a pharmaceutically acceptable salt thereof. A pharmaceutical composition comprises a compound selected from the structural formulae depicted in FIG. 1 or a pharmaceutically acceptable salt thereof, and a pharmaceutically acceptable carrier of excipient. A method of treating a HCV infection in a subject comprises administering to the subject a therapeutically effective amount of selected from the structural formulae depicted in FIG. 1 or a pharmaceutically acceptable salt thereof. A method of inhibiting or reducing the activity of HCV polymerase in a subject or in a biological in vitro sample comprises administering to the subject or to the sample a therapeutically effective amount of selected from the structural formulae depicted in FIG. 1 or a pharmaceutically acceptable salt thereof.

### RELATED APPLICATIONS

This application is a continuation of PCT Application Number PCT/US2011/048027, filed Aug. 17, 2011, which claims priority to U.S. Provisional Application, U.S. Ser. No. 61/374,396, filed on Aug. 17, 2010. The entire teachings of these applications are incorporated herein by reference.

### BACKGROUND OF THE INVENTION

Hepatitis C virus (HCV) is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

There is therefore a great need for the development of anti-viral agents for use in treating or preventing Flaviviridae infections.

### SUMMARY OF THE INVENTION

The present invention generally relates to compounds useful for treating or preventing Flaviviridae infections, such as HCV infections.

In one embodiment, the invention is directed to a compound selected from the structural formulae depicted below or in FIG. 1, or a pharmaceutically acceptable salt thereof:

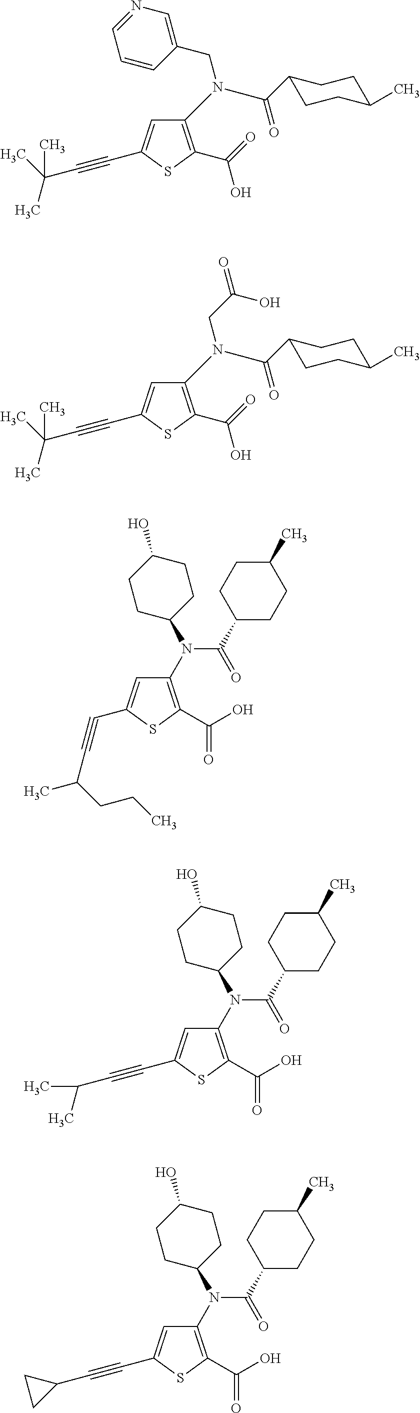

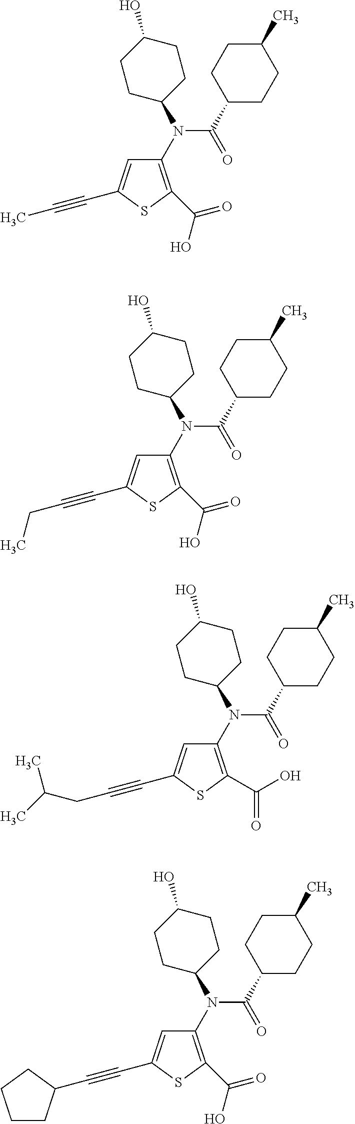

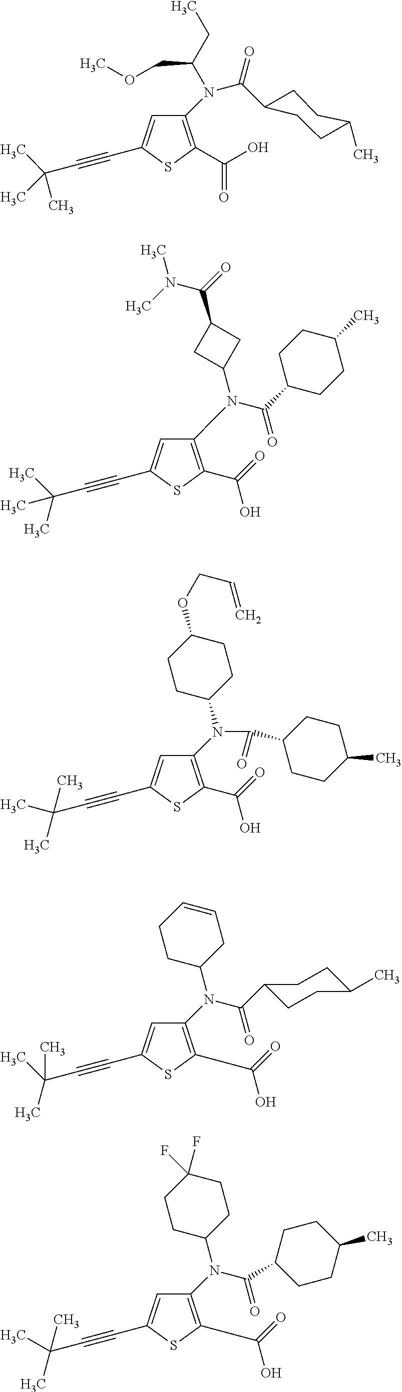

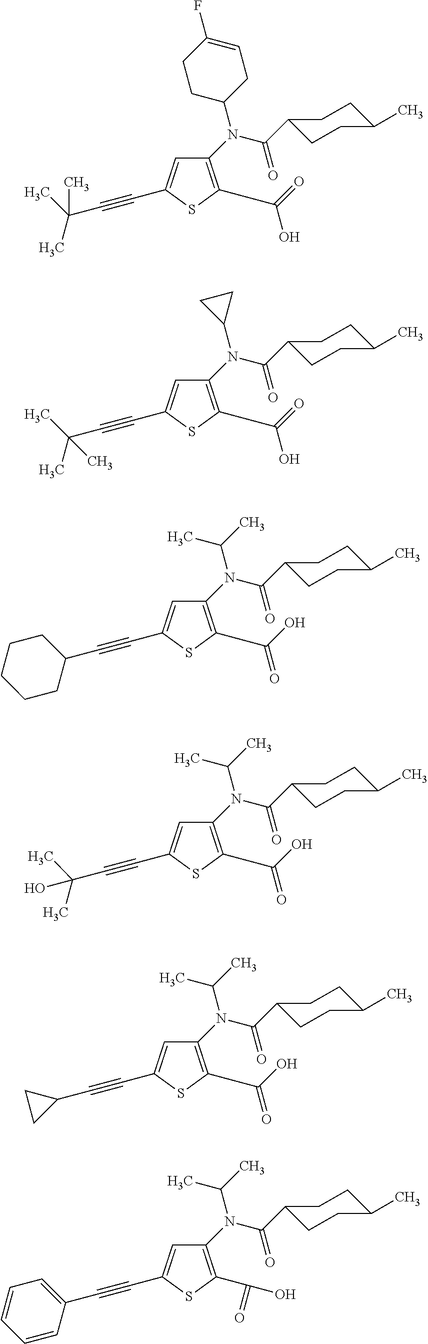

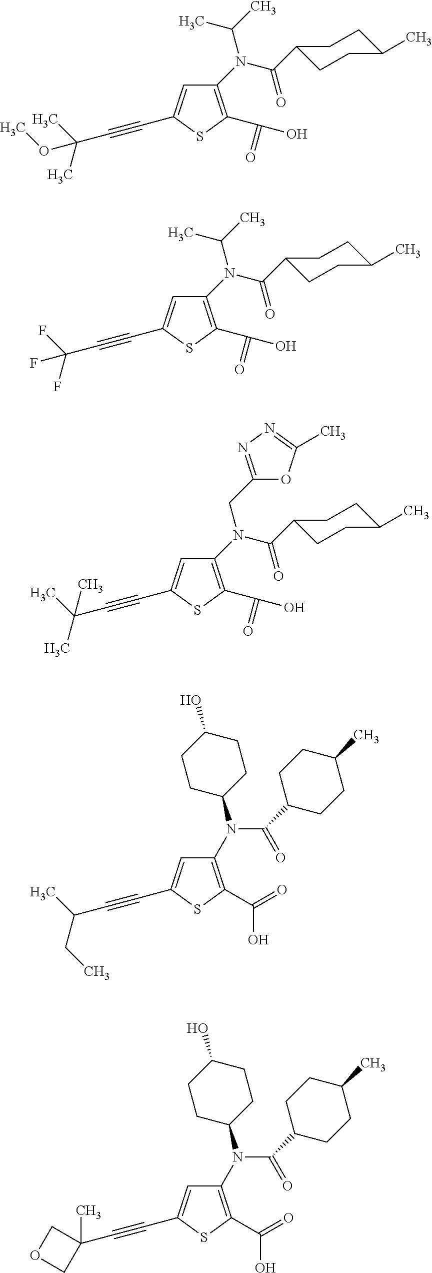

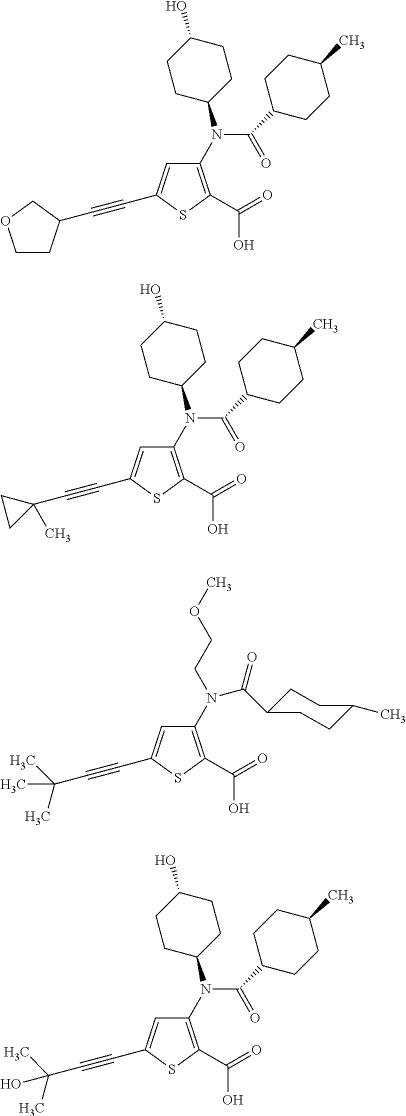

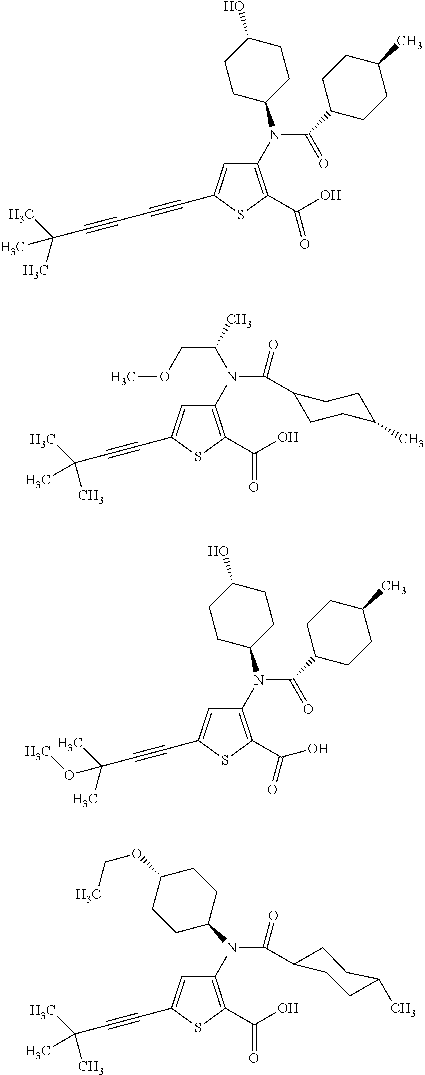

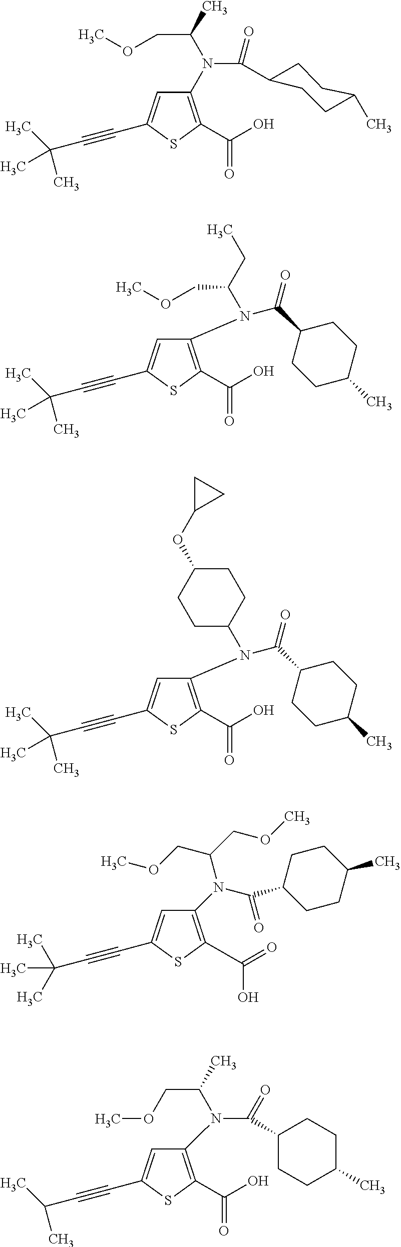

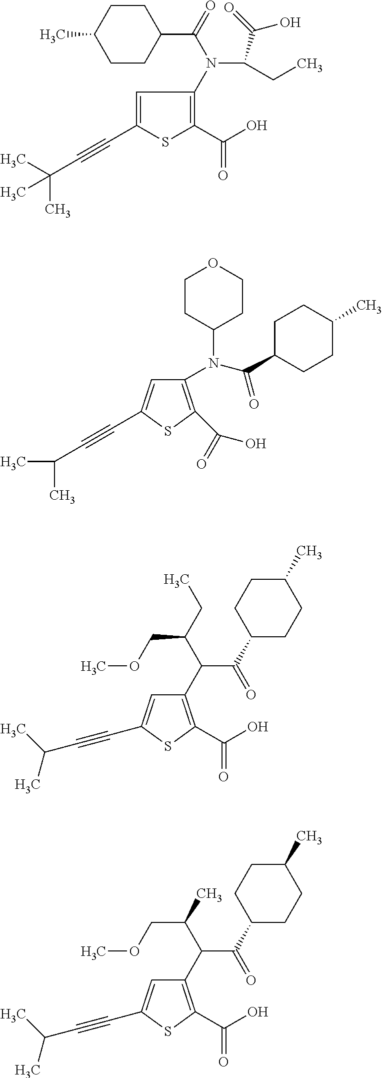

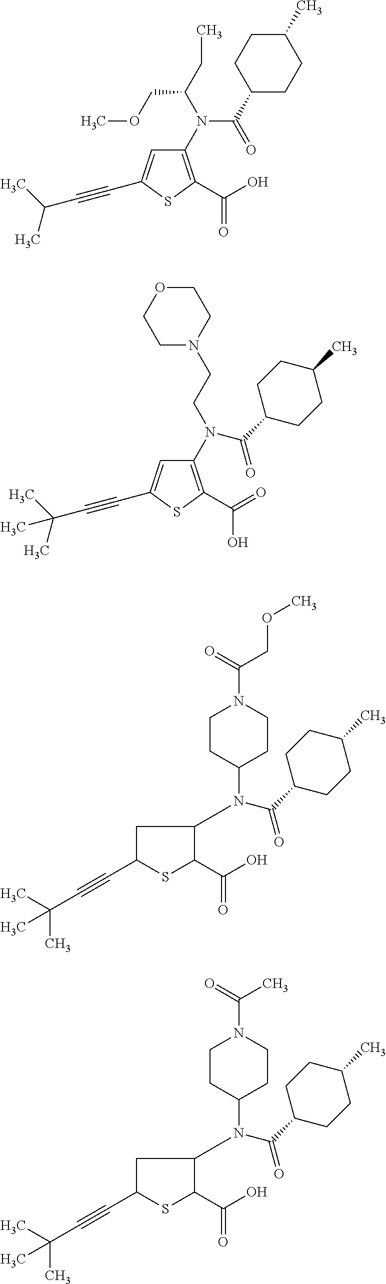

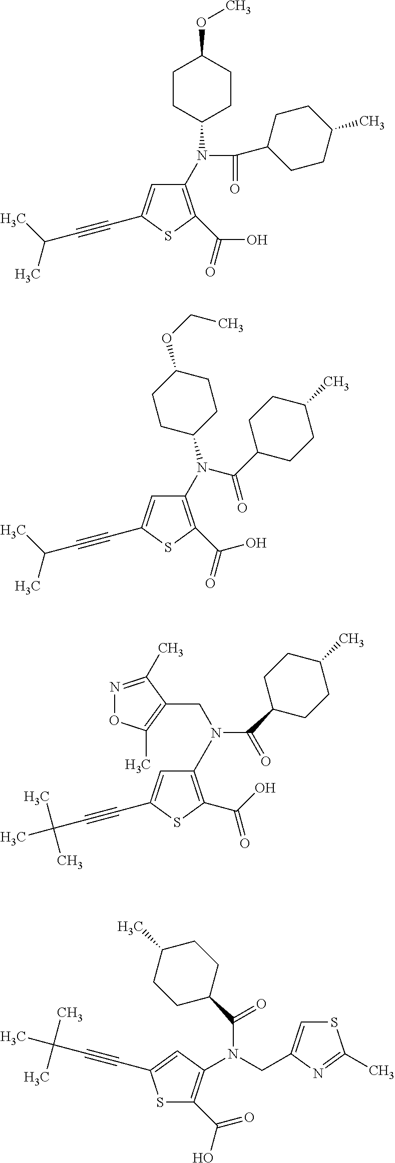

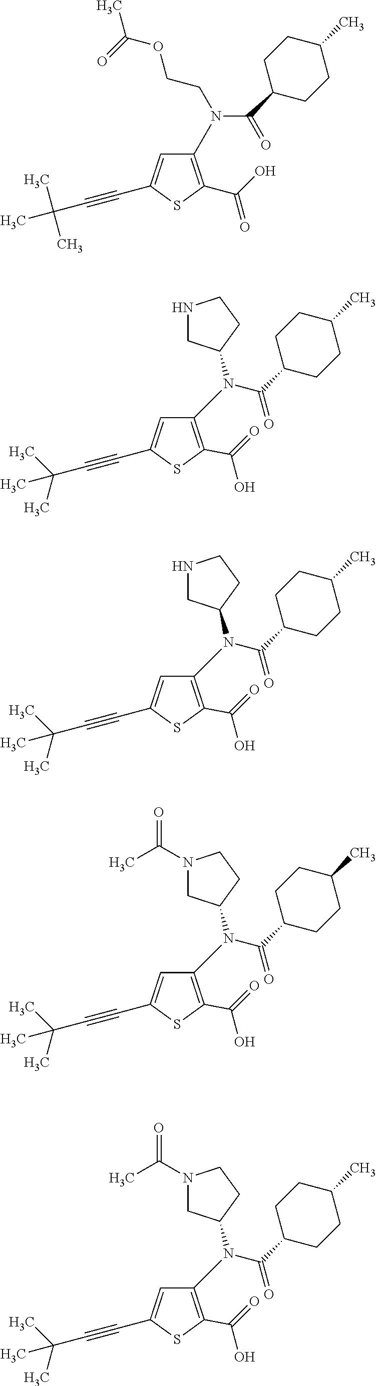

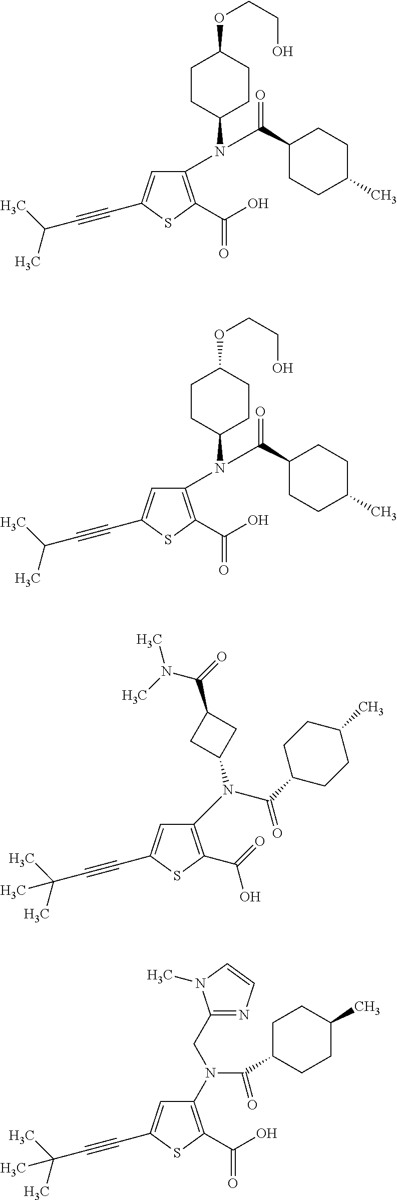

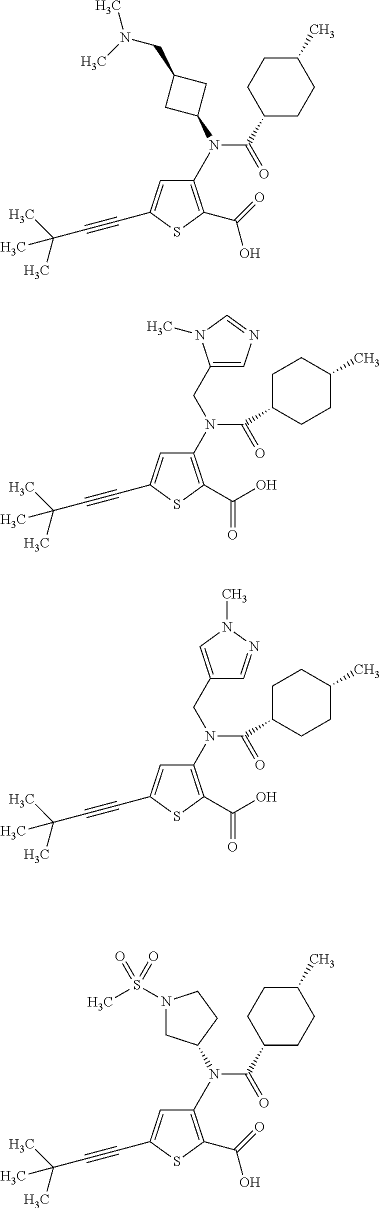

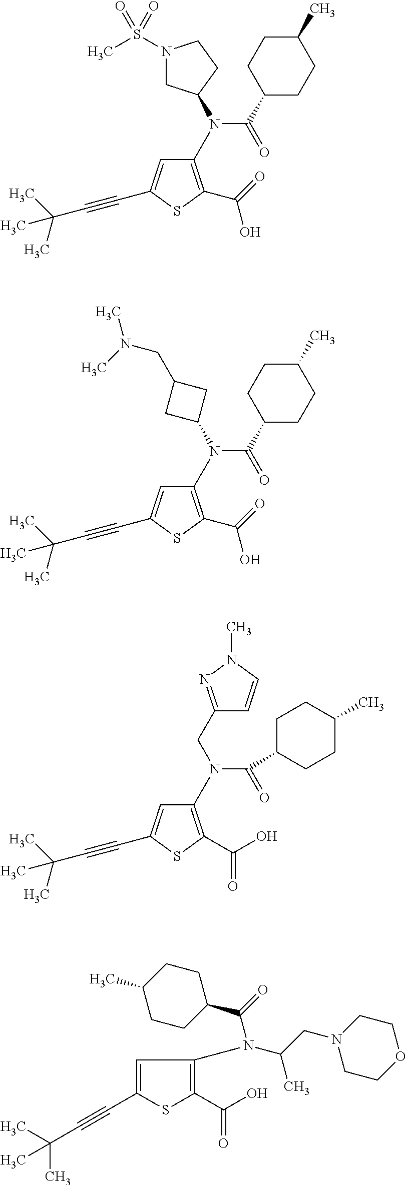

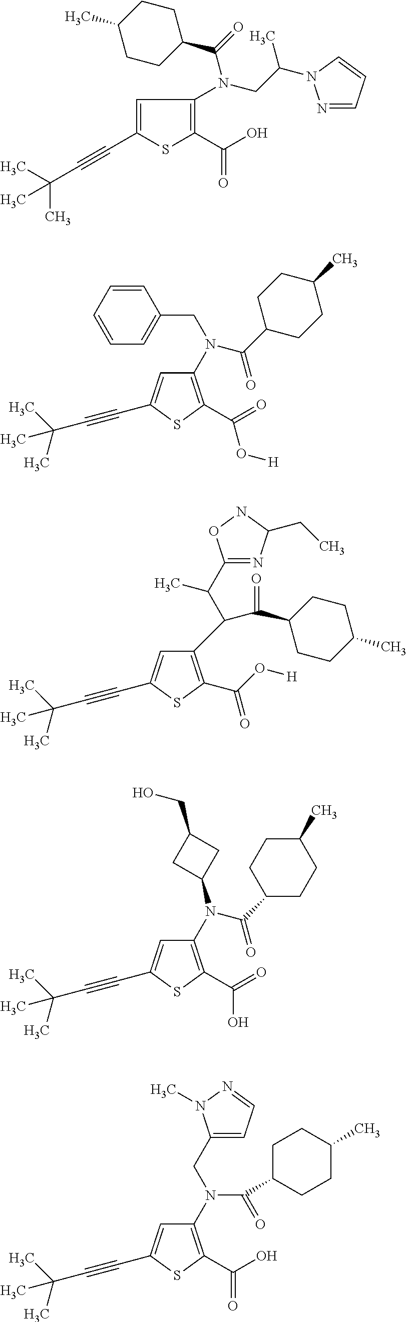

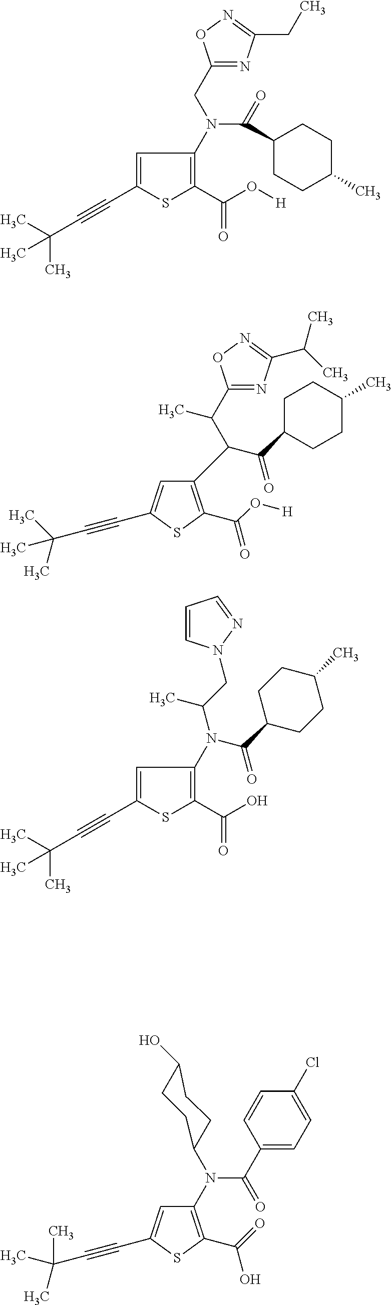

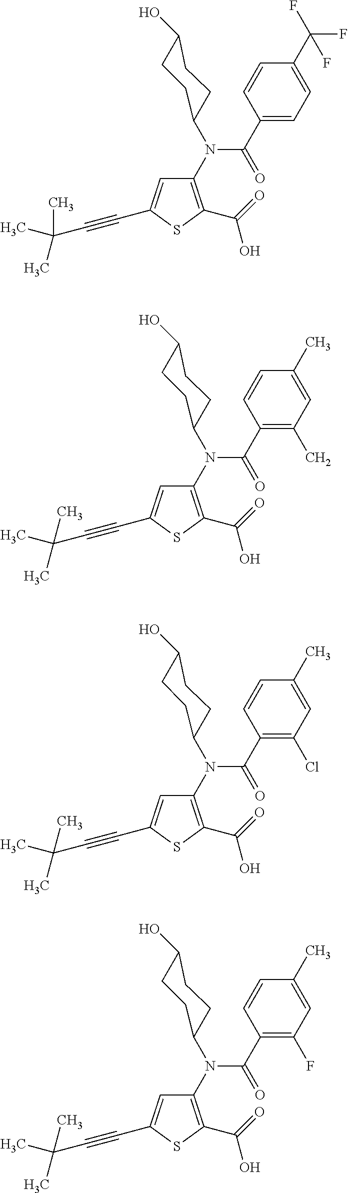

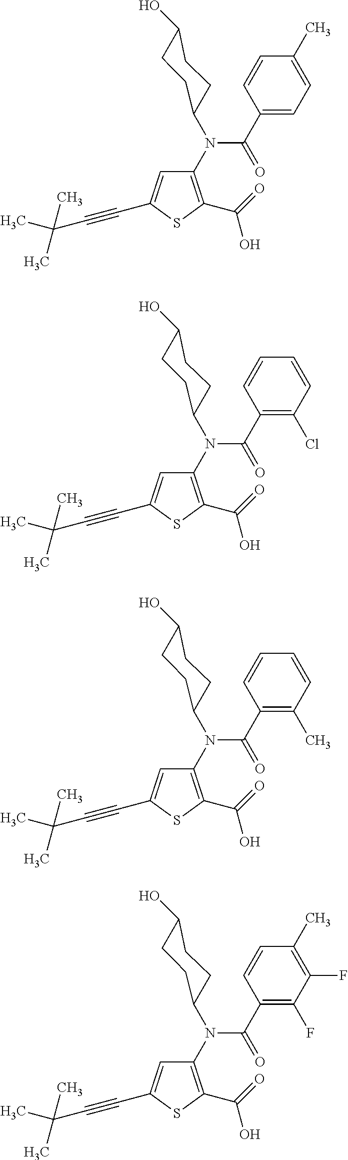

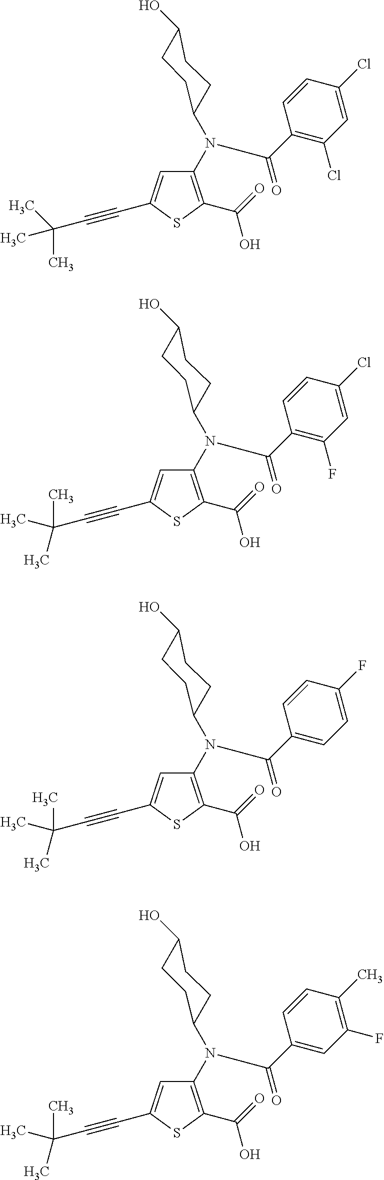 [SMILES: CC(C)(C)C#Cc1cc(N(C(=O)c2ccc(Cl)cc2Cl)C2CCC(O)CC2)c(C(=O)O)s1 ; CC(C)(C)C#Cc1cc(N(C(=O)c2ccc(Cl)cc2F)C2CCC(O)CC2)c(C(=O)O)s1 ; CC(C)(C)C#Cc1cc(N(C(=O)c2ccc(F)cc2)C2CCC(O)CC2)c(C(=O)O)s1 ; Cc1ccc(C(=O)N(c2cc(C#CC(C)(C)C)sc2C(=O)O)C2CCC(O)CC2)cc1F]

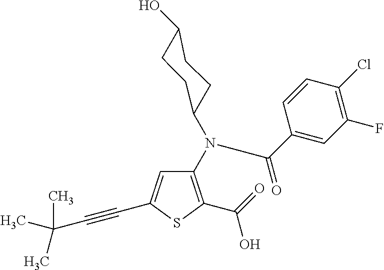 [SMILES: CC(C)(C)C#Cc1cc(N(C(=O)c2ccc(Cl)c(F)c2)C2CCC(O)CC2)c(C(=O)O)s1]

In another embodiment, the invention is directed to a compound depicted below or or a pharmaceutically acceptable salt thereof:

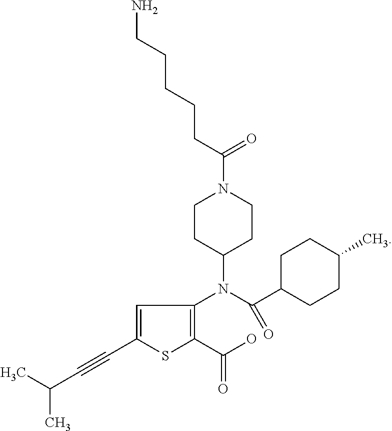

In another embodiment, the invention is directed to a pharmaceutical composition comprising a compound of the invention described herein and a pharmaceutically acceptable carrier or excipient.

In yet another embodiment, the invention provides methods of treating a HCV infection in a subject, comprising administering to the subject a therapeutically effective amount of a compound of the invention described herein.

In yet another embodiment, the invention is directed to a method of inhibiting or reducing the activity of HCV polymerase in a subject, comprising administering to the subject a therapeutically effective amount of a compound of the invention described herein.

In yet another embodiment, the invention is directed to a method of inhibiting or reducing the activity of HCV polymerase in a biological in vitro sample, comprising administering to the sample an effective amount of a compound of the invention described herein.

The present invention also provides use of the compounds of the invention described herein for the manufacture of the medicament for treating a HCV infection in a subject, or for inhibiting or reducing the activity of HCV polymerase in a subject.

Also provided herein is use of the compounds of the invention described herein for treating a HCV infection in a subject, or for inhibiting or reducing the activity of HCV polymerase in a subject.

### DESCRIPTION OF DRAWINGS

FIG. 1 shows certain compounds of the invention.

### DETAILED DESCRIPTION OF THE INVENTION

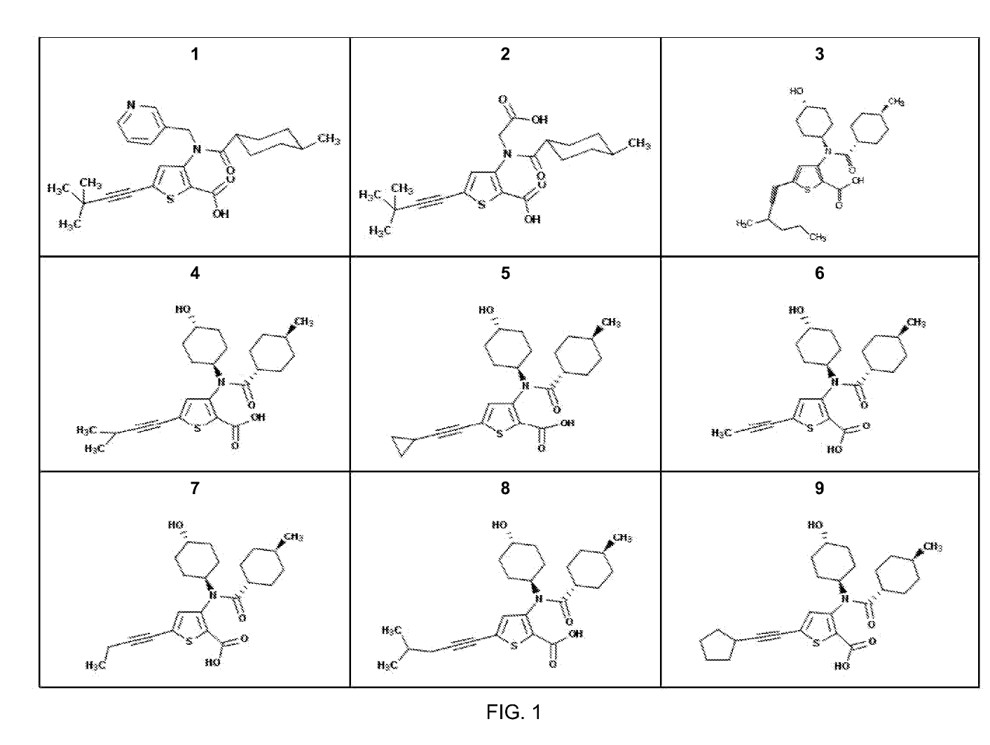

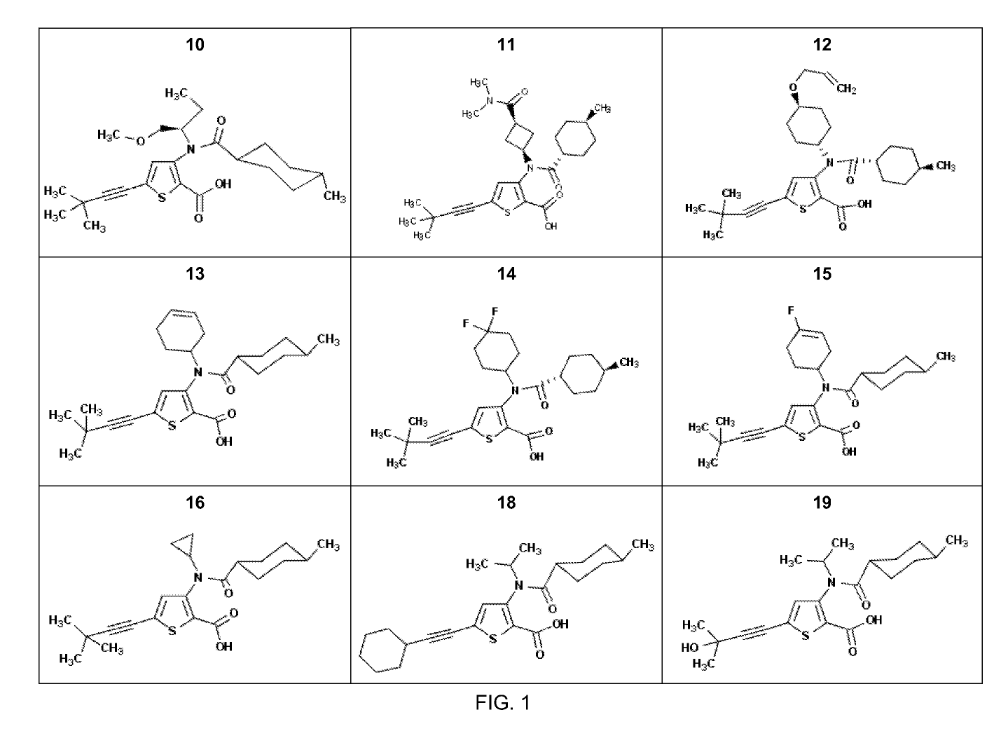

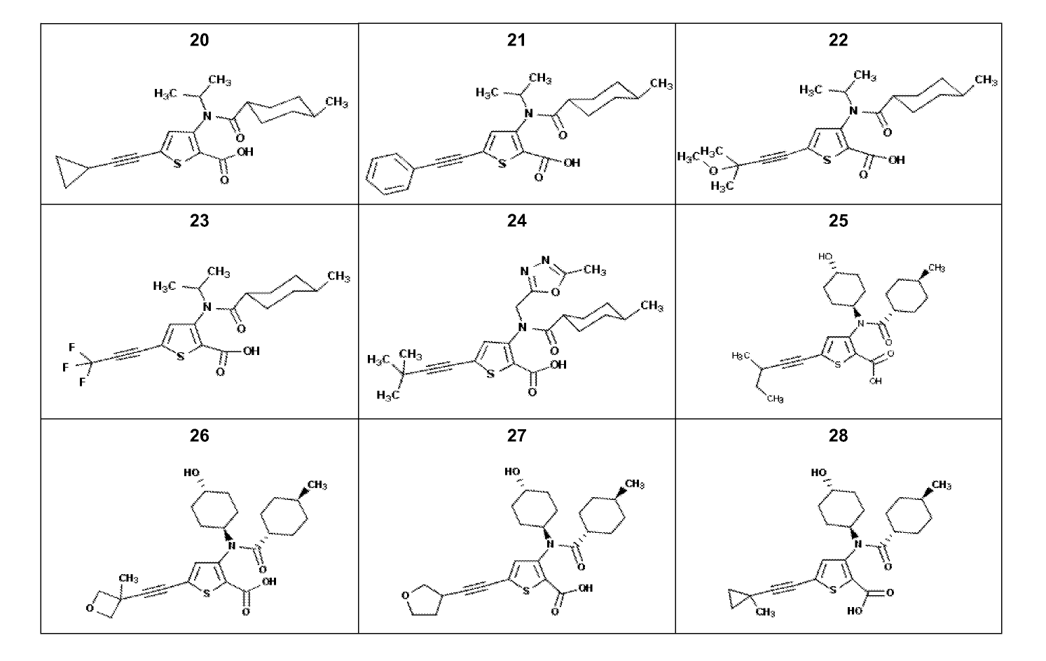

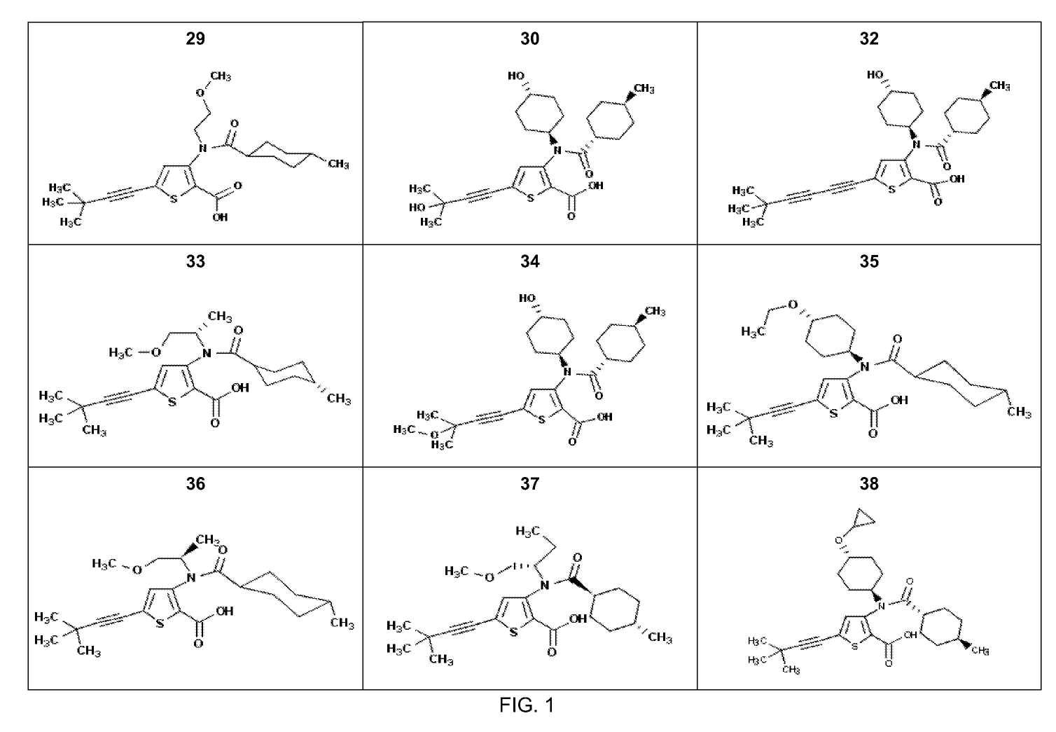

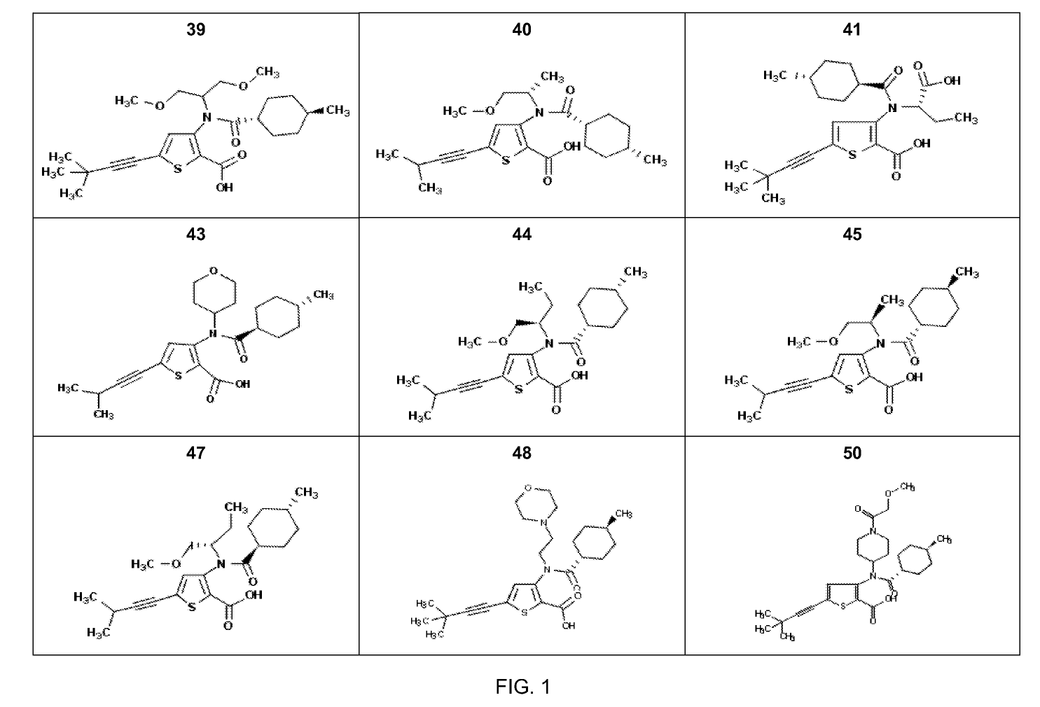

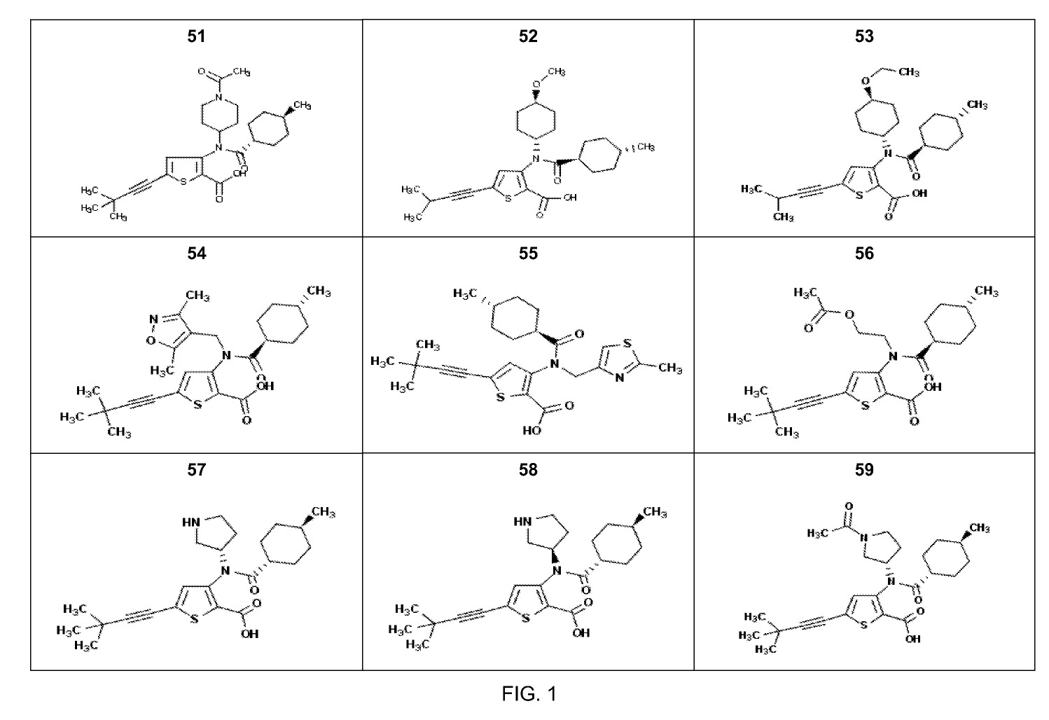

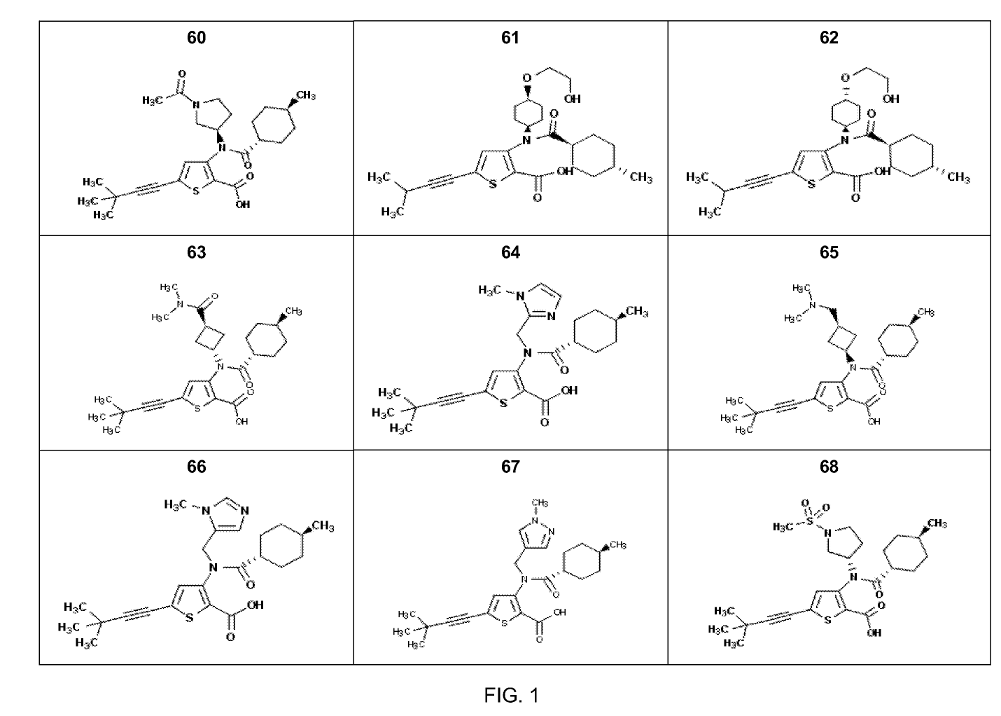

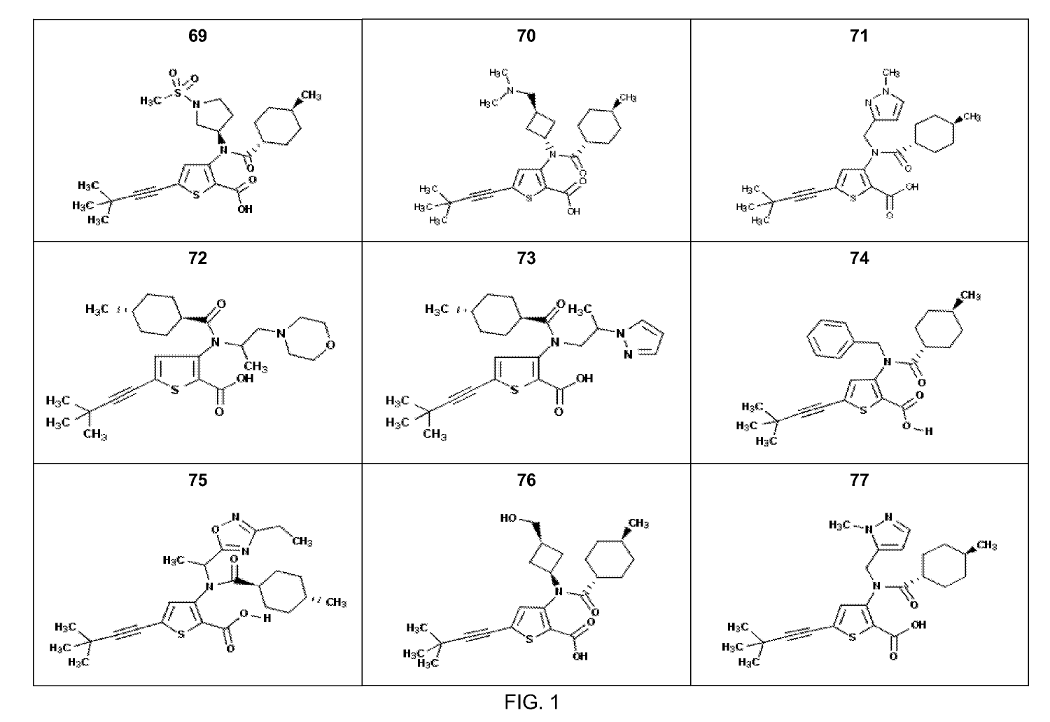

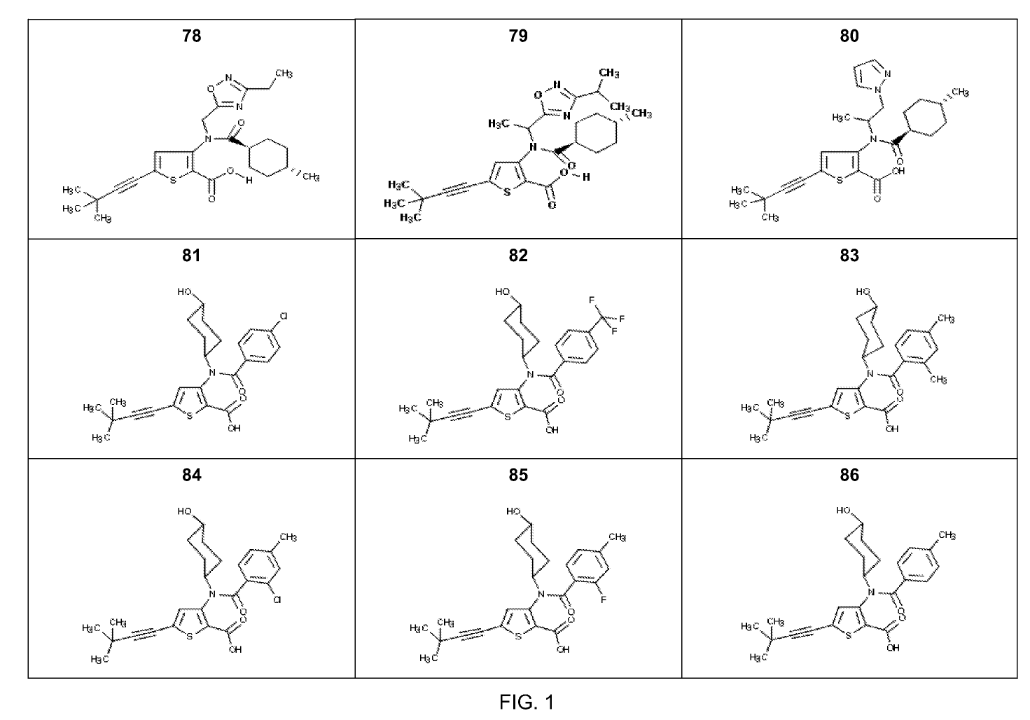

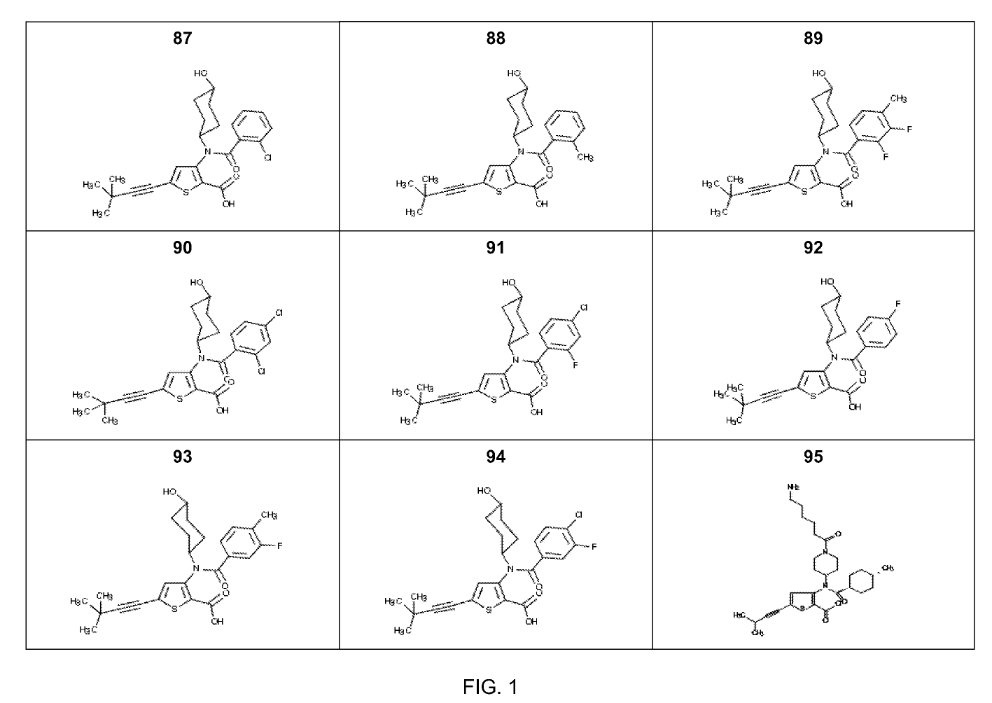

In one aspect, the present invention is directed to compounds represented by the structural formulae depicted in FIG. 1 and Exemplification below, or pharmaceutically acceptable salts thereof.

In a specific embodiment, the compounds are selected from the following structural formulae or pharmaceutically acceptable salts thereof:

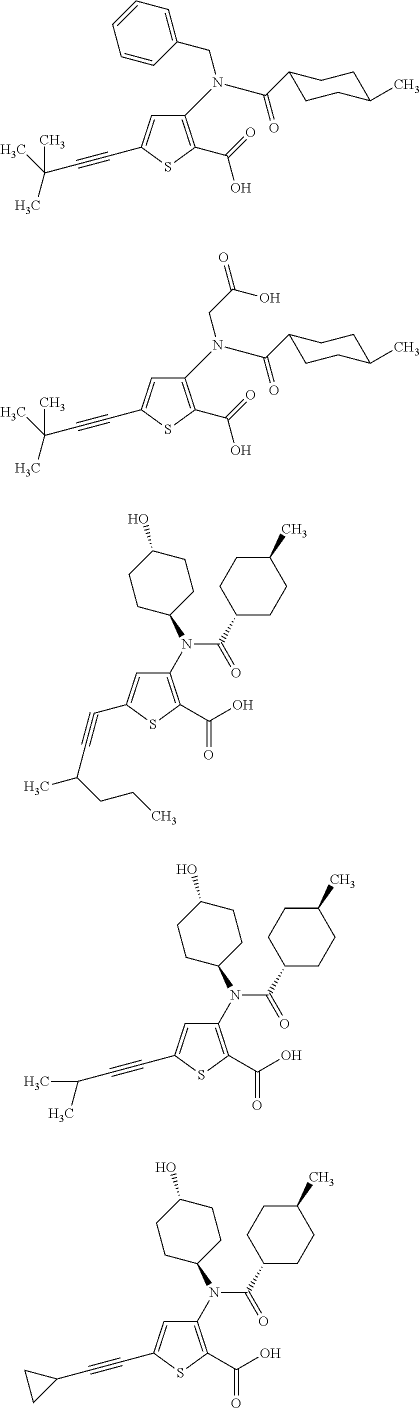

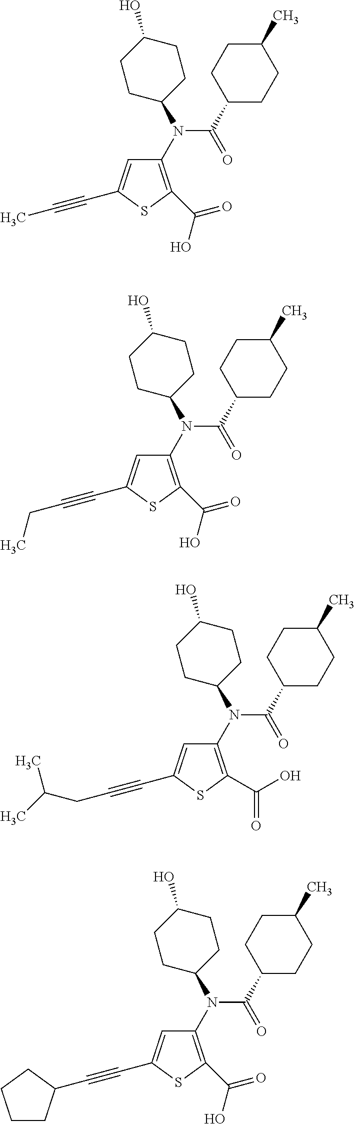

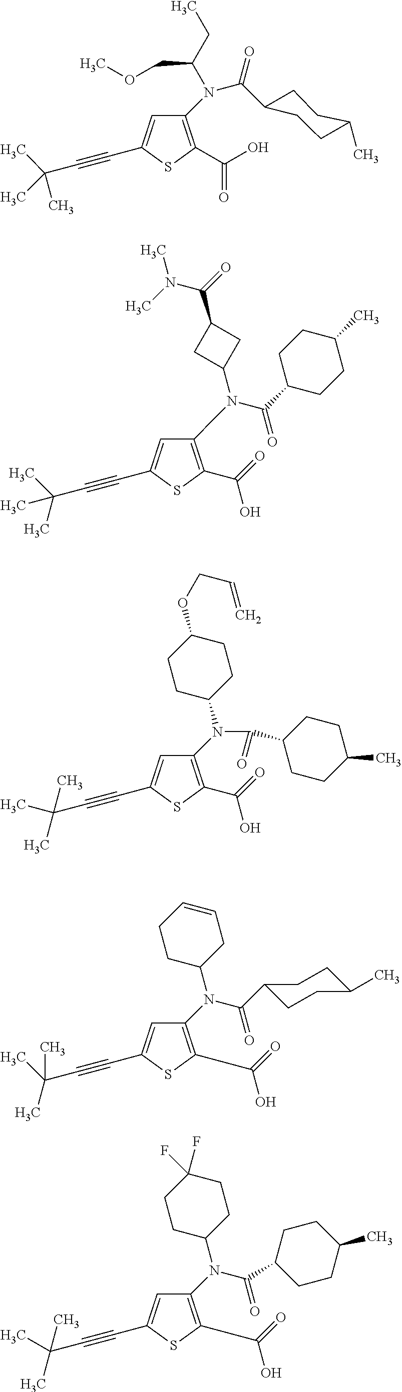

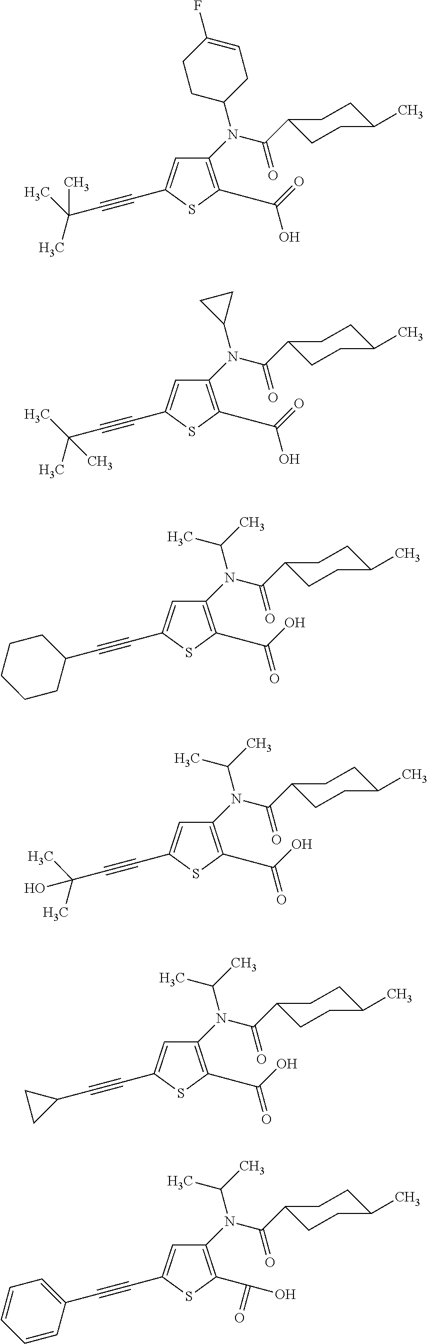

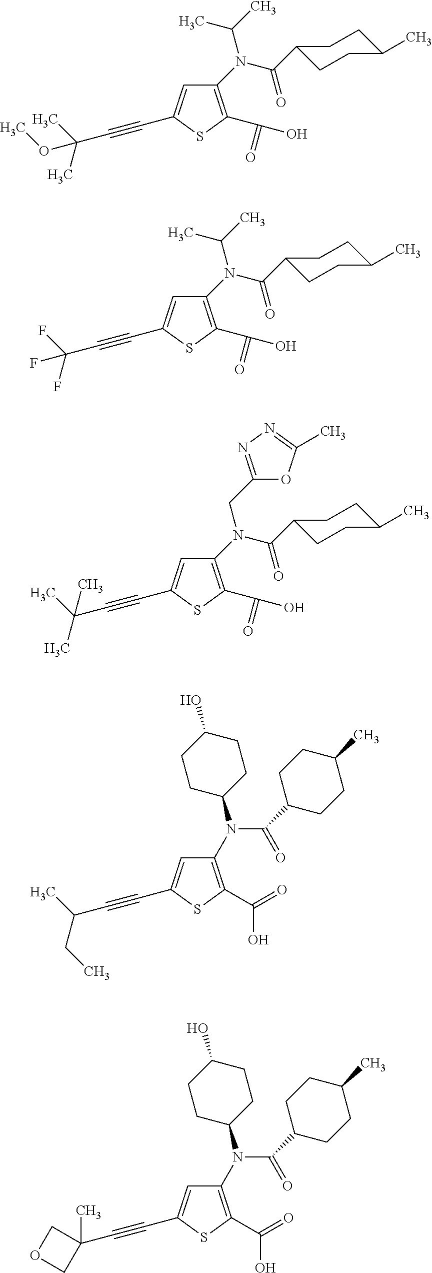

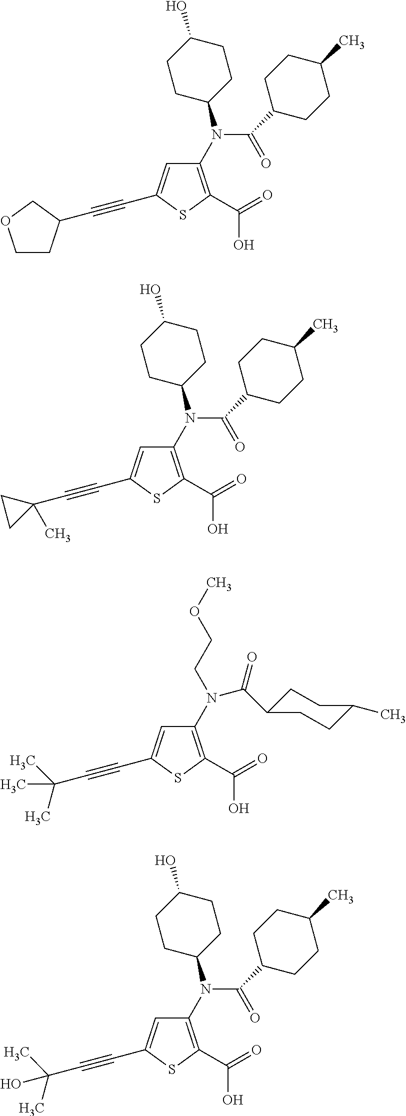

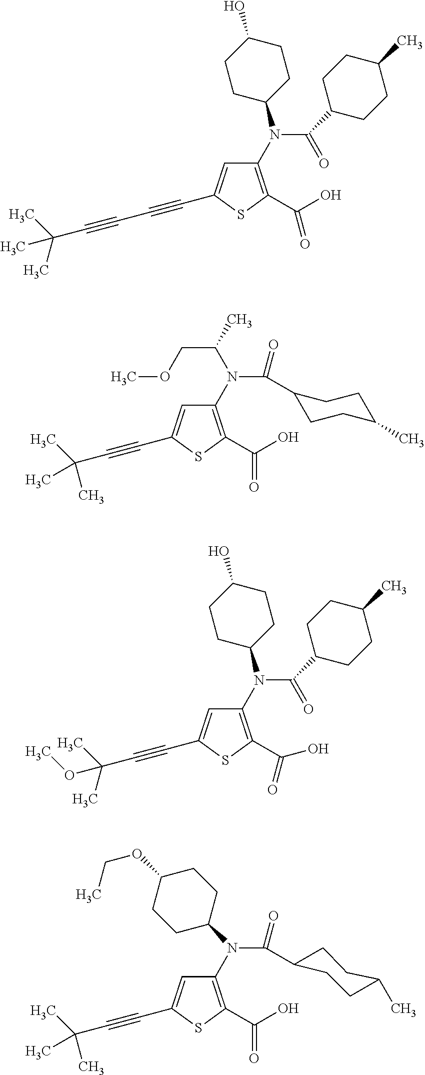

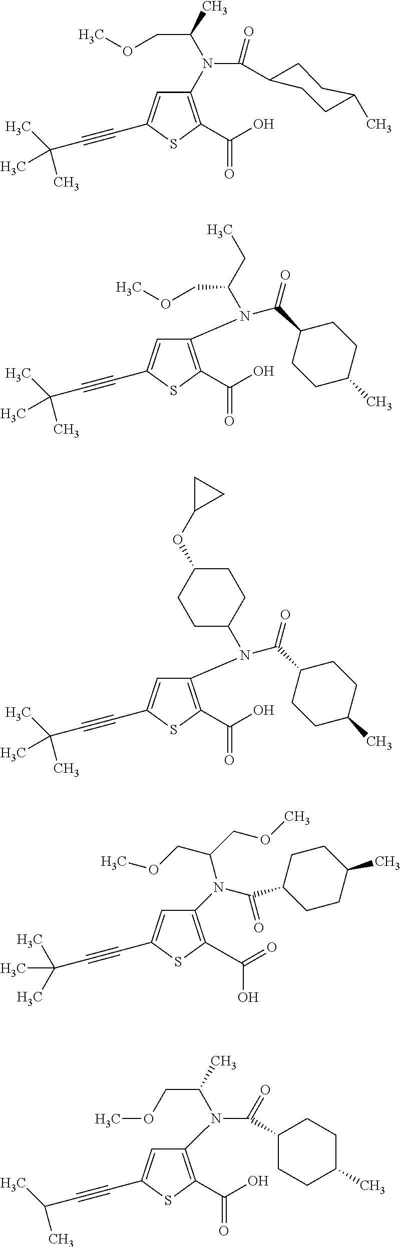

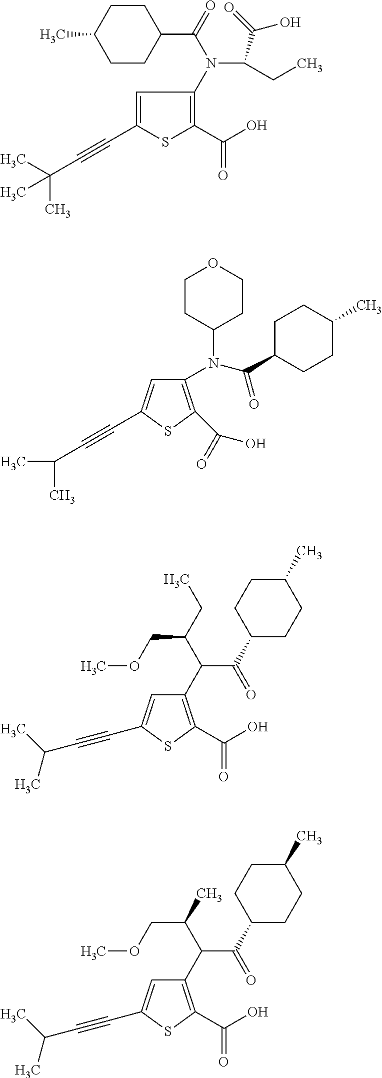

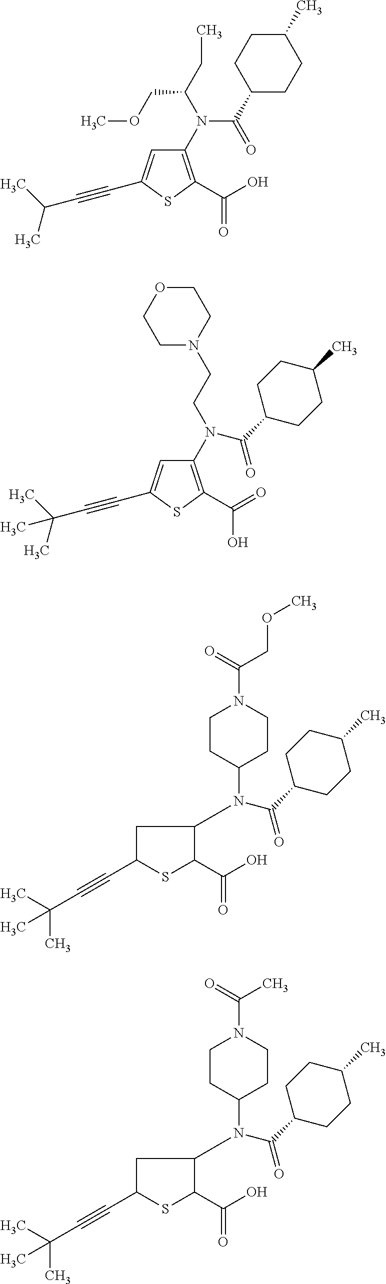

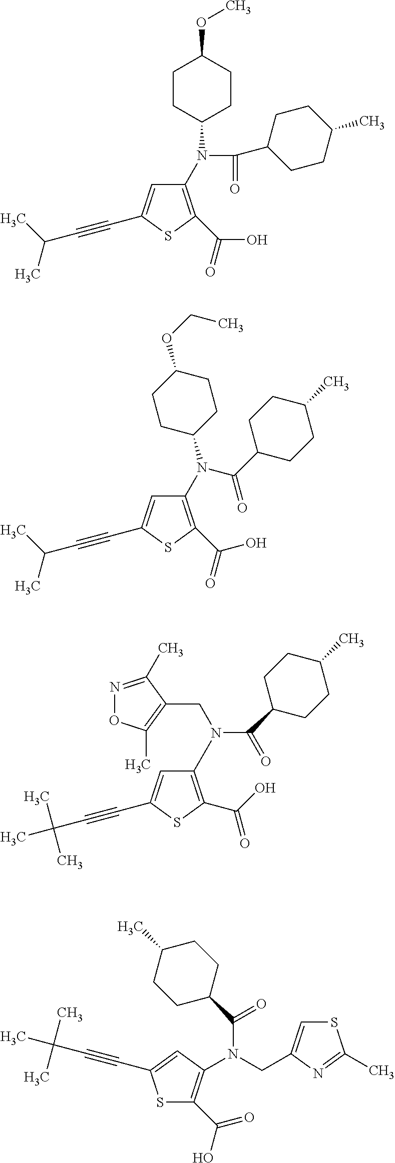

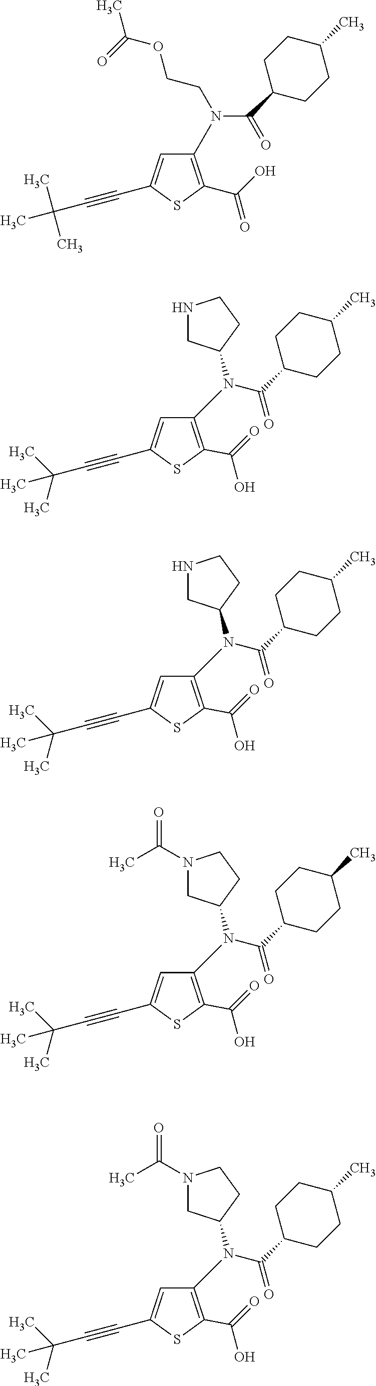

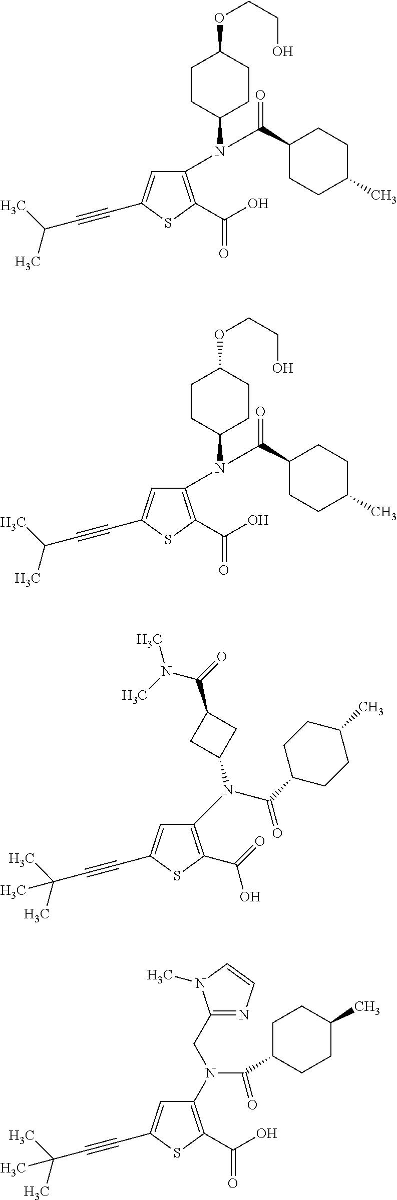

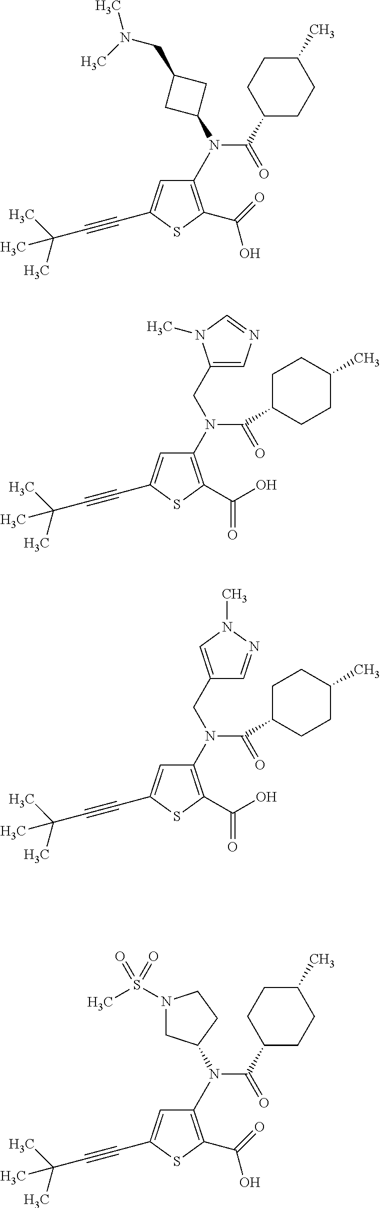

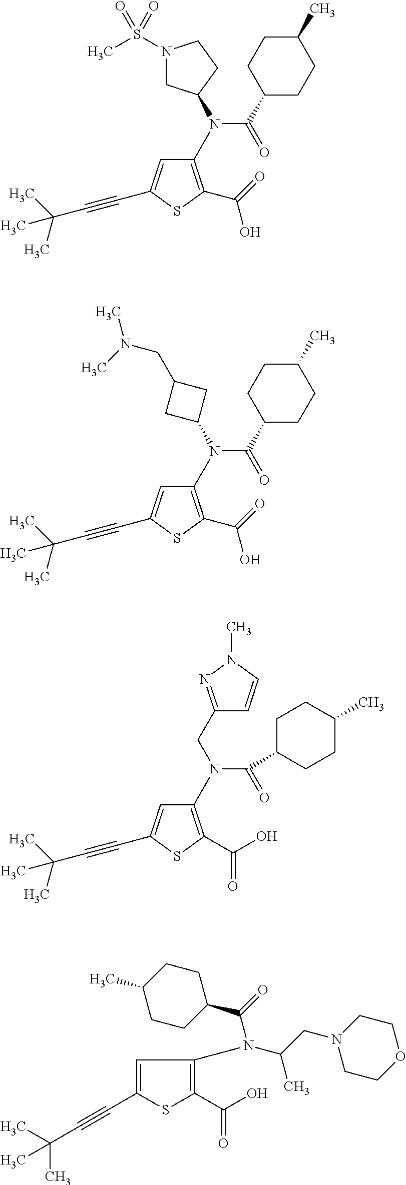

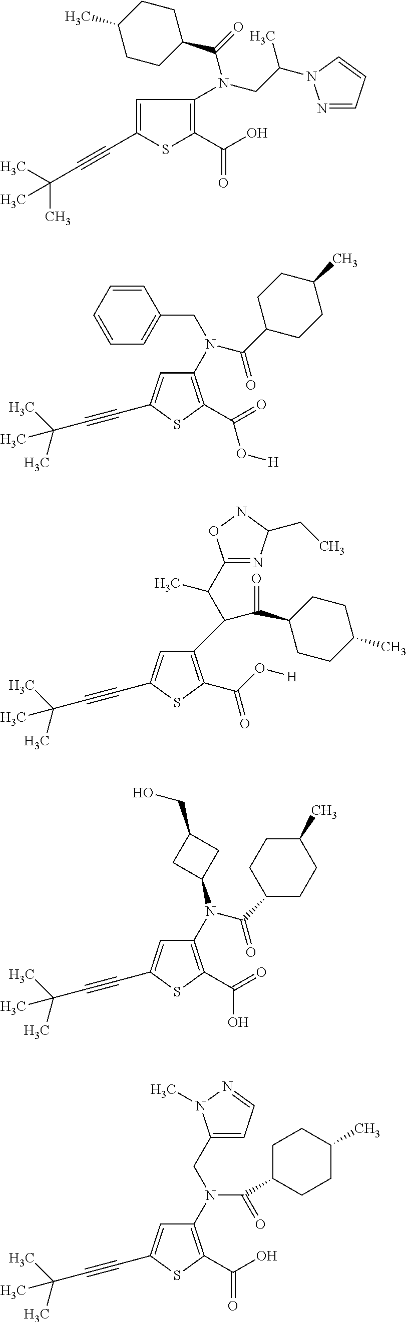

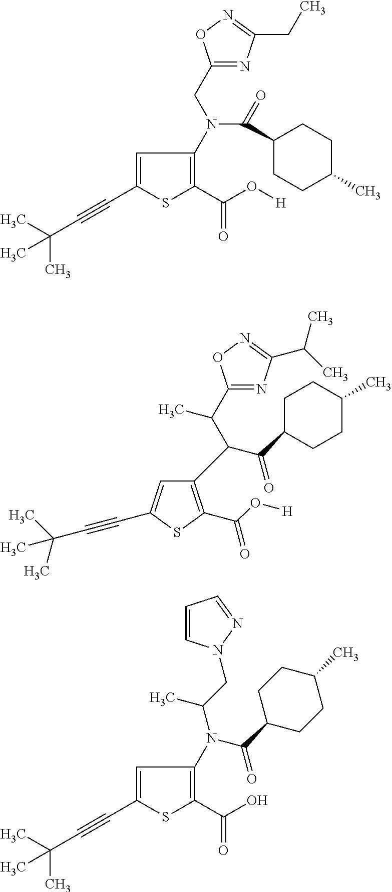

In another specific embodiment, the compounds are selected from the following structural formulae or pharmaceutically acceptable salts thereof:

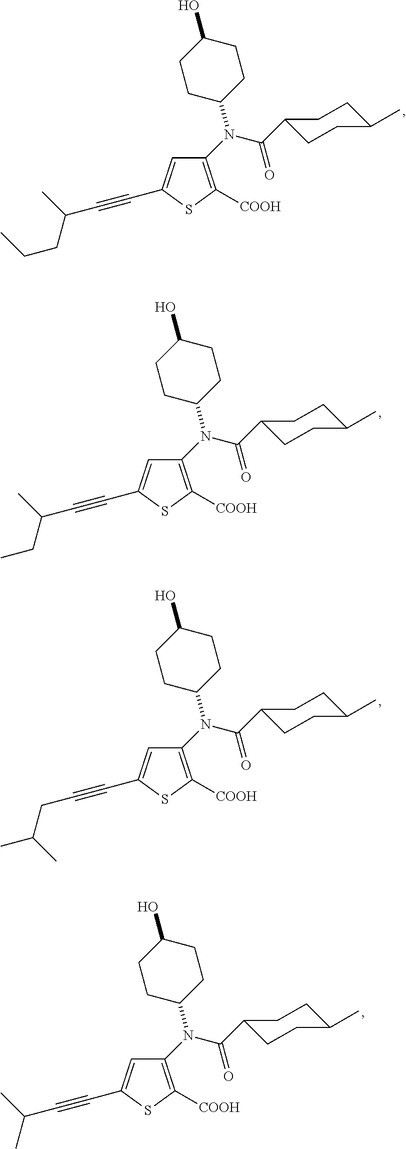

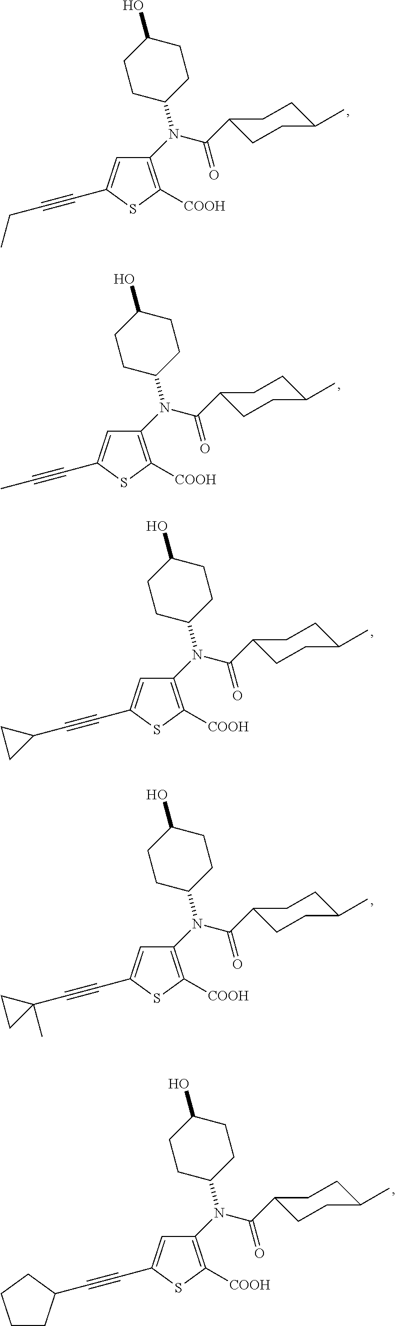

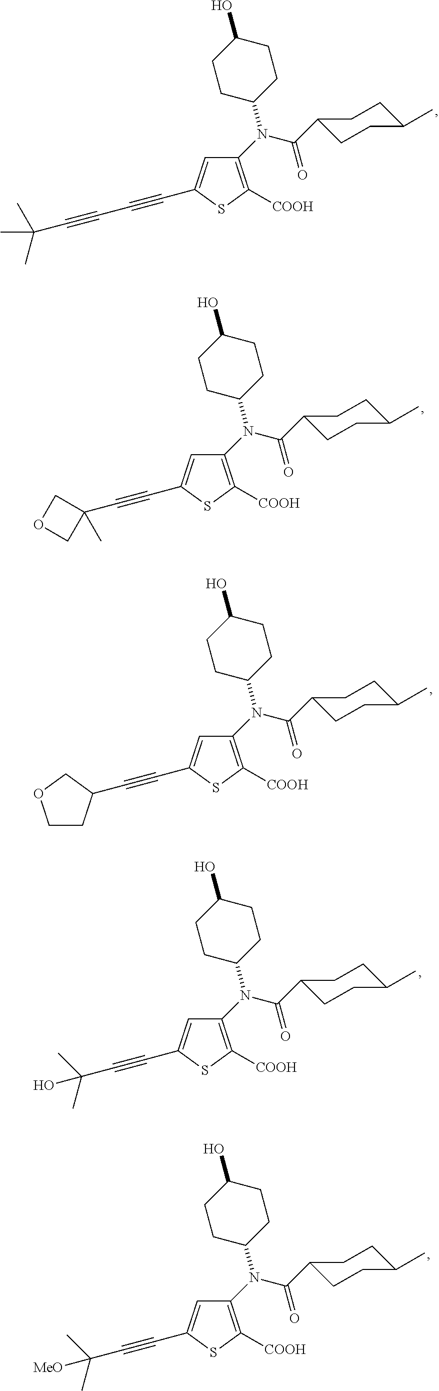

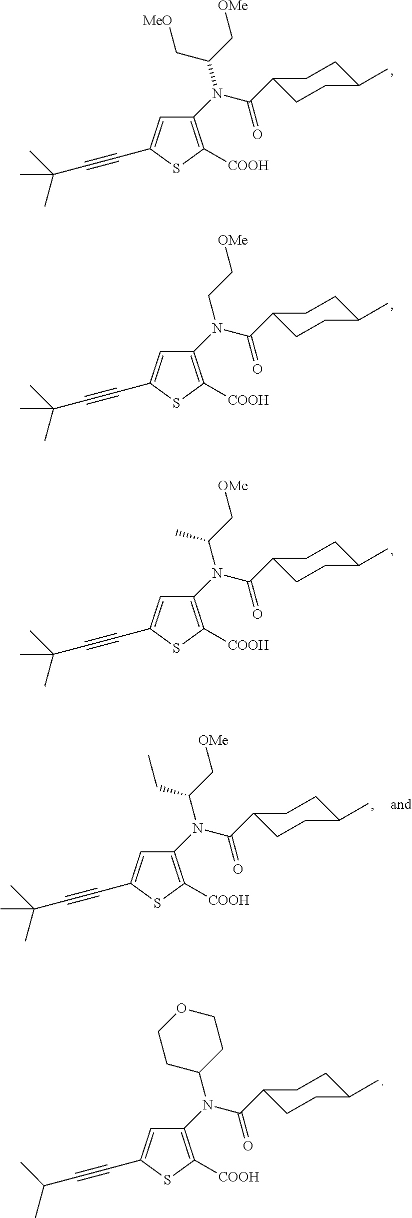

In another specific embodiment, the compound is selected from the following structural formula or a pharmaceutically acceptable salt thereof:

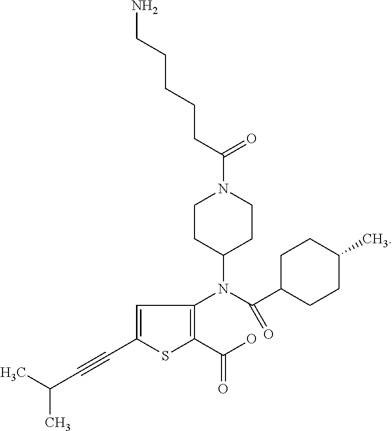

It will be appreciated by those skilled in the art that in the processes of the present invention certain functional groups such as hydroxyl or amino groups in the starting reagents or intermediate compounds may need to be protected by protecting groups. Thus, the preparation of the compounds may involve, at various stages, the addition and removal of one or more protecting groups. The protection and deprotection of functional groups is described in “Protective Groups in Organic Chemistry.” edited by J. W. F. McOmie, Plenum Press (1973) and “Protective Groups in Organic Synthesis,” 3rd edition, T. W. Greene and P. G. M. Wuts, Wiley Interscience, and “Protecting Groups,” 3rd edition, P. J. Kocienski, Thieme (2005)

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the invention may optionally be substituted with one or more substituents, such as illustrated generally below, or as exemplified by particular classes, subclasses, and species of the compounds described above. It will be appreciated that the phrase “optionally substituted” is used interchangeably with the phrase “substituted or unsubstituted.” In general, the term “substituted”, whether preceded by the term “optionally” or not, refers to the replacement of one or more hydrogen radicals in a given structure with the radical of a specified substituent. Unless otherwise indicated, an optionally substituted group may have a substituent at each substitutable position of the group. When more than one position in a given structure can be substituted with more than one substituent selected from a specified group, the substituent may be either the same or different at each position. When the term “optionally substituted” precedes a list, said term refers to all of the subsequent substitutable groups in that list. If a substituent radical or structure is not identified or defined as “optionally substituted”, the substituent radical or structure is unsubstituted. For example, if X is optionally substituted C₁-C₃alkyl or phenyl; X may be either optionally substituted C₁-C₃ alkyl or optionally substituted phenyl. Likewise, if the term “optionally substituted” follows a list, said term also refers to all of the substitutable groups in the prior list unless otherwise indicated. For example: if X is C₁-C₃alkyl or phenyl wherein X is optionally and independently substituted by J^{X}, then both C₁-C₃alkyl and phenyl may be optionally substituted by J^{X}. As is apparent to one having ordinary skill in the art, groups such as H, halogen, NO₂, CN, NH₂, OH, or OCF₃ would not be substitutable groups.

The phrase “up to”, as used herein, refers to zero or any integer number that is equal or less than the number following the phrase. For example, “up to 3” means any one of 0, 1, 2, and 3. As described herein, a specified number range of atoms includes any integer therein. For example, a group having from 1-4 atoms could have 1, 2, 3, or 4 atoms.

Selection of substituents and combinations of substituents envisioned by this invention are those that result in the formation of stable or chemically feasible compounds. The term “stable”, as used herein, refers to compounds that are not substantially altered when subjected to conditions to allow for their production, detection, and, specifically, their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. Only those choices and combinations of substituents that result in a stable structure are contemplated. Such choices and combinations will be apparent to those of ordinary skill in the art and may be determined without undue experimentation.

The term “aliphatic” or “aliphatic group”, as used herein, means a straight-chain (i.e., unbranched), or branched, hydrocarbon chain that is completely saturated or that contains one or more units of unsaturation but is non-aromatic. Unless otherwise specified, aliphatic groups contain 1-10 aliphatic carbon atoms. In some embodiments, aliphatic groups contain 1-6 aliphatic carbon atoms. In other embodiments, aliphatic groups contain 1-4 aliphatic carbon atoms. Aliphatic groups may be linear or branched, substituted or unsubstituted alkyl, alkenyl, or alkynyl groups. Specific examples include, but are not limited to, methyl, ethyl, isopropyl, n-propyl, sec-butyl, vinyl, n-butenyl, ethynyl, and tert-butyl and acetylene.

The term “alkyl” as used herein means a saturated straight or branched chain hydrocarbon. The term “alkenyl” as used herein means a straight or branched chain hydrocarbon comprising one or more double bonds. The term “alkynyl” as used herein means a straight or branched chain hydrocarbon comprising one or more triple bonds. Each of the “alkyl”, “alkenyl” or “alkynyl” as used herein can be optionally substituted as set forth below. In some embodiments, the “alkyl” is C₁-C₆ alkyl or C₁-C₄ alkyl. In some embodiments, the “alkenyl” is C₂-C₆ alkenyl or C₂-C₄ alkenyl. In some embodiments, the “alkynyl” is C₂-C₆ alkynyl or C₂-C₄ alkynyl.

The term “cycloaliphatic” (or “carbocycle” or “carbocyclyl” or “carbocyclic”) refers to a non-aromatic carbon only containing ring system which can be saturated or contains one or more units of unsaturation, having three to fourteen ring carbon atoms. In some embodiments, the number of carbon atoms is 3 to 10. In other embodiments, the number of carbon atoms is 4 to 7. In yet other embodiments, the number of carbon atoms is 5 or 6. The term includes monocyclic, bicyclic or polycyclic, fused, spiro or bridged carbocyclic ring systems. The term also includes polycyclic ring systems in which the carbocyclic ring can be “fused” to one or more non-aromatic carbocyclic or heterocyclic rings or one or more aromatic rings or combination thereof, wherein the radical or point of attachment is on the carbocyclic ring. “Fused” bicyclic ring systems comprise two rings which share two adjoining ring atoms. Bridged bicyclic group comprise two rings which share three or four adjacent ring atoms. Spiro bicyclic ring systems share one ring atom. Examples of cycloaliphatic groups include, but are not limited to, cycloalkyl and cycloalkenyl groups. Specific examples include, but are not limited to, cyclohexyl, cyclopropenyl, and cyclobutyl.

The term “heterocycle” (or “heterocyclyl,” or “heterocyclic” or “non-aromatic heterocycle”) as used herein refers to a non-aromatic ring system which can be saturated or contain one or more units of unsaturation, having three to fourteen ring atoms in which one or more ring carbons is replaced by a heteroatom such as, N, S, or O. In some embodiments, non-aromatic heterocyclic rings comprise up to three heteroatoms selected from N, S and O within the ring. In other embodiments, non-aromatic heterocyclic rings comprise up to two heteroatoms selected from N, S and O within the ring system. In yet other embodiments, non-aromatic heterocyclic rings comprise up to three heteroatoms selected from N and O within the ring system. In yet other embodiments, non-aromatic heterocyclic rings comprise up to two heteroatoms selected from N and O within the ring system. The term includes monocyclic, bicyclic or polycyclic fused, spiro or bridged heterocyclic ring systems. The term also includes polycyclic ring systems in which the heterocyclic ring can be fused to one or more non-aromatic carbocyclic or heterocyclic rings or one or more aromatic rings or combination thereof, wherein the radical or point of attachment is on the heterocyclic ring. Examples of heterocycles include, but are not limited to, piperidinyl, piperizinyl, pyrrolidinyl, pyrazolidinyl, imidazolidinyl, azepanyl, diazepanyl, triazepanyl, azocanyl, diazocanyl, triazocanyl, oxazolidinyl, isoxazolidinyl, thiazolidinyl, isothiazolidinyl, oxazocanyl, oxazepanyl, thiazepanyl, thiazocanyl, benzimidazolonyl, tetrahydrofuranyl, tetrahydrofuranyl, tetrahydrothiophenyl, tetrahydrothiophenyl, morpholino, including, for example, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, indolinyl, tetrahydroquinolinyl, tetrahydroisoquinolinyl, benzothiolanyl, benzodithianyl, 3-(1-alkyl)-benzimidazol-2-onyl, and 1,3-dihydro-imidazol-2-onyl.

The term “aryl” (or “aryl ring” or “aryl group”) used alone or as part of a larger moiety as in “aralkyl”, “aralkoxy”, “aryloxyalkyl”, or “heteroaryl” refers to carbocyclic aromatic ring systems. The term “aryl” may be used interchangeably with the terms “aryl ring” or “aryl group”. “Carbocyclic aromatic ring” groups have only carbon ring atoms (typically six to fourteen) and include monocyclic aromatic rings such as phenyl and fused polycyclic aromatic ring systems in which two or more carbocyclic aromatic rings are fused to one another. Examples include 1-naphthyl, 2-naphthyl, 1-anthracyl and 2-anthracyl. Also included within the scope of the term “carbocyclic aromatic ring” or “carbocyclic aromatic”, as it is used herein, is a group in which an aromatic ring is “fused” to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring.

The terms “heteroaryl”, “heteroaromatic”, “heteroaryl ring”, “heteroaryl group”, “aromatic heterocycle” or “heteroaromatic group”, used alone or as part of a larger moiety as in “heteroaralkyl” or “heteroarylalkoxy”, refer to heteroaromatic ring groups having five to fourteen members, in which one or more ring carbons is replaced by a heteroatom such as, N, S, or O. In some embodiments, heteroaryl rings comprise up to three heteroatoms selected from N, S and O within the ring. In other embodiments, heteroaryl rings comprise up to two heteroatoms selected from N, S and O within the ring system. In yet other embodiments, heteroaryl rings comprise up to three heteroatoms selected from N and O within the ring system. In yet other embodiments, heteroaryl rings comprise up to two heteroatoms selected from N and O within the ring system. Heteroaryl rings include monocyclic heteroaromatic rings and polycyclic aromatic rings in which a monocyclic aromatic ring is fused to one or more other aromatic rings. Also included within the scope of the term “heteroaryl”, as it is used herein, is a group in which an aromatic ring is “fused” to one or more non-aromatic rings (carbocyclic or heterocyclic), where the radical or point of attachment is on the aromatic ring. Bicyclic 6,5 heteroaromatic ring, as used herein, for example, is a six membered heteroaromatic ring fused to a second five membered ring, wherein the radical or point of attachment is on the six membered ring. Examples of heteroaryl groups include pyridyl, pyrazinyl, pyrimidinyl, pyridazinyl, imidazolyl, pyrrolyl, pyrazolyl, triazolyl, tetrazolyl, oxazolyl, isoxazolyl, oxadiazolyl, thiazolyl, isothiazolyl or thiadiazolyl including, for example, 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, benzimidazolyl, benzothienyl, benzofuranyl, indolyl, benzotriazolyl, benzothiazolyl, benzoxazolyl, benzimidazolyl, isoquinolinyl, indolyl, isoindolyl, acridinyl, benzisoxazolyl, isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

As used herein, “cyclo”, “cyclic”, “cyclic group” or “cyclic moiety”, include mono-, bi-, and tri-cyclic ring systems including cycloaliphatic, heterocycloaliphatic, aryl, or heteroaryl, each of which has been previously defined.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “bridged bicyclic ring system” refers to a bicyclic heterocycloalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “bridge” refers to a bond or an atom or an unbranched chain of atoms connecting two different parts of a molecule. The two atoms that are connected through the bridge (usually but not always, two tertiary carbon atoms) are denotated as “bridgeheads”.

As used herein, the term “spiro” refers to ring systems having one atom (usually a quaternary carbon) as the only common atom between two rings.

The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

As used herein an optionally substituted aralkyl can be substituted on both the alkyl and the aryl portion. Unless otherwise indicated as used herein optionally substituted aralkyl is optionally substituted on the aryl portion.

In some embodiments, an aliphatic group and a heterocyclic ring may independently contain one or more substituents. Suitable substituents on the saturated carbon of an aliphatic group or of a non-aromatic heterocyclic ring are selected from those described above. Other suitable substitutents include those listed as suitable for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, wherein each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R* is unsubstituted.

In some embodiments, optional substituents on the nitrogen of a heterocyclic ring include those described above. Examples of such suitable substituents include —OH, —NH₂, —NH(C₁-C₄ alkyl), —N(C₁-C₄ alkyl)₂, —CO(C₁-C₄ alkyl), —CO₂H, —CO₂(C₁-C₄ alkyl), —O(C₁-C₄ alkyl), and C₁-C₄ aliphatic that is optionally substituted with one or more substituents independently selected from the group consisting of halogen, oxo, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃₋₇ cycloalkyl, and C₃₋₇ cyclo(haloalkyl). Other suitable substituents include —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, or —NR⁺SO₂R⁺; wherein R⁺ is hydrogen, an optionally substituted C₁₋₆ aliphatic, optionally substituted phenyl, optionally substituted —O(Ph), optionally substituted —CH₂(Ph), optionally substituted —(CH₂)₂(Ph); optionally substituted —CH═CH(Ph); or an unsubstituted 5-6 membered heteroaryl or heterocyclic ring having one to four heteroatoms independently selected from oxygen, nitrogen, or sulfur, or, two independent occurrences of R⁺, on the same substituent or different substituents, taken together with the atom(s) to which each R⁺ group is bound, form a 5-8-membered heterocyclyl, aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R⁺ is unsubstituted.

In some embodiments, an aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl or heteroaryl group are selected from those described above. Specific examples include halogen, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), and C₁-C₄ aliphatic that is optionally substituted with one or more substituents independently selected from the group consisting of halogen, oxo, —CN, —OH, —NH₂, —NH(C₁-C₄ alkyl), —N(C₁-C₄ alkyl)₂, —OCO(C₁-C₄ alkyl), —CO(C₁-C₄ alkyl), —CO₂H, —CO₂(C₁-C₄ alkyl), —O(C₁-C₄ alkyl), C₃₋₇ cycloalkyl, and C₃₋₇ cyclo(haloalkyl). Other suitable substituents include: halogen; —R^{o}; —OR^{o}; —SR^{o}; 1,2-methylenedioxy; 1,2-ethylenedioxy; phenyl (Ph) optionally substituted with R^{o}; —O(Ph) optionally substituted with R^{o}; —(CH₂)₁₋₂(Ph), optionally substituted with R^{o}; —CH═CH(Ph), optionally substituted with R^{o}; —NO₂; —CN; —N(R^{o})₂; —NR^{o}C(O)R^{o}; —NR^{o}C(S)R^{o}; —NR^{o}C(O)N(R^{o})₂; —NR^{o}C(S)N(R^{o})₂; —NR^{o}CO₂R^{o}; —NR^{o}NR^{o}C(O)R^{o}; —NR^{o}NR^{o}C(O)N(R^{o})₂; —NR^{o}NR^{o}CO₂R^{o}; —C(O)C(O)R^{o}; —C(O)CH₂C(O)R^{o}; —CO₂R^{o}; —C(O)R^{o}; —C(S)R^{o}; —C(O)N(R^{o})₂; —C(S)N(R^{o})₂; —OC(O)N(R^{o})₂; —OC(O)R^{o}; —C(O)N(OR^{o})R^{o}; —C(NOR)^{o}R^{o}; —S(O)₂R^{o}; —S(O)₃R^{o}; —SO₂N(R^{o})₂; —S(O)R^{o}; —NR^{o}SO₂N(R^{o})₂; —NR^{o}SO₂R^{o}; —N(OR^{o}R^{o}; —C(═NH)—N(R^{o})₂; or —(CH₂)₀₋₂NHC(O)R^{o}; wherein each independent occurrence of R^{o} is selected from hydrogen, optionally substituted C₁₋₆ aliphatic, an unsubstituted 5-6 membered heteroaryl or heterocyclic ring, phenyl, —O(Ph), or —CH₂(Ph), or, two independent occurrences of R^{o}, on the same substituent or different substituents, taken together with the atom(s) to which each R^{o} group is bound, form a 5-8-membered heterocyclyl, aryl, or heteroaryl ring or a 3-8-membered cycloalkyl ring, wherein said heteroaryl or heterocyclyl ring has 1-3 heteroatoms independently selected from nitrogen, oxygen, or sulfur. Optional substituents on the aliphatic group of R^{o} are selected from NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, CHO, N(CO)(C₁₋₄ aliphatic), C(O)N(C₁₋₄ aliphatic), wherein each of the foregoing C₁₋₄aliphatic groups of R^{o} is unsubstituted.

Non-aromatic nitrogen containing heterocyclic rings that are substituted on a ring nitrogen and attached to the remainder of the molecule at a ring carbon atom are said to be N substituted. For example, an N alkyl piperidinyl group is attached to the remainder of the molecule at the two, three or four position of the piperidinyl ring and substituted at the ring nitrogen with an alkyl group. Non-aromatic nitrogen containing heterocyclic rings such as pyrazinyl that are substituted on a ring nitrogen and attached to the remainder of the molecule at a second ring nitrogen atom are said to be N′ substituted-N-heterocycles. For example, an N′ acyl N-pyrazinyl group is attached to the remainder of the molecule at one ring nitrogen atom and substituted at the second ring nitrogen atom with an acyl group.

The term “unsaturated”, as used herein, means that a moiety has one or more units of unsaturation.

As detailed above, in some embodiments, two independent occurrences of R^{o} (or R⁺, or any other variable similarly defined herein), may be taken together with the atom(s) to which each variable is bound to form a 5-8-membered heterocyclyl, aryl, or heteroaryl ring or a 3-8-membered cycloalkyl ring. Exemplary rings that are formed when two independent occurrences of R^{o} (or R⁺, or any other variable similarly defined herein) are taken together with the atom(s) to which each variable is bound include, but are not limited to the following: a) two independent occurrences of R^{o} (or R⁺, or any other variable similarly defined herein) that are bound to the same atom and are taken together with that atom to form a ring, for example, N(R^{o})₂, where both occurrences of R^{o} are taken together with the nitrogen atom to form a piperidin-1-yl, piperazin-1-yl, or morpholin-4-yl group; and b) two independent occurrences of R^{o} (or R⁺, or any other variable similarly defined herein) that are bound to different atoms and are taken together with both of those atoms to form a ring, for example where a phenyl group is substituted with two occurrences of OR^{o}

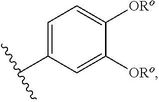

these two occurrences of R^{o} are taken together with the oxygen atoms to which they are bound to form a fused 6-membered oxygen containing ring:

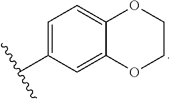 [SMILES: *c1ccc2c(c1)OCCO2]

It will be appreciated that a variety of other rings can be formed when two independent occurrences of R^{o} (or R⁺, or any other variable similarly defined herein) are taken together with the atom(s) to which each variable is bound and that the examples detailed above are not intended to be limiting.

As used herein, an “amino” group refers to —NH₂.

The term “hydroxyl” or “hydroxy” or “alcohol moiety” refers to —OH.

As used herein, an “oxo” refers to ═O.

As used herein, the term “alkoxy”, or “alkylthio”, as used herein, refers to an alkyl group, as previously defined, attached to the molecule through an oxygen (“alkoxy” e.g., —O-alkyl) or sulfur (“alkylthio” e.g., —S-alkyl) atom.

As used herein, the terms “halogen”, “halo”, and “hal” mean F, Cl, Br, or I.

As used herein, the term “cyano” or “nitrile” refer to —CN or —C≡N.

The terms “alkoxyalkyl”, “alkoxyalkenyl”, “alkoxyaliphatic”, and “alkoxyalkoxy” mean alkyl, alkenyl, aliphatic or alkoxy, as the case may be, substituted with one or more alkoxy groups.

The terms “haloalkyl”, “haloalkenyl”, “haloaliphatic”, “haloalkoxy”, and “cyclo(haloalkyl)” mean alkyl, alkenyl, aliphatic, alkoxy, or cycloalkyl, as the case may be, substituted with one or more halogen atoms. This term includes perfluorinated alkyl groups, such as —CF₃ and —CF₂CF₃.

The terms “cyanoalkyl”, “cyanoalkenyl”, “cyanoaliphatic”, and “cyanoalkoxy” mean alkyl, alkenyl, aliphatic or alkoxy, as the case may be, substituted with one or more cyano groups. In some embodiments, the cyanoalkyl is (NC)-alkyl-.

The terms “aminoalkyl”, “aminoalkenyl”, “aminoaliphatic”, and “aminoalkoxy” mean alkyl, alkenyl, aliphatic or alkoxy, as the case may be, substituted with one or more amino groups, wherein the amino group is as defined above.

The terms “hydroxyalkyl”, “hydroxyaliphatic”, and “hydroxyalkoxy” mean alkyl, aliphatic or alkoxy, as the case may be, substituted with one or more —OH groups.

The terms “alkoxyalkyl”, “alkoxyaliphatic”, and “alkoxyalkoxy” mean alkyl, aliphatic or alkoxy, as the case may be, substituted with one or more alkoxy groups. For example, an “alkoxyalkyl” refers to an alkyl group such as (alkyl-O)-alkyl-, wherein alkyl has been defined above.

The term “protecting group” and “protective group” as used herein, are interchangeable and refer to an agent used to temporarily block one or more desired functional groups in a compound with multiple reactive sites. In certain embodiments, a protecting group has one or more, or specifically all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

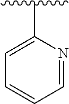 [SMILES: *c1ccccn1]

also represents

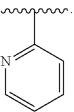 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays. Such compounds, especially deuterium (D) analogs, can also be therapeutically useful.

The terms “a bond” and “absent” are used interchangeably to indicate that a group is absent.

The compounds of the invention are defined herein by their chemical structures and/or chemical names. Where a compound is referred to by both a chemical structure and a chemical name, and the chemical structure and chemical name conflict, the chemical structure is determinative of the compound's identity.

The compounds described herein can exist in free form, or, where appropriate, as salts. Those salts that are pharmaceutically acceptable are of particular interest since they are useful in administering the compounds described above for medical purposes. Salts that are not pharmaceutically acceptable are useful in manufacturing processes, for isolation and purification purposes, and in some instances, for use in separating stereoisomeric forms of the compounds of the invention or intermediates thereof.

As used herein, the term “pharmaceutically acceptable salt” refers to salts of a compound, which are, within the scope of sound medical judgment, suitable for use in humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by, for example, 1) reacting the purified compound in its free-base form with a suitable organic or inorganic acid; and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by, for example, 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminium. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide and the like. Suitable amine base addition salts are prepared from amines which are frequently used in medicinal chemistry because of their low toxicity and acceptability for medical use Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

It should be understood that this invention includes mixtures/combinations of different pharmaceutically acceptable salts and also mixtures/combinations of compounds in free form and pharmaceutically acceptable salts.

In addition to the compounds described herein, the methods of the invention can be employed for preparing pharmaceutically acceptable solvates (e.g., hydrates) and clathrates of these compounds.

As used herein, the term “pharmaceutically acceptable solvate,” is a solvate formed from the association of one or more pharmaceutically acceptable solvent molecules to one of the compounds described herein. The term solvate includes hydrates (e.g., hemihydrate, monohydrate, dihydrate, trihydrate, tetrahydrate, and the like).

As used herein, the term “hydrate” means a compound described herein or a salt thereof that further includes a stoichiometric or non-stoichiometric amount of water bound by non-covalent intermolecular forces.

As used herein, he term “clathrate” means a compound described herein or a salt thereof in the form of a crystal lattice that contains spaces (e.g., channels) that have a guest molecule (e.g., a solvent or water) trapped within.

In addition to the compounds described herein, the methods of the invention can be employed for preparing pharmaceutically acceptable derivatives or prodrugs of these compounds.

A “pharmaceutically acceptable derivative or prodrug” includes any pharmaceutically acceptable ester, salt of an ester, or other derivative or salt thereof, of a compound described herein, which, upon administration to a recipient, is capable of providing, either directly or indirectly, a compound described herein or an inhibitorily active metabolite or residue thereof. Particularly favoured derivatives or prodrugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species.

As used herein and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds described herein that comprise —NO, —NO₂, —ONO, or —ONO₂ moieties. Prodrugs can typically be prepared using well-known methods, such as those described by BURGER'S MEDICINAL CHEMISTRY AND DRUG DISCOVERY (1995) 172-178, 949-982 (Manfred E. Wolff ed., 5th ed).

A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

Pharmaceutically acceptable prodrugs of the compounds described above include, without limitation, esters, amino acid esters, phosphate esters, metal salts and sulfonate esters.

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds of the invention are provided in the form of a single enantiomer at least 95%, at least 97% and at least 99% free of the corresponding enantiomer.

In a further embodiment, the compounds of the invention are in the form of the (+) enantiomer at least 95% free of the corresponding (−) enantiomer.

In a further embodiment, the compounds of the invention are in the form of the (+) enantiomer at least 97% free of the corresponding (−) enantiomer.

In a further embodiment, the compounds of the invention are in the form of the (+) enantiomer at least 99% free of the corresponding (−) enantiomer.

In a further embodiment, the compounds of the invention are in the form of the (−) enantiomer at least 95% free of the corresponding (+) enantiomer.

In a further embodiment, the compounds of the invention are in the form of the (−) enantiomer at least 97% free of the corresponding (+) enantiomer.

In a further embodiment the compounds of the invention are in the form of the (−) enantiomer at least 99% free of the corresponding (+) enantiomer.

In some embodiments, the compounds of the invention are provided as pharmaceutically acceptable salts e.g. Handbook of Pharmaceutical Salts Properties, Selection, and Use, Wiley, 2002, (P. Heinrich Stahl, Camille G. Wermuth, ed.)). As discussed above, such pharmaceutically acceptable salts can be derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. sodium, lithium, potassium), alkaline earth metals (e.g. calcium, magnesium), ammonium, NR₄₊ (where R is C₁₋₄ alkyl) salts, choline and tromethamine.

In one embodiment of the invention, the pharmaceutically acceptable salt is a sodium salt.

In one embodiment of the invention, the pharmaceutically acceptable salt is a potassium salt.

In one embodiment of the invention, the pharmaceutically acceptable salt is a lithium salt.

In one embodiment of the invention, the pharmaceutically acceptable salt is a tromethamine salt.

In one embodiment of the invention, the pharmaceutically acceptable salt is an L-arginine salt.

In one embodiment of the invention, the pharmaceutically acceptable salt is a calcium salt.

It will be appreciated by those skilled in the art that the compounds of the invention described herein can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the compounds of the invention described herein can exist in different solvate forms, for example hydrates. Solvates of the compounds of the invention may also form when solvent molecules are incorporated into the crystalline lattice structure of the compound molecule during the crystallization process.

The terms “subject,” “host,” or “patient” includes an animal and a human (e.g., male or female, for example, a child, an adolescent, or an adult). Preferably, the “subject,” “host,” or “patient” is a human.

In one embodiment, the present invention provides a method for treating or preventing a Flaviviridae viral infection in a host comprising administering to the host a therapeutically effective amount of at least one compound according to the invention described herein.

In one embodiment, the viral infection is chosen from Flaviviridae infections. In one embodiment, the Flaviviridae infection is Hepatitis C virus (HCV), bovine viral diarrhea virus (BVDV), hog cholera virus, dengue fever virus, Japanese encephalitis virus or yellow fever virus.

In one embodiment, the Flaviviridae viral infection is hepatitis C viral infection (HCV).

In one embodiment, the methods of the invention are directed for treatment of HCV genotype 1 infection. In another embodiment, the HCV is genotype 1a or genotype 1b.

In one embodiment, the present invention provides a method for treating or preventing a Flaviviridae viral infection in a host comprising administering to the host a therapeutically effective amount of at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In one embodiment, there is provided a method for inhibiting or reducing the activity of viral polymerase in a host comprising administering a therapeutically effective amount of a compound according to the invention described herein.

In one embodiment, there is provided a method for inhibiting or reducing the activity of viral polymerase in a host comprising administering a therapeutically effective amount of a compound according to the invention described herein and further comprising administering one or more viral polymerase inhibitors.

In one embodiment, viral polymerase is a Flaviviridae viral polymerase.

In one embodiment, viral polymerase is a RNA-dependant RNA-polymerase.

In one embodiment, viral polymerase is HCV polymerase.

In one embodiment, viral polymerase is HCV 5B polymerase.

In treating or preventing one or more conditions/diseases described above, the compounds described above can be formulated in pharmaceutically acceptable formulations that optionally further comprise a pharmaceutically acceptable carrier, adjuvant or vehicle.

In one embodiment, the present invention provides a pharmaceutical composition comprising at least one compound according to the invention described herein and at least one pharmaceutically acceptable carrier, adjuvant, or vehicle, which includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds described above, and pharmaceutically acceptable compositions thereof can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Any orally acceptable dosage form including, but not limited to, capsules, tablets, aqueous suspensions or solutions, can be used for the oral administration. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Liquid dosage forms for oral administration include, but are not limited to, pharmaceutically acceptable emulsions, microemulsions, solutions, suspensions, syrups and elixirs. In addition to the active compounds (the compounds described above), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Sterile injectable forms may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In order to prolong the effect of the active compounds administered, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

When desired the above described formulations adapted to give sustained release of the active ingredient may be employed.

Compositions for rectal or vaginal administration are specifically suppositories which can be prepared by mixing the active compound with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, transdermal patches, which have the added advantage of providing controlled delivery of a compound to the body, can also be used. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Alternatively, the compounds described above and pharmaceutically acceptable compositions thereof may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds described above and pharmaceutically acceptable compositions thereof can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration, for example, from 0.01 mg/kg body weight/day to 100 mg/kg body weight/day.

It will be appreciated that the amount of a compound according to the invention described herein required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

In one embodiment, the present invention provides a pharmaceutical composition comprising at least one compound according to the invention described herein, and further comprising one or more additional agents chosen from viral serine protease inhibitors, viral polymerase inhibitors, viral NS5A inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In another embodiment, there is provided a combination therapy of at least one compound according to the invention described herein in combination with one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

The additional agents for the compositions and combinations include, for example, ribavirin, amantadine, merimepodib, Levovirin, Viramidine, and maxamine.

In one combination embodiment, the compound and additional agent are administered sequentially.

In another combination embodiment, the compound and additional agent are administered simultaneously. The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The term “viral serine protease inhibitor” as used herein means an agent that is effective to inhibit the function of the viral serine protease including HCV serine protease in a mammal Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in: WO 03/010140 (Boehringer Ingelheim), WO 03/026587 (Bristol Myers Squibb); WO 02/100846 A1, WO 02/100851A2, WO 01/85172 AI (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121 A2 (Idenix), WO 02/069903 A2 (Biocryst Pharmaceuticals Inc.), and WO 02/057287 A2 (Merck/Isis) and WO 02/057425 A2 (Merck/lsis).

Specific examples of nucleoside inhibitors of an HCV polymerase, include R1626, R1479 (Roche), R7128 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190 (Gilead), and PF-868,554 (Pfizer).

The term “viral NS5A inhibitor” as used herein means an agent that is effective to inhibit the function of the viral NS5A protease in a mammal. Inhibitors of HCV NS5A include, for example, those compounds described in WO2010/117635, WO2010/117977, WO2010/117704, WO2010/1200621, WO2010/096302, WO2010/017401, WO2009/102633, WO2009/102568, WO2009/102325, WO2009/102318, WO2009020828, WO2009020825, WO2008144380, WO2008/021936, WO2008/021928, WO2008/021927, WO2006/133326, WO2004/014852, WO2004/014313, WO2010/096777, WO2010/065681, WO2010/065668, WO2010/065674, WO2010/062821, WO2010/099527, WO2010/096462, WO2010/091413, WO2010/094077, WO2010/111483, WO2010/120935, WO2010/126967, WO2010/132538, and WO2010/122162. Specific examples of HCV NS5A inhibitors include: EDP-239 (being developed by Enanta); ACH-2928 (being developed by Achillion); PPI-1301 (being developed by Presido Pharmaceuticals); PPI-461 (being developed by Presido Pharmaceuticals); AZD-7295 (being developed by AstraZeneca); GS-5885 (being developed by Gilead); BMS-824393 (being developed by Bristol-Myers Squibb); BMS-790052 (being developed by Bristol-Myers Squibb)

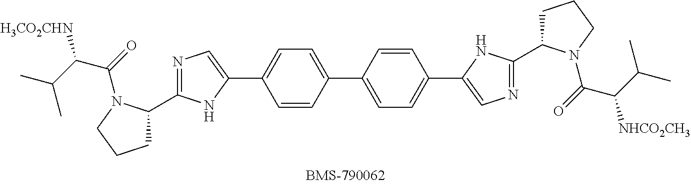

(Gao M. et al. Nature, 465, 96-100 (2010), nucleoside or nucleotide polymerase inhibitors, such as PSI-661 (being developed by Pharmasset), PSI-938 (being developed by Pharmasset), PSI-7977 (being developed by Pharmasset), INX-189 (being developed by Inhibitex), JTK-853 (being developed by Japan Tobacco), TMC-647055 (Tibotec Pharmaceuticals), RO-5303253 (being developed by Hoffmann-La Roche), and IDX-184 (being developed by Idenix Pharmaceuticals).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as alpha-, beta-, delta- and omega-interferons, x-interferons, consensus interferons and asialo-interferons), class II interferons (such as gamma-interferons) and pegylated interferons.

Exemplary immunomudulating agents, include, but are not limited to: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alpha 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.).

The term “class I interferon” as used herein means an interferon selected from a group of interferons that all bind to receptor type 1. This includes both naturally and synthetically produced class I interferons. Examples of class I interferons include alpha-, beta-, delta- and omega-interferons, tau-interferons, consensus interferons and asialo-interferons. The term “class II interferon” as used herein means an interferon selected from a group of interferons that all bind to receptor type II. Examples of class II interferons include gamma-interferons.

Antisense agents include, for example, ISIS-14803.

Specific examples of inhibitors of HCV NS3 protease, include BILN-2061 (Boehringer Ingelheim) SCH-6 and SCH-503034/Boceprevir (Schering-Plough), VX-950/telaprevir (Vertex) and ITMN-B (InterMune), GS9132 (Gilead), TMC-435350 (Tibotec/Medivir), ITMN-191 (InterMune), MK-7009 (Merck).

Inhibitor internal ribosome entry site (IRES) includes ISIS-14803 (ISIS Pharmaceuticals) and those compounds described in WO 2006019831 (PTC therapeutics).

In one embodiment, the additional agent is interferon alpha, ribavirin, silybum marianum, interleukine-12, amantadine, ribozyme, thymosin, N-acetyl cysteine or cyclosporin.

In one embodiment, the additional agent is interferon alpha 1A, interferon alpha 1B, interferon alpha 2A, or interferon alpha 2B. Interferon is available in pegylated and non pegylated forms. Pegylated interferons include PEGASYS™ and Peg-intron™.

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

The recommended dose of PEG-Intron™ regimen is 1.0 mg/kg/week subcutaneously for one year. The dose should be administered on the same day of the week.

When administered in combination with ribavirin, the recommended dose of PEG-Intron is 1.5 micrograms/kg/week.

In one embodiment, viral serine protease inhibitor is a flaviviridae serine protease inhibitor.

In one embodiment, viral polymerase inhibitor is a flaviviridae polymerase inhibitor.

In one embodiment, viral helicase inhibitor is a flaviviridae helicase inhibitor.

In further embodiments: viral serine protease inhibitor is HCV serine protease inhibitor; viral polymerase inhibitor is HCV polymerase inhibitor; viral helicase inhibitor is HCV helicase inhibitor.

In one embodiment, the present invention provides a pharmaceutical composition comprising at least one compound according to the invention described herein, one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin, and at least one pharmaceutically acceptable carrier or excipient.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention. The individual components for use in the method of the present invention or combinations of the present invention may be administered either sequentially or simultaneously in separate or combined pharmaceutical formulations.

In one embodiment, the present invention provides the use of a compound according to the invention described herein for treating or preventing Flaviviridae viral infection in a host.

In one embodiment, the present invention provides the use of a compound according to the invention described herein for the manufacture of a medicament for treating or preventing a viral Flaviviridae infection in a host.

In one embodiment, the present invention provides the use of a compound according to the invention described herein for inhibiting or reducing the activity of viral polymerase in a host.

In a further embodiment, the composition or combination according to the invention further comprises at least one compound according to the invention described herein; one or more additional agents select from non-nucleoside HCV polymerase inhibitors (e.g., HCV-796), nucleoside HCV polymerase inhibitors (e.g., R7128, R1626, R1479), and HCV NS3 protease inhibitors (e.g., VX-950/telaprevir and ITMN-191); and interferon and/or ribavirin.

In one embodiment, the additional agent is interferon α1A, interferon α1B, interferon α2A, or interferon α2B, and optionally ribavirin.

In one embodiment, the present invention provides a method for treating or preventing a HCV viral infection in a host comprising administering to the host a combined therapeutically effective amounts of at least one compound according to the invention described herein, and one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin.

In one combination embodiment, the compound and additional agent are administered sequentially.

In another combination embodiment, the compound and additional agent are administered simultaneously.

In one embodiment, there is provided a method for inhibiting or reducing the activity of HCV viral polymerase in a host comprising administering to the host a combined therapeutically effective amounts of at least one compound of the invention, and one or more additional agents select from non-nucleoside HCV polymerase inhibitors (e.g., HCV-796) and nucleoside HCV polymerase inhibitors (e.g., R7128, R1626, R1479), interferon and ribavirin.

The combinations referred to above may conveniently be presented for use in the form of a pharmaceutical formulation and thus pharmaceutical formulations or compositions comprising a combination as defined above together with a pharmaceutically acceptable carrier therefore comprise a further aspect of the invention.

The individual components for use in the method of the present invention or combinations of the present invention may be administered either sequentially or simultaneously in separate or combined pharmaceutical formulations.

In one embodiment, the present invention provides the use of at least one compound of the invention, in combination with the use of one or more additional agents select from non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon and ribavirin, for the manufacture of a medicament for treating or preventing a HCV infection in a host.

When the compounds of the invention described herein are used in combination with at least one second therapeutic agent active against the same virus, the dose of each compound may be either the same as or differ from that when the compound is used alone. Appropriate doses will be readily appreciated by those skilled in the art.

The ratio of the amount of a compound according to the invention described herein administered relative to the amount of the additional agent (non-nucleoside HCV polymerase inhibitors (e.g., HCV-796), nucleoside HCV polymerase inhibitors (e.g., R7128, R1626, R1479), HCV NS3 protease inhibitors (e.g., VX-950/telaprevir and ITMN-191), interferon or ribavirin) will vary dependent on the selection of the compound and additional agent.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

The compounds according to the invention described herein can be prepared by any suitable method known in the art. For example, the compounds can be prepared in accordance with procedures described in U.S. Pat. No. 6,881,741, US 2005/0009804, US 2006/0276533, WO 2002/100851, and WO 08/58393, the disclosures of which are hereby incorporated by reference. Specific exemplary preparation details are described below in the Exemplification section.

### EXEMPLIFICATION

### Example 1

### Synthesis of Compounds of the Invention

The compounds according to the invention described herein can be prepared by any suitable method known in the art, for example, U.S. Pat. No. 6,881,741, US 2005/0009804, US 2006/0276533, WO 2002/100851, and WO 08/58393. Preparation details of some exemplary compounds are described below. Syntheses of certain exemplary compounds of the invention are described below. Generally, the compounds of the invention can be prepared as shown in those syntheses optionally with any desired appropriate modification.

### A. General Analytical Methods

As used herein the term RT (min) refers to the LCMS retention time, in minutes, associated with the compound. Unless otherwise indicated, the method employed to obtain the reported retention times is as follows:

Column: YMC-Pack Pro C18, 50 mm×4.6 mm id

Gradient: 10-95% methanol/H₂O. Flow rate: 1.5 ml/min. UV-vis detection.

### B. General Analytical Methods and Methodology for Synthesis and Characterization of Compounds

As used herein the term RT (min) refers to the LCMS retention time, in minutes, associated with the compound. NMR and Mass Spectroscopy data of certain specific compounds are summarized in Table 1.

Purification by reverse phase HPLC was carried out under standard conditions using a Phenomenex Gemini C18 column, 21.2 mmID×250 mm, 5 m, 110 Å. Elution is performed using a linear gradient 20 to 90% (CH₃CN in water or CH₃CN in water with 0.02% HCl) with a flow rate of 5.0 mL/minute.

The following abbreviations may be used as follows:

aq aqueous
conc concentrate
DCM methylene chloride

### DIPEA Diisopropylethylamine

DMF dimethylformamide

### DMSO Dimethylsulfoxide

EtOAc Ethyl acetate
Mol molar

### MeOH Methanol

MTBE methyl ter-butyl ether
n-BuLi n-butyl lithium
PdCl₂dppf (1,1′-Bis-(diphenylphosphino)-ferrocene)palladium (II)dichloride
Pd(PPh₃)₂Cl₂ trans-dichlorobis(triphenyl phosphine) Palladium (II)
RT room temperature

### TEA Triethylamine

### THF Tetrahydrofuran

### C. Syntheses of Compounds

### Preparation of Compound 83: 3-[(2,4-Dimethyl-benzoyl)-(trans-4-hydroxy-cyclohexyl)-amino]-5-(3,3-dimethyl-but-1-ynyl)-thiophene-2-carboxylic acid

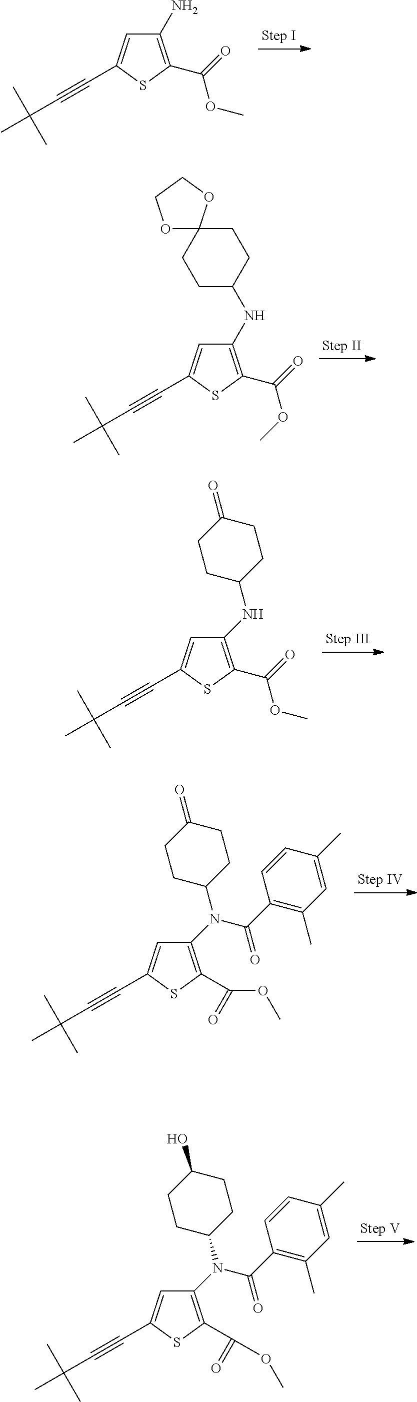

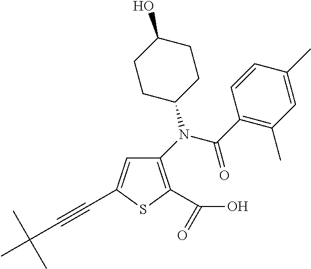 [SMILES: Cc1ccc(C(=O)N(c2cc(C#CC(C)(C)C)sc2C(=O)O)[C@H]2CC[C@H](O)CC2)c(C)c1]

### Step I

To a solution of 3-amino-5-(3,3-dimethyl-but-1-ynyl)-thiophene-2-carboxylic acid methyl ester (3.18 g, 13.1 mmol) in toluene (16 mL) was sequentially added 1,4-cyclohexanedione monoethylene ketal (4.09 g, 26.2 mmol), acetic acid (750 μL, 0.0131 mmol) and sodium triacetoxyborohydride (5.55 g, 26.2 mmol) under nitrogen atmosphere. The reaction mixture was stirred at RT overnight, filtered and washed with toluene (10 mL). The organic layer was washed with saturated sodium bicarbonate solution (1×10 mL), and EtOAc (1×20 mL) and concentrated to dryness. The residue was purified by flash column chromatography on silica gel (100% DCM) to give 5-(3,3-dimethyl-but-1-ynyl)-3-(1,4-dioxa-spiro[4.5]dec-8-ylamino)-thiophene-2-carboxylic acid methyl ester (4.1 g, 83%).

### Step II

To a solution of 5-(3,3-dimethyl-but-1-ynyl)-3-(1,4-dioxa-spiro[4.5]dec-8-ylamino)-thiophene-2-carboxylic acid methyl (4.0 g, 10.16 mmol) in THF (20 mL) was added aqueous HCl (20 mL, 3.6 N) under nitrogen atmosphere. The reaction mixture was stirred overnight at 40° C. Additional THF (30 mL) and HCl (5 mL, 12 N) were added and the mixture is stirred overnight at 40° C., cooled to RT and diluted with THF (10 mL). The organic layer was diluted with water (1×20 mL) and THF was evaporated to form a precipitate in H₂O. The precipitate was filtered, washed with H₂O and co-evaporated with toluene to give 5-(3,3-dimethyl-but-1-ynyl)-3-(4-oxo-cyclohexylamino)-thiophene-2-carboxylic acid methyl ester (2.75 g, 73%).

### Step III

To a solution of 5-(3,3-dimethyl-but-1-ynyl)-3-(4-oxo-cyclohexylamino)-thiophene-2-carboxylic acid methyl ester (200 mg, 0.539 mmol) in toluene (5 mL) was sequentially added pyridine (85 mL, 1.08 mmol) and 2,4-dimethylbenzoyl chloride (182 mL, 1.08 mmol) under nitrogen atmosphere. The reaction mixture was heated at 110° C. overnight in a sealed tube, cooled to RT, and diluted with EtOAc (10 mL). The reaction mixture was washed with saturated sodium bicarbonate solution (1×5 mL), dried over Na₂SO₄, filtered, and concentrated to dryness. The residue was purified by flash column chromatography on silica gel (0 to 50% EtOAc in Hexanes) to give 3-[(2,4-dimethyl-benzoyl)-(4-oxo-cyclohexyl)-amino]-5-(3,3-dimethyl-but-1-ynyl)-thiophene-2-carboxylic acid methyl ester (200 mg, 80%).

### Step IV

Sodium borohydride (16 mg, 0.43 mmol) was added to THF (2 mL) and H₂O (40 μL) at −20° C. To this mixture was added a solution of 3-[(2,4-dimethyl-benzoyl)-(4-oxo-cyclohexyl)-amino]-5-(3,3-dimethyl-but-1-ynyl)-thiophene-2-carboxylic acid methyl ester (200 mg, 0.43 mmol) in THF (4 mL) under nitrogen atmosphere. The reaction mixture was stirred at −20° C. for 30 minutes and aqueous HCl 1N (2 mL) was added. The reaction mixture was extracted with EtOAc (2×5 mL), and the organic layer was dried over Na₂SO₄, filtered, and concentrated to dryness. The residue was purified by flash column chromatography on silica gel (0 to 50% EtOAc in Hexanes) to give 3-[(2,4-dimethyl-benzoyl)-(trans-4-hydroxy-cyclohexyl)-amino]-5-(3,3-dimethyl-but-1-ynyl)-thiophene-2-carboxylic acid methyl ester (123 mg, 61%).

### Step V

To a solution of 3-[(2,4-dimethyl-benzoyl)-(trans-4-hydroxy-cyclohexyl)-amino]-5-(3,3-dimethyl-but-1-ynyl)-thiophene-2-carboxylic acid methyl ester (80 mg, 0.171 mmol) in a 3:2:1 mixture of THF:methanol:H₂O (2 mL) was added lithium hydroxide monohydride (20 mg, 0.856 mmol) under nitrogen atmosphere. The reaction mixture was stirred overnight and acidified to pH 3-4 with aqueous HCl 1N. The reaction mixture was extracted with EtOAc (2×5 mL), and the combined organic layers are dried over Na₂SO₄, filtered, and concentrated to dryness. The residue was purified by flash column chromatography on silica gel (0 to 10% methanol in DCM) to give 3-[(2,4-dimethyl-benzoyl)-(trans-4-hydroxy-cyclohexyl)-amino]-5-(3,3-dimethyl-but-1-ynyl)-thiophene-2-carboxylic acid (35 g, 45%).

¹H NMR (400 MHz, DMSO-d₆): δ 7.15-7.01 (m, 2H), 6.86 (s, 1H), 6.72 (d, J=7.7 Hz, 1H), 4.61-4.33 (m, 2H), 3.44-3.21 (m, 2H), 2.18 (d, J=17.2 Hz, 6H), 2.03-1.96 (d, J=11.9 Hz, 1H), 1.90-1.75 (m, 3H), 1.45-1.28 (m, 3H), 1.25 (s, 9H), 0.99-0.85 (m, 1H).

LC/MS: m/z=454.13 (M+H⁺).

### Preparation of Compounds 90, 91, 92 and 82

The following compounds were prepared using essentially the same procedure described above Compound 83:

### Compound 90: 3-[(2,4-Dichloro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 13.71 (s, 1H), 7.54 (d, 1H), 7.35 (dd, 1H), 7.25 (d, 1H), 7.21 (s, 1H), 4.58 (s, 1H), 4.44-4.34 (m, 1H), 3.30-3.23 (m, 1H), 2.05-1.99 (m, 1H), 1.93-1.69 (m, 4H), 1.53-1.41 (m, 2H), 1.27 (s, 9H), 1.02-0.90 (m, 2H).

LC/MS: m/z=494.03 (M+H⁺).

### Compound 91: 3-[(4-Chloro-2-fluoro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 7.51 (m, 1H), 7.26 (m, 1H), 7.08 (m, 1H), 6.84 (s, 1H), 4.53 (bs, 1H), 4.34 (m, 1H), 3.25 (m, 1H), 1.95-1.84 (m, 3H), 1.82-1.73 (m, 1H), 1.44-1.12 (m, 12H), 0.99-0.84 (m, 1H).

LC/MS: m/z=478.04 (M+H⁺)

### Compound 92: 5-(3,3-Dimethyl-but-1-ynyl)-3-[(4-fluoro-benzoyl)-(trans-4-hydroxy-cyclohexyl)-amino]-thiophene-2-carboxylic acid

LC/MS: m/z=444.09 (M+H⁺)

### Compound 82: 5-(3,3-Dimethyl-but-1-ynyl)-3-[(trans-4-hydroxy-cyclohexyl)-(4-trifluoromethyl-benzoyl)-amino]thiophene-2-carboxylic acid)

LC/MS: m/z=494.08 (M+H⁺)

### Preparation of Compound 84: 3-[(2-Chloro-4-methyl-benzoyl)-(trans-4-hydroxy-cyclohexyl)-amino]-5-(3,3-dimethyl-but-1-ynyl)-thiophene-2-carboxylic acid

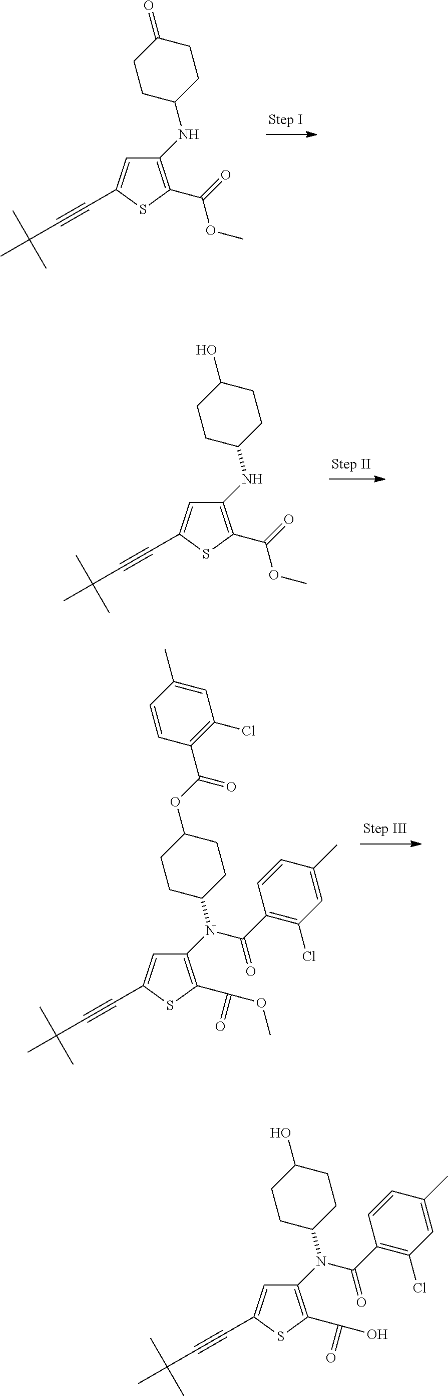

### Step I

Sodium borohydride (170 mg, 4.49 mmol) was added to a mixture of THF (15 mL) and H₂O (300 μL) at −15° C. To this mixture was added a solution of 5-(3,3-dimethyl-but-1-ynyl)-3-(4-oxo-cyclohexylamino)-thiophene-2-carboxylic acid methyl ester (1.58 g, 4.49 mmol) in THF (15 mL) under nitrogen atmosphere. The reaction mixture was stirred at −15° C. for 45 minutes, warmed to RT and aqueous HCl (2 mL, 1 N) was added. The reaction mixture was extracted by EtOAc (2×20 mL), and the organic layer was dried over Na₂SO₄, filtered, and concentrated to dryness. The residue was purified by flash column chromatography on silica gel (0 to 50% EtOAc in Hexanes) to give 5-(3,3-dimethyl-but-1-ynyl)-3-(trans-4-hydroxy-cyclohexylamino)-thiophene-2-carboxylic acid methyl ester (1.32 g, 88%).

### Step II

To a solution of 2-chloro-4-methylbenzoyl chloride (280 mg, 1.48 mmol) in toluene (2 mL) was sequentially added 5-(3,3-dimethyl-but-1-ynyl)-3-(trans-4-hydroxy-cyclohexylamino)-thiophene-2-carboxylic acid methyl ester (125 g, 0.37 mmol) and pyridine (140 μL, 1.74 mmol) under nitrogen atmosphere. The reaction mixture was stirred overnight at 100° C., cooled to RT, and concentrated to dryness. The residue was purified by flash column chromatography on silica gel (0 to 50% EtOAc in Hexanes) to give 3-{(2-chloro-4-methyl-benzoyl)-[trans-4-(2-chloro-4-methyl-benzoyloxy)-cyclohexyl]-amino}-5-(3,3-dimethyl-but-1-ynyl)-thiophene-2-carboxylic acid methyl ester (175 mg, 74%).

### Step III

To a solution of 3-{(2-chloro-4-methyl-benzoyl)-[trans-4-(2-chloro-4-methyl-benzoyloxy)-cyclohexyl]-amino}-5-(3,3-dimethyl-but-1-ynyl)-thiophene-2-carboxylic acid methyl ester (175 mg, 0.21 mmol) in a 3:2:1 mixture of THF:methanol:H₂O (2 mL) was added lithium hydroxide (170 mg, 2.7 mmol) under nitrogen atmosphere. The reaction mixture was stirred overnight and acidified to pH 3-4 with aqueous HCl 1N. The reaction mixture was extracted by EtOAc (2×3 mL), and the combined organic layers are dried over Na₂SO₄, filtered, and concentrated to dryness. The residue was purified by flash column chromatography on silica gel (0 to 10% methanol in DCM) to give 3-[(2-chloro-4-methyl-benzoyl)-(trans-4-hydroxy-cyclohexyl)-amino]-5-(3,3-dimethyl-but-1-ynyl)-thiophene-2-carboxylic acid (35 g, 45%).

¹H NMR (400 MHz, DMSO-d₆): δ 7.14 (d, 3H), 7.00 (d, 1H), 4.56 (bs, 1H), 4.45-4.33 (m, 1H), 3.30 (m, 1H), 2.21 (s, 3H), 2.06-1.98 (m, 1H), 1.93-1.70 (m, 4H), 1.52-1.39 (m, 2H), 1.34-1.28 (m, 2H), 1.26 (s, 9H), 1.24-1.12 (m, 2H), 1.01-0.88 (m, 1H).

LC/MS: m/z=474.07 (M+H⁺).

### Preparation of Compounds 85, 86, 87, 88, 89, 81, 93 and 94

The following compounds were prepared using essentially the same procedure described above for Compound 84:

### Compound 85: 5-(3,3-Dimethyl-but-1-ynyl)-3-[(2-fluoro-4-methyl-benzoyl)-(trans-4-hydroxy-cyclohexyl)-amino]thiophene-2-carboxylic acid

¹H NMR (400 MHz, DMSO-d₆): δ 13.48 (s, 1H), 7.17 (s, 1H), 7.10 (t, 1H), 6.86 (d, 2H), 4.55 (d, 1H), 4.45-4.32 (m, 1H), 3.31-3.20 (m, 1H), 2.22 (s, 3H), 2.00-1.69 (m, 5H), 1.55-1.39 (m, 2H), 1.28 (s, 9H), 1.07-0.89 (m, 2H).

LC/MS: m/z=458.11 (M+H⁺).

### Compound 86: 5-(3,3-Dimethyl-but-1-ynyl)-3-[(trans-4-hydroxy-cyclohexyl)-(4-methyl-benzoyl)-amino]-thiophene-2-carboxylic acid

LC/MS: m/z=440.13 (M+H⁺).

### Compound 87: 3-[(2-Chloro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 13.66 (s, 1H), 7.36-7.12 (m, 5H), 4.57 (s, 1H), 4.41 (s, 1H), 3.30-3.22 (m, 1H), 2.09-1.70 (m, 5H), 1.54-1.39 (m, 2H), 1.35-1.18 (m, 9H), 1.03-0.89 (m, 1H).

LC/MS: m/z=461.94 (M+H⁺).

### Compound 88: 5-(3,3-Dimethyl-but-1-ynyl)-3-[(trans-4-hydroxy-cyclohexyl)-(2-methyl-benzoyl)-amino]-thiophene-2-carboxylic acid

¹H NMR (400 MHz, DMSO-d₆): δ 13.49 (s, 1H), 7.24 (s, 1H), 7.13-6.92 (m, 4H), 4.56 (d, 1H), 4.48-4.36 (m, 1H), 3.31-3.22 (m, 1H), 2.23 (s, 3H), 2.06-1.72 (m, 5H), 1.54-1.39 (m, 2H), 1.33-1.31 (m, 1H), 1.26 (s, 9H), 1.04-0.90 (m, 2H).

LC/MS: m/z=439.98 (M+H⁺).

### Compound 89: 3-[(2,3-Difluoro-4-methyl-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 13.56 (s, 1H), 7.25 (s, 1H), 7.03-6.83 (m, 2H), 4.56 (d, 1H), 4.45-4.30 (m, 1H), 3.30-3.17 (m, 1H), 2.19 (s, 3H), 2.05-1.65 (m, 5H), 1.58-1.38 (m, 1H), 1.37-1.15 (m, 10H), 1.07-0.90 (m, 1H).

LC/MS: m/z=475.97 (M+H⁺).

### Compound 81: 3-[(4-Chloro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 7.29 (d, 2H), 7.20 (d, 2H), 6.99 (s, 1H), 4.60 (d, 1H), 4.39-4.28 (m, 1H), 4.28-4.19 (m, 1H), 3.29-3.17 (m, 1H), 3.15 (d, 2H), 1.97-1.82 (m, 3H), 1.82-1.71 (m, 1H), 1.29-1.20 (m, 9H), 1.01-0.86 (m, 2H).

LC/MS: m/z=460.01 (M+H⁺).

### Compound 93: 5-(3,3-Dimethyl-but-1-ynyl)-3-[(3-fluoro-4-methyl-benzoyl)-(trans-4-hydroxy-cyclohexyl)-amino]thiophene-2-carboxylic acid

LC/MS: m/z=458.11 (M+H⁺).

### Compound 94: 3-[(4-Chloro-3-fluoro-benzoyl)-(trans-4-hydroxy-cyclohexyl)-amino]-5-(3,3-dimethyl-but-1-ynyl)-thiophene-2-carboxylic acid

¹H NMR (400 MHz, DMSO-d₆): δ 13.59-13.40 (m, 1H), 7.48 (t, 1H), 7.38 (s, 1H), 7.20 (d, 1H), 7.03 (d, 1H), 4.55 (d, 1H), 4.43-4.32 (m, 1H), 3.31-3.21 (m, 1H), 2.02-1.71 (m, 5H), 1.51-1.39 (m, 2H), 1.29 (s, 9H), 1.27-1.17 (m, 1H), 1.08-0.94 (m, 1H).

LC/MS: m/z=478.06 (M+H⁺).

### Preparation of Compound 1: 5-(3,3-Dimethyl-but-1-ynyl)-3-[(trans-4-methyl-cyclohexanecarbonyl)-pyridin-3-yl-methyl-amino]-thiophene-2-carboxylic acid

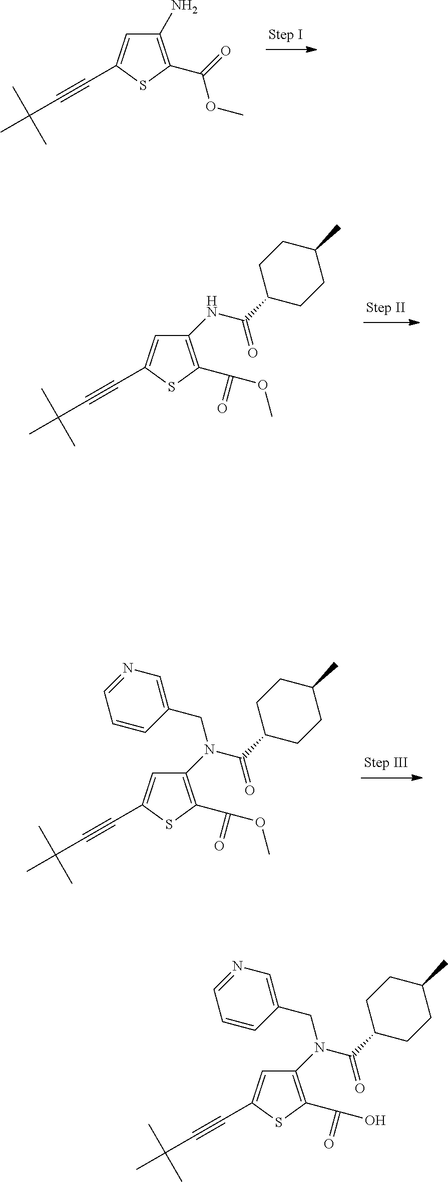

### Step I

To a solution of 3-amino-5-(3,3-dimethyl-but-1-ynyl)-thiophene-2-carboxylic acid methyl ester (280 mg, 1.05 mmol) in 1,2-dichloro-ethane (2 mL) was added trans-4-methylcyclohexanecarbonyl chloride (254 mg, 1.58 mmol) under nitrogen atmosphere. The reaction mixture was heated at 80° C. overnight, cooled to RT, and concentrated to dryness. The residue was purified by flash column chromatography on silica gel (0 to 20% EtOAc in Hexanes) to give 5-(3,3-dimethyl-but-1-ynyl)-3-[(trans-4-methyl-cyclohexanecarbonyl)-amino]-thiophene-2-carboxylic acid methyl ester (360 mg, 95%).

### Step II

To a solution of 5-(3,3-dimethyl-but-1-ynyl)-3-[(trans-4-methyl-cyclohexanecarbonyl)-amino]-thiophene-2-carboxylic acid methyl ester (100 mg, 0.277 mmol) in DMF (1 mL) at 0° C. was added sodium hydride 60% in mineral oil (3.8 mg, 0.9695 mmol) under nitrogen atmosphere. The mixture was stirred in an ice bath for 10 minutes and brought to RT. 3-(bromomethyl)pyridine hydrobromide (105 mg, 0.415 mmol) was added and the mixture is stirred for 2 hours, and quenched with H₂O (1 mL). The reaction mixture was extracted by EtOAc (2×5 mL), and the organic phase was dried over Na₂SO₄, filtered, and concentrated to dryness to give 5-(3,3-dimethyl-but-1-ynyl)-3-[(trans-4-methyl-cyclohexanecarbonyl)-pyridin-3-ylmethyl-amino]-thiophene-2-carboxylic acid methyl ester (60 mg) which was used for the next step.

### Step III

To a solution of 5-(3,3-dimethyl-but-1-ynyl)-3-[(trans-4-methyl-cyclohexanecarbonyl)-pyridin-3-ylmethyl-amino]-thiophene-2-carboxylic acid methyl ester (60 mg, 0.191 mmol) in a 3:2:1 mixture of THF:methanol:H₂O (0.6 mL) was added lithium hydroxide (80 mg, 1.91 mmol) under nitrogen atmosphere. The reaction mixture was stirred at RT overnight, acidified to pH 3-4 with aqueous HCl 1N and concentrated to dryness, co-evaporating with toluene. The residue was taken in H₂O and extracted with DCM (2×3 mL). The combined organic layers are dried over Na₂SO₄, filtered, and concentrated to dryness. The residue was purified by flash column chromatography on silica gel (0 to 10% methanol in DCM) to give 5-(3,3-dimethyl-but-1-ynyl)-3-[(trans-4-methyl-cyclohexanecarbonyl)-pyridin-3-ylmethyl-amino]-thiophene-2-carboxylic acid (15 mg, 25%).

LC/MS: m/z=439.00 (M+H⁺)

### Preparation of Compound 2: 3-[Carboxymethyl-(trans-4-methyl-cyclohexanecarbonyl)-amino]-5-(3,3-dimethyl-but-1-ynyl)-thiophene-2-carboxylic acid

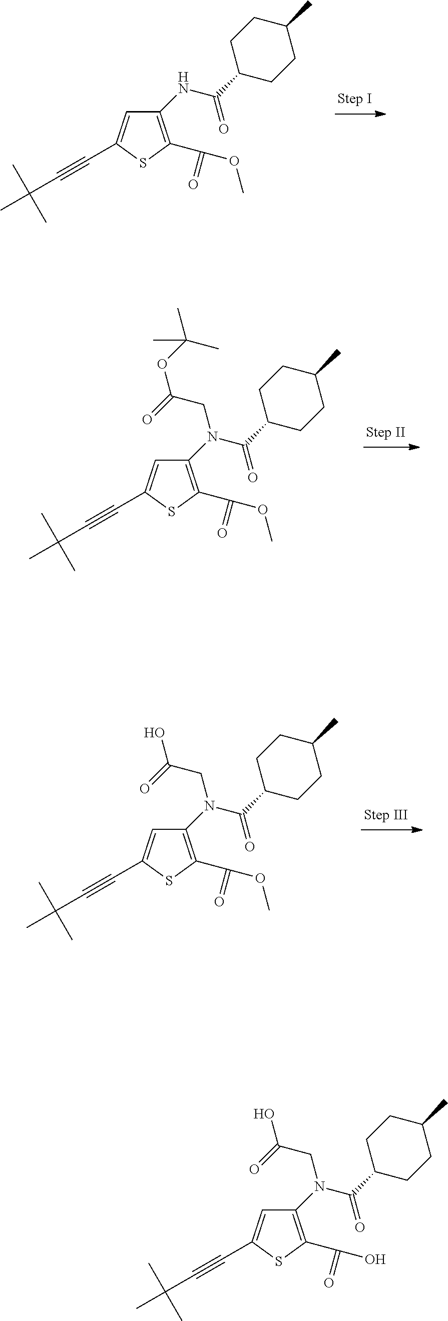

### Step I

To a solution of 5-(3,3-dimethyl-but-1-ynyl)-3-[(trans-4-methyl-cyclohexanecarbonyl)-amino]-thiophene-2-carboxylic acid methyl ester (100 mg, 0.276 mmol) in DMF (1 mL) at 0° C. was added sodium hydride 60% in mineral oil (27.6 mg, 0.69 mmol) under nitrogen atmosphere. The mixture was stirred in an ice bath for 10 minutes and brought to RT. tert-Butyl bromoacetate (61.3 mL, 0.415 mmol) was added dropwise and the mixture was stirred at RT for 40 minutes. The reaction mixture was quenched with H₂O (2 mL), and extracted by EtOAc (1×3 mL). The organic phase was washed with H₂O (2×2 mL), dried over Na₂SO₄, filtered, and concentrated to dryness to give 3-[tert-butoxycarbonylmethyl-(trans-4-methyl-cyclohexanecarbonyl)-amino]-5-(3,3-dimethyl-but-1-ynyl)-thiophene-2-carboxylic acid methyl ester (125 mg, 97%).

### Step II

To a solution of 3-[tert-butoxycarbonylmethyl-(trans-4-methyl-cyclohexanecarbonyl)-amino]-5-(3,3-dimethyl-but-1-ynyl)-thiophene-2-carboxylic acid methyl ester (91 mg, 0.29 mmol) in DCM (5 mL) at 0° C. was added trifluoroacetic acid (5 mL, 65.3 mmol) under nitrogen atmosphere. The mixture was stirred in an ice bath and brought to RT over 1 hour. The reaction mixture was concentrated to dryness, dissolved in H₂O (3 mL) and neutralized with saturated Na₂CO₃ solution. The reaction mixture was extracted by dichloromethane (2×2 mL), filtered, and the filtrate was concentrated to dryness to give 3-[carboxymethyl-(trans-4-methyl-cyclohexanecarbonyl)-amino]-5-(3,3-dimethyl-but-1-ynyl)-thiophene-2-carboxylic acid methyl ester (90.3 mg, 100%).

### Step III

To a solution of 3-[carboxymethyl-(trans-4-methyl-cyclohexanecarbonyl)-amino]-5-(3,3-dimethyl-but-1-ynyl)-thiophene-2-carboxylic acid methyl ester (13.4 mg, 0.032 mmol) in a 3:2:1 mixture of THF:methanol:H₂O (0.13 mL) was added lithium hydroxide (13.3 mg, 0.32 mmol) under nitrogen atmosphere. The reaction mixture was stirred at RT overnight and evaporated to dryness. The residue was purified by reverse phase preparative HPLC to give 3-[carboxymethyl-(trans-4-methyl-cyclohexanecarbonyl)-amino]-5-(3,3-dimethyl-but-1-ynyl)-thiophene-2-carboxylic acid (11.39 mg, 87%).

LC/MS: m/z=405.93 (M+H⁺).

### Preparation of Compound 24: 5-(3,3-Dimethyl-but-1-ynyl)-3-[(trans-4-methyl-cyclohexanecarbonyl)-(5-methyl-[1,3,4]oxadiazol-2-ylmethyl)-amino]-thiophene-2-carboxylic acid

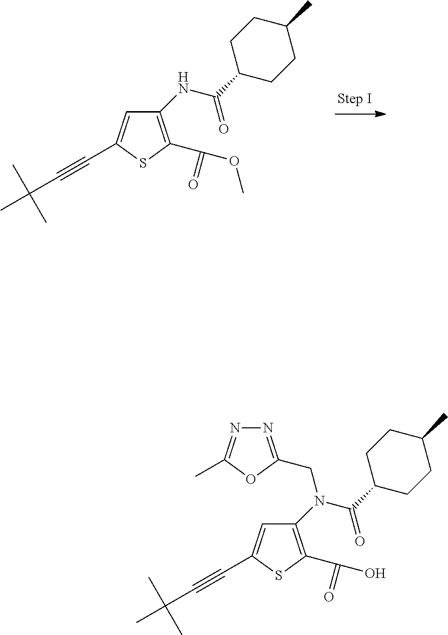

To a solution of 5-(3,3-dimethyl-but-1-ynyl)-3-[(trans-4-methyl-cyclohexanecarbonyl)-amino]-thiophene-2-carboxylic acid methyl ester (88.3 mg, 0.244 mmol) in acetonitrile (2.4 mL) was sequentially added 2-chloromethyl-5-methyl-[1,3,4]oxadiazole (97 mg, 0.733 mmol) and triethylamine (0.170 mL, 1.221 mmol) under nitrogen atmosphere. The reaction mixture was heated at 150° C. for 30 minutes under microwave irradiation, cooled to RT, and concentrated to dryness. The residue was purified by flash column chromatography on silica gel (0 to 35% EtOAc in Hexanes) and recrystallized to give 5-(3,3-dimethyl-but-1-ynyl)-3-[(trans-4-methyl-cyclohexanecarbonyl)-(5-methyl-[1,3,4]oxadiazol-2-ylmethyl)-amino]-thiophene-2-carboxylic acid (51 mg, 29%).

¹H NMR (400 MHz, CDCl₃): δ 10.02 (s, 1H), 8.13 (d, 1H), 5.45 (s, 2H), 2.58 (s, 3H), 2.30-2.19 (m, 1H), 2.05-1.98 (m, 2H), 1.84-1.77 (m, 2H), 1.60-1.47 (m, 2H), 1.29 (s, 9H), 1.06-0.94 (m, 3H), 0.91 (d, 3H).

LC/MS: m/z=444.05 (M+H⁺).

### Preparation of Compound 13: (3-[Cyclohex-3-enyl-(trans-4-methyl-cyclohexanecarbonyl)-amino]-5-(3,3-dimethyl-but-1-ynyl)-thiophene-2-carboxylic acid)

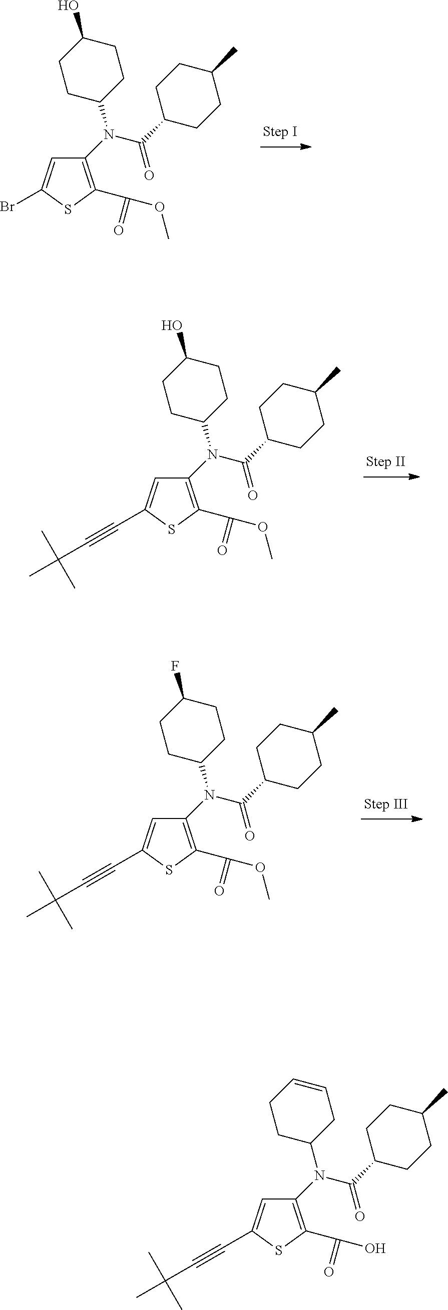

### Step I

To a solution of 5-bromo-3-[(trans-4-hydroxy-cyclohexyl)-(trans-4-methyl-cyclohexane-carbonyl)-amino]-thiophene-2-carboxylic acid methyl ester (1.273 g, 2.78 mmol) in DMF (15 mL) was sequentially added tris(dibenzylideneacetone)dipalladium(0) (127 mg, 0.14 mmol) and copper(I) iodide (11 mg, 0.06 mmol) under nitrogen atmosphere. The reaction mixture was deoxygenated by bubbling nitrogen gas for 10 minutes and tert-butyl acetylene (1.37 mL, 11.12 mmol), 2,2′-bis(diphenylphosphino)-1,1′-binaphthyl (156 mg, 0.25 mmol) and triethylamine (1.94 mL, 13.9 mmol) were sequentially added. The reaction mixture was heated at 60° C. overnight, diluted with DCM, filtered over Celite and washed with DCM. The filtrate was washed with brine, dried over Na₂SO₄, filtered, and concentrated to dryness. The residue was purified by flash column chromatography on silica gel (0 to 100% EtOAc in Hexanes) to give 5-(3,3-dimethyl-but-1-ynyl)-3-[(trans-4-hydroxy-cyclohexyl)-(trans-4-methyl-cyclohexanecarbonyl)-amino]-thiophene-2-carboxylic acid methyl ester (1.124 g, 88%).

### Step II

To a solution of 5-(3,3-dimethyl-but-1-ynyl)-3-[(trans-4-hydroxy-cyclohexyl)-(trans-4-methyl-cyclohexanecarbonyl)-amino]-thiophene-2-carboxylic acid methyl ester (191 mg, 0.41 mmol) in dichloromethane (3 mL) was added diethylaminosulphurtrifluoride (109 μL, 0.83 mmol) under nitrogen atmosphere. The reaction mixture was stirred for 1 hour at RT, diluted with dichloromethane, washed with brine and H₂O. The organic phase was dried over Na₂SO₄, filtered, and concentrated to dryness. The residue was purified by flash column chromatography on silica gel (0 to 50% EtOAc in Hexanes) to give 5-(3,3-dimethyl-but-1-ynyl)-3-[(trans-4-fluoro-cyclohexyl)-(trans-4-methyl-cyclohexanecarbonyl)-amino]-thiophene-2-carboxylic acid methyl ester (136 mg, 72%).

### Step III

To a solution of 5-(3,3-dimethyl-but-1-ynyl)-3-[(trans-4-fluoro-cyclohexyl)-(trans-4-methyl-cyclohexanecarbonyl)-amino]-thiophene-2-carboxylic acid methyl ester (136 g, 0.29 mmol) in THF:H₂O (5 mL, 4:1) was added lithium hydroxide monohydrate (37 mg, 0.88 mmol) under nitrogen atmosphere. The reaction mixture was heated at 50° C. for 3 hours, cooled to RT, and concentrated to 1/3 of its volume. The reaction mixture was diluted with DCM and acidified to pH 3 with HCl 1N. The reaction mixture was extracted by DCM, and the organic layer was washed with brine, dried over Na₂SO₄, filtered, and concentrated to dryness. The residue was purified by flash column chromatography on silica gel (0 to 5% methanol in DCM) to give 3-[cyclohex-3-enyl-(trans-4-methyl-cyclohexanecarbonyl)-amino]-5-(3,3-dimethyl-but-1-ynyl)-thiophene-2-carboxylic acid methyl ester (82 mg, 66%).

¹H NMR (400 MHz, DMSO-d₆): δ 13.55 (s, 1H), 7.27-7.17 (m, 1H), 5.66-5.47 (m, 2H), 4.66-4.28 (m, 2H), 3.95-3.83 (m, 1H), 2.16-1.42 (m, 12H), 1.26 (s, 9H), 0.82-0.70 (m, 3H), 0.67-0.54 (m, 2H).

LC/MS: m/z=442.36 (M+H⁺).

### Preparation of Compound 12: 3-[(trans-4-Allyloxy-cyclohexyl)-(trans-4-methyl-cyclohexanecarbonyl)-amino]-5-(3,3-dimethyl-but-1-ynyl)-thiophene-2-carboxylic acid

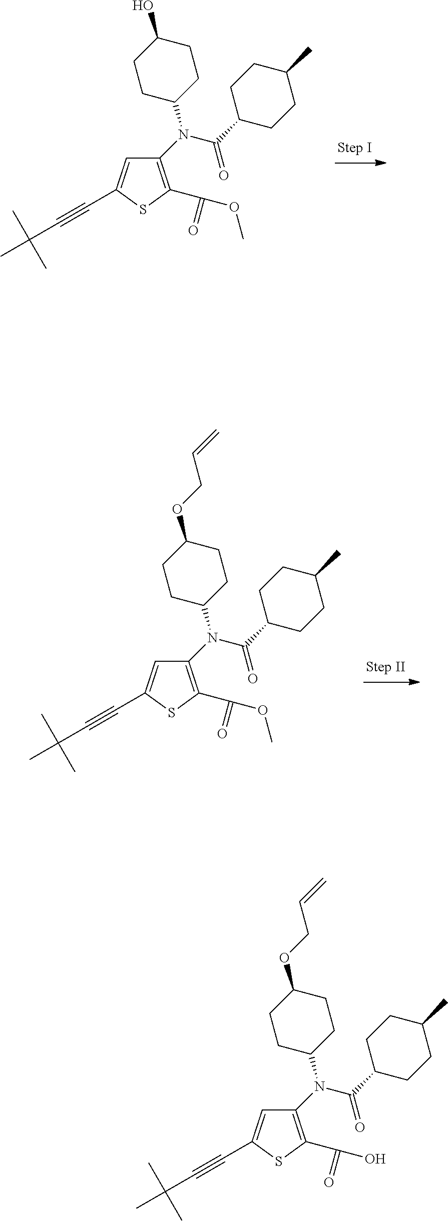

### Step I

To a degassed solution of 5-(3,3-dimethyl-but-1-ynyl)-3-[(trans-4-hydroxy-cyclohexyl)-(trans-4-methyl-cyclohexanecarbonyl)-amino]-thiophene-2-carboxylic acid methyl ester (1.87 g, 4.07 mmol) in THF (40 mL) was sequentially added allyl methyl carbonate (1042 μL, 9.17 mmol) and tetrakis(triphenylphosphine)palladium(0) (235 mg, 5 mol %) under nitrogen atmosphere. The reaction mixture was degassed and heated to 65° C. overnight. The reaction mixture was cooled to RT, and concentrated to dryness. The residue was purified by flash column chromatography on silica gel (0 to 30% EtOAc in Hexanes) to give 3-[(trans-4-allyloxy-cyclohexyl)-(trans-4-methyl-cyclohexanecarbonyl)-amino]-5-(3,3-dimethyl-but-1-ynyl)-thiophene-2-carboxylic acid methyl ester (470 mg, 23%).

### Step II

To a solution of 3-[(trans-4-allyloxy-cyclohexyl)-(trans-4-methyl-cyclohexanecarbonyl)-amino]-5-(3,3-dimethyl-but-1-ynyl)-thiophene-2-carboxylic acid methyl ester (220 mg, 0.44 mmol) in a 1:1:4 mixture of THF:H₂O:methanol (12 mL) was added lithium hydroxide monohydrate (74 mg, 1.76 mmol) under nitrogen atmosphere. The reaction mixture was heated at 50° C. overnight, cooled to RT, and concentrated. The aqueous solution was diluted with H₂O (10 mL) and acidified with aqueous HCl 2M to pH 2. The reaction mixture was extracted by DCM (3×10 mL), and the organic layer was dried over Na₂SO₄, filtered, and concentrated to dryness. The residue was purified by reverse phase preparative HPLC to give 3-[(trans-4-allyloxy-cyclohexyl)-(trans-4-methyl-cyclohexanecarbonyl)-amino]-5-(3,3-dimethyl-but-1-ynyl)-thiophene-2-carboxylic acid (210 mg, 98%).

LC/MS: m/z=486.18 (M+H⁺)

### Preparation of Compound 16: 3-[Cyclopropyl-(trans-4-methyl-cyclohexanecarbonyl)-amino]-5-(3,3-dimethyl-but-1-ynyl)-thiophene-2-carboxylic acid

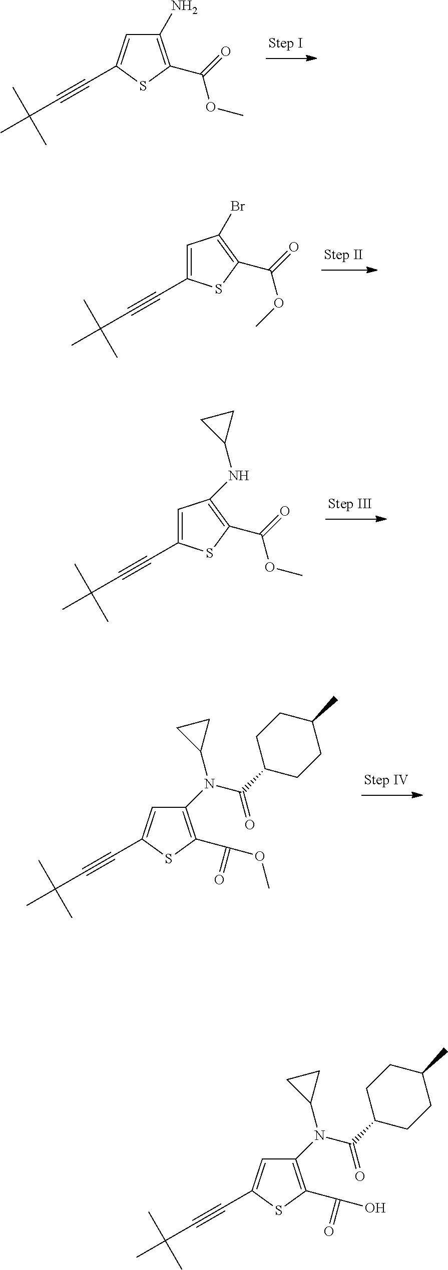

### Step I

To a suspension of copper bromide in acetonitrile (40 ml) cooled to 0° C. was added tert-butyl nitrite (1.24 mL, 1.073 mmol) under nitrogen atmosphere. The mixture was stirred for 15 minutes at 0° C. and 3-Amino-5-(3,3-dimethyl-but-1-ynyl)-thiophene-2-carboxylic acid methyl ester (2.272 g, 6.94 mmol) was added in portions over 25 minutes. The reaction mixture was protected from light, warmed to RT, stirred overnight and concentrated to dryness. The residue was dissolved in DCM (50 mL), HCl (50 mL, 1% aqueous) was added, and the mixture was stirred at RT for 30 minutes. The organic layer was washed with brine, dried over Na₂SO₄ and concentrated to dryness to give 3-Bromo-5-(3,3-dimethyl-but-1-ynyl)-thiophene-2-carboxylic acid methyl ester (2.905 g) and used as it is for the next step.

### Step II

To a solution of 3-bromo-5-(3,3-dimethyl-but-1-ynyl)-thiophene-2-carboxylic acid methyl ester (1.189 g, 3.75 mmol) in dioxane (30 mL) was sequentially added tris(dibenzylideneacetone)dipalladium(0) (343 mg, 0.375 mmol) and cesium carbonate (3.665 g, 11.25 mmol) under nitrogen atmosphere. The reaction mixture was deoxygenated by bubbling nitrogen gas for 10 minutes and cyclopropylamine (315 μL, 4.50 mmol) and 2,2′-bis(diphenylphosphino)-1,1′-binaphthyl (374 mg, 0.60 mmol) were sequentially added. The reaction mixture was heated at 60° C. for 24 hours and an additional equivalent of cyclopropylamine (265 μL, 3.75 mmol) was added after 18 hours. The reaction mixture was diluted with DCM, filtered over Celite and washed with DCM. The filtrate was concentrated to dryness and the residue was purified by flash column chromatography on silica gel (0 to 100% EtOAc in Hexanes) to give 3-cyclopropylamino-5-(3,3-dimethyl-but-1-ynyl)-thiophene-2-carboxylic acid methyl ester (742 mg, 67%) as a 3:2 mixture of starting material:product.

### Step III

To a solution of 3-cyclopropylamino-5-(3,3-dimethyl-but-1-ynyl)-thiophene-2-carboxylic acid methyl ester (742 mg, 2.53 mmol) in toluene (10 mL) was sequentially added pyridine (245 μL, 3.04 mmol) and trans-4-methylcyclohexanecarbonyl chloride (1.15 mL, 5.05 mmol) under nitrogen atmosphere. The reaction mixture was heated at 110° C. for 24 hours and pyridine and methanol were added. The reaction mixture was cooled to RT, and diluted with DCM. The organic layer was washed with brine, dried over Na₂SO₄, filtered, and concentrated to dryness. The residue was purified by flash column chromatography on silica gel (0 to 100% EtOAc in Hexanes) to give 3-[cyclopropyl-(trans-4-methyl-cyclohexanecarbonyl)-amino]-5-(3,3-dimethyl-but-1-ynyl)-thiophene-2-carboxylic acid methyl ester (448 mg, 42%).

### Step IV

To a solution of 3-[cyclopropyl-(trans-4-methyl-cyclohexanecarbonyl)-amino]-5-(3,3-dimethyl-but-1-ynyl)-thiophene-2-carboxylic acid methyl ester (435 mg, 1.04 mmol) in a 4:1 mixture of THF:H₂O (10 mL) was added lithium hydroxide monohydrate (262 mg, 6.25 mmol) under nitrogen atmosphere. The reaction mixture was heated at 60° C. for 3 hours, and cooled to RT. The reaction mixture was diluted with DCM and acidified to pH 2-3 with HCl 1N. The reaction mixture is extracted by DCM, and the organic layer was washed with brine, dried over Na₂SO₄, filtered, and concentrated to dryness. The residue was purified by flash column chromatography on silica gel (0 to 5% methanol in DCM) to give 3-[cyclopropyl-(trans-4-methyl-cyclohexanecarbonyl)-amino]-5-(3,3-dimethyl-but-1-ynyl)-thiophene-2-carboxylic acid (326 mg, 78%). LC/MS: m/z=388.26 (M+H⁺).

### Preparation of Compound 17: 5-(3,3-Dimethyl-but-1-ynyl)-3-[isopropyl-(trans-4-methyl-cyclohexanecarbonyl)-amino]-thiophene-2-carboxylic acid

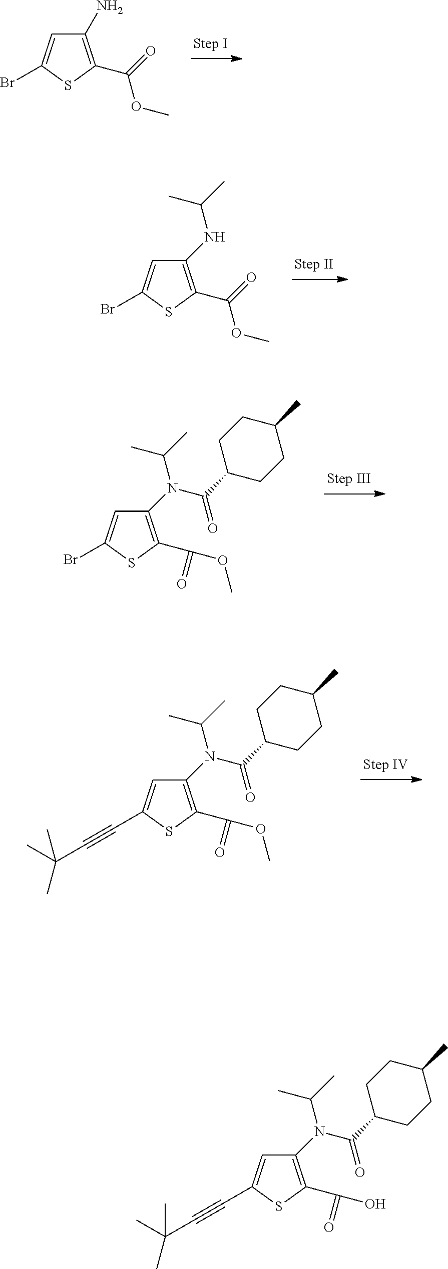

### Step I

To a solution of 3-amino-5-bromo-thiophene-2-carboxylic acid methyl ester (4.0 g, 16.95 mmol) in 1,2-dichloroethane (20 mL) was sequentially added 2-methoxypropene (6.5 mL, 67.79 mmol), acetic acid (3.8 mL, 67.79 mmol) and sodium triacetoxyborohydride (7.2 g, 67.79 mmol) under nitrogen atmosphere. The reaction mixture was stirred at RT overnight, and diluted with chloroform. The organic layer was washed with H₂O, dried over Na₂SO₄, filtered, and concentrated to dryness. The residue was purified by flash column chromatography on silica gel (2% EtOAc in hexanes) to give 5-bromo-3-isopropylamino-thiophene-2-carboxylic acid methyl (4.0 g, 85%).

### Step II

To a solution of 5-bromo-3-isopropylamino-thiophene-2-carboxylic acid methyl ester (4.0 g, 14.388 mmol) in toluene (50 mL) was sequentially added pyridine (1.3 mL, 15.83 mmol) and trans-4-methylcyclohexanecarbonyl chloride (4.6 g, 28.776 mmol) under nitrogen atmosphere. The reaction mixture was heated at 110° C. overnight, cooled to RT, and diluted with EtOAc. The organic layer was washed with saturated aqueous sodium bicarbonate, dried over Na₂SO₄, filtered, and concentrated to dryness. The residue was purified by flash column chromatography on silica gel (1 to 10% EtOAc in Hexanes) to give 5-bromo-3-[isopropyl-(trans-4-methyl-cyclohexanecarbonyl)-amino]-thiophene-2-carboxylic acid methyl ester (2.5 g, 44%).

### Step III

To a solution of 5-bromo-3-[isopropyl-(trans-4-methyl-cyclohexanecarbonyl)-amino]-thiophene-2-carboxylic acid methyl ester (150 mg, 0.387 mmol) in DMF (2 mL) was sequentially added tris(dibenzylideneacetone)dipalladium(0) (25 mg, 7 mol %) and copper(I) iodide (1.5 mg, 2 mol %) under nitrogen atmosphere. The reaction mixture was deoxygenated by bubbling nitrogen gas for 10 minutes and tert-butyl acetylene (136 mg, 1.55 mmol), triphenylphosphine (10 mg, 10 mol %) and triethylamine (381 μL, 2.75 mmol) were sequentially added. The reaction mixture was heated at 60° C. overnight, and concentrated to dryness. The reaction mixture was extracted with EtOAc, and the organic layer was washed with H₂O, dried over Na₂SO₄, filtered, and concentrated to dryness. The residue was purified by flash column chromatography on silica gel (0 to 60% EtOAc in Hexanes) to give 5-(3,3-dimethyl-but-1-ynyl)-3-[isopropyl-(trans-4-methyl-cyclohexanecarbonyl)-amino]-thiophene-2-carboxylic acid methyl ester (125 g, 80%).

### Step IV

To a solution of 5-(3,3-dimethyl-but-1-ynyl)-3-[isopropyl-(trans-4-methyl-cyclohexanecarbonyl)-amino]-thiophene-2-carboxylic acid methyl ester (125 mg, 0.310 mmol) in a 3:2:1 mixture of THF:methanol:H₂O (3 mL) was added lithium hydroxide monohydrate (930 μL, 1N) under nitrogen atmosphere. The reaction mixture was heated at 70° C. overnight, cooled to RT, concentrated to dryness and diluted with H₂O. The aqueous solution was acidified to pH 2-3 with aqueous HCl (1N). The reaction mixture was extracted with EtOAc, and the organic layer was dried over Na₂SO₄, filtered, and concentrated to dryness. The residue was purified by reverse phase preparative HPLC to give 5-(3,3-dimethyl-but-1-ynyl)-3-[isopropyl-(trans-4-methyl-cyclohexanecarbonyl)-amino]-thiophene-2-carboxylic acid (90 mg, 75%).

¹H NMR (400 MHz, CDCl₃): δ 6.8 (s, 1H), 4.9 (bs, 1H), 1.9 (m, 1H), 1.7-0.6 (m, 27H).

LC/MS: m/z=390.33 (M+H⁺).

### Preparation of Compounds 18, 19, 20, 21, and 22

The following compounds were prepared using essentially the same procedure described above for Compound 17:

### Compound 18: 5-Cyclohexylethynyl-3-[isopropyl-(trans-4-methyl-cyclohexanecarbonyl)-amino]-thiophene-2-carboxylic acid

¹H NMR (400 MHz, CDCl₃): δ 6.84 (s, 1H), 5.00-4.84 (m, 1H), 2.68-2.58 (m, 1H), 2.04-1.85 (m, 3H), 1.81-1.70 (m, 2H), 1.70-1.48 (m, 8H), 1.48-1.23 (m, 5H), 1.15 (d, 3H), 0.92 (d, 3H), 0.79 (d, 3H), 0.76-0.58 (d, 2H).

LC/MS: m/z=416.25 (M+H⁺).

### Compound 19: 5-(3-Hydroxy-3-methyl-but-1-ynyl)-3-[isopropyl-(trans-4-methyl-cyclohexanecarbonyl)-amino]-thiophene-2-carboxylic acid

LC/MS: m/z=392.22 (M+H⁺).

### Compound 20: 5-Cyclopropylethynyl-3-[isopropyl-(trans-4-methyl-cyclohexanecarbonyl)-amino]-thiophene-2-carboxylic acid

¹H NMR (400 MHz, CDCl₃): δ 6.83 (s, 1H), 4.96-4.85 (m, 1H), 1.95 (bs, 1H), 1.70-1.55 (m, 4H), 1.56-1.46 (m, 1H), 1.45-1.22 (m, 2H), 1.13 (d, 3H), 1.03-0.85 (m, 7H), 0.78 (d, 3H), 0.75-0.56 (s, 2H).

LC/MS: m/z=374.02 (M+H⁺).

### Compound 21: 3-[Isopropyl-(trans-4-methyl-cyclohexanecarbonyl)-amino]-5-phenylethynyl-thiophene-2-carboxylic acid

¹H NMR (400 MHz, CDCl₃): δ 9.91 (bs, 1H), 7.60-7.51 (m, 2H), 7.44-7.35 (m, 3H), 7.03 (s, 1H), 5.04-4.89 (m, 1H), 2.08-1.91 (m, 1H), 1.76-1.24 (m, 5H), 1.19 (d, J=6.6 Hz, 3H), 0.97 (d, J=6.8 Hz, 3H), 0.80 (d, J=6.5 Hz, 3H), 0.77-0.59 (m, 2H).

LC/MS: m/z=410.11 (M+H⁺).

### Compound 22: 3-[Isopropyl-(trans-4-methyl-cyclohexanecarbonyl)-amino]-5-(3-methoxy-3-methyl-but-1-ynyl)-thiophene-2-carboxylic acid

¹H NMR (400 MHz, CDCl₃): δ 8.00 (bs, 1H), 6.93 (s, 1H), 4.93 (s, 1H), 3.43 (s, 3H), 1.96 (bs, 1H), 1.74-1.58 (m, 4H), 1.56 (s, 6H), 1.52-1.20 (m, 3H), 1.16 (d, 3H), 0.93 (d, 3H), 0.79 (d, 3H), 0.73-0.58 (m, 2H).

LC/MS: m/z=406.15 (M+H⁺).

### Preparation of Compound 14: 3-[(4,4-Difluoro-cyclohexyl)-(trans-4-methyl-cyclohexanecarbonyl)-amino]-5-(3,3-dimethyl-but-1-ynyl)-thiophene-2-carboxylic acid

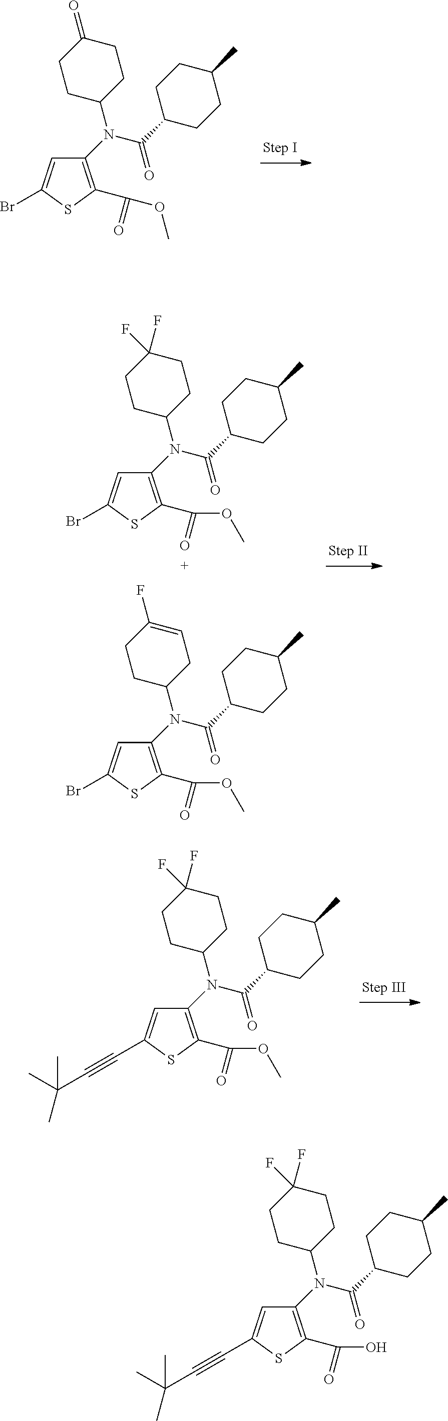

### Step I

To a solution of 5-bromo-3-[(trans-4-methyl-cyclohexanecarbonyl)-(4-oxo-cyclohexyl)-amino]-thiophene-2-carboxylic acid methyl ester (420 mg, 0.92 mmol) in toluene (5 mL) was added diethylaminosulphurtrifluoride (362 μL, 2.76 mmol) under nitrogen atmosphere. The reaction mixture was stirred at RT overnight, diluted with DCM, washed with brine, and H₂O. The organic phase was dried over Na₂SO₄, filtered, and concentrated to dryness. The residue was purified by flash column chromatography on silica gel (0 to 100% EtOAc in Hexanes) to give 5-Bromo-3-[4,4-difluoro-cyclohexyl)-(trans-4-methyl-cyclohexanecarbonyl)-amino]-thiophene-2-carboxylic acid methyl ester (196 mg, 44.5%) and 5-bromo-3-[(4-fluoro-cyclohex-3-enyl)-(trans-4-methyl-cyclohexanecarbonyl)-amino]-thiophene-2-carboxylic acid methyl ester (148 mg, 35%).

### Step II

To a solution of 5-bromo-3-[(4,4-difluoro-cyclohexyl)-(trans-4-methyl-cyclohexanecarbonyl)-amino]-thiophene-2-carboxylic acid methyl ester (193 mg, 0.40 mmol) in DMF (5 mL) was sequentially added tris(dibenzylideneacetone)dipalladium(0) (18 mg, 0.02 mmol) and copper(I) iodide (1.5 mg, 0.008 mmol) under nitrogen atmosphere. The reaction mixture was deoxygenated by bubbling nitrogen gas for 10 minutes and tert-butyl acetylene (199 μL, 1.61 mmol), 2,2′-bis(diphenylphosphino)-1,1′-binaphthyl (20 mg, 0.03 mmol) and triethylamine (279 μL, 2.0 mmol) are sequentially added. The reaction mixture was heated at 60° C. overnight, diluted with DCM, filtered over Celite and washed with DCM. The filtrate was washed with brine, dried over Na₂SO₄, filtered, and concentrated to dryness. The residue was purified by flash column chromatography on silica gel (0 to 100% EtOAc in Hexanes) to give 3-[(4,4-difluoro-cyclohexyl)-(trans-4-methyl-cyclohexanecarbonyl)-amino]-5-(3,3-dimethyl-but-1-ynyl)-thiophene-2-carboxylic acid methyl ester (183 mg, 95%).

### Step III

To a solution of 3-[4,4-difluoro-cyclohexyl)-(trans-4-methyl-cyclohexanecarbonyl)-amino]-5-(3,3-dimethyl-but-1-ynyl)-thiophene-2-carboxylic acid methyl ester (183 mg, 0.38 mmol) in a 4:1 mixture of THF:H₂O (5 mL) was added lithium hydroxide monohydrate (96 mg, 2.29 mmol) under nitrogen atmosphere. The reaction mixture was heated at 60° C. for 3.5 hours, cooled to RT and the THF was evaporated. The residue was diluted with DCM and acidified to pH 2-3 with aqueous HCl 1N. The reaction mixture is extracted by DCM and the organic layer was washed with brine, dried over Na₂SO₄, filtered, and concentrated to dryness. The residue was purified by reverse phase preparative HPLC to give 3-[(4,4-difluoro-cyclohexyl)-(trans-4-methyl-cyclohexanecarbonyl)-amino]-5-(3,3-dimethyl-but-1-ynyl)-thiophene-2-carboxylic acid (30 mg, 17%).

LC/MS: m/z=466.24 (M+H⁺).

### Preparation of Compound 15: 5-(3,3-Dimethyl-but-1-ynyl)-3-[(4-fluoro-cyclohex-3-enyl)-(trans-4-methyl-cyclohexanecarbonyl)-amino]-thiophene-2-carboxylic acid

Compound 15 was prepared using essentially the same procedure described above for Compound 14:

LC/MS: m/z=446.26 (M+H⁺).

### Preparation of Compound 23: 3-[Isopropyl-(trans-4-methyl-cyclohexanecarbonyl)-amino]-5-trifluoroprop-1-ynyl-thiophene-2-carboxylic acid

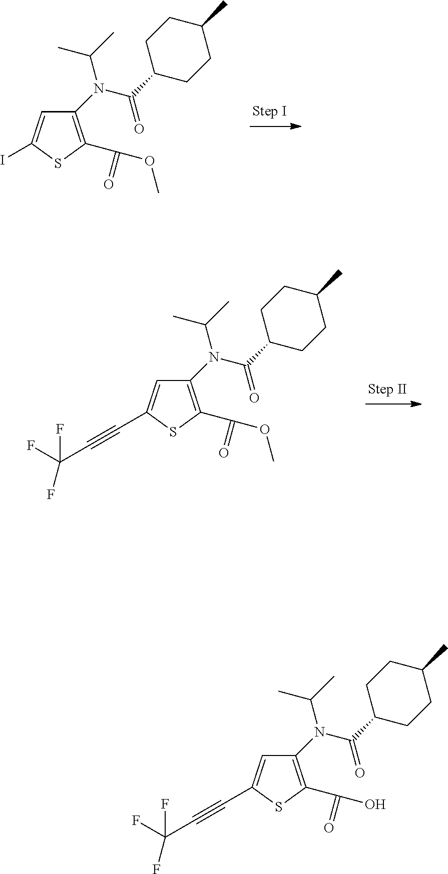 [SMILES: COC(=O)c1sc(I)cc1N(C(=O)[C@H]1CC[C@H](C)CC1)C(C)C ; COC(=O)c1sc(C#CC(F)(F)F)cc1N(C(=O)[C@H]1CC[C@H](C)CC1)C(C)C ; CC(C)N(c1cc(C#CC(F)(F)F)sc1C(=O)O)C(=O)[C@H]1CC[C@H](C)CC1]

### Step I

An excess of trifluoroacetylene is bubbled in THF (3 mL) at −78° C. and to this solution was added butyl lithium (1.5 mL, 1.5 M in hexanes) dropwise. The reaction mixture was stirred for 30 minutes at −78° C. and zinc chloride (13.4 mL, 0.5 M in THF) was added. The resulting solution was allowed to warm to RT over 1.5 hours, stirred for 30 minutes, cooled to 0° C. and tetrakis(triphenylphosphine)palladium(0) (64 mg, 0.055 mmol) and (5-iodo-3-[isopropyl-(trans-4-methyl-cyclohexanecarbonyl)-amino]-thiophene-2-carboxylic acid methyl ester (500 mg, 1.113 mmol) were sequentially added. The reaction mixture was stirred at RT for 30 minutes, at 50° C. for 4 hours and was diluted with H₂O. The reaction mixture was extracted by ether (1×50 mL), and the organic layer was dried over MgSO₄, filtered, and concentrated to dryness. The residue was purified by flash column chromatography on silica gel (0 to 12% EtOAc in DCM) to give 3-[Isopropyl-(trans-4-methyl-cyclohexanecarbonyl)-amino]-5-trifluoroprop-1-ynyl-thiophene-2-carboxylic acid methyl ester (51.2 mg, 11%).

### Step II

To a solution of 3-[isopropyl-(trans-4-methyl-cyclohexanecarbonyl)-amino]-5-trifluoroprop-1-ynyl-thiophene-2-carboxylic acid methyl ester (94 mg, 0.226 mmol) in a 1:1 mixture of THF:H₂O (2 mL) was added lithium hydroxide monohydrate (38 mg, 0.904 mmol) under nitrogen atmosphere. The reaction mixture was stirred at RT overnight, and acidified to pH 2-3 with aqueous HCl 1N. The reaction mixture was diluted with H₂O (10 mL), extracted by EtOAc (2×15 mL), and the organic layer was dried over Na₂SO₄, filtered, and concentrated to dryness. The residue was purified by reverse phase preparative HPLC to give 3-[isopropyl-(trans-4-methyl-cyclohexanecarbonyl)-amino]-5-trifluoroprop-1-ynyl-thiophene-2-carboxylic acid (10.5 mg, 11.6%).

¹H NMR (400 MHz, DMSO-d₆): δ 7.88 (s, 1H), 4.79-4.66 (m, 1H), 1.82 (t, 1H), 1.62-1.13 (m, 8H), 1.05 (d, 3H), 0.83 (d, 3H), 0.76 (d, 3H), 0.68-0.53 (m, 2H).

LC/MS: m/z=402.17 (M+H⁺).

### Example 1B

### Preparation of Compound 3

Compound 3 was prepared by the general methods described below in general scheme.

MS: m/z (obs.): 460.6 [M+H]⁺; Rt=6.05 min

1H NMR (300 MHz, MeOD) δ 6.99 (s, 1H), 4.39 (dd, J=15.9, 7.6 Hz, 1H), 2.75 (dd, J=13.4, 6.7 Hz, 1H), 2.05-1.84 (m, 4H), 1.56 (ddd, J=18.4, 12.9, 10.4 Hz, 10H), 1.32 (ddd, J=14.5, 11.3, 4.8 Hz, 8H), 1.13-0.85 (m, 5H), 0.76 (t, J=21.8 Hz, 5H).

### General Scheme

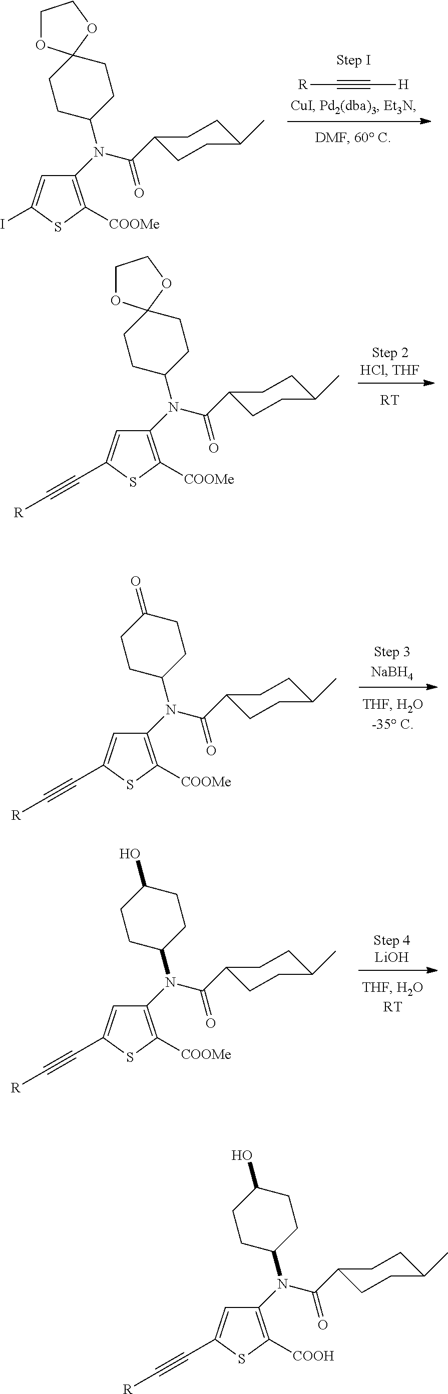

### Step 1

To a solution of 5-iodo-3-[(1,4-dioxaspiro[4.5]decan-8-yl)-(4-trans-methylcyclohexanecarbonyl)amino]thiophene-2-carboxylic acid methyl ester (1 mmol) in DMF (10-20 mL) is added Et₃N (1 mmol), CuI (0.1-0.25 mol %), tris(dibenzylideneacetone)dipalladium (0) (Pd₂(dba)₃) (0.01-0.05 mol %) and 2-substituted but-1-yne (1 mmol). The mixture is heated at 60° C. overnight, then diluted with ethyl acetate, washed with water and brine and dried (Na₂SO₄), then concentrated. The product is purified by silica gel chromatography (10-90% EtOAc in hexane) to give the desired 5-(2-substituted-ethyn-1-yl)-3-[(1,4-dioxaspiro[4.5]decan-8-yl)-(4-trans-methylcyclohexanecarbonyl)amino]thiophene-2-carboxylic acid methyl ester.

### Step 2

5-(2-Substituted-ethyn-1-yl)-3-[(1,4-dioxaspiro[4.5]decan-8-yl)-(4-trans-methylcyclohexanecarbonyl)amino]thiophene-2-carboxylic acid methyl ester (0.2 mmol) is dissolved in THF (10 mL) and added 3.6M HCl (5 mL) and stirred overnight. Then the reaction mixture is diluted with water and extracted with ethyl acetate. The organic layer is washed with brine, dried (Na₂SO₄), and concentrated. The product is purified by silica gel chromatography (10-90% ethylacetate in hexane) to give 5-(2-substituted-ethyn-1-yl)-3-[(4-oxocyclohexyl)-(4-trans-methylcyclohexanecarbonyl)amino]thiophene-2-carboxylic acid methyl ester

### Step 3

5-(2-Substituted-ethyn-1-yl)-3-[(4-oxocyclohexyl)-(4-trans-methylcyclohexanecarbonyl)amino]thiophene-2-carboxylic acid methyl ester (0.1 mmol) was taken in THF (10 mL) and water (2 drops) and the reaction mixture cooled to −25° C. Then added NaBH₄ (1 equiv.) and stirred for 2 hours. Then the reaction is quenched by addition of 1N HCl, then diluted with ethyl acetate and water. The organic layer is washed with brine and dried over Na₂SO₄ then concentrated to give the desired product, 5-(2-substituted-ethyn-1-yl)-3-[(4-trans-hydroxycyclohexyl)-(4-trans-methylcyclohexanecarbonyl)amino]thiophene-2-carboxylic acid methyl ester.

### Step 4

5-(2-Substituted-ethyn-1-yl)-3-[(4-trans-hydroxycyclohexyl)-(4-trans-methylcyclohexanecarbonyl)amino]thiophene-2-carboxylic acid methyl ester (0.1 mmol) is taken in THF (10 mL) and H₂O (2 mL) and LiOH (0.1 mmol) added. The reaction mixture is stirred at RT overnight, then washed with ethyl acetate. The aqueous layer is acidified with 1N HCl and extracted with ethyl acetate. The combined organic extracts are washed with brine and dried over Na₂SO₄ and concentrated. The product is isolated by purification by silica gel chromatography and reversed phase HPLC (60-95% methanol in H₂O (0.1% TFA) over 30 min) to give 5-(2-substituted-ethyn-1-yl)-3-[(4-trans-hydroxycyclohexyl)-(4-trans-methylcyclohexanecarbonyl)amino]thiophene-2-carboxylic acid.

### Preparation of Compound 4

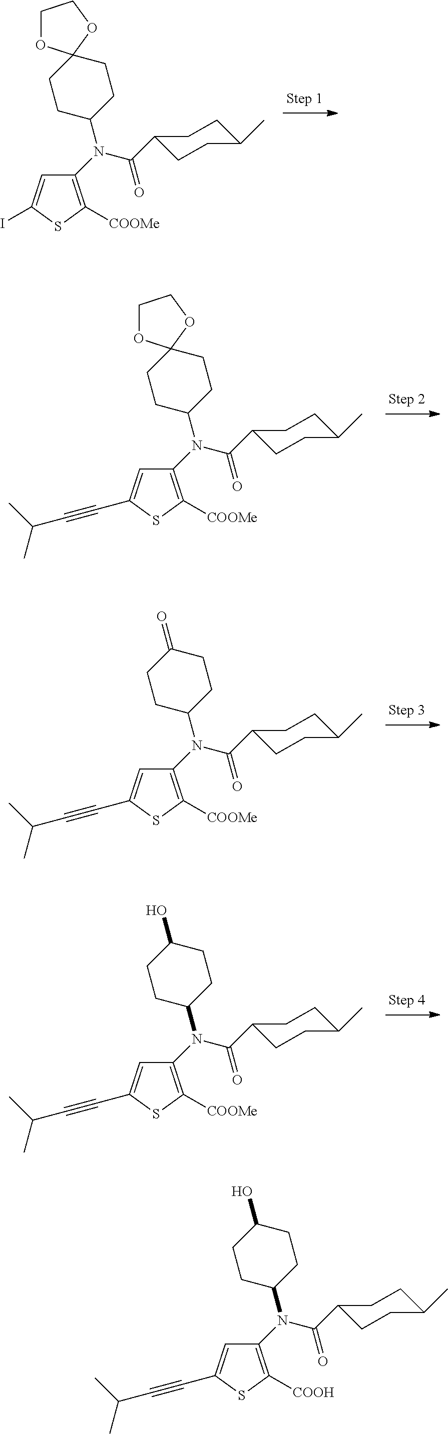

### Step 1

5-Iodo-3-[(1,4-dioxaspiro[4.5]decan-8-yl)-(4-trans-methylcyclohexanecarbonyl)amino]thiophene-2-carboxylic acid methyl ester (200 mg, 0.37 mmol) was taken in DMF (10 mL) and added Et₃N (127 μL, 0.91 mmol), CuI (17 mg, 0.09 mmol), tris(dibenzylideneacetone)dipalladium (0) (Pd₂(dba)₃) (3.3 mg, 0.0036 mmol) and 3-methylbut-1-yne (25 mg, 0.36 mmol). Then the reaction mixture was heated at 60° C. overnight. Then the reaction mixture was diluted with ethyl acetate washed with water and brine and dried (Na₂SO₄), then concentrated. The product was purified by silica gel chromatography (10-90% EtOAc in hexane) to give 5-(3-methylbut-1-yn-1-yl)-3-[(1,4-dioxaspiro[4.5]decan-8-yl)-(4-trans-methylcyclohexanecarbonyl)amino]thiophene-2-carboxylic acid methyl ester

### Step 2

5-(3-Methylbut-1-yn-1-yl)-3-[(1,4-dioxaspiro[4.5]decan-8-yl)-(4-trans-methylcyclohexanecarbonyl)amino]thiophene-2-carboxylic acid methyl ester (100 mg, 0.21 mmol) was taken in THF (10 mL) and added 3.6M HCl (5 mL) and stirred the reaction mixture overnight. Then the reaction mixture was diluted with water and extracted with ethylacetate. The organic layer was washed with brine and dried (Na₂SO₄), and concentrated to give light yellow oil. This was purified by silica gel chromatography (10-90% ethyl acetate in hexane) to give 5-(3-methylbut-1-ynyl)-3-[(4-oxocyclohexyl)-(4-trans-methylcyclohexanecarbonyl)amino]thiophene-2-carboxylic acid methyl ester

MS: m/z (obs.): 444.5 [M+H]⁺; Rt=5.62 min

### Step 3

5-(3-Methylbut-1-ynyl)-3-[(4-oxocyclohexyl)-(4-trans-methylcyclohexanecarbonyl)amino]thiophene-2-carboxylic acid methyl ester (50 mg, 0.11 mmol) was taken in THF (10 mL) and two drops of water, cooled the reaction mixture to −25° C. Then added NaBH₄ (4.2 mg, 0.11 mmol) and stirred for 2 h. Then the reaction was quenched by addition of 1N HCl, then diluted with ethyl acetate and water. The organic layer was washed with brine and dried over Na₂SO₄ then concentrated to give the desired product, 5-(3-methylbut-1-ynyl)-3-[(4-trans-hydroxycyclohexyl)-(4-trans-methylcyclohexanecarbonyl)amino]thiophene-2-carboxylic acid methyl ester.

MS: m/z (obs.): 446.5 [M+H]⁺; Rt=5.64 min

### Step 4

5-(3-Methylbut-1-ynyl)-3-[(4-trans-hydroxycyclohexyl)-(4-trans-methylcyclohexanecarbonyl)amino]thiophene-2-carboxylic acid methyl ester (30 mg, 0.067 mmol) was taken in THF (10 mL) added H₂O (2 mL) followed by LiOH (1.6 mg, 0.067 mmol). Then the reaction mixture was stirred at RT overnight. The reaction mixture was diluted with ethylacetate and extracted with water, then the aqueous layer was acidified 1N HCl and extracted with ethyl acetate. The organic phase was washed with brine and dried over Na₂SO₄ and concentrated to give a yellow oil. This was purified by silica gel chromatography and reversed phase HPLC (60-95% methanol in H2O (0.1% TFA) over 30 min) to give 5-(3-methylbut-1-ynyl)-3-[(4-trans-hydroxycyclohexyl)-(4-trans-methylcyclohexanecarbonyl)amino]thiophene-2-carboxylic acid.

MS: m/z (obs.): 432.4 [M+H]⁺; Rt=5.27 min

1H NMR (300 MHz, d6-DMSO) δ 13.44 (s, 1H), 7.18 (s, 1H), 4.46 (s, 1H), 4.27 (t, J=9.8 Hz, 1H), 3.19 (s, 1H), 2.89 (dt, J=13.7, 6.9 Hz, 1H), 1.78 (dd, J=19.7, 8.6 Hz, 4H), 1.67-1.34 (m, 6H), 1.31-1.00 (m, 11H), 0.97-0.68 (m, 4H), 0.59 (dd, J=22.2, 10.2 Hz, 2H).

### Preparation of Compounds 5, 6, 7, 8, 9 and 25

The following compounds were prepared by general methods described above for Compound 4.

### Compound 5

MS: m/z (obs.): 430.5 [M+H]⁺; Rt=4.97 min

1H NMR (300 MHz, MeOD) δ 6.99 (s, 1H), 4.39 (dd, J=15.9, 7.6 Hz, 1H), 2.75 (dd, J=13.4, 6.7 Hz, 1H), 2.09-1.43 (m, 10H), 1.32 (ddd, J=14.5, 11.3, 4.8 Hz, 8H), 1.15-0.85 (m, 5H), 0.76 (t, J=21.8 Hz, 5H).

### Compound 6

MS: m/z (obs.): 404.5 [M+H]⁺; Rt=4.80 min

1H NMR (300 MHz, d6-DMSO) δ 7.18 (s, 1H), 4.32 (dd, J=42.1, 30.5 Hz, 2H), 3.24 (d, J=35.7 Hz, 8H), 2.68-2.30 (m, 7H), 2.12 (s, 2H), 1.90-1.67 (m, 3H), 1.63-1.35 (m, 4H), 1.18 (d, J=8.7 Hz, 3H), 0.91-0.39 (m, 4H).

### Compound 7

MS: m/z (obs.): 418.5 [M+H]⁺; Rt=5.26 min

1H NMR (300 MHz, d6-DMSO) δ 13.40 (s, 2H), 7.18 (s, 1H), 4.26 (dd, J=15.1, 7.3 Hz, 2H), 3.64-3.03 (m, 8H), 2.19-1.67 (m, 6H), 1.77-1.38 (m, 10H), 1.38-1.04 (m, 10H), 2.05-0.24 (m, 32H), 0.96-0.35 (m, 9H).

### Compound 8

MS: m/z (obs.): 446.5 [M+H]⁺; Rt=5.69 min

1H NMR (300 MHz, MeOD) δ 7.00 (s, 1H), 4.38 (t, J=7.6 Hz, 1H), 3.41-3.30 (m, 1H obscured by solvent peak), 2.38 (d, J=6.5 Hz, 2H), 1.86 (dddd, J=21.4, 18.0, 9.9, 7.3 Hz, 6H), 1.59 (dd, J=29.1, 16.6 Hz, 4H), 1.32 (ddd, J=12.5, 10.6, 3.2 Hz, 6H), 1.03 (t, J=6.6 Hz, 6H), 1.00-0.49 (m, 7H).

### Compound 9

MS: m/z (obs.): 458.5 [M+H]⁺; Rt=5.86 min

1H NMR (300 MHz, MeOD) δ 6.98 (s, 1H), 4.54-4.27 (m, 1H), 3.31 (tt, J=6.1, 3.2 Hz, 1H and solvent), 2.93 (dd, J=14.8, 7.5 Hz, 1H), 2.25-1.83 (m, 8H), 1.83-1.47 (m, 8H), 1.46-1.15 (m, 8H), 1.12-0.51 (m, 5H).

### Compound 25

MS: m/z (obs.): 446.5 [M+H]⁺; Rt=5.72 min

### Preparation of Compound 26

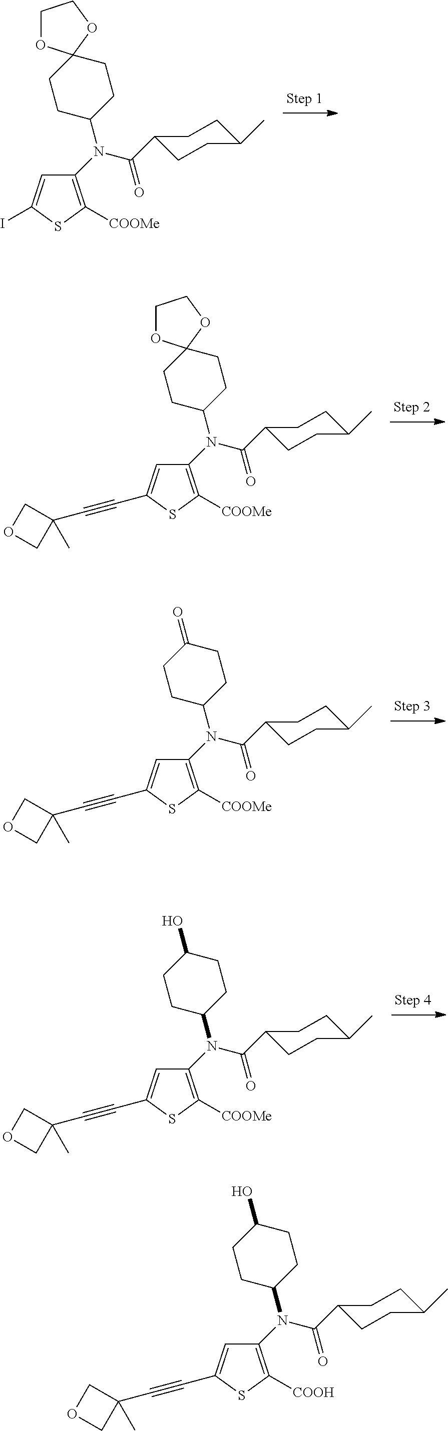 [SMILES: COC(=O)c1sc(I)cc1N(C(=O)C1CCC(C)CC1)C1CCC2(CC1)OCCO2 ; COC(=O)c1sc(C#CC2(C)COC2)cc1N(C(=O)C1CCC(C)CC1)C1CCC2(CC1)OCCO2 ; COC(=O)c1sc(C#CC2(C)COC2)cc1N(C(=O)C1CCC(C)CC1)C1CCC(=O)CC1 ; COC(=O)c1sc(C#CC2(C)COC2)cc1N(C(=O)C1CCC(C)CC1)[C@H]1CC[C@@H](O)CC1 ; CC1CCC(C(=O)N(c2cc(C#CC3(C)COC3)sc2C(=O)O)[C@H]2CC[C@@H](O)CC2)CC1]

As depicted in the general scheme for the preparation of Compound 26 above, Compound 26 was prepared in a similar manner as described above for the preparation of Compound 4 by utilizing 3-ethynyl-3-methyloxetane instead of 3-methylbut-1-yne.

MS: m/z (obs.): 460.6 [M+H]⁺; Rt=4.24 min

### 3-ethynyl-3-methyloxetane

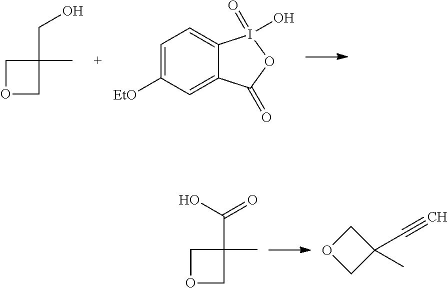

(3-Methyloxetan-3-yl)methanol (200 mg, 1.96 mmol) was taken in CH₂Cl₂ (30 mL) and IBX-polystyrene (Novabiochem, 3.2 g, 9.8 mmol) The reaction was stirred at room temperature overnight, then filtered. The solvent was evaporated and the product used directly in the next step.

3-methyloxetane-3-carboxaldehyde (1.9 mmol assumed) was taken in MeOH (20 mL) and added K₂CO₃ (1.38 g, 10 mmol) followed by dimethyl (diazomethyl)phosphonate (Bestmann's reagent, 384 mg, 1.99 mmol) at 0° C. Then the reaction mixture was warmed to RT and stirred overnight. The reaction mixture was passed through a short plug of silica gel eluted with diethyl ether. The solvent was evaporated and the product alkyne used directly in the next step.

### Preparation of Compound 27

3-Ethynyltetrahydrofuran was prepared from (tetrahydrofuran-3-yl)methanol by the methods described in compound 26, and used to prepare compound 27 by the general methods described above.

MS: m/z (obs.): 460.5 [M+H]⁺; Rt=4.08 min

1H NMR (300 MHz, MeOD) δ 7.08 (s, 1H), 4.98 (s, 4H), 4.59-4.27 (m, 1H), 4.16-3.70 (m, 5H), 3.57-3.23 (m, 3H), 2.44-2.25 (m, 1H), 2.18-1.30 (m, 20H), 1.26 (dd, J=9.5, 4.8 Hz, 2H), 1.19-0.83 (m, 3H), 0.83-0.12 (m, 5H).

### Preparation of Compound 28

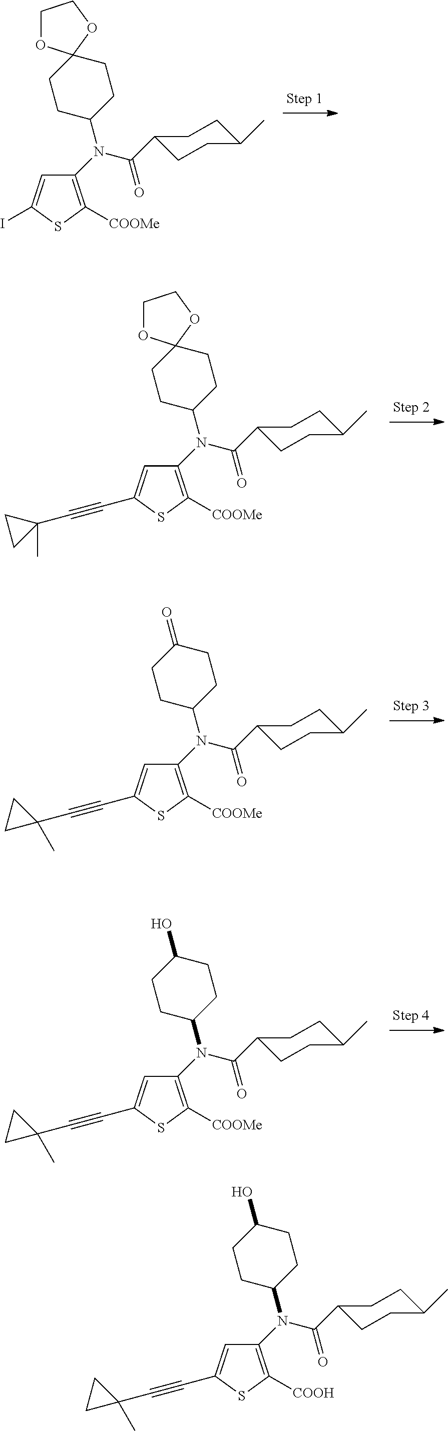 [SMILES: COC(=O)c1sc(I)cc1N(C(=O)C1CCC(C)CC1)C1CCC2(CC1)OCCO2 ; COC(=O)c1sc(C#CC2(C)CC2)cc1N(C(=O)C1CCC(C)CC1)C1CCC2(CC1)OCCO2 ; COC(=O)c1sc(C#CC2(C)CC2)cc1N(C(=O)C1CCC(C)CC1)C1CCC(=O)CC1 ; COC(=O)c1sc(C#CC2(C)CC2)cc1N(C(=O)C1CCC(C)CC1)[C@H]1CC[C@@H](O)CC1 ; CC1CCC(C(=O)N(c2cc(C#CC3(C)CC3)sc2C(=O)O)[C@H]2CC[C@@H](O)CC2)CC1]

As depicted in the general scheme for the preparation of Compound 28 above, Compound 28 was prepared in a similar manner as described above for the preparation of Compound 4 by utilizing 1-ethynyl-1-methylcyclopropane instead of 3-methylbut-1-yne.

MS: m/z (obs.): 444.5 [M+H]⁺; Rt=5.34 min

1H NMR (300 MHz, MeOD) δ 6.95 (s, 1H), 4.81 (s, 5H), 4.49-4.20 (m, 1H), 3.51-3.22 (m, 4H), 2.01-1.14 (m, 23H), 1.08-0.83 (m, 4H), 0.83-0.10 (m, 7H).

### 1-ethynyl-1-methylcyclopropane

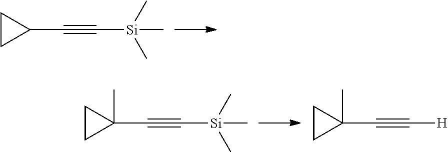

2-Cyclopropylethynyl-trimethylsilane (3 g, 21.69 mmol) was taken in diethyl ether (20 mL), cooled to 0° C. and 1.6 M n-BuLi in hexane (13.6 mL, 21.7 mmol) added dropwise at 0° C. The reaction mixture was stirred at RT overnight. Then dimethyl sulfate (6.62 g, 54.2 mmol) was added dropwise at −10° C. and the reaction maintained at 20° C. for 30 min. The reaction was quenched by adding saturated aq. NH₄Cl and 25% aq. ammonia solutions (1:3, 100 mL) and stirred at ambient temp for 1 hour. The aqueous phase was extracted with diethyl ether (3×50 mL) and the combined organic layers were washed with 5% HCl and 5% aq NaHCO₃ solution (100 mL) and water (100 mL), then dried over anhydrous Na₂SO₄ and carefully concentrated under a stream of nitrogen gas at ambient pressure. The product was taken forward without characterization.

(2-(1-Methylcyclopropyl)-ethynyl)-trimethylsilane (500 mg, 3.3 mmol) was taken in THF (20 mL) and 1M tetrabutylammonium fluoride in THF added (6.6 mL, 6.6 mmol). The reaction mixture was stirred overnight, then the reaction mixture was poured into water. The organic layer was separated, washed with brine, dried over Na₂SO₄ and partially evaporated (no heating). The product 1-ethynyl-1-methylcyclopropane was used as obtained as a concentrated solution in THF in the next step.

### Preparation of Compound 30

Compound 30 was prepared in a similar manner as described above for Compound 28. MS: m/z (obs.): 448.5 [M+H]⁺; Rt=3.67 min

1H NMR (300 MHz, DMSO) δ 13.50 (s, 1H), 7.22 (s, 1H), 5.63 (s, 1H), 4.47 (s, 1H), 4.28 (s, 1H), 3.18 (s, 1H), 1.92-1.70 (m, 4H), 1.71-1.36 (m, 11H), 1.33-1.03 (m, 6H), 0.97-0.82 (m, 1H), 0.76 (d, J=6.4 Hz, 3H), 0.68-0.40 (m, 2H).

### Preparation of Compound 32

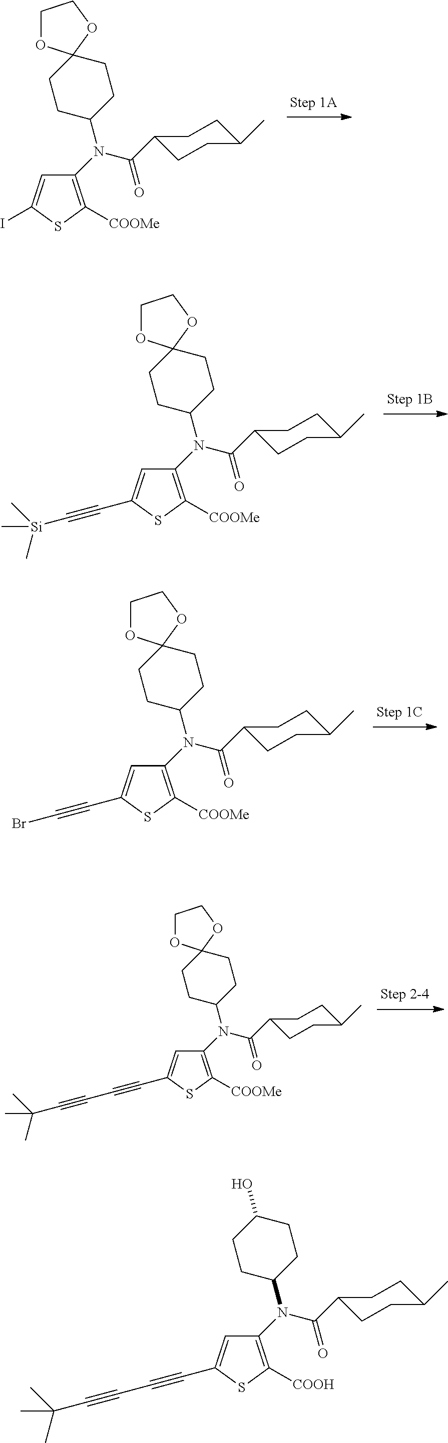

### Step 1A

5-Iodo-3-[(1,4-dioxaspiro[4.5]decan-8-yl)-(4-trans-methylcyclohexanecarbonyl)amino]thiophene-2-carboxylic acid methyl ester (500 mg, 0.91 mmol) was taken in DMF (20 mL) and copper (I) iodide (17 mg, 0.09 mmol), Pd₂(dba)₃ (84 mg, 0.09 mmol) and triethylamine (127 μL, 0.91 mmol) added. The reaction mixture was heated at 60° C. overnight. Then the reaction mixture was diluted with ethyl acetate, washed with water, and brine and dried (Na2SO4).

The solution was concentrated, and the residue purified by silica gel chromatography (10-90% ethyl acetate in hexane) to give 5-(trimethylsilylethyn-1-yl)-3-[(1,4-dioxaspiro[4.5]decan-8-yl)-(4-trans-methylcyclohexanecarbonyl)amino]thiophene-2-carboxylic acid methyl ester, which was used as obtained in the subsequent step.

MS: m/z (obs.): 518.5 [M+H]⁺; Rt=6.2 min

### Step 1B

5-(Trimethylsilylethyn-1-yl)-3-[(1,4-dioxaspiro[4.5]decan-8-yl)-(4-trans-methylcyclohexanecarbonyl)amino]thiophene-2-carboxylic acid methyl ester (350 mg, 0.68 mmol) was dissolved in acetone (20 mL) and added silver nitrate (73 mg, 0.68 mmol) followed by N-bromosuccinimde (120.3 mg, 0.68 mmol). The reaction was stirred room temperature for 2 hours, then cooled to 0° C. and quenched with water. The reaction mixture was extracted with ethyl acetate, dried and concentrated to give brown oil. This was taken to the next step without further purification.

MS: m/z (obs.): 524.3 [M+H]⁺; Rt=5.49 min

### Step 1C

5-(Bromoethyn-1-yl)-3-[(1,4-dioxaspiro[4.5]decan-8-yl)-(4-trans-methylcyclohexanecarbonyl)amino]thiophene-2-carboxylic acid methyl ester (300.0 mg, 0.57 mmol) was taken in DMF (15 mL) and added Pd₂(dba)₃ (174.6 mg, 0.19 mmol), copper (I) iodide (3.6 mg, 0.019 mmol), Et₃N (79 μL, 0.57 mmol) and 3,3-dimethylbut-1-yne (23 μL, 0.19 mmol). Then the reaction mixture was heated at 60° C. overnight. Then the reaction mixture was diluted with ethyl acetate washed with water and brine and dried (Na₂SO₄). The product was purified by silica gel chromatography (10-90% ethyl acetate in hexane) to give 5-(5,5-dimethylhexa-1,3-diyn-1-yl)-3-[(1,4-dioxaspiro[4.5]decan-8-yl)-(4-trans-methylcyclohexanecarbonyl)amino]thiophene-2-carboxylic acid methyl ester.

Compound 32 was prepared from 5-(5,5-dimethylhexa-1,3-diyn-1-yl)-3-[(1,4-dioxaspiro[4.5]decan-8-yl)-(4-trans-methylcyclohexanecarbonyl)amino]thiophene-2-carboxylic acid methyl ester using the general methods, steps 2-4 as described above.

MS: m/z (obs.): 470.5 [M+H]⁺; Rt=6.0 min

1H NMR (300 MHz, DMSO) δ 7.45 (s, 1H), 4.48 (d, J=4.1 Hz, 1H), 4.27 (s, 1H), 3.18 (s, 1H), 1.80 (d, J=10.1 Hz, 4H), 1.56 (d, J=11.8 Hz, 6H), 1.25 (d, J=10.0 Hz, 9H), 1.23-1.02 (m, 4H), 0.92-0.79 (m, 1H), 0.76 (d, J=6.4 Hz, 3H), 0.59 (dd, J=24.9, 12.5 Hz, 2H).

### Preparation of Compounds 34, 55, and 56

The following compounds were prepared in a similar manner as described above for the preparation of Compound 32.

### Compound 34

MS: m/z (obs.): 462.4 [M+H]⁺; Rt=4.67 min

1H NMR (300 MHz, DMSO) δ 13.54 (s, 1H), 7.30 (s, 1H), 4.47 (s, 1H), 4.28 (s, 1H), 3.30 (s, 3H), 3.20 (s, 1H), 1.79 (dd, J=18.5, 8.2 Hz, 4H), 1.67-1.31 (m, 12H), 1.21 (dd, J=21.6, 11.9 Hz, 5H), 0.92-0.44 (m, 6H).

### Compound 55

MS: m/z (obs.): 459.5 [M+H]⁺; Rt=0.83 min

### Compound 56

MS: m/z (obs.): 434.4 [M+H]⁺; Rt=3.21 min

### Preparation of Compound 29

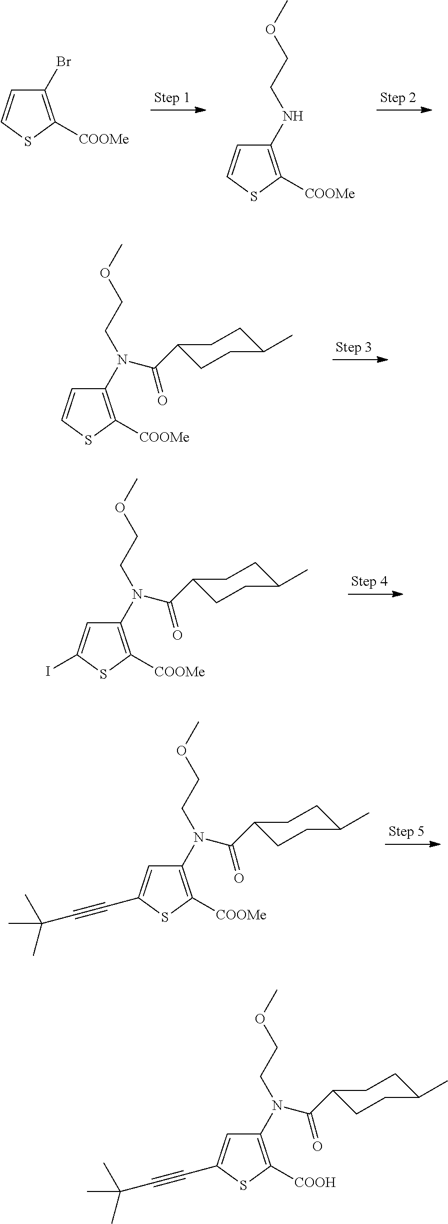

### Methyl 3-(2-methoxyethylamino)thiophene-2-carboxylate

At room temperature, to a solution of methyl-3-aminothiophene-2-carboxylate (3 g, 19.1 mmol, 1 eq) in DMF (100 mL) were added 2-bromoethyl methyl ether (26.5 g, 191 mmol, 10 eq), potassium iodide (31 g, 95.5 mmol, 10 eq) and followed by DIPEA (20 mL, 115 mmol, 6 eq) and the mixture was heated to 120° C. for about 20 h in a steel bomb, reaction progress was analyzed by TLC (20% EtOAc:pet ether, R_{f}=0.66). The reaction mixture was cooled to room temperature and DMF was evaporated at 50° C., added water (150 ml) and extracted with EtOAc (250 mL, 150 mL, 100 mL). The combined organic layer was washed with water (100 mL×3) and brine solution (50 mL×2), dried (Na₂SO₄) and evaporated at 45° C. to afford crude compound (5 g) that was purified by column chromatography over neutral alumina Gradient elution with 0-3% EtOAc/Pet ether gave methyl 3-((2-methoxyethyl)amino)thiophene-2-carboxylate (1.5 g, 36.5%) as a brown liquid along with unreacted starting material (1 g).

MS: m/z (obs.): 216 [M+H]⁺;

1H NMR CDCl3, 400 MHz: 7.33 (d, J=5.6 Hz, 1H), 6.92 (br.s, exchanged with D2O, 1H), 6.65 (d, J=5.6 Hz, 1H), 3.81 (s, 3H), 3.57 (t, J=6.0 Hz, 2H), 3.43-3.47 (q, 2H), 3.39 (s, 3H).

### Methyl 3-(N-(2-methoxyethyl)-4-trans-methyl-cyclohexanecarboxamido)thiophene-2-carboxylate

A solution of trans-4-methylcyclohexane carboxylic acid (132 mg, 0.93 mmol, 1 eq) in CH₂Cl₂ (4 mL) was added catalytic amount of DMF (1 drop), cooled to 0° C., added oxalyl chloride (0.1 ml, 1.02 mmol, 1.1 eq), stirred for 30 min at room temperature. In another flask, a solution of methyl 3-((2-methoxyethyl)amino)thiophene-2-carboxylate (200 mg, 0.93 mmol, 1 eq) in CH₂Cl₂ (4 mL) was added triethylamine (0.26 mL, 1.86 mmol, 2 eq), cooled to 0° C., to this solution was added above acid chloride solution drop wise, stirred to room temperature and the reaction progress was analyzed by TLC (20% EtOAc:CHCl₃, R_{f}: 0.5, 16 h, room temperature). The reaction mixture was quenched with saturated aq. NaHCO₃ solution (25 mL) and added water (20 mL), extracted with EtOAc (50 mL×4), the combined organic layer was washed with brine solution (20 mL), dried (Na₂SO₄) and evaporated at 45° C. to afford crude compound (261 mg) that was purified by column chromatography (100-200 mesh silica gel, 0-10% EtOAc:CHCl₃) to afford methyl 3-(N-(2-methoxyethyl)-4-trans-methyl-cyclohexane carboxamido)thiophene-2-carboxylate (180 mg, 57%) as an off white solid.

MS: m/z (obs.): 340 [M+H]⁺;

### Methyl 5-iodo-3-(N-(2-methoxyethyl)-4-trans-methyl-cyclohexane carboxamido)thiophene-2-carboxylate

A solution of methyl 3-(N-(2-methoxyethyl)-4-trans-methyl-cyclohexane carboxamido)thiophene-2-carboxylate (136 mg, 0.40 mmol, 1 eq) in THF (3 mL) was cooled to −78° C., added LDA (2M solution in THF) (0.6 ml, 1.20 mmol, 3 eq) at −78° C. (light green clear solution was observed), slowly stirred to −20° C. for 45 min (thick brown solution was observed) and again cooled to −78° C. then added a solution of I₂ (305 mg, 1.20 mmol, 3 eq) in THF (2 ml) at −78° C., stirred to room temperature. The reaction mixture was quenched with ice water (10 mL), extracted with EtOAc (25 mL×5), the combined organic layer was washed with brine solution (20 ml), dried (Na₂SO₄) and evaporated at 40° C. to afford crude compound (150 mg) that was purified by column chromatography using (100-200 mesh silica gel, eluted with 0-15% EtOAc:Pet ether) to afford methyl 5-iodo-3-(N-(2-methoxyethyl)-4-trans-methyl-cyclohexane carboxamido)thiophene-2-carboxylate (30 mg, 16%, R_{f}: 0.55 (30% EtOAc:Pet ether) as an off white solid.

MS: m/z (obs.): 466 [M+H]⁺;

1H NMR CDCl3, 400 MHz: 7.17 (s, 1H), 4.10-4.04 (m, 1H), 3.83 (s, 3H), 3.58-3.54 (m, 1H), 3.47-3.41 (m, 2H), 3.25 (s, 1H), 2.04-2.02 (m, 1H), 1.66-1.60 (m, 4H), 1.51-1.44 (m, 2H), 1.35-1.27 (m, 1H), 0.81 (d, J=6.4 Hz, 3H), 0.72-0.68 (m, 2H)

### Methyl 5-(3,3-dimethylbut-1-ynyl)-3-(N-(2-methoxyethyl)-4-trans-methylcyclohexanecarboxamido)thiophene-2-carboxylate

A solution of methyl 5-iodo-3-(N-(2-methoxyethyl)-4-trans-methyl-cyclohexane carboxamido)thiophene-2-carboxylate (100 mg, 0.21 mmol, 1 eq) in THF (5 mL) was cooled to −10° C., deoxygenated by bubbling with a stream of Argon for 10 min, added TEA (26 mg, 0.25 mmol, 1.2 eq) and copper iodide (1.23 mg, 0.065 mmol, 0.03 eq), purged with Argon for 30 min at −10° C., added Pd(PPh₃)₂Cl₂ (4.5 mg, 0.0065 mmol, 0.03 eq) at −10° C., again purged Argon for 10 min. and finally add 3,3-dimethyl butyne (0.1 ml, 0.32 mmol, 1.5 eq) at −10° C. for 5 h. To the reaction mixture added water (20 ml), extracted with EtOAc (50 mL×3), the combined organic layer was washed with water (10 ml) and brine solution (20 ml), dried (Na₂SO₄) and evaporated at 40° C. to afford crude compound (100 mg) that was purified by column chromatography (100-200 mesh silica gel, eluted with 25% EtOAc:Pet ether) to afford methyl 5-(3,3-dimethylbut-1-ynyl)-3-(N-(2-methoxyethyl)-4-trans-methylcyclohexanecarboxamido)thiophene-2-carboxylate (50 mg, 55.5%, TLC(R_{f}: 0.54 (40% EtOAc:pet ether)) as a brown color liquid.

MS: m/z (obs.): 420 [M+H]⁺;

1H NMR CDCl3, 400 MHz: 6.94 (s, 1H), 4.09-4.06 (m, 1H), 3.83 (s 3H), 3.55-3.43 (m, 3H), 3.25 (s, 3H), 2.04-2.02 (m, 1H), 1.65-1.59 (m, 5H), 1.33 (s, 9H), 1.30-1.25 (m, 2H), 0.80 (d, J=6.8 Hz, 3H), 0.72-0.69 (m, 2H).

### 5-(3,3-Dimethylbut-1-ynyl)-3-(N-(2-methoxyethyl)-4-trans-methylcyclohexanecarboxamido)thiophene-2-carboxylic acid

A solution of methyl 5-(3,3-dimethylbut-1-ynyl)-3-(N-(2-methoxyethyl)-4-trans-methylcyclohexanecarboxamido)thiophene-2-carboxylate (450 mg, 1.073 mmol, 1 eq) in THF:MeOH (10 ml: 10 ml) was cooled to 0° C., added LiOH.H₂O (270 mg, 6.44 mmol, 6 eq) at 0° C. and stirred at room temperature for 16 h. The reaction progress was analyzed by TLC (20% MeOH:CHCl₃, R_{f}: 0.4). The solvent was evaporated completely at 35° C. to afford crude compound (400 mg) that was purified by column chromatography (100-200 mesh silica gel, eluted with 5% MeOH:CHCl₃) and HPLC to afford 5-(3,3-dimethylbut-1-ynyl)-3-(N-(2-methoxyethyl)-4-trans-methylcyclohexanecarboxamido)thiophene-2-carboxylic acid (180 mg, 41%) as an off white solid.

MS: m/z (obs.): 460 [M+H]⁺;

1H NMR CDCl3, 400 MHz: 6.92 (s, 1H), 3.90-3.75 (m, 2H), 3.8-3.40 (m, 2H), 3.30 (s, 3H), 2.20-2.05 (m, 1H), 1.65-1.58 (m, 7H), 1.33 (s, 9H), 0.80 (d, J=6.8 Hz, 3H), 0.72 (m, 2H)

### Preparation of Compound 39

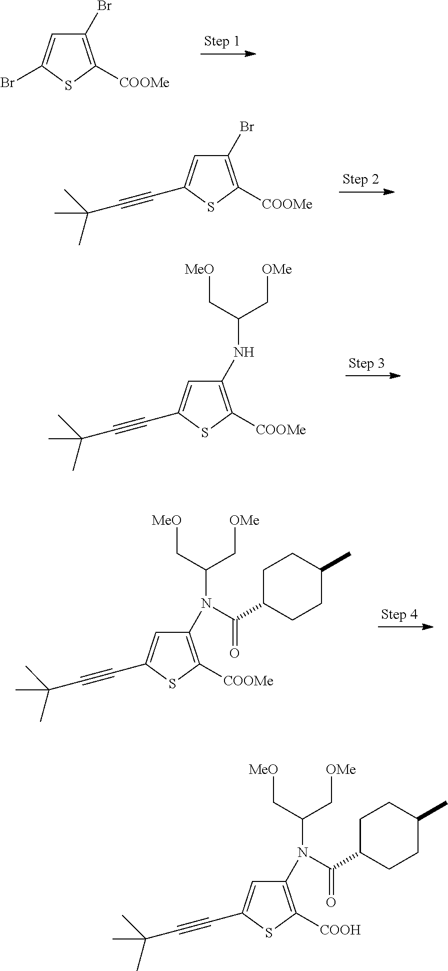

### Step 1: Methyl 3-bromo-5-(3,3-dimethylbut-1-yn-1-yl)thiophene-2-carboxylate

A suspension of CuI (0.95 g, 4.99 mmol) in 1,4 dioxane (300 mL) was deoxygenated by purging with argon for 30 min at room temperature and Pd (PPh₃)₂Cl₂ (3.5 g, 4.99 mmol) was added after which the purging was continued. After 15 min diisopropylamine (35.5 mL, 250 mmol) was added followed by methyl 3,5-dibromothiophene-2-carboxylate (50 g, 166.6 mmol). After stirring for 15 min at room temperature the reaction mixture was cooled and 3,3-dimethyl-1-butyne (22.1 mL, 183 mmol) in dioxane (360 mL) was added at 5-10° C. —(initially 10 mL). The reaction was stirred for 15 min and then remaining quantity of 3-methyl-1-butyne in dioxane was added (maintaining the internal temperature below 20° C.). After addition the reaction mixture was stirred at room temperature for 2 h. The reaction progress was monitored by TLC and GC (GC shows complete consumption of SM). The reaction mixture was diluted with diethyl ether (800 mL), filtered through celite and the cake was washed with ether (2×50 mL). The combined filtrate was concentrated and the obtained crude compound was purified by column chromatography (100-200 mesh silica gel) using 4% EtOAc in hexane as eluent to afford methyl 3-bromo-5-(3,3-dimethylbut-1-yn-1-yl)thiophene-2-carboxylate (48.0 gm, 95.8%) as a white solid. (TLC system: 3% CH₂Cl₂ in pet ether, Rf: 0.35)

MS: m/z (obs.): 301, 303 [M+H]⁺;

1H NMR CDCl3, 400 MHz: 7.04 (s, 1H), 3.87 (s, 3H), 1.30 (s, 9H).

### Step 2: Methyl 3-(1,3-dimethoxypropan-2-ylamino)-5-(3,3-dimethylbut-1-ynyl)thiophene-2-carboxylate

To a stirred solution of methyl 3-bromo-5-(3,3-dimethylbut-1-yn-1-yl)thiophene-2-carboxylate (0.1 g, 0.332 mmol, 1 eq) in 1,4-dioxane (4 ml), added Cs₂CO₃ (0.32 g, 0.99 mmol, 3 eq) and BINAP (35 mg, 0.05 mmol, 0.17 eq) at room temperature. The reaction mixture was deoxygenated by bubbling argon for 15 min before the addition of Pd₂(dba)₃ (30.3 mg, 0.03 mmol, 0.1 eq) and continued for another 10 min at room temperature. 2-Amino-1,3-dimethoxy propane (55 mg, 0.46 mmol, 1.4 eq) at room temperature. The reaction mixture was stirred at 90° C. for 20 h. Reaction progress was monitored by TLC (10% EtOAc in pet ether, R_{f}: 0.5). The reaction mixture was concentrated to obtain crude compound; which was purified by column chromatography (silica gel 100-200 mesh) by using 8% EtOAc in pet ether to afford methyl 3-(1,3-dimethoxypropan-2-ylamino)-5-(3,3-dimethylbut-1-ynyl)thiophene-2-carboxylate (40 mg, 36.3%) as pale yellow liquid.

MS: m/z (obs.): 340 [M+H]⁺;

1H NMR CDCl3, 400 MHz: 7.01 (br d, D₂O exchangeable, 1H), 6.64 (s, 1H), 3.79 (s, 3H), 3.63-3.60 (m, 1H), 3.50-3.49 (m, 4H), 3.38 (s, 6H), 1.30 (s, 9H).

### Step 3: Methyl 3-(-N-(1,3-dimethoxypropan-2-yl)-4-trans-methylcyclohexanecarboxamido)-5-(3,3-dimethylbut-1-yn-1-yl)thiophene-2-carboxylate

To a solution of trans-4-methyl cyclohexanecarboxylic acid (0.5 g, 3.52 mmol, 1 eq) in 1,2-dichloroethane (4.5 mL), added DMF (0.005 mL, 0.07 mmol, 0.02 eq) followed by oxalyl chloride (0.32 mL, 3.87 mmole, 1.1 eq) at room temperature. After addition the reaction mixture was stirred for 1 h at room temperature. In another flask a solution of methyl 3-(1,3-dimethoxypropan-2-ylamino)-5-(3,3-dimethylbut-1-ynyl)thiophene-2-carboxylate (50m g, 0.14 mmole, 1 eq) in 1,2-dichloroethane (2.3 mL) was added DMAP (6 mg, 0.04 mmol, 0.3 eq) and pyridine (0.13 mL, 1.62 mmol, 11 eq) at room temperature. To this solution the above prepared acid chloride solution was added dropwise at room temperature. After addition, the reaction mixture was stirred at 85° C. for 8 h. The reaction progress was monitored by TLC (20% EtOAc in CHCl₃, R_{f}: 0.3). The reaction mixture was cooled to room temperature and quenched with water (25 ml) and extracted with EtOAc (20 ml×5). The combined organic layer was washed with 2N HCl (10 ml), brine (10 ml), dried over with Na₂SO₄ and concentrated to obtain crude compound (60 mg); which was purified by column chromatography (silica gel 100-200 mesh) by using 5-8% EtOAc in pet ether as eluent to afford methyl 3-(-N-(1,3-dimethoxypropan-2-yl)-4-trans-methylcyclohexanecarboxamido)-5-(3,3-dimethylbut-1-yn-1-yl)thiophene-2-carboxylate (20 mg, 29.4%) as colorless liquid.

MS: m/z (obs.): 464 [M+H]⁺;

1H NMR CDCl3, 400 MHz: 7.01 (s, 1H), 4.70-4.64 (m, 1H), 3.82 (s, 3H), 3.60-3.43 (m, 3H), 3.38-3.34 (m, 1H), 3.31 (s, 3H), 3.16 (s, 3H), 2.27-2.20 (m, 1H), 2.04-1.97 (m, 3H), 1.78-1.74 (m, 2H), 1.67-1.60 (m, 5H), 1.49-1.39 (m, 3H), 1.33 (s, 9H), 0.96

### Step 4: 3-(-N-(1,3-Dimethoxypropan-2-yl)-4-trans-methylcyclohexanecarboxamido)-5-(3,3-dimethylbut-1-yn-1-yl)thiophene-2-carboxylic acid (Compound 39)

Title compound was prepared by hydrolysis of methyl 3-(-N-(1,3-dimethoxypropan-2-yl)-4-trans-methylcyclohexanecarboxamido)-5-(3,3-dimethylbut-1-yn-1-yl)thiophene-2-carboxylate (80 mg) as described for the preparation of Compound 29. Yield 31 mg, 40%.

MS: m/z (obs.): 450.6 [M+H]⁺;

1H NMR DMSO, 400 MHz (80° C.): 6.86 (s, 1H), 4.54-4.48 (m, 1H), 3.60-3.39 (m, 4H), 3.20 (s, 3H), 3.12 (s, 3H), 2.04 (m, 1H), 1.76-1.73 (m, 1H), 1.58-1.35 (m, 5H), 1.29 (s, 9H), 0.76 (d, J=6.8 Hz, 3H), 0.64-0.61 (m, 2H)

### Preparation of Compound 33

Compound 33 was prepared from S-1-methoxypropyl-2-amine in the manner described for compound 39.

MS: m/z (obs.): 420.5 [M+H]⁺;

1H NMR DMSO, 400 MHz (80° C.): 6.94 (s, 1H), 4.74 (s, 1H), 3.45 (s, 1H), 3.24 (s, 3H), 3.12 (s, 2H), 1.93-1.84 (m, 2H), 1.65-1.56 (m, 4H), 1.30 (s, 9H), 0.96-0.93 (m, 3H), 0.77 (d, J=6.4 Hz, 3H), 0.63 (s, 2H)

### Preparation of Compound 36

Compound 36 was prepared from R-1-methoxypropyl-2-amine in the manner described for compound 39.

MS: m/z (obs.): 418.0 [M−H]⁻;

1H NMR CDCl3, 400 MHz: 6.89 (s, 1H), 6.83 (s, 1H), 4.99 (s, 1H), 4.80 (br. s, 1H), 3.80-3.70 (m, 1H), 3.52-3.40 (m, 2H), 3.36 (s, 3H), 1.98-1.84 (m, 2H), 1.71-1.49 (m, 4H), 1.33 (s, 9H), 0.98-0.86 (m, 2H), 0.77 (s, 3H), 0.67 (s, 2H)

### Preparation of Compound 37

Compound 37 was prepared from S-1-methoxybutyl-2-amine in the manner described for compound 39.

MS: m/z (obs.): 434.5 [M+H]⁺;

1H NMR CDCl3, 400 MHz: 6.81 (s, 1H), 4.9 (brs, 1H), 4.2 (br s, 1H), 3.48 (m, 1H), 3.27 (s, 3H), 2.07-1.97 (m, 2H), 1.79-1.48 (m, 9H), 1.32 (s, 9H), 1.0-0.65 (series of m, 7H)

### Preparation of Compound 10

Compound 10 was prepared from R-1-methoxybutyl-2-amine in the manner described for compound 39.

MS: m/z (obs.): 434.1 [M+H]⁺;

1H NMR CDCl3, 400 MHz: 6.92 (s, 1H), 4.9 (Bs, 1H), 3.74 (m, 1H), 3.48 (m, 1H), 3.35 (s, 3H), 2.07-1.97 (m, 2H), 1.79-1.48 (m, 5H), 1.32 (s, 9H), 0.88 (d, j=6.4 Hz, 3H), 0.78 (d, j=5.2 Hz, 3H), 0.73 (bs, 2H)

### Preparation of Compound 38

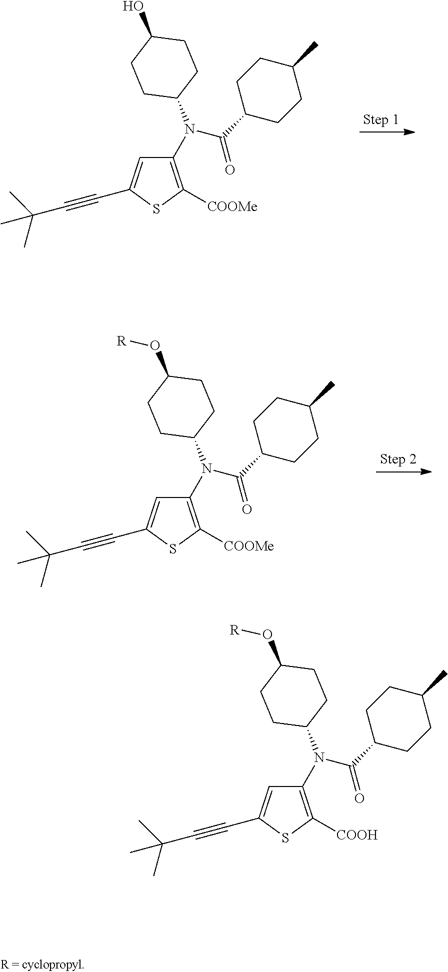

### Step 1

To a stirred solution of compound methyl 5-(3,3-dimethylbut-1-yn-1-yl)-3-((N-4-trans-hydroxycyclohexyl)-4-trans-methylcyclohexanecarboxamido)thiophene-2-carboxylate (80 mg, 0.174 mmol, 1 eq) in Cyclopropyl bromide (5 mL) was added silver oxide (806 mg, 3.48 mmol, 20 eq), molecular sieves 4 Å (200 mg) and sodium iodide (521 mg, 3.48 mmol, 20 eq) at room temperature, warmed to 75° C. for 24 h while monitoring reaction progress by TLC analysis (10% MeOH in CHCl₃, R_{F}:0.65). The reaction mixture was filtered, washed with ethyl acetate (60 mL). The combined organic layer was concentrated at 45° C. under reduced pressure to give residue (65 mg) that was purified by column chromatography (100-200 mesh silica gel, 1% MeOH:CHCl₃) to afford methyl 5-(3,3-dimethylbut-1-yn-1-yl)-3-((N-4-trans-cyclopropoxycyclohexyl)-4-trans-methylcyclohexanecarboxamido)thiophene-2-carboxylate (10 mg, 11.6%) as off white solid.

MS: m/z (obs.): 3 [M+H]⁺;

1H NMR CDCl₃, 400 MHz: 6.78 (s, 1H), 5.90-5.82 (s, 1H, impurity), 5.2-5.1 (m, 2H, impurity) 4.57-4.49 (m, 1H), 4.30-4.24 (m, 2H), 4.24-4.18 (m, 2H), 3.95 (d, J=8.0 Hz, impurity), 3.45*(s, 3H), 2.06-0.61 (series of m, 24H)

### Step 2: 5-(3,3-Dimethylbut-1-yn-1-yl)-3-((N-4-trans-cyclopropoxycyclohexyl)-4-trans-methylcyclohexanecarboxamido)thiophene-2-carboxylic acid

To the stirred solution of methyl 5-(3,3-dimethylbut-1-yn-1-yl)-3-((N-4-trans-cyclopropoxycyclohexyl)-4-trans-methylcyclohexanecarboxamido)thiophene-2-carboxylate (10 mg, 0.02 mmol, 1 eq) in 1:1 THF/MeOH mixture (2 mL) at room temperature was added LiOH.H₂O (8.4 mg, 0.2 mmol, 10 eq) at room temperature while monitoring reaction progress by TLC analysis (10% MeOH:CHCl₃, R_{f}: 0.32). The reaction mixture was concentrated under reduced pressure at 35° C. to give residue (20 mg) that was purified by Prep TLC (5% MeOH:CHCl₃) to get compound 38 (1.9 mg, 19.5%) as an off white solid.

MS: m/z (obs.): 486.0 [M+H]⁺;

1H NMR CDCl₃, 400 MHz: 6.78 (br.s, 1H), 5.95-5.83 (m, 1H impurity), 5.22 (d, J=17 Hz, impurity, 1H), 5.13 (d, J=10.0 Hz, impurity, 1H), 4.49 (s, 1H), 3.93 (s, 1H), 3.07 (s, 1H), 2.03-0.68 (series of m, 26H)

### Preparation of Compound 35

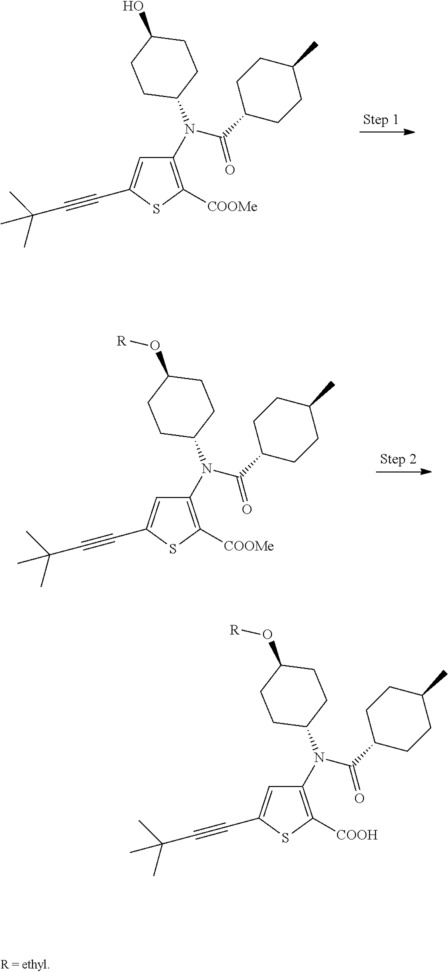

Compound 35 was prepared in two steps as described for Compound 38.

### Step 1: Methyl 5-(3,3-dimethylbut-1-yn-1-yl)-3-((N-4-trans-ethoxycyclohexyl)-4-trans-methylcyclohexanecarboxamido)thiophene-2-carboxylate

Isolated in 57% yield (60 mg):

MS: m/z (obs.): 488.1 [M+H]⁺;

1H NMR CDCl₃, 400 MHz: 6.78 (s, 1H), 4.57-4.51 (m, 1H), 3.82 (s, 3H), 3.45-3.41 (m, 2H), 3.06-3.00 (m, 1H), 2.06-1.91 (m, 4H), 1.77-1.74 (m, 1H), 1.66-1.59 (m, 4H), 1.34 (s, 9H), 1.29-1.22 (m, 2H), 1.15 (t, J=6.8, 6.8 Hz, 3H), 0.95-0.86 (m, 2H), 0.79 (d, J=6.4 Hz, 3H), 0.70-0.60 (m, 2H)

### Step 2: 5-(3,3-Dimethylbut-1-yn-1-yl)-3-((N-4-trans-ethoxycyclohexyl)-4-trans-methylcyclohexanecarboxamido)thiophene-2-carboxylic acid

Yield 13 mg, off-white solid.

MS: m/z (obs.): 474.0 [M+H]⁺;

1H NMR CDCl₃, 400 MHz: 6.89 (s, 1H), 4.50 (m, 1H), 3.50-3.42 (m, 2H), 3.02 (m, 1H), 2.01-1.84 (m, 6H), 1.59-1.40 (m, 4H) 1.32 (s, 9H), 1.25 (m, 2H), 1.15 (t, J=6.8, 7.2 Hz, 3H), 0.89-0.65 (m, 6H).

### Preparation of Compound 43

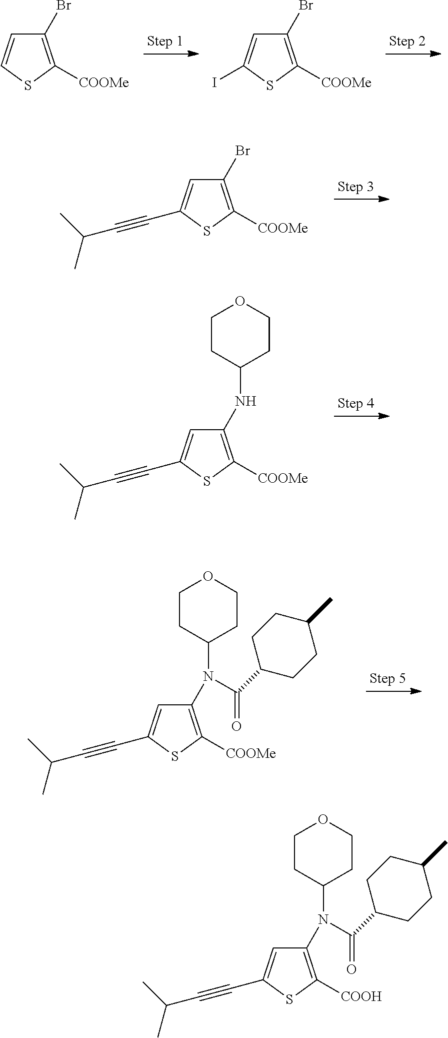

### Step 1: Methyl 3-Bromo-5-iodo-thiophene-2-carboxylate

To a stirred solution of diisopropylamine (29.76 g, 41.22 mL, 294.1 mmol) in 2-MeTHF (400 mL) was cooled to 0° C., n-BuLi (108.6 mL of 2.5 M, 271.4 mmol) was added drop wise, stirred for 30 minutes. The reaction mixture was cooled to −70° C., methyl 3-bromothiophene-2-carboxylate (50 g, 226.2 mmol) in 2-MeTHF (200 mL) was added drop wise over 30 minutes, after the addition the reaction mixture was stirred for 30 minutes, at which point HPLC-analysis revealed 5-10% starting material. Iodine (63.15 g, 12.81 mL, 248.8 mmol) in 2-MeTHF (100 mL) was added drop wise over 30 minutes, maintained the internal temperature −60° C. and stirred at this temperature for 45 minutes, allowed to 0° C., quenched with sat. aqueous NH4Cl solution (300 mL), diluted with MTBE (1.0 L), organic layer was separated. The organic layer was washed with 1 M aqueous Na₂S₂O₃ solution (200 mL), water (200 mL), brine (200 mL), dried over Na₂SO₄, filter and concentrated under reduced pressure. The crude product was purified by silica-gel plug using 10% ethyl acetate in hexanes as eluent to afford product ˜85% purity, which was triturated with methanol to afford methyl 3-bromo-5-iodo-thiophene-2-carboxylate (35.4 g, 45% yield) as a light yellow solid.

1H NMR CDCl₃, 300 MHz: 7.26 (s, 1H), 3.90 (s, 3H)

### Step 2: Methyl 3-Bromo-5-(3-methylbut-1-yn-1-yl)thiophene-2-carboxylate

To a stirred solution of methyl 3-bromo-5-iodo-thiophene-2-carboxylate (3.0 g, 10.0 mmol) in THF (45 ml) at 0° C. to −5° C., added CuI (76 mg, 0.4 mmol) followed by Et₃N (2.1 mL, 15.0 mmol). The reaction mixture was deoxygenated by purging with a stream of argon for 30 min at −5° C. Added Pd (PPh₃)₂Cl₂ (0.28 g, 0.4 mmol) and purging was continued. After 10 min added 3-methyl 1-butyene (0.81 g, 12.0 mmol) at −5° C. and stirred at RT for 16 h. The reaction progress was monitored by TLC. (The color of the reaction mass changes from brown colour solution to thick black indicates the completion of reaction). The reaction mixture was diluted with diethyl ether (50 mL), filtered through celite bed, washed with ether (2×10 mL). The filtrate was concentrated. The obtained crude compound was purified by column chromatography (100-200 mesh silica gel) using 3% EtOAc in hexane to afford methyl 3-bromo-5-(3-methylbut-1-yn-1-yl)thiophene-2-carboxylate (1.0 g, 35%) as a yellow liquid.

MS: m/z (obs.): 289.0 [M+H]⁺;

1H NMR CDCl₃, 400 MHz: 7.04 (s, 1H), 3.88 (s, 3H), 2.83-2.76 (m, 1H), 1.25 (d, J=6.8 Hz; 6H).

### Step 3: Methyl 5-(3-methylbut-1-yn-1-yl)-3-((tetrahydro-2H-pyran-4-yl)amino)thiophene-2-carboxylate

A suspension of methyl 3-bromo-5-(3-methylbut-1-yn-1-yl)thiophene-2-carboxylate (1.0 mmol), Cs₂CO₃ (3.0 mmol), BINAP (0.17 mmol) in dry toluene (5-10 vol) was deoxygenated by bubbling a stream of argon for 30 min at RT. Added Pd(OAc)₂ (0.1 mmol) and purging was continued for 10 min and added tetrahydro-2H-pyran-4-amine (1.2 mmol) at RT. The reaction mixture was stirred at 95° C. for 16 h. The reaction progress was monitored by TLC. Cooled to RT, diluted with EtOAc (50 mL), filtered through celite and the filtrate was concentrated. The obtained crude compound was purified by column chromatography (100-200 mesh silica gel) by using 6% EtOAc:pet ether as eluent to afford methyl 5-(3-methylbut-1-yn-1-yl)-3-((tetrahydro-2H-pyran-4-yl)amino)thiophene-2-carboxylate (800 mg, 38%).

MS: m/z (obs.): 308.1 [M+H]⁺;

1H NMR CDCl₃, 400 MHz: 6.75 (br d, D₂O exchangeable, 1H), 6.62 (s, 1H), 3.99-3.96 (m, 2H), 3.79 (s, 3H), 3.51-3.46 (m, 3H), 2.82-2.75 (m, 1H), 1.97 (br d, J=13.2 Hz; 2H), 1.62-1.53 (m, 2H), 1.25 (d, J=6.8 Hz; 6H).

### Step 4: Methyl 3-(N-(tetrahydro-2H-pyran-4-yl)-4-trans-methyl-cyclohexanecarboxamido)-5-(3-methylbut-1-yn-1-yl)thiophene-2-carboxylate

Methyl 5-(3-methylbut-1-yn-1-yl)-3-((tetrahydro-2H-pyran-4-yl)amino)thiophene-2-carboxylate was acylated with trans-4-methyl cyclohexanecarbonyl chloride by the methods described above to afford the desired product, isolated after silica gel chromatography (30% EtOAc in pet. ether), 100 mg (48%).

MS: m/z (obs.): 432.5 [M+H]⁺;

1H NMR CDCl₃, 400 MHz: 6.81 (s, 1H), 4.80-4.79 (m, 1H), 3.97-3.86 (m, 2H), 3.81 (s, 3H), 3.51-3.41 (m, 2H), 2.84-2.81 (m, 1H), 2.04-1.91 (m, 2H), 1.80-1.75 (m, 2H), 1.66-1.50 (m, 7H), 1.46-1.39 (m, 2H), 1.29 (d, J=6.8 Hz, 6H), 1.24-1.18 (m, 1H)

### Step 5: 3-(N-(tetrahydro-2H-pyran-4-yl)-4-trans-methyl-cyclohexanecarboxamido)-5-(3-methylbut-1-yn-1-yl)thiophene-2-carboxylic acid

Methyl 3-(N-(tetrahydro-2H-pyran-4-yl)-4-trans-methyl-cyclohexanecarboxamido)-5-(3-methylbut-1-yn-1-yl)thiophene-2-carboxylate was hydrolyzed using LiOH by the methods described above to afford the desired product, compound 43, isolated following silica gel chromatography (11% MeOH in CHCl₃), 290 mg (61%).

MS: m/z (obs.): 418.1 [M+H]⁺;

1H NMR CDCl₃, 400 MHz: 6.82 (s, 1H), 4.49-4.43 (m, 1H), 3.82-3.73 (m, 2H), 3.31-3.23 (m, 2H), 2.86-2.79 (m, 1H), 2.03-1.97 (m, 1H), 1.79-1.48 (m, 6H), 1.37-1.28 (m, 2H), 1.2 (d, J=7.2 Hz; 6H), 1.18-1.01 (m, 3H), 0.75 (d, J=6.0 Hz; 3H), 0.63-0.50 (m, 2H).

### Preparation of Compound 52

Compound 52 was prepared from trans-4-methylcyclohexylamine and methyl 3-bromo-5-(3-methylbut-1-yn-1-yl)thiophene-2-carboxylate according to the scheme described for compound 43.

MS: m/z (obs.): 446.1 [M+H]⁺;

1H NMR CDCl₃, 400 MHz: 6.82 (s, 1H), 4.50 (m, 1H), 3.28 (s, 3H), 2.95-2.94 (m, 1H), 2.85-2.82 (m, 1H), 2.09-2.06 (m, 1H), 2.01-1.93 (m, 2H), 1.84-1.81 (m, 1H), 1.65-1.55 (m, 5H), 1.42-1.33 (m, 4H), 1.29 (d, J=6.4 Hz; 6H), 0.94-0.88 (m, 1H), 0.79 (d, 3H), 0.75-0.60 (m, 2H).

### Preparation of Compound 61 and Compound 62

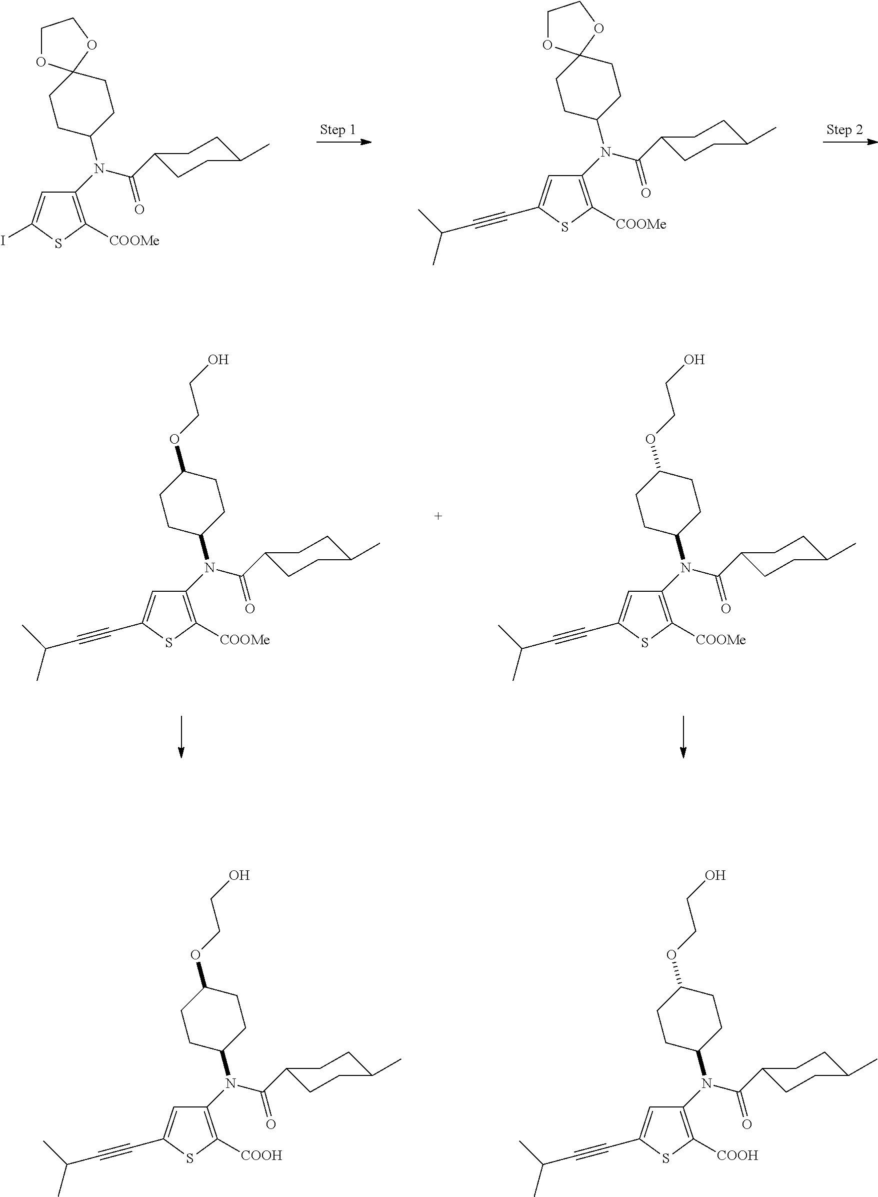

### Step 1: 5-(3-Methylbut-1-yn-1-yl)-3-[(1,4-dioxaspiro[4.5]decan-8-yl)-(4-trans-methylcyclohexanecarbonyl)amino]thiophene-2-carboxylic acid methyl ester

A suspension of compound 5-iodo-3-[(1,4-dioxaspiro[4.5]decan-8-yl)-(4-trans-methylcyclohexanecarbonyl)amino]thiophene-2-carboxylic acid methyl ester (300 mg, 0.548 mmol), Et₃N (66.4 mg, 0.658 mmol), CuI (5.2 mg, 0.027 mmol) in THF (7 mL) at 0-10° C. was deoxygenated by bubbling Ar for 20 min, added Pd(PPh₃)₂Cl₂ (19 mg, 0.027 mmol) and purging was continued for 10 min followed by the addition of 3-methyl-1-butyne (0.16 mL, 1.64 mmol). After addition the brown colored reaction mixture was warmed to RT and stirred for 2 h to get dark color. The reaction progress was monitored by TLC. The reaction mixture was diluted with diethyl ether (100 mL), filtered through celite bed, washed with excess of diethyl ether (2×50 mL). The filtrate was concentrated; the obtained crude compound was purified by column chromatography (100-200 mesh silica gel) using 10% EtOAc/Pet ether as eluent to afford 5-(3-methylbut-1-yn-1-yl)-3-[(1,4-dioxaspiro[4.5]decan-8-yl)-(4-trans-methylcyclohexanecarbonyl)-amino]-thiophene-2-carboxylic acid methyl ester (150 mg, 56%) as pale yellow solid.

MS: m/z (obs.): 488.2 [M+H]⁺;

1H NMR CDCl₃, 400 MHz: 6.81 (s, 1H), 4.68-4.56 (m, 1H), 3.89-3.86 (m, 4H), 3.81 (s, 3H), 2.83-2.76 (m, 1H), 1.87-1.80 (m, 2H), 1.75-1.55 (m, 7H), 1.52-1.36 (m, 3H), 1.36-1.22 (m, 7H), 0.79 (d, J=6.6 Hz; 3H), 0.72-0.58 (m, 2H).

### Step 2

### Methyl 5-(3-methylbut-1-yn-1-yl)-3-((N-4-cis-(2-hydroxyethoxy)-cyclohexyl)-4-trans-methylcyclohexanecarboxamido)thiophene-2-carboxylate and

### Methyl 5-(3-methylbut-1-yn-1-yl)-3-((N-4-trans-(2-hydroxyethoxy)-cyclohexyl)-4-trans-methylcyclohexanecarboxamido)thiophene-2-carboxylate

To a suspension of NaCNBH₃ (1.5 g, 24.63 mmol) in THF (40 mL) at 0° C. added Boron Trifluoride etherate (1.4 ml, 12.31 mmol) slowly and stirred for 1 h. To the obtained mixture, a solution of 5-(3-methylbut-1-yn-1-yl)-3-[(1,4-dioxaspiro[4.5]decan-8-yl)-(4-trans-methylcyclohexanecarbonyl)-amino]-thiophene-2-carboxylic acid methyl ester (1 g, 2.05 mmol) in THF (10 mL) was added and the reaction mixture was stirred at RT for 20 h. The reaction progress was monitored by TLC and observed the un reacted SM. The reaction mixture was quenched with ice water (100 mL), extracted with dichloromethane (4×100 mL). The combined organic layer was washed with NaHCO₃ (3×50 mL), brine (50 mL), dried over with NaSO₄. and concentrated. The obtained crude compound was purified by column chromatography (100-200 mesh silica gel) using 50% EtoAc/Pet ether as eluent to afford cis isomer methyl 5-(3-methylbut-1-yn-1-yl)-3-((N-4-cis-(2-hydroxyethoxy)-cyclohexyl)-4-trans-methylcyclohexanecarboxamido)thiophene-2-carboxylate (110 mg) and 70% EtOAc/Pet ether as eluent to afford trans isomer methyl 5-(3-methylbut-1-yn-1-yl)-3-((N-4-trans-(2-hydroxyethoxy)-cyclohexyl)-4-trans-methylcyclohexanecarboxamido)thiophene-2-carboxylate (275 mg). (TLC system: 80% EtOAC/Pet ether, R_{f} spot A (cis): 0.4, spot B (trans): 0.2).

### Cis Product:

MS: m/z (obs.): 490.2 [M+H]⁺;

1H NMR DMSO-d6, 400 MHz: 7.25 (s, 1H), 4.45-4.35 (m, 2H), 3.75 (s, 3H), 3.42-3.36 (m, 4H), 3.32-3.25 (m, 2H), 2.90 (m, 1H), 1.86-1.77 (m, 2H), 1.57-1.50 (m, 5H), 1.47-1.33 (m, 4H), 1.23-1.18 (m, 6H), 1.08-1.04 (m, 2H), 0.88-0.84 (m, 1H), 0.76

### Trans Product:

MS: m/z (obs.): 490.2 [M+H]⁺;

1H NMR DMSO-d6, 400 MHz: 7.25 (s, 1H), 4.45-4.35 (m, 2H), 3.75 (s, 3H), 3.42-3.36 (m, 4H), 3.32-3.25 (m, 2H), 2.90 (m, 1H), 1.86-1.77 (m, 2H), 1.57-1.50 (m, 5H), 1.47-1.33 (m, 4H), 1.23-1.18 (m, 6H), 1.08-1.04 (m, 2H), 0.88-0.84 (m, 1H), 0.76

### Step 3A: 5-(3-Methylbut-1-yn-1-yl)-3-((N-4-cis-(2-hydroxy)-ethoxycyclohexyl)-(4-trans-methylcyclohexane)carboxamido)thiophene-2-carboxylic acid (compound 61)

Ester hydrolysis was performed as described above. 60 mg, 57%.

MS: m/z (obs.): 476.2 [M+H]⁺;

1H NMR DMSO-d6, 400 MHz: 6.90 (s, 1H), 4.49 (m, 1H), 4.29 (m, 1H), 3.39-3.16 (m, 5H), 2.88-2.81 (m, 1H), 1.99-1.71 (m, 4H), 1.6-1.40 (m, 2H), 1.37-1.30 (m, 2H), 1.21-1.16 (m, 8H), 1.12-1.00 (m, 1H), 0.75 (d, J=6.6 Hz; 3H), 0.62-0.49 (m, 2H).

### Step 3B: 5-(3-Methylbut-1-yn-1-yl)-3-((N-4-trans-(2-hydroxy)-ethoxycyclohexyl)-(4-trans-methylcyclohexane)carboxamido)thiophene-2-carboxylic acid (compound 62)

Ester hydrolysis was performed as described above. 60 mg, 57%.

MS: m/z (obs.): 476.1 [M+H]⁺;

1H NMR DMSO-d6, 400 MHz: 6.98 (s, 1H), 4.51 (brs, 1H), 4.28-4.25 (m, 1H), 3.41-3.34 (m, 4H), 3.02-2.99 (m, 1H), 2.87-2.84 (m, 1H), 1.95-1.88 (m, 2H), 1.77-1.65 (m, 2H), 1.56-1.35 (m, 4H), 1.23-1.05 (m, 8H), 0.85-0.78 (m, 1H), 0.75 (d, J=6.6 Hz; 3H), 0.63-0.52 (m, 2H).

### Preparation of Compound 45

Compound 45 was prepared as described for compound 36.

MS: m/z (obs.): 406.2 [M+H]⁺;

1H NMR CDCl₃, 400 MHz: 13.6 (br), 7.02 (s, 1H), 4.81-4.78 (m, 1H), 3.23 (s, 3H), 3.22-3.20 (m, 2H), 3.09 (s, 2H), 2.91-2.84 (m, 2H), 1.92-1.84 (m, 2H), 1.70-1.4 (m, 6H), 1.21 (d, J=7.2 Hz, 9H), 0.76 (d, J=7.2 Hz, 3H).

### Preparation of Compound 40

Compound 40 was prepared as described for compound 33.

MS: m/z (obs.): 406.2 [M+H]⁺;

1H NMR DMSO-d6, 400 MHz: 6.93-6.88 (m, 1H), 4.74-4.72 (m, 1H), 4.38 (b s, 1H), 3.54 (b s, 1H), 3.26-3.07 (m, 6H), 2.89-2.81 (m, 1H), 2.08-1.98 (b s, 1H), 1.70-1.32 (m, 4H), 1.29-1.17 (m, 6H), 1.05 (2H, m), 0.96-0.50 (m, 10H).

### Preparation of Compound 44

Compound 44 was prepared as described for compound 927251.

MS: m/z (obs.): 420.1 [M+H]⁺;

1H NMR DMSO-d6, 400 MHz: 6.85 (s, 1H), 4.4 (br s, 1H), 3.2 (s, 3H), 3.05 (s, 2H), 2.9-2.8 (m, 1H), 2.08-1.98 (br s, 1H), 1.70-1.4 (m, 7H), 1.21 (d, J=6.8 Hz, 6H), 1.2-1.14 (m, 2H), 0.9-0.5 (m, 9H).

### Preparation of Compound 47

Compound 47 was prepared as described for compound 37 from S-1-methoxybutyl-2-amine

MS: m/z (obs.): 420.1 [M+H]⁺;

1H NMR DMSO-d6, 400 MHz: 6.85 (s, 1H), 4.4 (br s, 1H), 3.2 (s, 3H), 3.05 (s, 2H), 2.9-2.8 (m, 1H), 2.08-1.98 (br s, 1H), 1.70-1.4 (m, 7H), 1.21 (d, J=6.8 Hz, 6H), 1.2-1.14 (m, 2H), 0.9-0.5 (m, 9H)

### Preparation of Compound 53

Compound 53 was prepared as described for compound 35.

MS: m/z (obs.): 460.2 [M+H]⁺;

1H NMR DMSO-d6, 400 MHz: 6.89 (s, 1H), 4.23 (m, 1H), 3.37 (q, J=7.2 Hz; 2H), 2.99 (m, 1H), 2.87-2.80 (m, 1H), 1.95-1.72 (m, 6H), 1.55-1.48 (m, 3H), 1.40-1.31 (m, 1H), 1.20 (d, J=6.8 Hz; 6H), 1.16-1.08 (m, 3H), 1.04 (t, J=6.8 Hz; 3H), 0.86-0.74 (m, 4H)

### Preparation of Compound 50

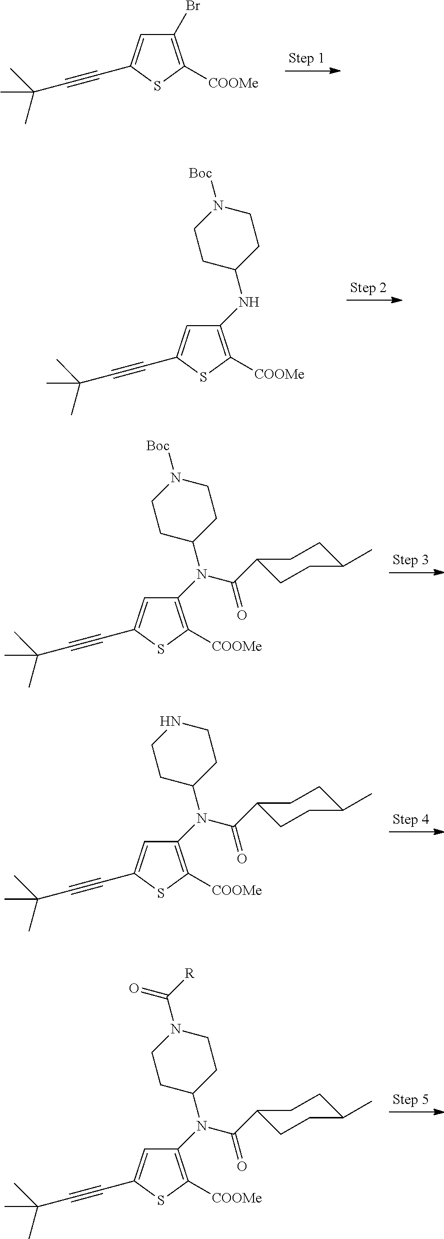

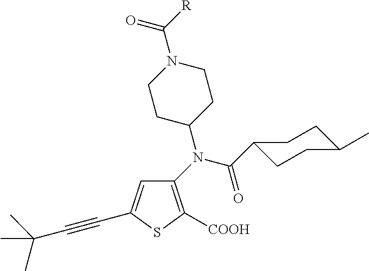

### Step 1

Methyl 3-bromo-5-(3,3-dimethylbut-1-ynyl)thiophene-2-carboxylate (1000 mg, 3.320 mmol), tert-butyl 4-aminopiperidine-1-carboxylate (797.9 mg, 3.984 mmol), cesium carbonate (3.245 g, 9.960 mmol), and dicyclohexyl-[2-(2,6-dimethoxyphenyl)phenyl]phosphane (136.3 mg, 0.3320 mmol) were taken into 15 mL of 1,4-dioxane. Heated at 100° C. for 18 h, then cooled and diluted with ethyl acetate. The mixture was washed with saturated sodium bicarbonate, water, and brine. The ethyl acetate extract was dried over anhydrous sodium sulfate, filtered and evaporated in vacuo. The crude product was purified by column chromatography (40 g SiO2 column) eluting with a gradient of hexane to 50% ethyl acetate in hexane. The desired fractions were combined and evaporated in vacuo to afford 870 mg of the desired product.

MS: m/z (obs.): 365.3 [M+H]⁺;

### Step 2

tert-Butyl 4-[[5-(3,3-dimethylbut-1-ynyl)-2-methoxycarbonyl-3-thienyl]amino]piperidine-1-carboxylate (870 mg, 2.069 mmol) was taken into toluene (10 mL) and pyridine (840 μL, 10.3 mmol). trans-4-Methylcyclohexanecarbonyl chloride (1.1 g, 6.85 mmol) was added to the mixture and heated to 110 C for 48 hours.

The mixture was cooled to room temperature and 1 mL of pyridine was added to the mixture followed by the addition of 1 mL of methanol. The reaction was diluted with dichloromethane and washed with brine, dried over anhydrous sodium sulfate, filtered and evaporated in vacuo to afford yellow solid that was purified by column chromatography eluting with a gradient of hexanes to 70% ethyl acetate in hexanes. The desired fractions were combined and evaporated and the resulting crystalline solid was dried under vacuum to afford 870 mg of the desired product.

MS: m/z (obs.): 545.4 [M+H]⁺;

### Step 3

tert-Butyl 4-[[5-(3,3-dimethylbut-1-ynyl)-2-methoxycarbonyl-3-thienyl]-(4-trans-methylcyclohexanecarbonyl)amino]piperidine-1-carboxylate (860 mg, 1.579 mmol) was dissolved in 5 mL of dichloromethane and 1 mL of TFA was added. The reaction was stirred at room temperature for 1 hour. The reaction was evaporated in vacuo and the residue dissolved in dichloromethane and washed with saturated sodium bicarbonate and brine. The organic dried over anhydrous sodium sulfate, filtered, and evaporated in vacuo to afford 679 mg of the desired product.

MS: m/z (obs.): 445.3 [M+H]⁺;

### Step 4 (R═CH₃OCH₂—)

Methyl 5-(3,3-dimethylbut-1-ynyl)-3-[(4-trans-methylcyclohexanecarbonyl)-(4-piperidyl)amino]thiophene-2-carboxylate (350.0 mg, 0.7872 mmol) was taken into triethylamine (143 μL, 1.02 mmol) and dichloromethane (5 mL). The mixture was cooled to 0° C. with an ice bath and 2-methoxyacetyl chloride (85.4 mg, 0.79 mmol) was added to the mixture and stirred overnight allowing the temperature to warm to room temperature. The reaction was diluted with dichloromethane and washed with water, saturated sodium bicarbonate soln. and brine. The organic was dried over anhydrous sodium sulfate, filtered and evaporated in vacuo to afford a gum that was purified by chromatography (SiO2) eluting with a gradient of dichloromethane to ethyl acetate. The desired fractions were combined and evaporated to afford 370 mg of the desired product.

MS: m/z (obs.): 517.4 [M+H]⁺;

### Step 5 (R═CH₃OCH₂—) 5-(3,3-dimethylbut-1-ynyl)-3-[[1-(2-methoxyacetyl)-4-piperidyl]-(4-trans-methylcyclohexanecarbonyl)amino]thiophene-2-carboxylic acid (compound 50)

Methyl 5-(3,3-dimethylbut-1-ynyl)-3-[[1-(2-methoxyacetyl)-4-piperidyl]-(4-trans-methylcyclohexanecarbonyl)amino]thiophene-2-carboxylate (360 mg, 0.70 mmol) was dissolved in 1:1 mixture of MeOH and water (5 ml). To the solution was added Lithium hydroxide (33.4 mg, 1.39 mmol) and the mixture was stirred overnight. Inspection of the LC/MS showed product and also slight hydrolysis of the amide.

Compound was purified by column chromatography eluting with a gradient of dichloromethane to 10% methanol and 0.5% formic acid in dichloromethane. Desired fractions were combined and evaporated to give the desired product.

MS: m/z (obs.): 503.4 [M+H]⁺;

1H NMR DMSO-d₆, 300 MHz: 6.78 (d, J=8.9 Hz, 1H), 4.85 (d, J=19.3 Hz, 1H), 4.71 (m, 2H), 4.43 (br s, 1H), 4.11-4.02 (m, 2H), 3.87 (dd, J=25.7, 14.0 Hz, 1H, 3.39 (d, J=6.2 Hz, 3H), 3.11 (dd, J=26.7, 12.9 Hz, 1H), 2.66 (dd, J=24.0, 11.5 Hz, 1H), 2.05-1.78 (m, 3H), 1.65-1.5 (m, 5H), 1.49-1.39 (m, 2H), 1.36 (s, 9H), 1.110 (m, 1H), 0.82 (d, J=6.5 Hz, 3H), 0.76 (m, 2H).

### Preparation of Compound 51 (R═CH₃—)

Compound 51 was prepared by the methods described for compound 50.

MS: m/z (obs.): 473.3 [M+H]⁺;

1H NMR DMSO-d6, 300 MHz: 13.64 (br s, 1H), 7.19 (d, J=7.2 Hz, 1H), 4.64-4.26 (m, 2H), 3.79 (m, 1H), 3.04 (m, 1H), 1.93 (d, J=9.4 Hz, 3H), 1.84 (d, J=11.5 Hz, 2H), 1.70-1.35 (m, 5H), 1.30 (s, 9H), 1.25-1.04 (m, 4H), 1.04-0.81 (m, 1H), 0.76 (d, J=6.4 Hz, 3H), 0.59 (dd, J=26.9, 14.8 Hz, 3H).

### Preparation of Compound 54

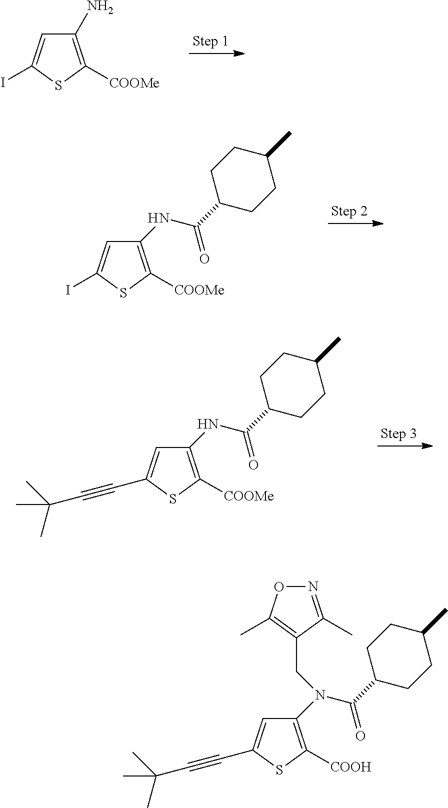

### Step 1. Methyl 5-iodo-3-(trans-4-methylcyclohexanecarboxamido)thiophene-2-carboxylate

Methyl 3-amino-5-iodo-thiophene-2-carboxylate (50 g, 174.9 mmol) was dissolved in CH2Cl2 (500 mL) and pyridine (30 mL) and cooled to 0° C. and then added trans-4-methyl cyclohexanecarbonyl chloride (60.2 g, 210 mmol) dropwise (neat), and rinsed with a small amount of DCM. After 5 min, removed bath, and stirred as reaction came to RT. After 1.25 hours, took an aliquot from the reaction, diluted with DCM, and check for disappearance of SM by TLC (5% EtOAc/Hexane); Reaction complete. 1.) Work up by adding brine, and then extraction with DCM (2×500 mL), combined and washed with 1N HCl (500 mL), washed with 1:1-1N NaOH (50 mL) brine (500 mL); back extracted 1×, then dried over sodium sulfate, filtered and evaporated, then triturated product with hexane. 67.3 g, (93%).

MS: m/z (obs.): 407.95 [M+H]⁺;

### Step 2. Methyl 5-(3,3-dimethylbut-1-yn-1-yl)-3-(trans-4-methylcyclohexane-carboxamido)-thiophene-2-carboxylate

In a dry flask under nitrogen atmosphere, mixed methyl 5-iodo-3-[(4-trans-methylcyclohexanecarbonyl)amino]thiophene-2-carboxylate (52 g, 128 mmol), 3,3-dimethylbut-1-yne (12.58 g, 18.3 mL, 153.2 mmol), triethylamine (53.4 mL, 383 mmol), and then cooled with ice bath to at 0° C. before adding copper iodide (2.95 g, 15.5 mmol), and Pd₂(dba)₃ (1.27 g, 1.39 mmol); Removed bath and stirred as reaction came to RT over several hours. Added brine (800 mL) and DI water (200 mL), and isopropyl acetate (1000 mL), stirred and filtered through celite. Organic phase dried over sodium sulfate, filtered and evaporated, then purified over silica gel using 5% EtOAc/Hexane to give desired product, 46 g (97%).

MS: m/z (obs.): 362.4 [M+H]⁺;

### Step 3. 5-(3,3-Dimethylbut-1-yn-1-yl)-3-(N-((3,5-dimethylisoxazol-4-yl)methyl)-4-trans-methylcyclohexanecarboxamido)thiophene-2-carboxylic acid

Methyl 5-(3,3-dimethylbut-1-yn-1-yl)-3-(trans-4-methylcyclohexane-carboxamido)-thiophene-2-carboxylate (50 mg) in DMF (5 mL) was treated with NaH (1 equiv.) and 4-(chloromethyl)-3,5-dimethylisoxazole (1 equiv). Stirred for 1 h at room temperature, then LiOH and H2O added and stirred for 24 h. The desired product was isolated by preparative HPLC.

MS: m/z (obs.): 457.41 [M+H]⁺.

### Preparation of Compound 41

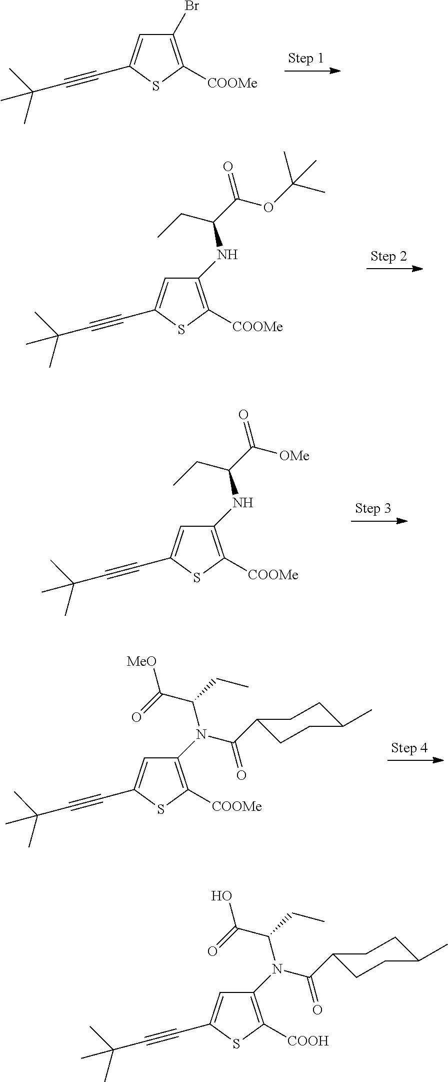

### Step 1. (S)-methyl 3-((1-(tert-butoxy)-1-oxobutan-2-yl)amino)-5-(3,3-dimethylbut-1-yn-1-yl)thiophene-2-carboxylate

1,4-Dioxane solvent used in reaction was anhydrous and deoxygenated by purging with nitrogen gas for 30 mins. Methyl 3-bromo-5-(3,3-dimethylbut-1-ynyl)thiophene-2-carboxylate (500 mg, 1.660 mmol), dicyclohexyl-[2-(2,6-dimethoxyphenyl)phenyl]phosphane (68 mg, 0.17 mmol) and cesium carbonate (1.62 g, 4.98 mmol) were taken into 5 mL of toluene and argon was bubbled to the mixture for 5 minutes. The catalyst Pd₂dba₃ (76 mg, 0.083 mmol) was added to the mixture and the reaction was sealed and heated at 90° C. overnight for 18 hrs. Cooled, diluted with EtOAc, and washed with sat NaHCO₃, and water, dried over MgSO4, filtered, evaporated with silica gel and purified by silica gel chromatography eluted with 0-35% EtOAc in hexane over 35 min to afford desired product as an oil (67%).

MS: m/z (obs.): 380.3 [M+H]⁺;

1H NMR (300 MHz, CDCl3) δ 7.01 (d, J=8.3 Hz, 1H), 6.47 (s, 1H), 3.82 (dt, J=8.4, 6.1 Hz, 1H), 3.73 (s, 3H), 1.89-1.65 (m, 3H), 1.38 (s, 9H), 1.23 (s, 9H), 0.91 (t, J=7.4 Hz, 3H).

### Step 2. (S)-methyl 5-(3,3-dimethylbut-1-yn-1-yl)-3-((1-methoxy-1-oxobutan-2-yl)-amino)thiophene-2-carboxylate

To a solution of methyl 3-[[(1S)-1-tert-butoxycarbonylpropyl]amino]-5-(3,3-dimethylbut-1-ynyl)thiophene-2-carboxylate (200 mg, 0.53 mmol) in dry MeOH (4 mL) was added 6 M hydrogen chloride in MeOH (880 μL, 5.3 mmol) and the mixture was stirred at room temperature for 15 h. LCMS showed major product was the methyl ester. Evaporated the solvent, the residue was used directly for the next step.

MS: m/z (obs.): 338.2 [M+H]⁺;

### Step 3. Methyl 5-(3,3-dimethylbut-1-yn-1-yl)-3-(N—((S)-1-methoxy-1-oxobutan-2-yl)-(4-trans-methylcyclohexane)-carboxamido)thiophene-2-carboxylate

To a solution of crude (S)-methyl 5-(3,3-dimethylbut-1-yn-1-yl)-3-((1-methoxy-1-oxobutan-2-yl)amino)thiophene-2-carboxylate (200 mg, 0.59 mmol) in DCE (4 mL) was added pyridine (57 μL, 0.71 mmol) and followed by adding 4-methylcyclohexane-carbonyl chloride (143 mg, 0.89 mmol); the mixture was heated at 90° C. overnight. The crude product was purified by silica gel chromatography eluted with 5-35% EtOAc in hexane to give a clear oil (55%).

MS: m/z (obs.): 462.3 [M+H]⁺;

### Step 4. 3-(N—((S)-1-carboxypropyl)-(4-trans-methylcyclohexane)-carboxamido)-5-(3,3-dimethylbut-1-yn-1-yl)thiophene-2-carboxylic acid

To a solution of methyl 5-(3,3-dimethylbut-1-yn-1-yl)-3-(N—((S)-1-methoxy-1-oxobutan-2-yl)-(4-trans-methylcyclohexane)-carboxamido)thiophene-2-carboxylate (150 mg, 0.32 mmol) in water (3 mL) and THF (3 mL) was added LiOH (78 mg, 3.25 mmol), stirred at room temperature for 12 hrs. Acidified with 6N HCl to pH 1, blowing off the THF using nitrogen, filtered to obtain a gummy solid, left to dryness in filter funnel with vacuum, the gummy solid turned to off white powder, washed with water and then dried again on vacuum (95%).

MS: m/z (obs.): 434.3 [M+H]⁺;

1H NMR (300 MHz, CDCl3) δ 7.21 (s, 0.6H), 7.18 (s, 0.4H), 4. 71 (t, 0.6H), 4.20 (t, 0.4H), 2.20-1.15 (m, 11H), 1.28 (s, 9H), 0.91-0.50 (m, 7H).

### Preparation of Compound 72

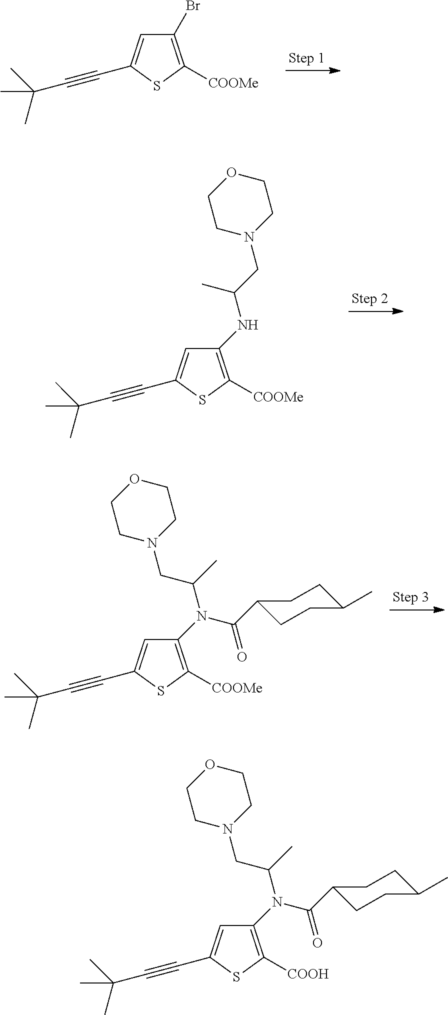

### Step 1. Methyl 5-(3,3-dimethylbut-1-yn-1-yl)-3-((1-morpholinopropan-2-yl)amino)thiophene-2-carboxylate

Title compound was prepared in the manner described for compound 54.

MS: m/z (obs.): 365.2 [M+H]⁺;

### Step 2. Methyl 5-(3,3-dimethylbut-1-yn-1-yl)-3-(trans-4-methyl-N-(1-morpholinopropan-2-yl)cyclohexanecarboxamido)thiophene-2-carboxylate

Title compound was prepared in the manner described for compound 54.

MS: m/z (obs.): 489.3 [M+H]⁺;

### Step 3. 5-(3,3-Dimethylbut-1-yn-1-yl)-3-(trans-4-methyl-N-(1-morpholinopropan-2-yl)cyclohexanecarboxamido)thiophene-2-carboxylic acid

Title compound 72 was prepared using procedures described for compound 54.

MS: m/z (obs.): 475.24 [M+H]⁺;

1H NMR (300 MHz, CDCl3) δ 6.75 (s, 1H), 5.65 (br, 1H), 4.25-3.80 (m, 4H), 3.15-2.65 (m, 6H), 2.20-1.48 (m, 7H), 1.40-1.28 (m, 14H), 0.85-0.60 (m, 4H).

### Preparation of Compound 64

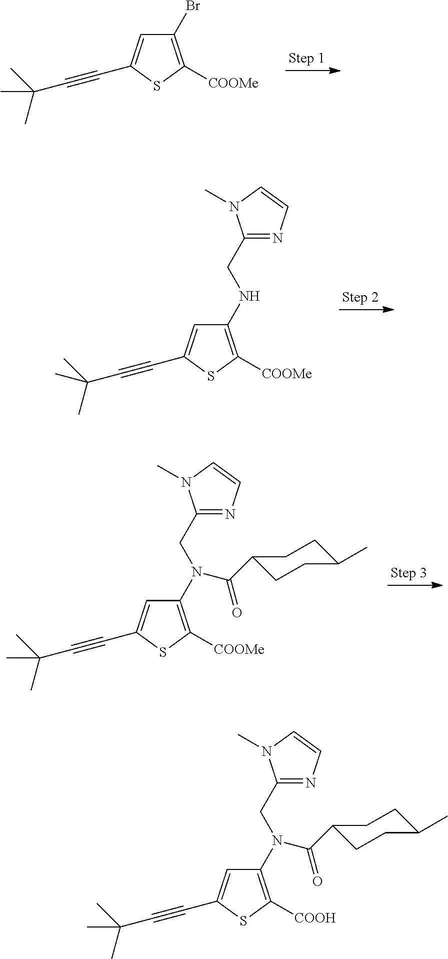

### Step 1. Methyl 5-(3,3-dimethylbut-1-ynyl)-3-((1-methyl-1H-imidazol-2-yl)methylamino)thiophene-2-carboxylate

To a solution of methyl 3-amino-5-(3,3-dimethylbut-1-ynyl)thiophene-2-carboxylate (125 mg, 0.53 mmol) and glacial acetic acid (120 μL, 2.10 mmol) in 1,2-dichloroethane (1.5 mL) was added 1-methylimidazole-2-carbaldehyde (174 mg, 1.58 mmol) and allowed to stir for 1 hr. Sodium triacetoxyborohydride (279 mg, 1.32 mmol) was added and allowed to stir overnight using LC/MS to monitor progress. The reaction was made basic with the addition of saturated aq. NaHCO₃ solution (10 mL) and stirring for 20 min. Extracted with DCM (2×10 mL) and washed combined organics with brine, dried over sodium sulfate and concentrated under vacuum. 168 mg of desired product obtained, 96% yield.

MS: m/z (obs.): 332 [M+H]⁺

### Step 2. Methyl 5-(3,3-dimethylbut-1-ynyl)-3-(N-((1-methyl-1H-imidazole-2-yl)methyl)-4-trans-4-methylcyclohexanecarboxamido)-thiophene-2-carboxylate

To a mixture of methyl 5-(3,3-dimethylbut-1-ynyl)-3-((1-methyl-1H-imidazol-2-yl)methylamino)thiophene-2-carboxylate (168 mg, 0.51 mmol) in dichloroethane (5 mL) was added pyridine (410 μL, 5.07 mmol), N,N-dimethylaminopyridine (6 mg, 0.05 mmol), and 4-methylcyclohexanecarbonyl chloride (407 mg, 2.5 mmol). The mixture was reflux for 24 h and monitored with LC/MS. The reaction was cooled to room temperature and diluted with ethyl acetate (30 mL), washed with water (15 mL) and saturated aq. NaHCO₃ solution (15 mL). Concentrated organic phase under vacuum and. purified by chromatography over silica gel using a 0-50% ethyl acetate/hexanes gradient as eluant. 150 mg obtained, 65% yield.

MS: m/z (obs.): 456 [M+H]⁺

### Step 3. 5-(3,3-Dimethylbut-1-ynyl)-3-((trans)-4-methyl-N-((1-methyl-1H-imidazol-2-yl)methyl)cyclohexanecarboxamido)thiophene-2-carboxylic acid

To a solution of methyl 5-(3,3-dimethylbut-1-ynyl)-3-(N-((1-methyl-1H-imidazole-2-yl)methyl)-4-trans-4-methylcyclohexanecarboxamido)-thiophene-2-carboxylate (150 mg, 0.33 mmol) in methanol (5 mL) was added sodium hydroxide (1.65 mmol, 1M) and allowed to stir overnight. Starting material consumed by LC/MS. The reaction mixture was acidified to pH˜6 with 1N HCl and extracted with dichloromethane (2×15 mL). The combined organics were dried over sodium sulfate and concentrated under vacuum. The resulting residue was chromatographed over 12 g silica gel using a 0 to 15% MeOH/DCM gradient as eluant. 60 mg obtained, 38% yield.

MS: m/z (obs.): 442 [M+H]⁺;

1H NMR (300 MHz, CDCl3) d 7.03 (s, 1H), 6.90 (s, 1H), 6.77 (s, 1H), 5.53 (d, J=16.4 Hz, 1H), 4.47 (d, J=16.1 Hz, 1H), 3.72 (s, 3H), 2.42 (t, J=11.8 Hz, 1H), 1.93 (d, J=13.0 Hz, 1H), 1.71-1.13 (m, 15H), 0.96-0.68 (m, 5H).

### Preparation of Compound 66

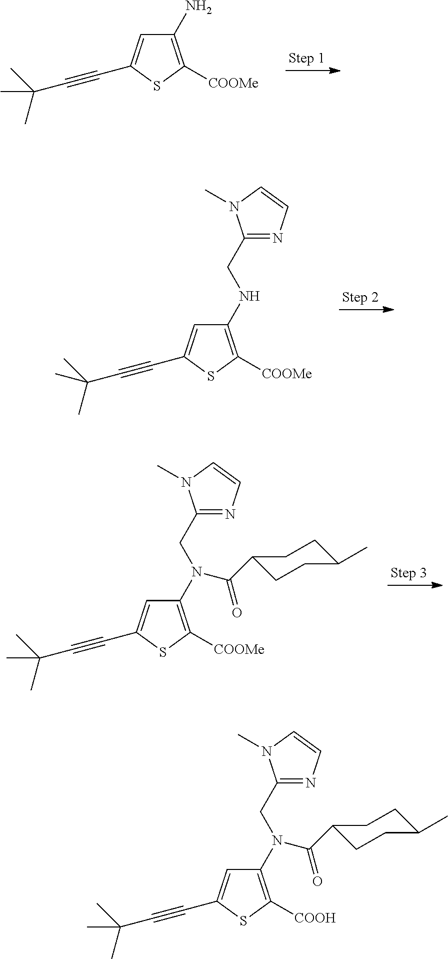

### Step 1. Methyl 5-(3,3-dimethylbut-1-ynyl)-3-((1-methyl-1H-imidazol-5-yl)methylamino)thiophene-2-carboxylate

To a solution of methyl 3-amino-5-(3,3-dimethylbut-1-ynyl)thiophene-2-carboxylate (125 mg, 0.53 mmol) and glacial acetic acid (126 mg, 120 μL, 2.1 mmol) in 1,2-dichloroethane (5 mL) was added 3-methylimidazole-4-carbaldehyde (174 mg, 1.58 mmol) and allowed to stir for 1 hr. sodium triacetoxyborohydride (279 mg, 1.32 mmol) was added and allowed to stir overnight using LC/MS to monitor progress. The reaction was made basic with the addition of saturated aq. NaHCO₃ solution (10 mL) and stirring for 20 min. Extracted with DCM (2×10 mL) and washed combined organics with brine, dried over sodium sulfate and concentrated under vacuum. 175 mg of desired product obtained, 100% yield.

MS: m/z (obs.): 332 [M+H]⁺

### Step 2. Methyl 5-(3,3-dimethylbut-1-ynyl)-3-((trans)-4-methyl-N-((1-methyl-1H-imidazol-5-yl)methyl)cyclohexanecarboxamido)thiophene-2-carboxylate

To a mixture of methyl 5-(3,3-dimethylbut-1-ynyl)-3-((1-methyl-1H-imidazol-5-yl)methylamino)thiophene-2-carboxylate (175 mg, 0.53 mmol)) in dichloroethane (5 mL) was added pyridine (430 μL, 5.310 mmol), N,N-dimethylaminopyridine (6.5 mg, 0.05 mmol), and 4-methylcyclohexanecarbonyl chloride (427 mg, 2.65 mmol). The mixture was reflux for 24 h and monitored with LC/MS. The reaction was cooled to room temperature and diluted with ethyl acetate (30 mL), washed with water (15 mL) and saturated aq. NaHCO₃ solution (15 mL). The organic phase was concentrated under vacuum. 230 mg obtained, 92% yield.

MS: m/z (obs.): 456 [M+H]⁺

### Step 3. 5-(3,3-Dimethylbut-1-ynyl)-3-((trans)-4-methyl-N-((1-methyl-1H-imidazol-5-yl)methyl)cyclohexanecarboxamido)thiophene-2-carboxylic acid

To a solution of methyl 5-(3,3-dimethylbut-1-ynyl)-3-((trans)-4-methyl-N-((1-methyl-1H-imidazol-5-yl)methyl)cyclohexanecarboxamido)thiophene-2-carboxylate (230 mg, 0.50 mmol) in methanol (5 mL) was added 1 M sodium hydroxide (2.5 mL) and allowed to stir overnight. Starting material consumed by LC/MS. The reaction mixture was acidified to pH˜6 with 1N HCl and extracted with dichloromethane (2×15 mL). The combined organics were dried over sodium sulfate and concentrated under vacuum. The resulting residue was chromatographed over 12 g silica gel using a 0 to 20% MeOH/DCM gradient as eluant. 51 mg obtained, 20% yield.

MS: m/z (obs.): 442 [M+H]⁺;

1H NMR (300 MHz, CDCl3) δ 11.84 (s, 1H), 8.00 (s, 1H), 6.78 (s, 1H), 6.58 (s, 1H), 5.56 (d, J=13.6 Hz, 1H), 4.16-3.93 (m, 1H), 3.73 (s, 3H), 2.20 (t, J=11.5 Hz, 1H), 1.91-1.14 (m, 15H), 0.98-0.52 (m, 5H).

### Preparation of Compound 67

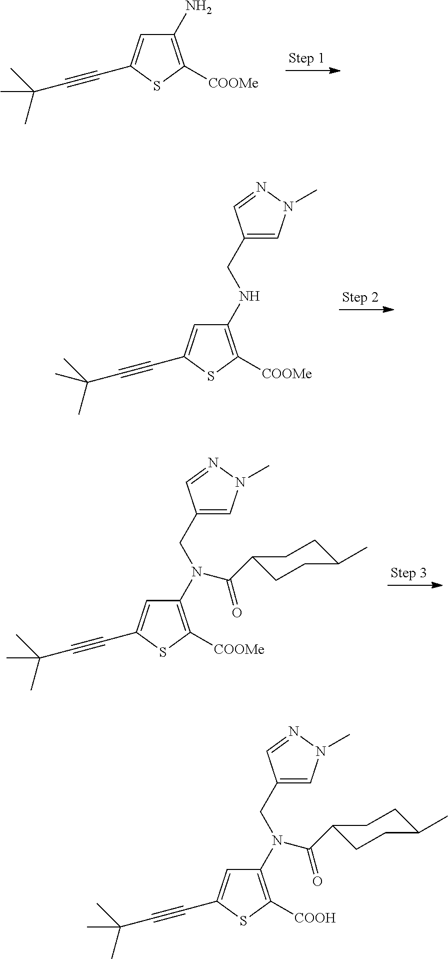

### Step 1. Methyl 5-(3,3-dimethylbut-1-ynyl)-3-((1-methyl-1H-pyrazol-4-yl)methylamino)thiophene-2-carboxylate

To a solution of methyl 3-amino-5-(3,3-dimethylbut-1-ynyl)thiophene-2-carboxylate (130 mg, 0.55 mmol) and glacial acetic acid (125 μL, 2.19 mmol) in 1,2-dichloroethane (5 mL) was added 1-methylpyrazole-4-carbaldehyde (181 mg, 1.64 mmol) and allowed to stir for 1 h. Sodium triacetoxyborohydride (290 mg, 1.37 mmol) was added and allowed to stir overnight using LC/MS to monitor progress. The reaction was made basic with the addition of saturated aq. NaHCO₃ solution (10 mL) and stirring for 20 min. Extracted with DCM (2×10 mL), dried combined organics over sodium sulfate and concentrated under vacuum. The resulting residue was chromatographed over 12 g silica gel using a 0 to 50% EtOAc/Hex gradient as eluant. 142 mg obtained.

MS: m/z (obs.): 332 [M+H]⁺

1H NMR (300 MHz, CDCl3) δ 7.42 (s, 1H), 7.29 (s, 1H), 6.87 (s, 1H), 6.63 (s, 1H), 4.28 (d, J=5.7 Hz, 2H), 3.86 (d, J=4.7 Hz, 3H), 3.78 (s, 3H), 1.35-1.24 (m, 9H).

### Step 2. Methyl 5-(3,3-dimethylbut-1-ynyl)-3-((trans)-4-methyl-N-((1-methyl-1H-pyrazol-4-yl)methyl)cyclohexanecarboxamido)thiophene-2-carboxylate

To a mixture of methyl 5-(3,3-dimethylbut-1-ynyl)-3-[(1-methylpyrazol-4-yl)methylamino]thiophene-2-carboxylate (142 mg, 0.43 mmol) in dichloroethane (5 mL) was added pyridine (350 μL, 4.28 mmol), dimethylaminopyridine (5 mg, 0.04 mmol), and 4-methylcyclohexanecarbonyl chloride (344 mg, 2.1 mmol). The mixture was reflux for 24 h and monitored with LC/MS. The reaction was cooled to room temperature and diluted with ethyl acetate (30 mL), washed with water (15 mL) and saturated aq. NaHCO₃ solution (15 mL). The organic phase was concentrated under vacuum and the resulting residue was chromatographed over 12 g silica gel using 0-50% EtOAc/hex as eluant. Recovered desired product 152 mg, 78% yield.

MS: m/z (obs.): 456 [M+H]⁺

### Step 3. 5-(3,3-Dimethylbut-1-ynyl)-3-((trans)-4-methyl-N-((1-methyl-1H-pyrazol-4-yl)methyl)cyclohexanecarboxamido)thiophene-2-carboxylic acid

To a solution of methyl 5-(3,3-dimethylbut-1-ynyl)-3-((trans)-4-methyl-N-((1-methyl-1H-pyrazol-4-yl)methyl)cyclohexanecarboxamido)thiophene-2-carboxylate (152 mg, 0.33 mmol) in methanol (3.3 mL) was added 1 M sodium hydroxide (1.7 mL) and allowed to stir overnight. Starting material consumed by LC/MS. The reaction mixture was acidified to pH˜6 with 1N HCl and extracted with dichloromethane (2×15 mL). The combined organics were dried over sodium sulfate and concentrated under vacuum and the resulting residue was chromatographed over 12 g silica gel using a 0 to 15% MeOH/DCM gradient as eluant.

MS: m/z (obs.): 442 [M+H]⁺;

1H NMR (300 MHz, CDCl3) δ 10.97 (s, 1H), 7.34 (s, 1H), 7.29 (s, 1H), 6.75 (s, 1H), 4.61 (dd, J=32.1, 14.7 Hz, 2H), 3.82 (s, 3H), 2.10 (t, J=11.5 Hz, 1H), 1.81-1.36 (m, 6H), 1.40-1.20 (m, 10H), 0.89-0.58 (m, 5H).

### Preparation of Compound 71

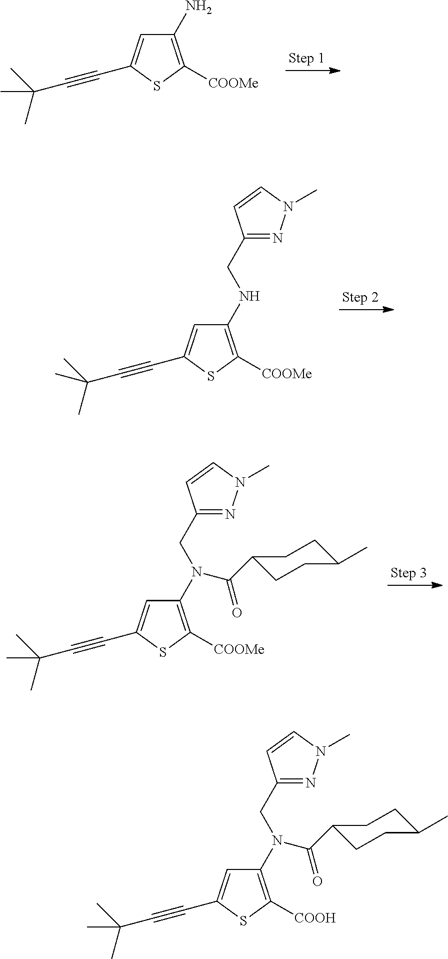

### Step 1. Methyl 5-(3,3-dimethylbut-1-ynyl)-3-((1-methyl-1H-pyrazol-3-yl)methylamino)thiophene-2-carboxylate

To a solution of methyl 3-amino-5-(3,3-dimethylbut-1-ynyl)thiophene-2-carboxylate (125 mg, 0.53 mmol) and glacial acetic acid (120 μL, 2.1 mmol) in 1,2-dichloroethane (5 mL) was added 1-methylpyrazole-3-carbaldehyde (174 mg, 1.58 mmol) and allowed to stir for 30 min. sodium triacetoxyborohydride (279 mg, 1.32 mmol) was added and allowed to stir overnight. The reaction was made basic with the addition of saturated aq. NaHCO₃ solution (10 mL) and stirring for 20 min. Extracted with DCM (2×10 mL), dried combined organics over sodium sulfate and concentrated under vacuum. The resulting residue was chromatographed over 12 g silica gel using a 0 to 40% EtOAc/Hex gradient as eluant. 164 mg obtained, 93% yield.

MS: m/z (obs.): 332 [M+H]⁺

### Step 2. Methyl 5-(3,3-dimethylbut-1-ynyl)-3-((trans)-4-methyl-N-((1-methyl-1H-pyrazol-3-yl)methyl)cyclohexanecarboxamido)thiophene-2-carboxylate

To a mixture of methyl 5-(3,3-dimethylbut-1-ynyl)-3-((1-methyl-1H-pyrazol-3-yl)methylamino)thiophene-2-carboxylate (160 mg, 0.48 mmol) in dichloroethane (5 mL) was added pyridine (390 μL, 4.8 mmol), N,N-dimethylaminopyridine (6 mg, 0.05 mmol), and 4-methylcyclohexanecarbonyl chloride (388 mg, 2.4 mmol). The mixture was reflux for 24 h and monitored with LC/MS. The reaction was cooled to room temperature and diluted with ethyl acetate (30 mL), washed with water (15 mL) and saturated aq. NaHCO₃ solution (15 mL). The organic phase was concentrated under vacuum and the resulting residue was chromatographed over 12 g silica gel using 0-50% EtOAc/hex as eluant. 127 mg obtained, 58% yield.

MS: m/z (obs.): 456 [M+H]⁺

1H NMR (300 MHz, CDCl3) δ 7.23 (d, J=2.1 Hz, 1H), 6.73 (s, 1H), 6.18 (d, J=2.2 Hz, 1H), 5.13 (d, J=14.8 Hz, 1H), 4.43 (d, J=14.8 Hz, 1H), 3.78 (d, J=7.1 Hz, 6H), 2.05 (s, 1H), 1.64 (d, J=7.6 Hz, 6H), 1.38-1.18 (m, 10H), 0.80 (d, J=6.5 Hz, 3H), 0.70 (d, J=11.2 Hz, 2H).

### Step 3. 5-(3,3-Dimethylbut-1-ynyl)-3-((trans)-4-methyl-N-((1-methyl-1H-pyrazol-3-yl)methyl)cyclohexanecarboxamido)thiophene-2-carboxylic acid

To a solution of methyl 5-(3,3-dimethylbut-1-ynyl)-3-((trans)-4-methyl-N-((1-methyl-1H-pyrazol-3-yl)methyl)cyclohexanecarboxamido)thiophene-2-carboxylate (122 mg, 0.27 mmol) in methanol (3 mL) was added 1 M sodium hydroxide (1.4 mL) and allowed to stir for 60 h. The starting material was consumed by LC/MS. The reaction mixture was acidified to pH˜4 with 1N HCl and extracted with dichloromethane (2×15 mL). The combined organics were dried over sodium sulfate and concentrated under vacuum. The resulting solid was diluted with EtOAc (˜1 mL) and added hexanes dropwise until product precipitated. The white solid was filtered to give 87 mg of product, 70% yield.

MS: m/z (obs.): 442 [M+H]⁺;

1H NMR (300 MHz, CDCl3) d 7.29 (d, J=2.3 Hz, 1H), 6.89 (s, 1H), 6.09 (d, J=2.3 Hz, 1H), 5.56 (d, J=16.4 Hz, 1H), 4.38 (d, J=16.3 Hz, 1H), 3.83 (s, 3H), 2.35 (t, J=11.3 Hz, 1H), 1.88 (d, J=11.5 Hz, 1H), 1.75-1.46 (m, 4H), 1.45-1.14 (m, 10H), 0.91-0.58 (m, 5H).

### Preparation of Compound 77

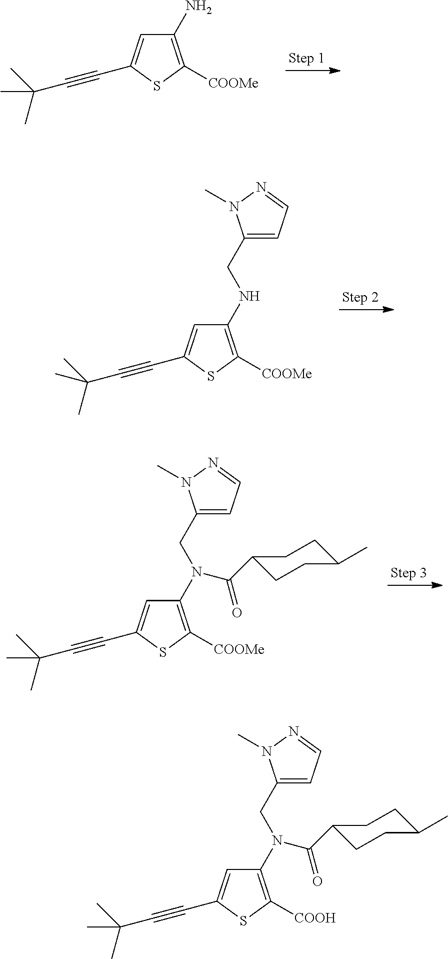

### Step 1. Methyl 5-(3,3-dimethylbut-1-ynyl)-3-((1-methyl-1H-pyrazol-5-yl)methylamino)thiophene-2-carboxylate

To a solution of methyl 3-amino-5-(3,3-dimethylbut-1-ynyl)thiophene-2-carboxylate (130 mg, 0.55 mmol) and glacial acetic acid (125 μL, 2.19 mmol) in 1,2-dichloroethane (5 mL) was added and allowed to stir for 30 min. sodium triacetoxyborohydride (290 mg, 1.37 mmol) was added and allowed to stir overnight. The reaction was made basic with the addition of saturated aq. NaHCO₃ solution (10 mL) and stirring for 20 min. Extracted with DCM (2×10 mL), dried combined organics over sodium sulfate and concentrated under vacuum. The resulting residue was chromatographed over 12 g silica gel using a 0 to 50% EtOAc/Hex gradient as eluant. 139 mg obtained, 76% yield.

MS: m/z (obs.): 332 [M+H]⁺

1H NMR (300 MHz, CDCl3) δ 7.39 (d, J=1.8 Hz, 1H), 6.94 (s, 1H), 6.61 (s, 1H), 6.19 (d, J=1.7 Hz, 1H), 4.41 (d, J=5.7 Hz, 2H), 3.85 (s, 3H), 3.79 (s, 3H), 1.30 (s, 9H).

### Step 2. Methyl 5-(3,3-dimethylbut-1-ynyl)-3-((trans)-4-methyl-N-((1-methyl-1H-pyrazol-5-yl)methyl)cyclohexanecarboxamido)thiophene-2-carboxylate

To a mixture of methyl 5-(3,3-dimethylbut-1-ynyl)-3-((1-methyl-1H-pyrazol-5-yl)methylamino)thiophene-2-carboxylate (136 mg, 0.41 mmol) in DCE (4 mL) was added pyridine (330 μL, 4.1 mmol), N,N-dimethylaminopyridine (5 mg, 0.04 mmol), and 4-methylcyclohexanecarbonyl chloride (330 mg, 2.05 mmol). The mixture was reflux for 24 h and monitored with LC/MS. The reaction was cooled to room temperature and diluted with ethyl acetate (30 mL), washed with water (15 mL) and saturated aq. NaHCO₃ solution (15 mL). The organic phase was concentrated under vacuum and the resulting residue was chromatographed over 12 g silica gel using 0 to 40% EtOAc/hex as eluant. 174 mg obtained, 93% yield.

MS: m/z (obs.): 456 [M+H]⁺

1H NMR (300 MHz, CDCl3) δ 7.29 (t, J=3.5 Hz, 1H), 6.60 (s, 1H), 5.81 (d, J=1.8 Hz, 1H), 5.02 (d, J=15.2 Hz, 1H), 4.73 (d, J=15.2 Hz, 1H), 3.83 (s, 3H), 3.74 (s, 3H), 2.03 (d, J=6.6 Hz, 1H), 1.71-1.52 (m, 6H), 1.58-1.38 (m, 1H), 1.34-1.21 (m, 10H), 0.87-0.60 (m, 4H).

### Step 3. 5-(3,3-Dimethylbut-1-ynyl)-3-((trans)-4-methyl-N-((1-methyl-1H-pyrazol-5-yl)methyl)cyclohexanecarboxamido)thiophene-2-carboxylic acid

To a solution of methyl 5-(3,3-dimethylbut-1-ynyl)-3-((trans)-4-methyl-N-((1-methyl-1H-pyrazol-5-yl)methyl)cyclohexanecarboxamido)thiophene-2-carboxylate (174 mg, 0.38 mmol) in methanol (5 mL) was added 1M sodium hydroxide (1.9 mL) and allowed to stir overnight. The starting material was consumed by LC/MS. The reaction mixture was acidified to pH˜6 with 1N HCl and extracted with dichloromethane (2×20 mL). The combined organics were dried over sodium sulfate and concentrated under vacuum. The resulting solid was diluted with EtOAc (˜1 mL) and added hexanes dropwise until product precipitated. The white solid was filtered to give 123 mg, 68% yield.

MS: m/z (obs.): 442 [M+H]⁺;

1H NMR (300 MHz, CDCl3) d 11.02 (s, 1H), 7.27 (d, J=2.1 Hz, 1H), 6.87 (s, 1H), 5.81-5.57 (m, J=9.2 Hz, 2H), 4.04 (d, J=15.0 Hz, 1H), 3.91 (s, 3H), 2.30-2.00 (m, 1H), 1.81 (d, J=11.1 Hz, 1H), 1.76-1.43 (m, 5H), 1.43-1.16 (m, 10H), 0.93-0.54 (m, 5H).

### Preparation of Compound 80

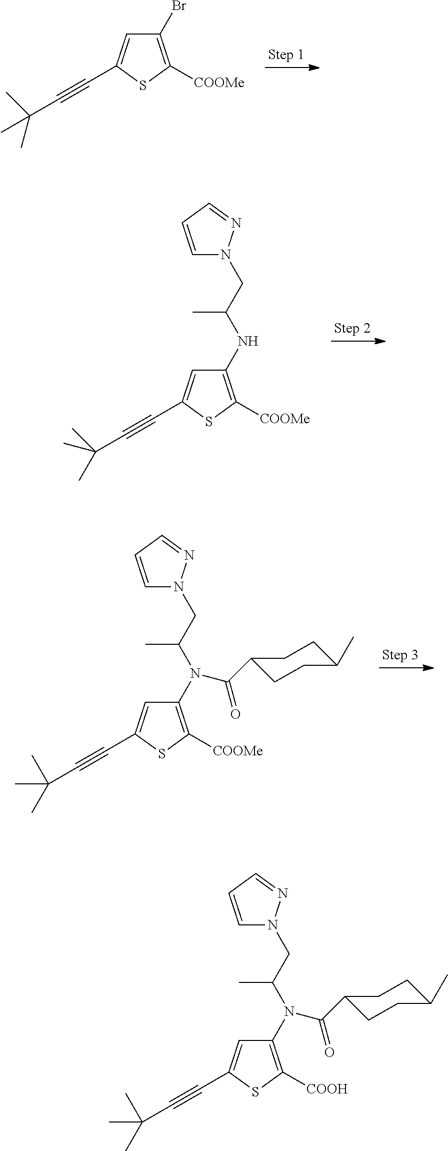

### Step 1. Methyl 5-(3,3-dimethylbut-1-ynyl)-3-[(1-methyl-2-pyrazol-1-yl-ethyl)amino]thiophene-2-carboxylate

Methyl 3-bromo-5-(3,3-dimethylbut-1-ynyl)thiophene-2-carboxylate (430 mg, 1.43 mmol), cesium carbonate (1.39 g, 4.28 mmol), and 1-pyrazol-1-ylpropan-2-amine (215 mg, 1.71 mmol) were taken into 2 mL of anhydrous 1,4-dioxane that was degassed with argon. After 15 mins., S-PHOS (dicyclohexyl-[2-(2,6-dimethoxyphenyl) phenyl]phosphane (117 mg, 0.29 mmol)) and Pd₂dba₃ (131 mg, 0.14 mmol) were added and mixture degassed for 5 more minutes then sealed and heated to 100° C. for 16 hours. LC/MS of reaction mixture confirmed formation of product. The reaction mixture was diluted with ethyl acetate and washed with water and brine. The organic layer was dried over anhydrous sodium sulfate, filtered, and evaporated to afford the crude product. This was purified by silica gel column chromatography eluting with a gradient of hexane to 70% ethyl acetate in hexane. Evaporation of the desired fractions afforded 291 mg of the title compound. Yield 291 mg, 59%_{.}

MS: m/z (obs): 346.04 [M+H]⁺

1H NMR (300 MHz, CDCl3) δ 7.57 (d, J=1.7 Hz, 1H), 7.36 (d, J=2.1 Hz, 1H), 6.49 (s, 1H), 6.23-6.20 (m, 1H), 4.19 (d, J=6.1 Hz, 2H), 4.05-3.91 (m, 2H), 3.82 (s, 3H), 1.31 (d, J=4.1 Hz, 9H), 1.23 (d, J=6.6 Hz, 3H).

### Step 2. Methyl 5-(3,3-dimethylbut-1-ynyl)-3-[(trans-4-methylcyclohexanecarbonyl)-(1-methyl-2-pyrazol-1-yl-ethyl)amino]thiophene-2-carboxylate

Methyl 5-(3,3-dimethylbut-1-ynyl)-3-[(1-methyl-2-pyrazol-1-yl-ethyl)amino]thiophene-2-carboxylate (285 mg, 0.83 mmol) was taken into 1,2-dichloroethane (5 mL) and pyridine (170 mL, 2.06 mmol). 4-trans-Methylcyclohexanecarbonyl chloride (331 mg, 2.06 mmol) was added to the solution at room temperature. After addition, the reaction was heated in a sealed tube at 110 C overnight. The reaction was cooled to room temperature. Triethylamine (2 mL) was added to the mixture followed by the addition of 1 mL of methanol. The mixture was stirred at room temperature for 1 hour then evaporated in vacuo. The crude gum was dissolved in dichloromethane and absorbed onto SiO2. This was purified by silica gel column chromatography eluting with a gradient of hexanes to 100% ethyl acetate. The desired fractions were combined and evaporated in vacuo to afford the desired product as a clear oil that crystallized upon setting at room temperature. Yield 150 mg, 38.7%.

### Step 3. 5-(3,3-Dimethylbut-1-ynyl)-3-[(4-trans-methylcyclohexanecarbonyl)-(1-methyl-2-pyrazol-1-yl-ethyl)amino]thiophene-2-carboxylic acid

Title compound was prepared by the hydrolysis of methyl 5-(3,3-dimethylbut-1-ynyl)-3-[(4-trans-methylcyclohexanecarbonyl)-(1-methyl-2-pyrazol-1-yl-ethyl)amino]thiophene-2-carboxylate (170 mg, 0.3620 mmol) as described for the preparation of Compound 29. Yield 157 mg, 90%.

MS: m/z (obs): 456.26 [M+H]⁺;

1H NMR (300 MHz, CDCl3) δ 7.65-7.48 (m, 2H), 6.90 & 6.40 (2×s, 1H), 6.29 (dd, J=5.3, 2.2 Hz, 1H), 4.98-4.76 (m, 1H), 4.59-4.40 (m, 1.5H), 4.10 (dd, J=13.5, 7.6 Hz, 0.5H), 1.96 (q, J=11.6 Hz, 1H), 1.76-1.39 (m, 6H), 1.34 (2×s, 9H), 1.24 (d, J=11.1 Hz, 1H), 1.15 & 1.05 (2×d, J=6.9 Hz, 3H), 0.84-0.73 (m, 3H), 0.73-0.51 (m, 2H).

### Preparation of Compound 79

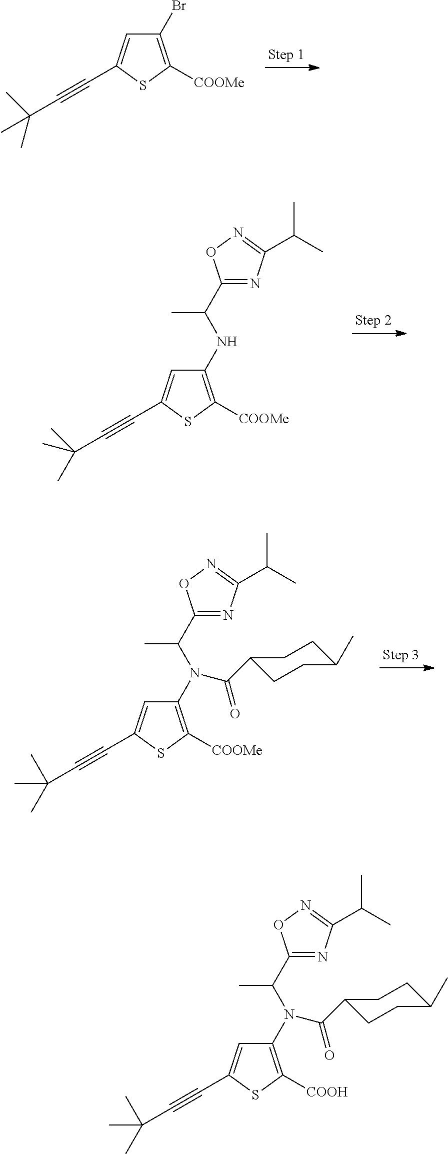

### Step 1. Methyl 5-(3,3-dimethylbut-1-ynyl)-3-[(1-(3-isopropyl-1,2,4-oxadiazol-5-yl)ethylamino]thiophene-2-carboxylate

Methyl 3-bromo-5-(3,3-dimethylbut-1-ynyl)thiophene-2-carboxylate (373 mg, 1.24 mmol), 1-(3-isopropyl-1,2,4-oxadiazol-5-yl)ethanamine (400 mg, 1.49 mmol), and cesium carbonate (1.61 g, 4.95 mmol) were taken into 12 mL of anhydrous dioxane and degassed with argon. After 30 mins, S-PHOS (dicyclohexyl-[2-(2,6-dimethoxyphenyl)phenyl]phosphane (102 mg, 0.25 mmol)) and Pd₂dba₃ (113 mg, 0.12 mmol) were added and the reaction degassed for 5 minutes, then sealed and heated at 100° C. overnight. The reaction was diluted with ethyl acetate, washed with water and brine, dried over anhydrous sodium sulfate, filtered and evaporated in vacuo to afford a brown residue. This was purified by silica gel column chromatography eluting with a gradient of hexane to ethyl acetate to afford 278 mg of the desired product. NMR confirms the structure.

1H NMR (300 MHz, CDCl3) δ 7.14 (t, J=10.0 Hz, 1H), 6.64 (s, 1H), 4.85 (dq, J=14.1, 7.0 Hz, 1H), 3.83 (s, 3H), 3.09 (dq, J=13.9, 7.0 Hz, 1H), 1.73 (d, J=7.0 Hz, 3H), 1.35 (d, J=7.0 Hz, 6H), 1.31 (s, 9H).

### Step 2. Methyl 5-(3,3-dimethylbut-1-ynyl)-3-[(1-(3-isopropyl-1,2,4-oxadiazol-5-yl)ethyl-(4-trans-methylcyclohexanecarbonyl)amino]thiophene-2-carboxylate

Methyl 5-(3,3-dimethylbut-1-ynyl)-3-[1-(3-isopropyl-1,2,4-oxadiazol-5-yl)ethylamino]thiophene-2-carboxylate (265 mg, 0.7058 mmol) was taken into 1,2-dichloroethane (5 mL) and pyridine (150 μL, 1.77 mmol). 4-trans-Methyl-cyclohexanecarbonyl chloride (283 mg, 1.76 mmol) was added to the mixture and heated at 100° C. in a sealed tube overnight.

Reaction was cooled to room temperature and diluted with dichloromethane. The solution was washed with saturated sodium bicarbonate, water, and brine and dried over anhydrous sodium sulfate, and evaporated in vacuo to afford the crude product. This was purified by silica gel column chromatography eluting with a gradient of hexane to 100% ethyl acetate. The desired fractions were combined and evaporated in vacuo to afford a clear viscous gum.

wt. 191 mg, 54%

MS: m/z (obs): 500.28 [M+H]⁺.

### Step 3. 5-(3,3-Dimethylbut-1-ynyl)-3-[(1-(3-isopropyl-1,2,4-oxadiazol-5-yl)ethyl-(4-trans-methylcyclohexanecarbonyl)amino]thiophene-2-carboxylic acid

The title compound was prepared by the hydrolysis of methyl 5-(3,3-dimethylbut-1-ynyl)-3-[1-(3-isopropyl-1,2,4-oxadiazol-5-yl)ethyl-(4-trans-methylcyclohexanecarbonyl)amino]thiophene-2-carboxylate (191 mg, 0.38 mmol) as described for the preparation of Compound 29. Yield 171 mg, 87.5%.

MS: m/z (obs): 486.27 [M+H]⁺.

1H NMR (300 MHz, CDCl3) δ 7.03 & 6.89 (2×s, 1H), 6.07 (q, J=7.3 Hz) & 5.75 (q, J=7.0 Hz) (2×q, 1H), 3.21-2.97 (m, 1H), 2.08 (dd, J=14.8, 7.7 Hz, 1H), 1.79-1.52 (m, 8H), 1.45-1.24 (m, 18H), 0.82 (d, J=6.5 Hz, 3H), 0.71 (m, 1H).

### Preparation of Compound 78

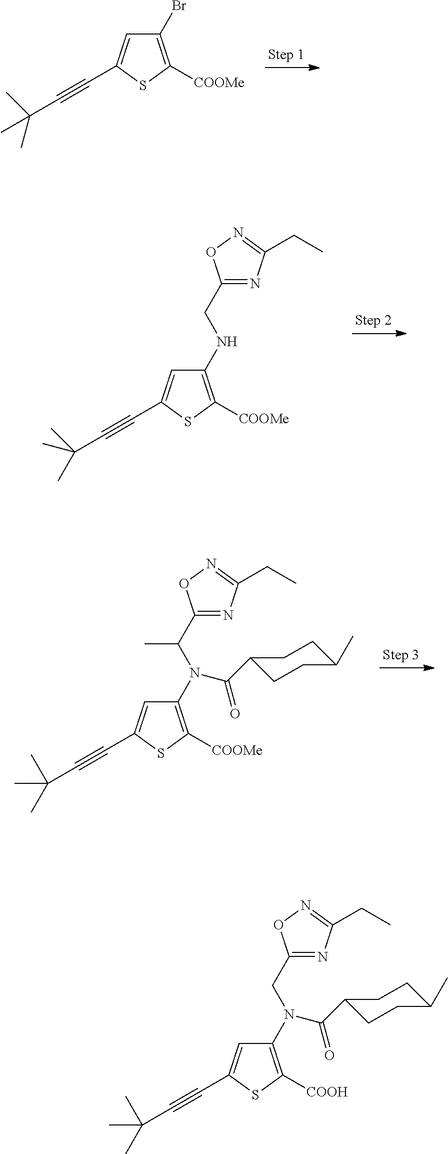

### Step 1. Methyl 5-(3,3-dimethylbut-1-ynyl)-3-[(3-ethyl-1,2,4-oxadiazol-5-yl)methylamino]thiophene-2-carboxylate

Methyl 3-bromo-5-(3,3-dimethylbut-1-ynyl)thiophene-2-carboxylate (400 mg, 1.33 mmol), (3-ethyl-1,2,4-oxadiazol-5-yl)methanamine (261 mg, 1.59 mmol), and cesium carbonate (1.73 g, 5.31 mmol) were taken into 12 ml of anhydrous dioxane and degassed with argon. After 30 minutes, S-PHOS (dicyclohexyl-[2-(2,6-dimethoxyphenyl)phenyl]phosphane (109.0 mg, 0.2656 mmol)) and Pd₂dba₃ (121.6 mg, 0.1328 mmol) were added to the mixture and degassed for an additional 10 mins. The reaction was sealed and heated at 100° C. overnight. The reaction was diluted with ethyl acetate, washed with water and brine, dried over anhydrous sodium sulfate, filtered, and evaporated to afford the crude product. This was purified by silica gel column chromatography eluting with a gradient of hexane to 100% ethyl acetate to afford the 89 mg of the desired product. Yield 89 mg, 19.3%.

MS: m/z (obs): 348.07 [M+H]⁺.

1H NMR (300 MHz, CDCl3) δ 6.57 (s, 1H), 4.55 (d, J=6.5 Hz, 2H), 3.75 (s, 3H), 2.70 (q, J=7.6 Hz, 2H), 1.26 (t, J=6.0 Hz, 3H), 1.23 (s, 9H).

### Step 2. Methyl 5-(3,3-dimethylbut-1-ynyl)-3-[(3-ethyl-1,2,4-oxadiazol-5-yl)methyl-(4-methylcyclohexanecarbonyl)amino]thiophene-2-carboxylate

Methyl 5-(3,3-dimethylbut-1-ynyl)-3-[(3-ethyl-1,2,4-oxadiazol-5-yl)methylamino]thiophene-2-carboxylate (86 mg, 0.25 mmol) was taken into 1,2-dichloroethane (4 mL) and pyridine (50 μL, 0.62 mmol). 4-trans-Methyl-cyclohexanecarbonyl chloride (100 mg, 0.62 mmol) was added to the mixture and heated at 100° C. in a sealed tube. The reaction was diluted with dichloromethane, washed with saturated sodium bicarbonate, water and brine. The organic layer was dried over anhydrous sodium sulfate, filtered, and evaporated in vacuo to afford a golden oil. This was purified by silica gel column chromatography eluting with a gradient of hexane to 100% ethyl acetate. The desired fractions were combined and evaporated in vacuo to afford 40 mg of the title compound. Yield 40 mg, 34%. Product was used without further characterization.

MS: m/z (obs): 472.25 [M+H]⁺.

### Step 3. 5-(3,3-dimethylbut-1-ynyl)-3-[(3-ethyl-1,2,4-oxadiazol-5-yl)methyl-(4-trans-methylcyclohexanecarbonyl)amino]thiophene-2-carboxylic acid

The title compound was prepared by the hydrolysis of methyl 5-(3,3-dimethylbut-1-ynyl)-3-[(3-ethyl-1,2,4-oxadiazol-5-yl)methyl-(4-trans-methylcyclohexanecarbonyl)amino]thiophene-2-carboxylate (90 mg, 0.1908 mmol) as described for the preparation of Compound 29. Yield 77 mg, 84%.

MS: m/z (obs): 458.24 [M+H]⁺.

1H NMR (300 MHz, CDCl3) δ 7.09 (s, 1H), 5.44 (d, J=17.0 Hz, 1H), 4.66 (d, J=17.0 Hz, 1H), 2.77 (q, J=7.6 Hz, 2H), 2.19 (dd, J=15.7, 7.4 Hz, 1H), 1.84-1.43 (m, 8H), 1.35 (s, 9H), 1.33-1.27 (t, 3H), 0.84 (d, J=6.5 Hz, 3H), 0.75 (m, 1H).

### Preparation of Compound 75

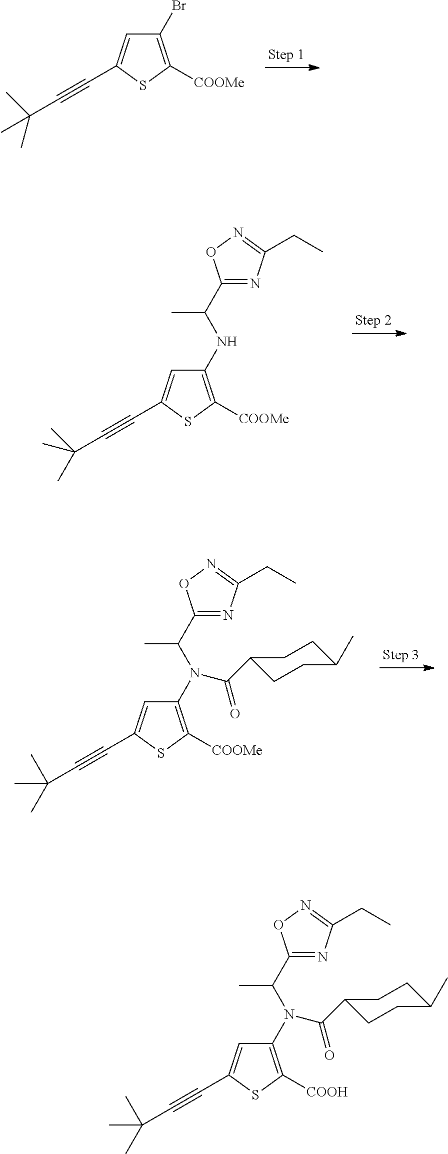

### Step 1. Methyl 5-(3,3-dimethylbut-1-ynyl)-3-[(1-(3-ethyl-1,2,4-oxadiazol-5-yl)ethylamino]thiophene-2-carboxylate

Methyl 3-bromo-5-(3,3-dimethylbut-1-ynyl)thiophene-2-carboxylate (393 mg, 1.30 mmol), 1-(3-ethyl-1,2,4-oxadiazol-5-yl)ethanamine (400 mg, 1.57 mmol), and cesium carbonate (1.70 g, 5.2 mmol) were taken into 10 mL of anhydrous dioxane and degassed with argon. After 30 minutes, S-PHOS (dicyclohexyl-[2-(2,6-dimethoxyphenyl)phenyl]phosphane (107 mg, 0.26 mmol)) and Pd₂dba₃ (119 mg, 0.13 mmol) were added to the mixture and degassed for an additional 5 minutes. The reaction was sealed and heated to 100° C. overnight. The reaction was diluted with ethyl acetate, washed with water and brine, dried over anhydrous sodium sulfate, and evaporated in vacuo to afford a crude brown semi-solid. This was purified by silica gel column chromatography eluting with a gradient of hexane to 100% ethyl acetate to afford the title compound as a crystalline solid. Yield 211 mg, 44.7%.

MS: m/z (obs): 362.00 [M+H]⁺.

1H NMR (300 MHz, CDCl3) δ 7.08 (d, J=7.9 Hz, 1H), 6.53 (s, 1H), 4.82-4.66 (m, 1H), 3.78-3.69 (m, 3H), 2.69 (q, J=7.6 Hz, 2H), 1.64 (d, J=7.0 Hz, 3H), 1.26 (t, J=6.3 Hz, 3H), 1.22 (s, 9H).

### Step 2. Methyl 5-(3,3-dimethylbut-1-ynyl)-3-[(1-(3-ethyl-1,2,4-oxadiazol-5-yl)ethyl-(4-trans-methylcyclohexanecarbonyl)amino]thiophene-2-carboxylate

Methyl 5-(3,3-dimethylbut-1-ynyl)-3-[1-(3-ethyl-1,2,4-oxadiazol-5-yl)ethylamino]thiophene-2-carboxylate (207 mg, 0.57 mmol) was taken into 1,2-dichloroethane (5 mL) and pyridine (115 μL, 1.43 mmol). 4-trans-Methyl-cyclohexanecarbonyl chloride (230 mg, 1.43 mmol) was added to the mixture and heated at 100° C. in a sealed tube overnight. The reaction was evaporated in vacuo. The crude was purified by silica gel column chromatography eluting with a gradient of hexane to 100% ethyl acetate. The desired fractions were combined and evaporated in vacuo to afford the title compound. Yield 125 mg, 45%.

MS: m/z (obs): 486.26 [M+H]⁺.

### Step 3. 5-(3,3-Dimethylbut-1-ynyl)-3-[(1-(3-ethyl-1,2,4-oxadiazol-5-yl)ethyl-(4-trans-methylcyclohexanecarbonyl)amino]thiophene-2-carboxylic acid

The title compound was prepared by the hydrolysis of methyl 5-(3,3-dimethylbut-1-ynyl)-3-[1-(3-ethyl-1,2,4-oxadiazol-5-yl)ethyl-(4-trans-methylcyclohexanecarbonyl)amino]thiophene-2-carboxylate (120 mg, 0.2471 mmol as described for the preparation of Compound 29. Yield 111 mg, 90.5%.

MS: m/z (obs): 472.32 [M+H]⁺.

1H NMR (300 MHz, CDCl3) d 7.05 (s, 0.33H), 6.94 (s, 0.67H), 6.05 (q, J=7.3 Hz, 0.67H), 5.73-5.58 (q, 0.33H), 2.87-2.65 (2×q, 2H), 2.07 (m, 1H), 1.80-1.56 (m, 6H), 1.48-1.18 (m & 2×s, 16H), 0.82 (2×d, 3H). 0.74 (m, 2H)

### Preparation of Compound 73

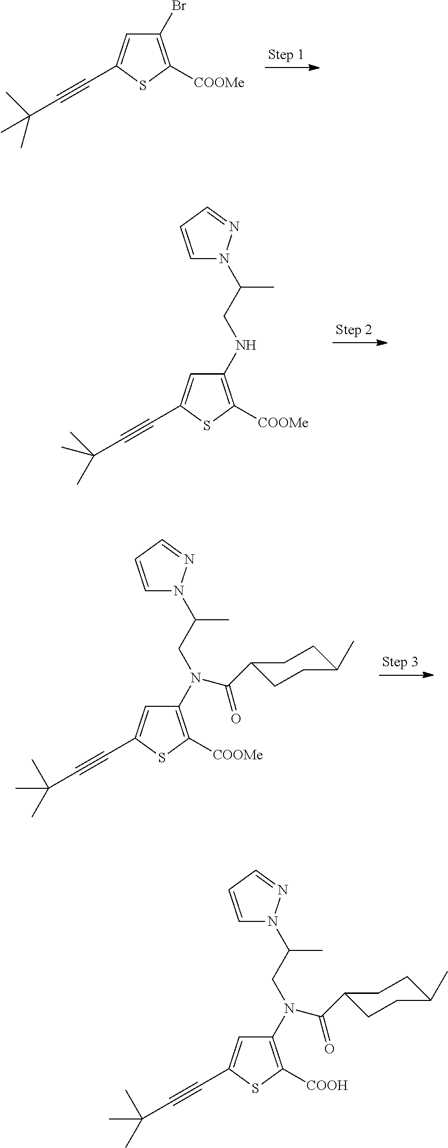

### Step 1. Methyl 5-(3,3-dimethylbut-1-ynyl)-3-(2-pyrazol-1-ylpropylamino)thiophene-2-carboxylate

Methyl 3-bromo-5-(3,3-dimethylbut-1-ynyl)thiophene-2-carboxylate (400 mg, 1.33 mmol), 2-pyrazol-1-ylpropan-1-amine (200 mg, 1.59 mmol), and cesium carbonate (1.08 g, 3.32 mmol) were taken into anhydrous 1,4-dioxane (10 mL) and degassed with argon. After 30 minutes, S-PHOS (dicyclohexyl-[2-(2,6-dimethoxyphenyl)phenyl]phosphane) (109.0 mg, 0.2656 mmol) and Pd₂dba₃ (122 mg, 0.13 mmol) were added and mixture degassed for an additional 5 minutes then sealed and heated to 100° C. for 16 hours. The reaction was diluted with ethyl acetate, washed with water, saturated sodium bicarbonate, and brine, dried over anhydrous sodium sulfate, and evaporated in vacuo to afford a golden colored gum. This was purified by column chromatography eluting with a gradient of hexane to 100% ethyl acetate to afford the title compound. Yield 301 mg, 65.6%.

MS: m/z (obs): 346.04 [M+H]⁺.

1H NMR (300 MHz, CDCl3) δ 7.59 (d, J=1.7 Hz, 0H), 7.39 (d, J=2.1 Hz, 0H), 6.49 (s, 0H), 6.24 (t, J=2.1 Hz, 0H), 4.57-4.42 (m, 0H), 3.78 (s, 0H), 3.73-3.46 (m, 0H), 1.61 (d, J=6.9 Hz, 0H), 1.32 (s, 1H).

### Step 2. Methyl 5-(3,3-dimethylbut-1-ynyl)-3-[(4-trans-methylcyclohexanecarbonyl)-(2-pyrazol-1-ylpropyl)amino]thiophene-2-carboxylate

Methyl 5-(3,3-dimethylbut-1-ynyl)-3-(2-pyrazol-1-ylpropylamino)thiophene-2-carboxylate (240 mg, 0.69 mmol) was taken into pyridine (110 μL, 1.38 mmol) and 1,2-dichloroethane (5 mL). 4-trans-Methylcyclohexanecarbonyl chloride (279 mg, 1.73 mmol) was added to the mixture and was heated at 90° C. overnight in a sealed tube. The reaction was cooled to room temperature. Methanol was added to the reaction and the reaction was evaporated in vacuo. The resulting crude product was purified by column chromatography eluting with a gradient of hexanes to 70% ethyl acetate, Evaporation of the desired fractions afforded the title product. Yield 310 mg, 95%.

MS: m/z (obs): 470.27 [M+H]⁺.

### Step 3. 5-(3,3-Dimethylbut-1-ynyl)-3-[(4-trans-methylcyclohexanecarbonyl)-(2-pyrazol-1-ylpropyl)amino]thiophene-2-carboxylic acid

The title compound was prepared by the hydrolysis of methyl 5-(3,3-dimethylbut-1-ynyl)-3-[(4-trans-methylcyclohexanecarbonyl)-(2-pyrazol-1-ylpropyl)amino]thiophene-2-carboxylate (350 mg, 0.75 mmol) as described for the preparation of Compound 29. Yield 335 mg, 94%.

MS: m/z (obs): 456.26 [M+H]⁺.

1H NMR (300 MHz, CDCl3) d 7.48 (d, J=3.8 Hz, 0.7H), 7.40 (d, J=1.4 Hz, 0.3H), 7.35 (s, 0.3H), 6.76 (d, J=6.6 Hz, 0.7H), 6.24 & 6.15 (d, J=2.0 & J=1.9 Hz, 1H), 5.64 (d, J=6.8 Hz, 1H), 4.89 (s, 1H), 4.70-4.46 (m, 1H), 4.38 (m, 1H), 3.55 (d, J=8.9 Hz, 1H), 3.26 (dd, J=13.8, 9.8 Hz, 1H), 2.58 (t, J=6.6 Hz, 1H), 1.93 (d, J=45.9 Hz, 1H), 1.50 (m, 7H), 1.32 (2×s, & m, 10H), 1.28-1.05 (m, 3H), 0.84-0.46 (m, 2H).

### Preparation of Compound 74

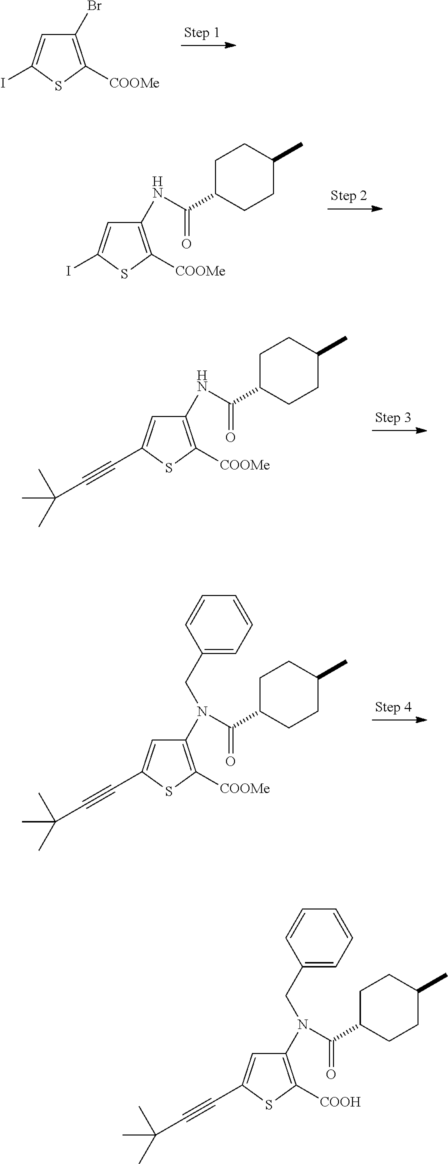

### Step 1. Methyl 5-iodo-3-[(4-methylcyclohexanecarbonyl)amino]thiophene-2-carboxylate

4-trans-Methylcyclohexanecarbonyl chloride (6.24 g, 38.9 mmol) was added dropwise to a cooled solution (0° C.) of methyl 3-amino-5-iodo-thiophene-2-carboxylate (10 g, 35.3 mmol) in dichloromethane (90 mL) and pyridine (6 mL). The reaction was warmed to room temperature and stirred overnight. The reaction mixture was washed with water, 1N HCl, and brine. The organic layer was dried over anhydrous sodium sulfate and concentrated in vacuo to provide the title compound as a pale yellow solid. Yield 14.45 g, 99%.

MS: m/z (obs): 408.14 [M+H]⁺.

### Step 2. Methyl 5-(3,3-dimethylbut-1-ynyl)-3-[(4-trans-methylcyclohexanecarbonyl)amino]thiophene-2-carboxylate

Methyl 5-iodo-3-[(4-trans-methylcyclohexanecarbonyl)amino]thiophene-2-carboxylate (14.4 g, 35.4 mmol) and N,N-diisopropylamine (7.5 mL, 53 mmol) were taken into 100 mL of dioxane and degassed by bubbling nitrogen into the mixture for 10 minutes.

Bis(triphenylphosphine)palladium(II)dichloride (995.3 mg, 1.414 mmol) and copper(I)iodide (269.3 mg, 1.414 mmol) was added to the mixture followed by the dropwise addition of a solution of 3,3-dimethylbut-1-yne (3.195 g, 38.9 mmol) in 40 mL 1,4-dioxane at room temperature. The reaction was stirred at room temperature for 60 hours. The reaction was diluted with ethyl acetate and washed with water and 1N HCl. The organic layer was dried over anhydrous sodium sulfate and concentrated in vacuo to afford a curde yellow solid. This was purified by silica gel column chromatography eluting with a gradient of 5-20% ethyl acetate in hexanes. The desired fractions were combined and evaporated in vacuo to afford an off-white solid. Yield 9 g, 70%.

MS: m/z (obs): 362.33 [M+H]⁺.

### Step 3. Methyl 3-[benzyl-(4-trans-methylcyclohexanecarbonyl)amino]-5-(3,3-dimethylbut-1-ynyl)thiophene-2-carboxylate

Methyl 5-(3,3-dimethylbut-1-ynyl)-3-[(4-trans-methylcyclohexane carbonyl)amino]thiophene-2-carboxylate (100 mg, 0.28 mmol) was taken into anhydrous THF (10 mL) and cooled to 0° C. under argon followed by the addition of 60% sodium hydride (11 mg, 0.28 mmol) in mineral oil. The reaction was stirred for 30 minutes followed by the addition of benzyl chloride (35 μL, 0.30 mmol). After 1 hour, the reaction was heated at 50 C overnight. The reaction was diluted with ethyl acetate and washed with water and brine. The organic layer was dried over anhydrous sodium sulfate, filtered, and evaporated in vacuo to afford the crude product. This was purified by silica gel column chromatography eluting with a gradient of hexane to 50% ethyl acetate. The desired fractions were evaporated to afford 49 mg of the title compound as a foam. Yield 49.6 mg, 39.7%.

MS: m/z (obs): 452.0 [M+H]⁺.

1H NMR (300 MHz, CDCl3) δ 7.28-7.24 (m, 3H), 7.19-7.16 (m, 2H), 6.64 (s, 1H), 5.12 (d, 1H), 4.47 (d, 1H), 3.72 (s, 3H), 2.10-2.02 (m, 1H), 1.69-1.48 (m, 6H), 1.32 (s, 9H), 1.31-1.28 (m, 1H), 0.83 (d, 3H), 0.74-0.61 (m, 2H).

### Step 4. 3-[Benzyl-(4-trans-methylcyclohexanecarbonyl)amino]-5-(3,3-dimethylbut-1-ynyl)thiophene-2-carboxylic acid

The title compound was prepared by the hydrolysis of methyl 3-[benzyl-(4-methylcyclohexanecarbonyl)amino]-5-(3,3-dimethylbut-1-ynyl)thiophene-2-carboxylate (49 mg, 0.108 mmol) as described for the preparation of Compound 29. Yield 41 mg, 83%.

MS: m/z (obs): 438.28 [M+H]⁺

1H NMR (300 MHz, CDCl3) δ 7.29-7.24 (m, 3H), 7.19 (d, J=7.7 Hz, 2H), 6.60 (s, 1H), 5.37 (d, J=14.9 Hz, 1H), 4.30 (d, J=14.2 Hz, 1H), 2.11 (s, 1H), 1.78-1.46 (m, 7H), 1.32 (s, 9H), 0.83 (d, J=6.5 Hz, 3H), 0.75 (d, J=12.1 Hz, 2H).

### Preparation of Compound 48

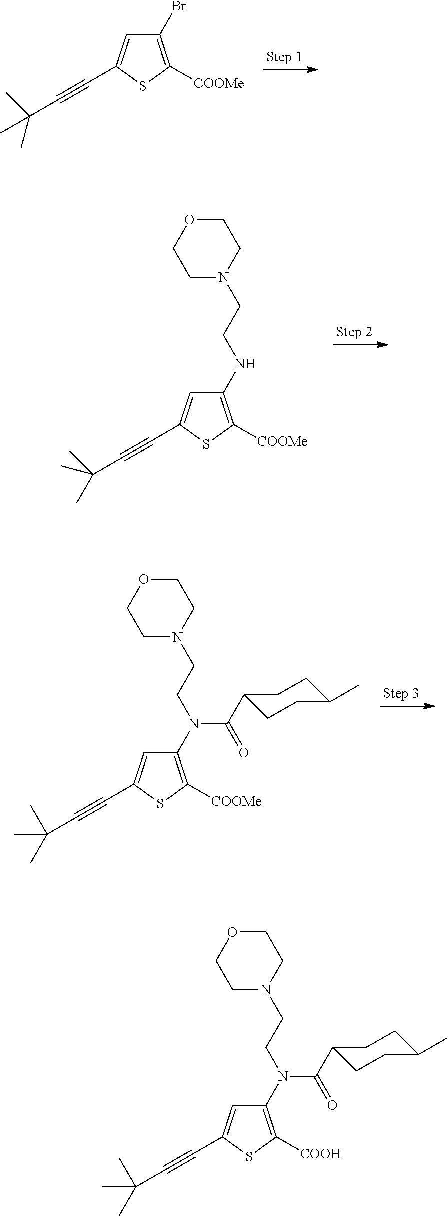

### Step 1. Methyl 5-(3,3-dimethylbut-1-yn-1-yl)-3-((2-morpholinoethyl)amino)thiophene-2-carboxylate

To methyl 3-bromo-5-(3,3-dimethylbut-1-ynyl)thiophene-2-carboxylate (900 mg, 3.0 mmol), 2-morpholinoethanamine (390 μL, 3.0 mmol) and cesium carbonate (2.92 g, 9.0 mmol) in a microwave tube was added degassed 1,4-dioxane (11 mL). Argon was bubbled into the solution for 5 minutes. Pd₂(dba)₃ (137 mg, 0.15 mmol) and S-Phos (dicyclohexyl-[2-(2,6-dimethoxyphenyl)phenyl]phosphane) (123 mg, 0.3 mmol) were added. The tube was sealed and stirred overnight at 90° C. The mixture was filtered and the filtrate concentrated then purified via silica gel chromatography, eluting with 10-100% EtOAc in hexanes over 20 minutes to give 220 mg of desired product as a yellow oil.

MS: m/z (obs.): 351.3 [M+H]⁺;

### Step 2. Methyl 5-(3,3-dimethylbut-1-yn-1-yl)-3-(trans-4-methyl-N-(2-morpholinoethyl)cyclohexanecarboxamido)thiophene-2-carboxylate

Prepared as described for compound 73.

MS: m/z (obs.): 475.4 [M+H]⁺;

### Step 3. 5-(3,3-Dimethylbut-1-yn-1-yl)-3-(trans-4-methyl-N-(2-morpholinoethyl)cyclohexanecarboxamido)thiophene-2-carboxylic acid

Prepared by hydrolysis as described for compound 29. Yield 97%.

MS: m/z (obs.): 461.0 [M+H]⁺;

1H NMR (300 MHz, DMSO) δ 7.22 (s, 1H), 4.60 (s, 1H), 3.87-2.80 (m, 12H), 2.14 (t, J=11.6 Hz, 1H), 1.73-1.00 (m, 16H), 0.77 (d, J=6.5 Hz, 5H).

### Preparation of Compound 57

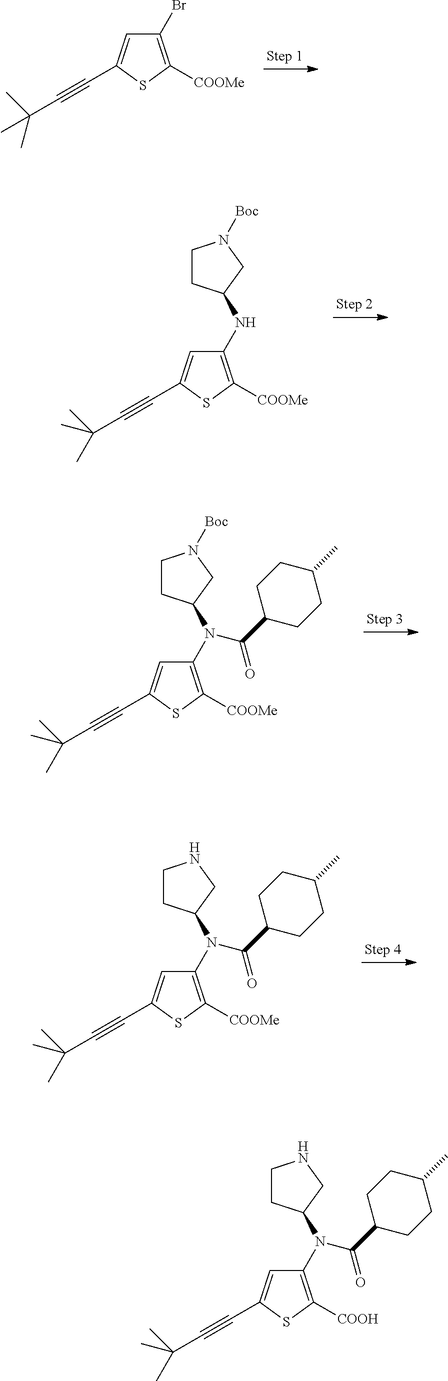

### Step 1. (S)-tert-Butyl 3-((5-(3,3-dimethylbut-1-yn-1-yl)-2-(methoxycarbonyl)thiophen-3-yl)amino)pyrrolidine-1-carboxylate

Buchwald coupling of 3-bromo-5-(3,3-dimethylbut-1-ynyl)thiophene-2-carboxylate (1 g, 3.3 mmol) with (S)-tert-butyl 3-aminopyrrolidine-1-carboxylate (620 mg, 3.2 mmol) was performed as described for compound 48. Yield 1.11 g.

### Step 2. (S)-tert-Butyl 3-(-N-(5-(3,3-dimethylbut-1-yn-1-yl)-2-(methoxycarbonyl)thiophen-3-yl)-4-trans-methylcyclohexanecarboxamido)pyrrolidine-1-carboxylate

To (S)-tert-butyl 3-((5-(3,3-dimethylbut-1-yn-1-yl)-2-(methoxycarbonyl)thiophen-3-yl)amino)pyrrolidine-1-carboxylate (1.1 g, 2.7 mmol) and trans-4-methylcyclohexanecarbonyl chloride (1.15 g, 7.16 mmol) in dichloroethane was added pyridine (1.1 mL, 13.5 mmol). The reaction was heated overnight in a sealed tube at 90° C. LCMS confirms product plus some byproduct resulting from loss of N-Boc and acylation of pyrrolidine nitrogen. The reaction mixture was diluted with water and washed with 1 M HCl. The organic layer was dried and concentrated then purified via flash chromatography, eluting with 15-70% EtOAc in hexanes over 20 minutes. (Rf product 0.55 and byproduct 0.15 in 1:1 hex/EtOAc). Combined and concentrated pure fractions to give 900 mg of desired product as a white foam.

MS: m/z (obs.): 531.4 [M+H]⁺;

### Step 3. Methyl 5-(3,3-dimethylbut-1-yn-1-yl)-3-(trans-4-methyl-N-((S)-pyrrolidin-3-yl)cyclohexanecarboxamido)thiophene-2-carboxylate

To the product of step 2 (900 mg, 1.7 mmol) in dichloromethane (5 mL) was added trifluoroacetic acid (5 mL). The reaction was stirred for 30 minutes and solvent was removed. Diluted with DCM and washed 2× with saturated sodium bicarbonate. The organic layer was dried and concentrated to give 723 mg of desired product as an off-white solid.

MS: m/z (obs.): 431.3 [M+H]⁺;

### Step 4. 5-(3,3-Dimethylbut-1-yn-1-yl)-3-((trans)-4-methyl-N-((S)-pyrrolidin-3-yl)cyclohexanecarboxamido)thiophene-2-carboxylic acid

To methyl 5-(3,3-dimethylbut-1-yn-1-yl)-3-(trans-4-methyl-N-((S)-pyrrolidin-3-yl)cyclohexanecarboxamido)thiophene-2-carboxylate (100 mg, 0.23 mmol) in 3:1 tetrahydrofuran and water was added lithium hydroxide monohydrate (97 mg, 2.3 mmol). The reaction was stirred overnight at room temperature, acidified to pH ˜4 using 2 M HCl, then the organic solvent was removed. A white precipitate was filtered, dissolved in MeOH and dropped into stirring ether. The white solid was filtered again and dried overnight under vacuum at 50° C.

MS: m/z (obs.): 417.0 [M+H]⁺;

1H NMR DMSO-d6, 300 MHz: δ 7.35 (d, J=29.1 Hz, 1H), 4.79-4.50 (m, 1H), 3.53-2.86 (m, 7H), 2.20-1.96 (m, 1H), 1.94-1.03 (m, 16H), 0.75-0.52 (m, 4H).

### Preparation of Compound 58

Compound 58 (5-(3,3-Dimethylbut-1-yn-1-yl)-3-((trans)-4-methyl-N—((R)-pyrrolidin-3-yl)cyclohexanecarboxamido)thiophene-2-carboxylic acid) was prepared as described for compound 57.

MS: m/z (obs.): 417.0 [M+H]⁺;

1H NMR DMSO-d6, 300 MHz: δ 7.35 (d, J=29.1 Hz, 1H), 4.79-4.50 (m, 1H), 3.53-2.86 (m, 7H), 2.20-1.96 (m, 1H), 1.94-1.03 (m, 16H), 0.75-0.52 (m, 4H).

### Preparation of Compound 59

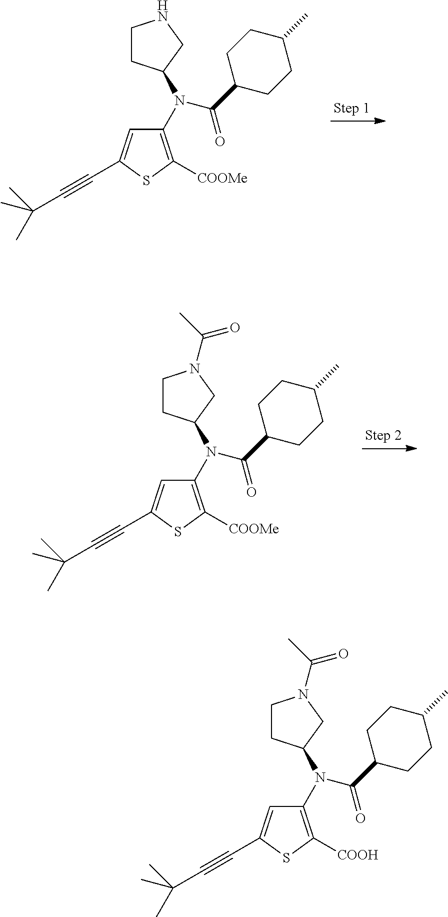

### Step 1. Methyl 3-(N—((S)-1-acetylpyrrolidin-3-yl)-4-trans-methylcyclohexanecarboxamido)-5-(3,3-dimethylbut-1-yn-1-yl)thiophene-2-carboxylate

To methyl 5-(3,3-dimethylbut-1-yn-1-yl)-3-(trans-4-methyl-N-((S)-pyrrolidin-3-yl)cyclohexanecarboxamido)thiophene-2-carboxylate (150 mg, 0.35 mmol) in N,N-dimethylformamide (2 mL) was added acetic anhydride (50 μL, 0.52 mmol) and pyridine (85 μL, 1.04 mmol). The reaction was stirred overnight, diluted with DCM and washed with 1 M HCl. The organic layer was dried, concentrated, and purified via flash chromatography (4 g ISCO column) eluting with 1-10% MeOH in DCM over 15 minutes. Pure fractions were combined and concentrated to give 140 mg of desired product as a pale yellow oil.

MS: m/z (obs.): 473.4 [M+H]⁺;

### Step 2. 3-(N—((S)-1-Acetylpyrrolidin-3-yl)-4-trans-methylcyclohexanecarboxamido)-5-(3,3-dimethylbut-1-yn-1-yl)thiophene-2-carboxylic acid

To methyl 3-(N—((S)-1-acetylpyrrolidin-3-yl)-4-trans-methylcyclohexanecarboxamido)-5-(3,3-dimethylbut-1-yn-1-yl)thiophene-2-carboxylate (145 mg, 0.31 mmol) in tetrahydrofuran and water was added lithium hydroxide monohydrate (64 mg, 1.53 mmol). The reaction was stirred overnight at room temperature then acidified using 1 M HCl. Solvent was removed and the resulting white ppt was filtered, washed 2× water, and dried overnight under vacuum at 50° C.

MS: m/z (obs.): 459.0 [M+H]⁺;

1H NMR DMSO-d6, 300 MHz: δ 7.31 (dd, J=9.9, 6.6 Hz, 1H), 4.95-4.72 (m, 1H), 3.30 (s, 7H), 2.22-1.10 (m, 19H), 0.70-0.52 (m, 4H).

### Preparation of Compound 60

Compound 60 (3-(N—((R)-1-Acetylpyrrolidin-3-yl)-4-trans-methylcyclohexane-carboxamido)-5-(3,3-dimethylbut-1-yn-1-yl)thiophene-2-carboxylic acid) was prepared as described for compound 59.

MS: m/z (obs.): 459.0 [M+H]⁺;

1H NMR DMSO-d6, 300 MHz: δ 7.31 (dd, J=9.9, 6.6 Hz, 1H), 4.95-4.72 (m, 1H), 3.30 (s, 7H), 2.22-1.10 (m, 19H), 0.70-0.52 (m, 4H).

### Preparation of Compound 63

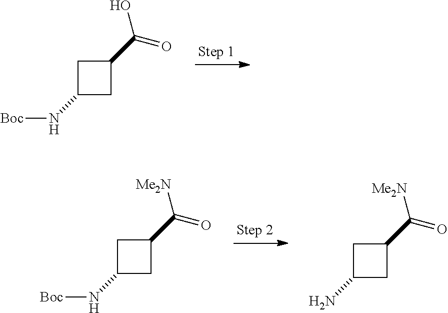 [SMILES: CC(C)(C)OC(=O)N[C@H]1C[C@H](C(=O)O)C1 ; CN(C)C(=O)[C@H]1C[C@H](NC(=O)OC(C)(C)C)C1 ; CN(C)C(=O)[C@H]1C[C@H](N)C1]

### Step 1. trans-(3-t-Butoxycarbonylamino)-cyclobutane carboxylic acid dimethylamide

To trans-(3-t-Butoxycarbonylamino)-cyclobutane carboxylic acid (500 mg, 2.32 mmol), EDC (535 mg, 2.79 mmol), and HOBt (377 mg, 2.79 mmol) was added a solution of dimethylamine (560 μL, 11.6 mmol) in THF. The reaction was diluted with 10 mL of DCM and stirred overnight at room temperature. The mixture was diluted with 10 mL DCM then washed with water, 1 M HCl, and saturated sodium bicarbonate. The organic layer was dried and concentrated to give 360 mg of desired product as a white solid.

### Step 2. trans-3-Aminocyclobutane carboxylic acid dimethylamide

To trans-(3-t-Butoxycarbonylamino)-cyclobutane carboxylic acid dimethylamide (360 mg, 2.14 mmol) was added 4M HCl in 1,4-dioxane (5 mL). The reaction was stirred for 3 h. The solvent was removed and the resulting material was dissolved in a minimal amount of MeOH then slowly dropped into stirring ether. A white precipitate formed and was filtered and dried overnight under vacuum at 50° C. to give desired product.

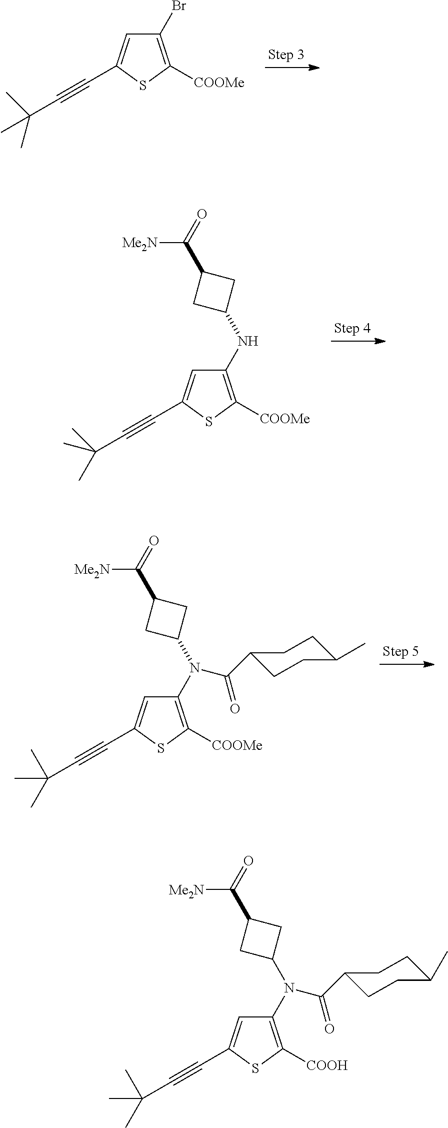

### Step 3. Methyl 5-(3,3-dimethylbut-1-yn-1-yl)-3-((trans-3-(dimethylcarbamoyl)-cyclobutyl)amino)thiophene-2-carboxylate

Coupling was performed as described in the preparation of compound 48 from 235 mg trans-3-aminocyclobutane carboxylic acid dimethylamide to give 285 mg of desired product as an orange solid.

MS: m/z (obs.): 363.2 [M+H]⁺;

### Step 4. Methyl 5-(3,3-dimethylbut-1-yn-1-yl)-3-(N-(3-trans-(dimethylcarbamoyl)-cyclobutyl)-4-trans-methylcyclohexanecarboxamido)thiophene-2-carboxylate

Acylation was performed as described in the preparation of compound 48. Product was purified via flash chromatography, eluting with 0-3% MeOH in DCM. Pure fractions were combined and concentrated to afford 314 mg of desired product as a light yellow oil.

MS: m/z (obs.): 487.3 [M+H]⁺;

### Step 5. 5-(3,3-Dimethylbut-1-yn-1-yl)-3-(N-(3-trans-(dimethylcarbamoyl)-cyclobutyl)-4-trans-methylcyclohexanecarboxamido)thiophene-2-carboxylic acid

Ester hydrolysis was performed as described in the preparation of compound 48.

MS: m/z (obs.): 473.0 [M+H]⁺;

1H NMR DMSO-d6, 300 MHz: δ 7.28 (s, 1H), 4.92-4.70 (m, 1H), 3.30 (s, 2H), 2.80 (d, J=8.3 Hz, 7H), 2.35-1.75 (m, 5H), 1.64-1.14 (m, 15H), 0.76 (d, J=6.5 Hz, 4H).

### Preparation of Compound 11

Compound 11 (5-(3,3-dimethylbut-1-yn-1-yl)-3-(trans-4-methyl-N-(cis-3-(dimethylcarbamoyl)cyclobutyl)cyclohexanecarboxamido)thiophene-2-carboxylic acid) was prepared as described for compound 63.

MS: m/z (obs.): 473.3 [M+H]⁺

1H NMR DMSO-d6, 300 MHz: δ 7.21 (s, 1H), 4.73 (s, 1H), 3.60 (s, 0.66H), 3.30 (s, 1.33H), 2.83 (t, J=29.7 Hz, 7H), 2.36-2.21 (m, 1H), 2.16-2.00 (m, 1H), 1.92-1.11 (m, 18H), 0.72-0.51 (m, 4H).

### Preparation of Compound 68

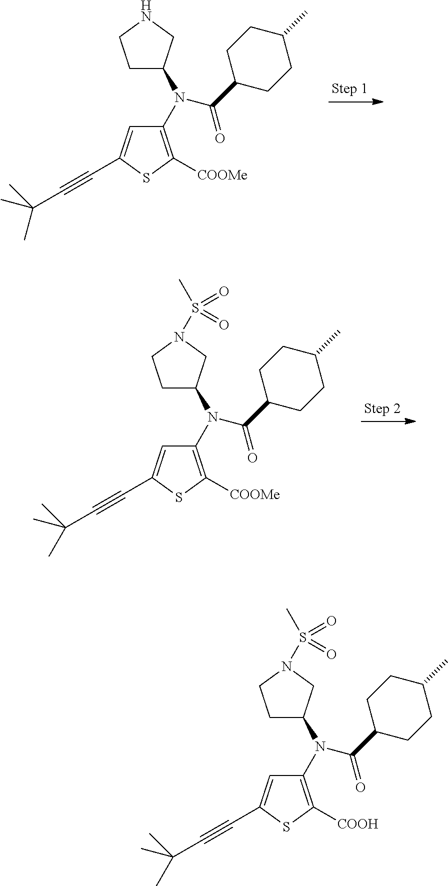

### Step 1. Methyl 5-(3,3-dimethylbut-1-yn-1-yl)-3-(trans-4-methyl-N-((S)-1-methylsulfonyl)pyrrolidin-3-yl)cyclohexanecarboxamido)thiophene-2-carboxylate

To methyl 5-(3,3-dimethylbut-1-yn-1-yl)-3-(trans-4-methyl-N-((S)-pyrrolidin-3-yl)cyclohexanecarboxamido)thiophene-2-carboxylate (100 mg, 0.23 mmol) in DCM (2 mL) with triethylamine (36 μL, 0.26 mmol) was added methanesulfonyl chloride (20 μL, 0.26 mmol). The reaction was stirred overnight at room temperature, washed with 1 M HCl, concentrated and purified via flash chromatography eluting with 25-60% EtOAc in hexanes. Pure fractions were combined and concentrated to give 54 mg of desired product as a colorless film.

MS: m/z (obs.): 509.2 [M+H]⁺

### Step 2. 5-(3,3-dimethylbut-1-yn-1-yl)-3-(trans-4-methyl-N-((S)-1-methylsulfonyl)pyrrolidin-3-yl)thiophene-2-carboxylic acid

To methyl 5-(3,3-dimethylbut-1-yn-1-yl)-3-(trans-4-methyl-N—((S)-1-methylsulfonyl)pyrrolidin-3-yl)cyclohexanecarboxamido)thiophene-2-carboxylate (54 mg, 0.11 mmol) in a 3:1 mixture of THF and water was added lithium hydroxide monohydrate (45 mg, 1.06 mmol). The reaction was stirred overnight at room temperature and concentrated to remove organic solvent. The resulting precipitate was filtered, washed with water, and dried overnight at 50° C. to obtain 20 mg of desired product.

MS: m/z (obs.): 495.2 [M+H]⁺

1H NMR DMSO-d6, 300 MHz: δ 7.36 (d, J=12.4 Hz, 1H), 4.96-4.67 (m, 1H), 3.59 (d, J=6.5 Hz, 2H), 3.22-2.98 (m, 2H), 2.87 (d, J=18.7, 12.4 Hz, 3H), 2.18-1.08 (m, 19H), 0.88-0.50 (m, 5H).

### Preparation of Compound 69

Compound (5-(3,3-dimethylbut-1-yn-1-yl)-3-(trans-4-methyl-N—((R)-1-methylsulfonyl)pyrrolidin-3-yl)thiophene-2-carboxylic acid) was prepared as described for compound 68.

MS: m/z (obs.): 495.2 [M+H]⁺

1H NMR DMSO-d6, 300 MHz: δ 7.36 (d, J=12.4 Hz, 1H), 4.96-4.67 (m, 1H), 3.59 (d, J=6.5 Hz, 2H), 3.22-2.98 (m, 2H), 2.87 (d, J=18.7, 12.4 Hz, 3H), 2.18-1.08 (m, 19H), 0.88-0.50 (m, 5H).

### Preparation of Compound 70

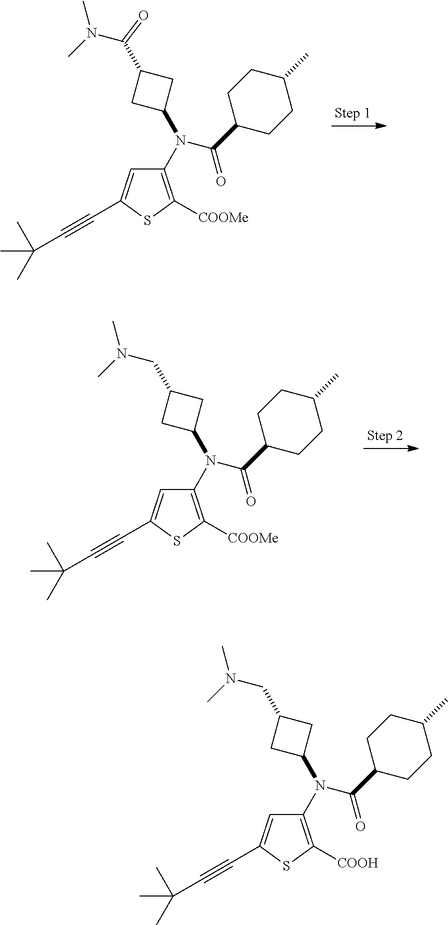

### Step 1: Methyl-5-(3,3-dimethylbut-1-yn-1-yl)-3-(trans-4-methyl-N-((trans-3-(dimethylamino)methyl)cyclobutyl)cyclohexanecarboxamido)thiophene-2 carboxylate

To Methyl-5-(3,3-dimethylbut-1-yn-1-yl)-3-(trans-4-methyl-N-(trans-3-(dimethylcarbamoyl)cyclobutyl)cyclohexanecarboxamido)thiophene-2 carboxylate in DCM (2 mL) at room temperature was added triflic anhydride (60 μL, 0.36 mmol). The mixture was stirred at room temperature for 5 minutes and then diethyl 2,6-dimethyl-1,4-dihydropyridine-3,5-dicarboxylate (225 μL, 0.82 mmol) was added. After stirring for 30 minutes, saturated sodium bicarbonate was added and the mixture was extracted with diethyl ether (2×5 mL). The organic layers were combined, concentrated, and purified via flash chromatography eluting with 1-20% ammonia/MeOH in DCM over 15 minutes. Pure fractions were combined and concentrated to give 73 mg of desired product as a light yellow oil.

MS: m/z (obs.): 473.3 [M+H]⁺

### Step 2: 5-(3,3-Dimethylbut-1-yn-1-yl)-3-(trans-4-methyl-N((trans-3-(dimethylamino)methyl)cyclobutyl)cyclohexanecarboxamido)thiophene-2 carboxylic acid

To methyl-5-(3,3-dimethylbut-1-yn-1-yl)-3-(trans-4-methyl-N-((trans-3-(dimethylamino)methyl)cyclobutyl)cyclohexanecarboxamido)thiophene-2 carboxylate (73 mg, 0.15 mmol) in a 3:1 mixture of THF and water (1 mL) was added lithium hydroxide monohydrate (65 mg, 1.54 mmol). The reaction was stirred overnight at room temperature and organic solvent was removed under reduced pressure. The resulting precipitate was filtered, washed with water, and dried under vacuum at 50° C. to afford 44 mg of desired product as a hydrochloride salt.

MS: m/z (obs.): 459.3 [M+H]⁺

1H NMR DMSO-d6, 300 MHz: δ 7.25 (d, J=25.5 Hz, 1H), 5.04-4.87 (m, 0.65H), 4.68-4.52 (m, 0.35H), 3.22 (d, J=7.7 Hz, 1H), 2.93 (d, J=6.2 Hz, 1H), 2.63 (d, J=11.1 Hz, 6H), 1.97 (dd, J=64.8, 9.0 Hz, 6H), 1.67-1.11 (m, 16H), 0.69 (dd, J=43.4, 9.1 Hz, 5H).

### Preparation of Compound 65

Compound 65 (Methyl-5-(3,3-dimethylbut-1-yn-1-yl)-3-(trans-4-methyl-N-(cis-3-((dimethylamino)methyl)cyclobutyl)cyclohexanecarboxamido)thiophene-2 carboxylate was prepared as described for compound 70.

MS: m/z (obs.): 459.3 [M+H]⁺

1H NMR DMSO-d6, 300 MHz: δ 7.21 (s, 1H), 4.71-4.51 (m, 1H), 3.34 (s, 2H), 2.93 (d, J=6.3 Hz, 2H), 2.62 (s, 6H), 2.31 (s, 3H), 1.85 (dd, J=22.9, 11.0 Hz, 1H), 1.67-1.08 (m, 16H), 0.69-0.51 (m, 5H).

### Preparation of Compound 76

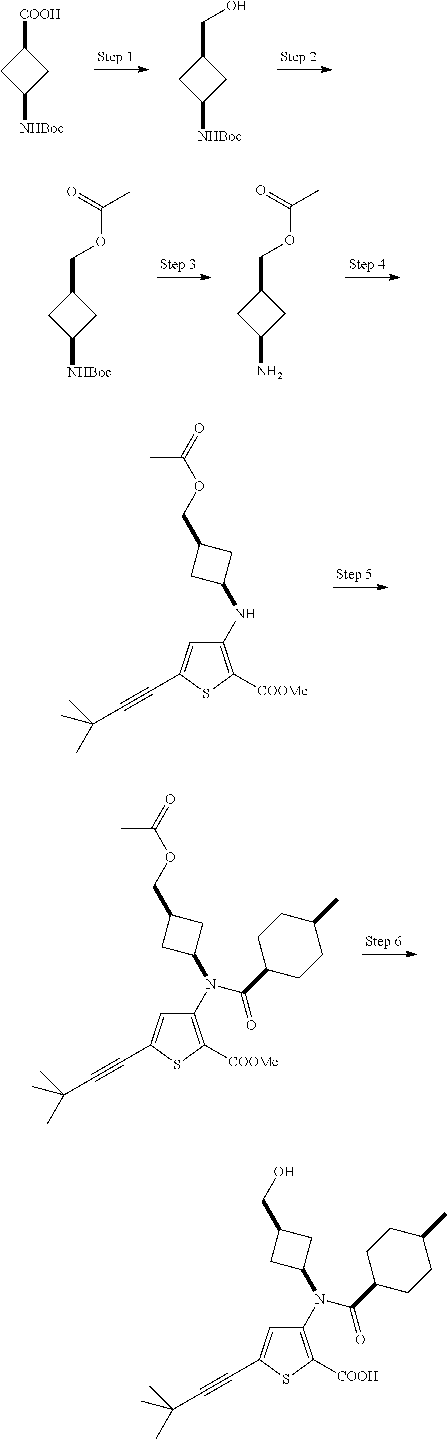

### Step 1. tert-Butyl cis-3-(hydroxymethyl)cyclobutylcarbamate

To cis-3-(tert-butoxycarbonylamino)cyclobutanecarboxylic acid (440 mg, 2.04 mmol) in THF (10 mL) at room temperature was added dropwise 10M BH₃-DMS (410 μL). The reaction was stirred overnight, quenched with dropwise addition of 1 M HCl, and extracted with EtOAc (3×10 mL). The crude material was purified via flash chromatography, eluting with 20-60% EtOAc in hexanes. Pure fractions were combined and concentrated to give 270 mg (65%) of desired product as a white solid.

### Step 2: (cis-3-(tert-Butoxycarbonylamino)cyclobutyl)methyl ethanoate

To tert-butyl cis-3-(hydroxymethyl)cyclobutylcarbamate (270 mg, 1.34 mmol) in DCM (7 mL) with DIEA (260 μL, 1.48 mmol) at room temperature was added acetic anhydride (135 μL, 1.4 mmol). The reaction was stirred at room temperature overnight, concentrated to approximately one third of the reaction volume, and purified via silica gel chromatography eluting with 0-50% EtOAc in hexanes. Pure fractions were combined and concentrated to give 250 mg (77%) of desired product as a colorless oil.

### Step 3: (cis-3-Aminocyclobutyl)methyl ethanoate

To (cis-3-(tert-butoxycarbonylamino)cyclobutyl)methyl ethanoate (250 mg, 1.03 mmol) was added 4M HCl in dioxane (2.6 mL). The reaction was stirred overnight at room temperature, concentrated to dryness, and dried overnight under vacuum to afford 184 mg of desired product which was used without further purification as the hydrochloride salt.

### Step 4: Methyl 5-(3,3-dimethylbut-1-yn-1-yl)-3-((cis-3-ethanoyloxymethyl)cyclobutylamino)thiophene-2-carboxylate

To a high-pressure tube containing a suspension of methyl 3-bromo-5-(3,3-dimethylbut-1-yn-1-yl)thiophene-2-carboxylate (308 mg, 1.02 mmol), (cis-3-aminocyclobutyl)methyl ethanoate (184 mg, 1.02 mmol), cesium carbonate (1.00 g, 3.07 mmol) and S-Phos (42.0 mg, 0.10 mmol) in anhydrous dioxane (10 mL) was added Pd₂(dba)₃. The suspension was degassed with an argon stream for 5 minutes then the tube was sealed and heated overnight at 90° C. The crude mixture was cooled to room temperature, filtered over Celite, concentrated to dryness and purified via flash chromatography eluting with EtOAc and hexanes to afford 200 mg (54%) of desired product.

MS: m/z (obs.): 364.2 [M+H]⁺

### Step 5: Methyl 5-(3,3-dimethylbut-1-yn-1-yl)-3-(trans-4-methyl-N-((cis-3-ethanoyloxymethyl)cyclobutyl)cyclohexanecarboxamido)thiophene-2-carboxylate

To Methyl 5-(3,3-dimethylbut-1-yn-1-yl)-3-((cis-3-ethanoyloxymethyl)-cyclobutylamino)thiophene-2-carboxylate (200 mg, 0.55 mmol) in dichloroethane with pyridine (225 μL, 2.75 mmol) was added at room temperature trans-4-methylcyclohexanecarbonyl chloride (265 mg, 1.65 mmol). The reaction was heated overnight at 90 deg C., washed with 1 M HCl, concentrated and then purified via flash chromatography. Pure fractions were combined and concentrated to give 52 mg (19%) of desired product as a colorless film.

MS: m/z (obs.): 488.3 [M+H]⁺

### Step 6: 5-(3,3-dimethylbut-1-yn-1-yl)-3-(trans-4-methyl-N-(cis-3-(hydroxymethyl)cyclobutyl)cyclohexanecarboxamido)thiophene-2-carboxylic acid

To methyl 5-(3,3-dimethylbut-1-yn-1-yl)-3-(trans-4-methyl-N-((cis-3-ethanoyloxymethyl)cyclobutyl)cyclohexanecarboxamido)thiophene-2-carboxylate (52 mg, 0.11 mmol) in a 3:1 mixture of THF and water (1 mL) was added lithium hydroxide monohydrate (45 mg, 1.07 mmol). The reaction was stirred overnight at room temperature, acidified with 1 M HCl, then concentrated to remove organic solvent. The resulting precipitate was filtered, washed with water, and dried overnight under vacuum to afford 38 mg (83%) of desired product.

MS: m/z (obs.): 432.3 [M+H]⁺

1H NMR DMSO-d6, 300 MHz: δ 13.41 (s, 1H), 7.17 (s, 1H), 4.63 (ddd, J=17.3, 9.7, 7.5 Hz, 1H), 4.34 (s, 1H), 3.14 (s, 2H), 2.10 (dt, J=17.2, 7.0 Hz, 1H), 2.02-1.13 (m, 21H), 0.72 (t, J=20.6 Hz, 5H).

### Preparation of Compound 95

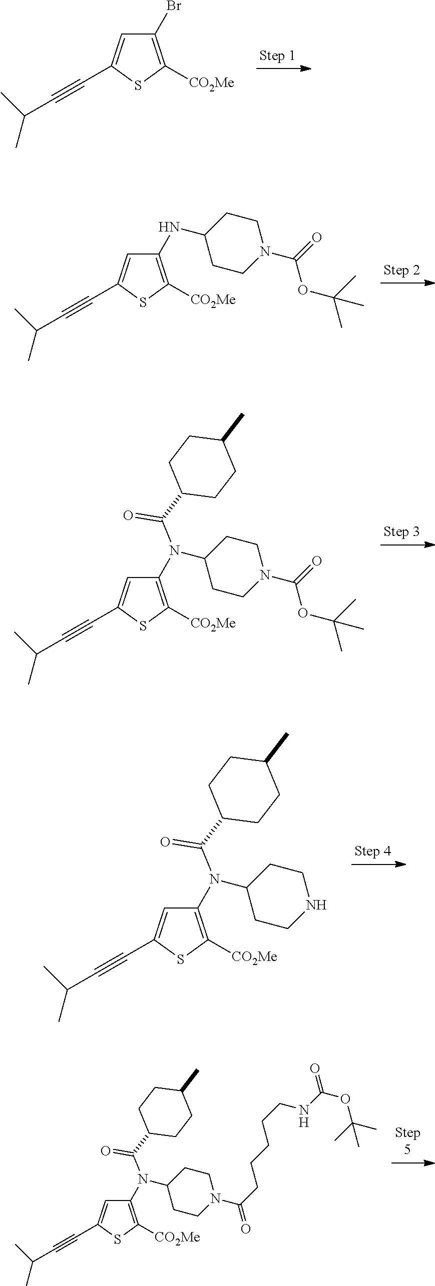

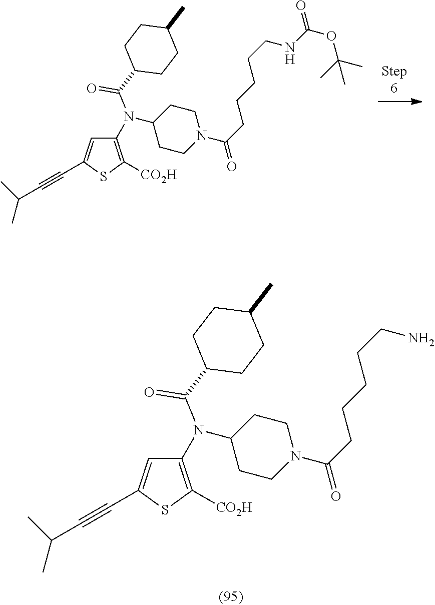

### Step 1. Preparation of Methyl 3-((1-t-butoxycarbonyl-piperidin-4-yl)amino)-5-(3-methylbut-1-ynyl)thiophene-2-carboxylate

Methyl 3-bromo-5-(3-methylbut-1-ynyl)thiophene-2-carboxylate (500 mg, 1.74 mmol), tert-butyl 4-aminopiperidine-1-carboxylate (418.4 mg, 2.09 mmol), cesium carbonate (1.702 g, 5.2 mmol), and dicyclohexyl-[2-(2,6-diisopropoxyphenyl)phenyl]phosphane (81 mg, 0.17 mmol) were taken into 15 mL of degassed 1,4-dioxane. The reaction was heated at 90° C. for 24 h. The reaction was diluted with ethyl acetate and washed with saturated sodium bicarbonate, water, and brine. The ethyl acetate extract was dried over anhydrous sodium sulfate, filtered and evaporated in vacuo. The crude product was purified by column chromatography (40 g SiO₂ column) eluting with a gradient of hexane to 50% ethyl acetate in hexane. The desired fractions were combined and evaporated in vacuo to afford 360 mg of the desired product.

MS: m/z (obs.): 407.3 [M+H]⁺; Rt=5.82 min

1H NMR (300 MHz, CDCl3) d 6.76 (d, 1H), 6.63 (s, 1H), 3.98 (d, 2H), 3.82 (d, 3H), 3.44 (dt, 1H), 2.98 (dd, 2H), 2.80 (dq, 1H), 2.02-1.89 (m, 2H), 1.48 (s, 10H), 1.27 (d, 7H).

### Step 2. Preparation of Methyl 3-((trans-4-methylcyclohexanecarbonyl)-(1-t-butoxycarbonyl-piperidin-4-yl)amino)-5-(3-methylbut-1-ynyl)thiophene-2-carboxylate

Methyl 3-bromo-5-(3-methylbut-1-ynyl)thiophene-2-carboxylate (500 mg, 1.74 mmol), tert-butyl 4-aminopiperidine-1-carboxylate (360 mg, 0.89 mmol) and pyridine (215 μL, 2.6 mmol) were taken into toluene (5 mL); 4-methylcyclohexanecarbonyl chloride (285 mg, 1.77 mmol) was added to the mixture and heated to 90° C. for 24 hours. Little product formation observed via HPLC. Reaction heated to 110 C for 24 hours and most of the starting material was consumed. The reaction was cooled to room temperature and 0.5 mL of pyridine was added to the reaction followed by 1 ml of methanol.

The reaction was washed with brine and dried over anhydrous sodium sulfate, filtered and evaporated in vacuo to afford a crude yellow gum. This was purified by column chromatography (SiO₂) and eluting with Hexane to 50% ethyl acetate in hexane. The desired fractions were combined and evaporated in vacuo to afford 238 mg of the desired product.

MS: m/z (obs.): 531.4 [M+H]⁺; Rt=3.20 min

### Step 3. Preparation of Methyl 3-((trans-4-methylcyclohexanecarbonyl)-(piperidin-4-yl)amino)-5-(3-methylbut-1-ynyl)thiophene-2-carboxylate

Methyl 3-((trans-4-methylcyclohexanecarbonyl)-(1-t-butoxycarbonyl-piperidin-4-yl)amino)-5-(3-methylbut-1-ynyl)thiophene-2-carboxylate (238 mg, 0.45 mmol) was taken into 5 mL of dichloromethane and 0.5 mL of trifluoroacetic acid (500 μL, 6.49 mmol). The reaction was stirred at room temperature for 1 hour, evaporated in vacuo and dissolved in dichoromethane and washed with saturated sodium bicarbonate (2×) and once with brine. Dried over anhydrous sodium sulfate, filtered and evaporated to afford 190 mg of the desired product.

MS: m/z (obs.): 431.4 [M+H]⁺; Rt=2.20 min

### Step 4. Preparation of Methyl 3-((trans-4-methylcyclohexanecarbonyl)-(1-(6-(t-butoxycarbonylamino)-hexanoyl)-piperidin-4-yl)amino)-5-(3-methylbut-1-ynyl)thiophene-2-carboxylate

6-(t-Butoxycarbonylamino)hexanoic acid (112 mg, 0.48 mmol), and methyl 3-((trans-4-methylcyclohexanecarbonyl)-(piperidin-4-yl)amino)-5-(3-methylbut-1-ynyl)thiophene-2-carboxylate (190 mg, 0.44 mmol) were taken into 4 mL of DMF and diisopropylethylamine (230 μL, 1.32 mmol). To the solution was added HBTU ((benzotriazol-1-yloxy-dimethylamino-methylene)-dimethyl-ammonium hexafluorophosphate (251 mg, 0.66 mmol)) and the reaction was stirred at room temperature overnight. The reaction was diluted with ethyl acetate (50 mL) and washed with saturated sodium bicarbonate, water, and brine. The organic layer was dried over anhydrous sodium sulfate, filtered and evaporated to afford a yellow orange gum. This was purified by column chromatography on silica gel eluting with a gradient of hexanes to ethyl acetate. The desired fractions were combined and evaporated in vacuo to afford 259 mg of the desired product.

MS: m/z (obs.): 644.4 [M+H]⁺; Rt=2.53 min

### Step 5. Preparation of 3-((trans-4-Methylcyclohexanecarbonyl)-(1-(6-(t-butoxycarbonylamino)-hexanoyl)-piperidin-4-yl)amino)-5-(3-methylbut-1-ynyl)thiophene-2-carboxylic acid

Methyl 3-((trans-4-methylcyclohexanecarbonyl)-(1-(6-(t-butoxycarbonylamino)-hexanoyl)-piperidin-4-yl)amino)-5-(3-methylbut-1-ynyl)thiophene-2-carboxylate (259 mg, 0.40 mmol) and lithium hydroxide (48 mg, 2.01 mmol) was taken into 4 mL of a 1:1 mixture of methanol:water. The reaction was stirred at room temperature for 2.5 hours. Once complete, the reaction was evaporated in vacuo and the residue was dissolved in water and acidified with 10% sodium bisulfate and extracted with dichloromethane. The organic was dried over sodium sulfate, filtered and evaporated to afford the product (235 mg) as a white solid.

MS: m/z (obs.): 630.4 [M+H]⁺; Rt=1.64 min

### Step 6. Preparation of 3-((trans-4-Methylcyclohexanecarbonyl)-(1-(6-amino-hexanoyl)-piperidin-4-yl)amino)-5-(3-methylbut-1-ynyl)thiophene-2-carboxylic acid (Compound 96)

3-((trans-4-Methylcyclohexanecarbonyl)-(1-(6-(t-butoxycarbonylamino)-hexanoyl)-piperidin-4-yl)amino)-5-(3-methylbut-1-ynyl)thiophene-2-carboxylic acid (50 mg, 0.079 mmol) was taken into ethyl acetate and cooled (0° C.). Anhydrous HCl gas was bubbled into the solution for 1 minute. The reaction was stirred for 1 hour and the product was evaporated at room temperature. The material was dissolved in diethyl ether, then the solvent was evaporated and the residue dried in vacuum oven to give 36 mg of the product.

MS: m/z (obs.): 530.4 [M+H]⁺; Rt=2.10 min

1H NMR (300 MHz, DMSO) 7.94 (br s, 3H), 7.21 (d, J=5.5 Hz, 1H), 4.65-4.27 (m, 2H), 3.81 (d, 1H), 3.38 (m, 3H), 2.98 (m, 1H), 2.88 (dd, 1H), 2.72 (s, 2H), 2.24 (m, 2H), 1.83 (m, 2H), 1.76-1.40 (m, 9H), 1.3-1.15 (m, 4H), 1.20 (t, 6H), 0.89 (m, 1H), 0.75 (d, 3H), 0.61 (m, 2H).

### Example 2

### HCV Replicon Assay

### A. Principle

This procedure below describes the HCV replicon assay using a Huh7 hepatoma cell line harboring a highly cell culture-adapted replicon (genotype 1b) (hereafter named cell line ET). The ET cells contained the highly cell culture-adapted replicon I₃₈₉luc-ubi-neo/NS3-3′/5.1 construct that carried, in addition to the neomycin gene, an integrated copy to the firefly luciferase gene (Krieger, N; Lohmann, V; Bartenschlager, R. Enhancement of hepatitis C virus RNA replication by cell culture-adaptive mutations. J. Virol. 2001, 75, 4614-4624). A replicon cell line W11.8, containing the 1a genotype of HCV was also used. These two cell lines (genotype 1b and 1a) allowed measurement of RNA replication and translation by measuring luciferase activity (against genotype 1b) or by measuring the NS5A level using the ELISA assay (against genotype 1a). It was shown that the luciferase activity tightly followed the replicon RNA level in the ET cells. ET cell lines were maintained in cultures at a sub-confluent level (<85%). The culture media used for cell passaging consisted of DMEM (Gibco BRL Laboratories, Mississauga, ON, Canada) supplemented with 10% fetal bovine serum with 1% penicillin/streptomycin, 1% glutamine, 1% sodium pyruvate, 1% non-essential amino acids, and 180 μg/ml of G418 final concentration.

### B. Measurement of Luciferase Activity (Luci-ET-1b)

For the treatment of the cells with the testing drug, the culture medium was removed from the 175 cm² T-flask by aspiration. Cell monolayer was rinsed with 10 mL of PBS 1× at room temperature. PBS was removed by aspiration. Cells were trypsinized using 1 mL of Trypsin/EDTA. Flask were incubated at 37° C. (incubator) for 7 minutes. Complete medium (9 mL) with no G418 and no phenol red was then added. Cell clumps were disrupted by pipetting up and down several times. The cell suspension was then transferred to a 50 mL Falcon polypropylene tube. Cells were then counted several times using the hemacytometer. Cells were diluted at 30 000 cells/mL with complete DMEM with no G418 and no phenol red, then transferred into a sterile reservoir. Using a multichannel pipet, approximately 3000 viable cells (100 μL) were plated per well in a white opaque 96-well microtiter plate. After an incubation period of 2-4 hours at 37° C. in a 5% CO₂ incubator, compounds were added at various concentrations.

Compounds under testing were resuspended in DMSO at a stock concentration of 100 mM. Then, they were diluted at twice the final concentration in the same medium (without G418) described earlier, in sterile 96-deep well plate and according to a particular template. One volume (100 μL) of each compound dilution was then added to each well that contains cells or in control wells with no cells. Final drug concentrations were usually between 200 μM and 0.0001 μM. Ten wells were used as positive control without drug. Cells were further incubated for 4 days at 37° C. in a 5% CO₂ incubator. A control compound was used as an internal standard at the same concentrations described above.

Following the incubation time of four days, the culture media was removed and quickly dried upside down on a stack of sterile absorbing papers. Cells were then lysed by the addition of 95 μL of the luciferase buffer A using a multichannel pipet, sealed using TopSeal™ adhesive sealing film and the reaction mixture was incubated at room temperature and protected from direct light for at least 10 minutes. Plates were read for luciferase counts using a luminometer (Wallac MicroBeta Trilux, Perkin Elmer™, MA, USA).

The percentage of inhibition at each drug concentration tested (in duplicate) was calculated. The concentration required to reduce viral replication by 50% (IC₅₀) was then determined from dose response curves using nonlinear regression analysis (e.g., GraphPad Prism software, version 2.0 (GraphPad Software Inc., San Diego, Calif., USA)). The IC₅₀ values are summarized in Tables 1-3:
A: IC₅₀ value (mean)≦0.1 μM;
B: 0.1 μM<IC₅₀ value (mean)≦1 μM;
C: 1 μM<IC₅₀ value (mean)≦10 μM;
D: IC₅₀ value (mean)>10 μM.

### C. Elisa Assay (ELISA W 11.8-1a)

Replicon cell lines W11.8 containing a sub-genomic replicon of genotype 1a was used for the HCV Replicon Cell-Based detection using the ELISA. The RNA replication in presence of different drug concentrations was indirectly measured in these cell lines by the level of NS5A protein content upon drug treatment for four days. The NS5A is a non-structural protein of HCV and is used as marker of HCV replication in this assay.

For the treatment of the cells with the testing drug, Culture medium was removed from the 175 cm² T-flask by aspiration. Cell monolayer was rinsed with 10-20 mL of PBS 1× at room temperature. PBS was removed by aspiration. Cells were trypsinized using 3 mL of Trypsin (0.25%)/EDTA (0.1%) solution. Flasks were incubated at 37° C. (incubator) for 7 minutes. Complete medium (9 mL) without G418 is then added. Cell clumps were disrupted by pipetting up and down several times.

The cell suspension was then transferred to a 50 mL Falcon polypropylene tube. Cells were then counted several times using the haemocytometer. Cells were diluted at 50,000 cells/mL with complete DMEM without G418, then transferred into a sterile reservoir. Using a multichannel pipet, approximately 5,000 viable cells (100 μL) were plated per well in a white opaque 96-well microtiter plate. After an incubation period of 2-4 hours at 37° C. in a 5% CO₂ incubator, compounds were added at various concentrations.

Drugs were resuspended in DMSO at a stock concentration of 100 mM or 10 mM. In some cases (drugs with a potency below nmolar values), it was necessary to dilute compounds in DMSO at 1 mM or 100 μM as a starting solution. Then, drugs were diluted at twice the final concentration in the same medium (without G418) described earlier, in sterile 96-deep well plate and according to a particular template (see Appendix). One volume (100 μL) of each drug dilution was then added to each well that contains cells.

Sixteen wells were used as control (0% inhibition) without drug. Eight wells were used as background control (100% inhibition) containing 2 μM (final concentration) of the reference compound. The reference compound at 2 μM was shown to inhibit the NS5A expression at ≈100% and is nontoxic to the cells. Values from 100% inhibited wells were averaged and used as the background value. Cells are further incubated for 4 days at 37° C. in a 5% CO₂ incubator.

For the measurement of NS5A protein content, following the incubation time of four days, the media was throwed into an appropriate waste container by inverting the plate. Any residual liquid was removed by tapping gently on absorbent paper several times. The plates were then washed once with 150 μL of PBS per well, and then incubated for 5 minutes at room temperature on a shaker (500 rpm). 150 μL per well of cold (−20° C.) fixative solution (50% methanol/50% acetone mix) was added into the plates, and the plates was incubated for 5 minutes at room temperature. The plates were then inverted, and any residual liquid was removed by tapping gently on absorbent paper several times. The plates were then washed twice with 150 μL of PBS per well, and incubated for 5 minutes at room temperature on a shaker (500 rpm) for each wash. 150 μL of blocking solution per well was added into the plates. The plates were then sealed using TopSeal™ adhesive sealing films and incubated for one hour at 37° C. or at 4° C. overnight to block non-specific sites.

The plates were inverted and the blocking solution was dumped into an appropriate waste container. Any residual liquid was removed by tapping gently on absorbent paper several times. The plates were then washed twice with 150 μL of PBS per well and once with 150 μL of PBSTS solution per well, and then incubated for 5 minutes at room temperature on a shaker (500 rpm) for each wash. Then, was add into the plates 50 μL per well of anti-human NS5A antibody (Ab1) diluted 1/1,000 in the blocking solution. The plates were then sealed using TopSeal™ adhesive sealing films and incubate at 4° C. overnight.

Next day, the plates were inverted to dump solution into an appropriate waste container. The plates then were gently tapped on absorbent paper several times to remove residual liquid. The plates were washed five times with 150 μL of PBS per well, and incubated for 5 minutes at room temperature on a shaker (500 rpm) for each wash. Then was add into the plates 50 μL per well of peroxidase-conjugated donkey anti-mouse antibody (Ab2) diluted 1/10,000 in the blocking solution. The plates were then sealed using TopSeal™ adhesive sealing films and incubate at room temperature for 3 hours on a shaker (500 rpm). Towards the end of the 3 hours incubation, the commercially available chemiluminescent substrate solution was prepared. A mixture of equal volumes of the luminol/enhancer and stable peroxide reagents was prepared and protected from light. The plates were then inverted to dump solution into an appropriate waste container. Any residual liquid was removed by tapping gently on absorbent paper several times. The plates were washed four times with 150 μL of PBSTS solution per well and once with 150 μL of PBS, and then incubated for 5 minutes at room temperature on a shaker (500 rpm) for each wash. 100 μL of substrate solution per well was then added into the plates. The plates were then sealed using TopSeal™ adhesive sealing films and incubate for 1 minute at room temperature on a shaker (500 rpm), and then incubated between 30 and 60 minutes at room temperature (protect from light) prior to reading the luminescence (relative light units) on the Analyst HT plate reader (LJL Default Luminescence Method).

The percentage of inhibition at each drug concentration tested (in duplicate) was calculated. The concentration required to reduce viral replication by 50% (IC₅₀) was then determined from dose response curves using nonlinear regression analysis (e.g., GraphPad Prism software, version 2.0 (GraphPad Software Inc., San Diego, Calif., USA)). The IC50 values are summarized in Table 1:
A: IC₅₀ value (mean)≦0.1 μM;
B: 0.1 μM<IC₅₀ value (mean)≦1 μM;
C: 1 μM<IC₅₀ value (mean)≦10 μM;
D: IC₅₀ value (mean)>10 μM.

### Example 3

### [³H]Thymidine Incorporation Assay

A total of 2,000 cells/well were seeded in 96-well cluster dishes in a volume of 100 [mu]l of DMEM (Wisent., St Bruno, QC) supplemented with 10% FBS (Wisent., St Bruno, QC) and 2 mM glutamine (Life Technologies, Inc.). Penicillin and streptomycin (Life Technologies, Inc.) are added to 500 U/mL and 50 μg/mL final concentrations, respectively. After an incubation of at least 3 h at 37° C. in an atmosphere of 5% CO₂, compounds, prepared at twice the final concentration, are added to the cells. Eleven serial two to four-fold dilutions of drugs are tested in duplicate plates. After 72-h incubation, a volume of 20 μL of a 10 μCi/mL solution of [3H] methyl thymidine (Amersham Life Science, Inc., Arlington Heights, III; 2 Ci/mmol) in culture medium is added and the plates are incubated for a further a 24 h at 37° C. Cells are then washed with phosphate-buffered saline (PBS), trypsinized for 2 min, and collected onto a fiberglass filter using a Tomtec cell harvester (Tomtec, Orange, Conn.). Filters are dried at 37° C. for 1 h and placed into a bag with 4.5 mL of liquid scintillation cocktail (Wallac Oy, Turku, Finland). The accumulation of [3H] methyl thymidine, representing viable replicating cells, is measured using a liquid scintillation counter (1450-Microbeta; Wallac Oy). Ref. SOP: 265-162-03. For this experiment, the cell lines used are; Huh-7 ET (cells derived from the Huh-7 cell line (hepatocellular carcinoma, human) and containing a HCV sub-genomic replicon), Molt-4 (peripheral blood, acute lymphoblastic leukemia, human), DU-145 (prostate carcinoma, metastasis to brain, human), Hep-G2 (hepatocellular carcinoma, human), and SH-SY5Y (neuroblastoma, human) cells.

The 50% cytotoxic concentrations (CC₅₀) for cell toxicity were determined from dose response curves using six to eight concentrations per compound in triplicate. Curves were fitted to data points using non-linear regression analysis, and IC₅₀ values were interpolated from the resulting curve using GraphPad Prism software, version 2.0 (GraphPad Software Inc., San Diego, Calif., USA).

CC₅₀ values of compounds of the invention are summaries in Table 1:
A: CC₅₀ value (mean)≧100 μM;
B: 10 μM≦CC₅₀ value (mean)<100 μM;
C: CC₅₀ value (mean)≦10 μM.

TABLE 1
IC₅₀, CC₅₀, LCMS and NMR data of the compounds described in FIG. 1.

| Compound | HCV-Replicon- | HCV-Replicon- | | LCMS | LCMS | |
|---|---|---|---|---|---|---|
| Nos. | 1b_IC50 | ELISA-1a IC50 | CC50 | [M + H]⁺ | RT | NMR |
| 1 | | A | B | 439 | 2.9 | |
| 2 | | B | A | 405.93 | | |
| 3 | | B | B | 460.55 | 6.05 | |
| 4 | A | A | B | 432.52 | 5.27 | 1H NMR (300 MHz, DMSO) d 13.44 (s, 7H), |
| | | | | | | 7.18 (s, 4H), 4.46 (s, 6H), 4.27 (t, J = 9.8 Hz, |
| | | | | | | 6H), 3.24 (d, J = 33.4 Hz, 14H), 3.16-2.89 |
| | | | | | | (m, 4H), 2.85 (d, J = 6.8 Hz, 2H), 2.50 (dt, J = |
| | | | | | | 3.6, 1.8 Hz, 22H), 2.28-1.63 (m, 20H), 1.63- |
| | | | | | | 1.33 (m, 23H), 1.38 (s, 1H), 1.30 (s, 2H), 1.27- |
| | | | | | | 1.06 (m, 42H), 0.92-0.68 (m, 17H), 0.68-−0.00 |
| | | | | | | (m, 19H), −0.01 (s, 2H). |
| 5 | B | B | B | 430.47 | 4.97 | |
| 6 | | C | B | 404.49 | 4.8 | 1H NMR (300 MHz, DMSO) d 7.18 (s, 1H), |
| | | | | | | 4.32 (dd, J = 42.1, 30.5 Hz, 2H), 3.24 (d, J = |
| | | | | | | 35.7 Hz, 8H), 2.68-2.30 (m, 7H), 2.12 (s, |
| | | | | | | 2H), 1.90-1.67 (m, 3H), 1.63-1.35 (m, 4H), |
| | | | | | | 1.18 (d, J = 8.7 Hz, 3H), 0.91-0.39 (m, 4H). |
| 7 | | A | B | 418.51 | 5.26 | 1H NMR (300 MHz, DMSO) d 13.40 (s, 2H), |
| | | | | | | 7.18 (s, 1H), 4.26 (dd, J = 15.1, 7.3 Hz, 2H), |
| | | | | | | 3.64-3.03 (m, 8H), 2.19-1.67 (m, 6H), 1.77- |
| | | | | | | 1.38 (m, 10H), 1.38-1.04 (m, 10H), 2.05- |
| | | | | | | 0.24 (m, 32H), 0.96-0.35 (m, 9H). |
| 8 | | A | B | 446.53 | 5.69 | 1H NMR (300 MHz, MeOD) d 7.00 (s, 3H), |
| | | | | | | 4.81 (s, 18H), 4.39 (d, J = 3.4 Hz, 2H), 4.09 |
| | | | | | | (d, J = 7.1 Hz, 1H), 3.55-3.25 (m, 18H), 2.38 |
| | | | | | | (d, J = 6.5 Hz, 6H), 2.03-1.45 (m, 34H), 1.45- |
| | | | | | | 1.17 (m, 16H), 1.09-0.86 (m, 21H), 0.80 (d, |
| | | | | | | J = 6.5 Hz, 9H), 0.73-0.12 (m, 5H), −0.01 (s, |
| | | | | | | 3H). |
| 9 | | A | B | 458.5 | 5.86 | 1H NMR (300 MHz, MeOD) d 7.30-6.96 (m, |
| | | | | | | 3H), 4.82 (s, 17H), 4.72-4.27 (m, 3H), 4.09 |
| | | | | | | (q, J = 7.1 Hz, 1H), 4.04-3.24 (m, 16H), 3.24- |
| | | | | | | 2.83 (m, 3H), 2.80-1.45 (m, 63H), 1.42- |
| | | | | | | 0.35 (m, 37H), 0.22-−0.31 (m, 3H). |
| 10 | | A | B | 434.1 | 2.87 | 1H NMR CDCl3, 400 MHz: 6.92 (s, 1H), 4.9 |
| | | | | | | (Bs, 1H), 3.74 (m, 1H), 3.48 (m, 1H), 3.35 (s, |
| | | | | | | 3H), 2.07-1.97 (m, 2H), 1.79-1.48 (m, 5H), |
| | | | | | | 1.32 (s, 9H), 0.88 (d, j = 6.4 Hz, 3H), 0.78 |
| | | | | | | (d, j = 5.2 Hz, 3H), 0.73 (bs, 2H) |
| 11 | | A | B | 473.3 | 1.34 | 1H NMR DMSO-d6, 300 MHz: δ 7.21 (s, 1H), |
| | | | | | | 4.73 (s, 1H), 3.60 (s, 0.66H), 3.30 (s, 1.33H), |
| | | | | | | 2.83 (t, J = 29.7 Hz, 7H), 2.36-2.21 (m, 1H), |
| | | | | | | 2.16-2.00 (m, 1H), 1.92-1.11 (m, 18H), |
| | | | | | | 0.72-0.51 (m, 4H) |
| 12 | | A | B | 486.18 | | |
| 13 | | C | B | 442.36 | | ¹H NMR (400 MHz, DMSO-d₆): δ 13.55 (s, |
| | | | | | | 1H), 7.27-7.17 (m, 1H), 5.66-5.47 (m, 2H), |
| | | | | | | 4.66-4.28 (m, 2H), 3.95-3.83 (m, 1H), 2.16- |
| | | | | | | 1.42 (m, 12H), 1.26 (s, 9H), 0.82-0.70 (m, |
| | | | | | | 3H), 0.67-0.54 (m, 2H) |
| 14 | | A | B | 466.24 | | |
| 15 | | A | B | 446.26 | | |
| 16 | | B | B | 388.26 | | |
| 18 | | C | B | 416.25 | | ¹H NMR (400 MHz, CDCl₃): δ 6.84 (s, 1H), |
| | | | | | | 5.00-4.84 (m, 1H), 2.68-2.58 (m, 1H), 2.04- |
| | | | | | | 1.85 (m, 3H), 1.81-1.70 (m, 2H), 1.70-1.48 |
| | | | | | | (m, 8H), 1.48-1.23 (m, 5H), 1.15 (d, 3H), |
| | | | | | | 0.92 (d, 3H), 0.79 (d, 3H), 0.76-0.58 (d, 2H) |
| 19 | | B | A | 392.22 | | |
| 20 | | B | B | 374.02 | | ¹H NMR (400 MHz, CDCl₃): δ 6.83 (s, 1H), |
| | | | | | | 4.96-4.85 (m, 1H), 1.95 (bs, 1H), 1.70-1.55 |
| | | | | | | (m, 4H), 1.56-1.46 (m, 1H), 1.45-1.22 (m, |
| | | | | | | 2H), 1.13 (d, 3H), 1.03-0.85 (m, 7H), 0.78 |
| | | | | | | (d, 3H), 0.75-0.56 (s, 2H) |
| 21 | | C | B | 410.11 | | ¹H NMR (400 MHz, CDCl₃): δ 9.91 (bs, 1H), |
| | | | | | | 7.60-7.51 (m, 2H), 7.44-7.35 (m, 3H), 7.03 |
| | | | | | | (s, 1H), 5.04-4.89 (m, 1H), 2.08-1.91 (m, |
| | | | | | | 1H), 1.76-1.24 (m, 5H), 1.19 (d, J = 6.6 Hz, |
| | | | | | | 3H), 0.97 (d, J = 6.8 Hz, 3H), 0.80 (d, J = 6.5 |
| | | | | | | Hz, 3H), 0.77-0.59 (m, 2H) |
| 22 | | B | B | 406.15 | | ¹H NMR (400 MHz, CDCl₃): δ 8.00 (bs, 1H), |
| | | | | | | 6.93 (s, 1H), 4.93 (s, 1H), 3.43 (s, 3H), 1.96 |
| | | | | | | (bs, 1H), 1.74-1.58 (m, 4H), 1.56 (s, 6H), 1.52- |
| | | | | | | 1.20 (m, 3H), 1.16 (d, 3H), 0.93 (d, 3H), 0.79 |
| | | | | | | (d, 3H), 0.73-0.58 (m, 2H) |
| 23 | | B | B | 402.17 | | ¹H NMR (400 MHz, DMSO-d₆): δ 7.88 (s, 1H), |
| | | | | | | 4.79-4.66 (m, 1H), 1.82 (t, 1H), 1.62-1.13 |
| | | | | | | (m, 8H), 1.05 (d, 3H), 0.83 (d, 3H), 0.76 (d, |
| | | | | | | 3H), 0.68-0.53 (m, 2H) |
| 24 | | | C | 444.05 | | ¹H NMR (400 MHz, CDCl₃): δ 10.02 (s, 1H), |
| | | | | | | 8.13 (d, 1H), 5.45 (s, 2H), 2.58 (s, 3H), 2.30- |
| | | | | | | 2.19 (m, 1H), 2.05-1.98 (m, 2H), 1.84-1.77 |
| | | | | | | (m, 2H), 1.60-1.47 (m, 2H), 1.29 (s, 9H), |
| | | | | | | 1.06-0.94 (m, 3H), 0.91 (d, 3H) |
| 25 | | A | B | 446.53 | 5.72 | 1H NMR (300 MHz, MeOD) d 7.08 (s, 1H), |
| | | | | | | 4.98 (s, 4H), 4.59-4.27 (m, 1H), 4.16-3.70 |
| | | | | | | (m, 5H), 3.57-3.23 (m, 3H), 2.44-2.25 (m, |
| | | | | | | 1H), 2.18-1.30 (m, 20H), 1.26 (dd, J = 9.5, |
| | | | | | | 4.8 Hz, 2H), 1.19-0.83 (m, 3H), 0.83-0.12 |
| | | | | | | (m, 5H). |
| 26 | | B | A | 460.55 | 4.24 | 1H NMR (300 MHz, MeOD) d 6.95 (s, 1H), |
| | | | | | | 4.81 (s, 5H), 4.49-4.20 (m, 1H), 3.51-3.22 |
| | | | | | | (m, 4H), 2.01-1.14 (m, 23H), 1.08-0.83 (m, |
| | | | | | | 4H), 0.83-0.10 (m, 7H). |
| 27 | | B | A | 460.48 | 4.08 | (CDCl3, 400 MHz): 6.92 (s, 1H), 3.90-3.75 (m, |
| | | | | | | 2H), 3.8-3.40 (m, 2H), 3.30 (s, 3H), 2.20-2.05 |
| | | | | | | (m, 1H), 1.65-1.58 (m, 7H), 1.33 (s, 9H), 0.80 |
| | | | | | | (d, J = 6.8 Hz, 3 H), 0.72 (m, 2H) |
| 28 | A | A | B | 444.49 | 5.34 | 1H NMR (300 MHz, DMSO) d 7.22 (s, 1H), |
| | | | | | | 5.63 (s, 1H), 4.47 (s, 2H), 4.28 (s, 1H), 3.30 |
| | | | | | | (s, 4H), 3.18 (s, 1H), 2.50 (dt, J = 3.6, 1.8 Hz, |
| | | | | | | 8H), 2.01-1.60 (m, 6H), 1.57 (d, J = 11.7 Hz, |
| | | | | | | 3H), 1.44 (d, J = 14.6 Hz, 7H), 1.33-0.87 (m, |
| | | | | | | 6H), 0.87-0.69 (m, 4H), 0.69-0.54 (m, 2H), |
| | | | | | | 0.09-−0.09 (m, 4H). |
| 29 | | A | B | 406.5 | 4.66 | |
| 30 | | B | B | 448.45 | 3.67 | (DMSO, 400 MHz, at 800 C.): 6.94 (s, 1H), |
| | | | | | | 4.74 (s, 1H), 3.45 (s, 1H), 3.24 (s, 3H), 3.12 |
| | | | | | | (s, 2H), 1.93-1.84 (m, 2H), 1.65-1.56 (m, 4H), |
| | | | | | | 1.30 (s, 9H), 0.96-0.93 (m, 3H), 0.77 (d, |
| | | | | | | J = 6.4 Hz, 3H), 0.63 (s, 2H) |
| 32 | | D | B | 470.46 | 6 | 1H NMR (300 MHz, DMSO) d 7.30 (s, 1H), |
| | | | | | | 4.28 (s, 2H), 3.30 (s, 4H), 3.20 (s, 1H), 2.56- |
| | | | | | | 2.43 (m, 3H), 2.25-1.55 (m, 9H), 1.55-1.22 |
| | | | | | | (m, 12H), 1.17 (d, J = 9.4 Hz, 4H), 0.84 (dd, |
| | | | | | | J = 12.8, 2.9 Hz, 2H), 0.76 (d, J = 6.4 Hz, 3H), |
| | | | | | | 0.61 (d, J = 11.8 Hz, 2H), −0.00 (s, 1H). 1H |
| | | | | | | NMR (300 MHz, DMSO) d 7.30 (s, 1H), 4.28 |
| | | | | | | (s, 2H), 3.30 (s, 4H), 3.20 (s, 1H), 2.56-2.43 |
| | | | | | | (m, 3H), 2.25-1.55 (m, 9H), 1.55-1.22 (m, |
| | | | | | | 12H), 1.17 (d, J = 9.4 Hz, 4H), 0.84 (dd, J = |
| | | | | | | 12.8, 2.9 Hz, 2H), 0.76 (d, J = 6.4 Hz, 3H), |
| | | | | | | 0.61 (d, J = 11.8 Hz, 2H), −0.00 (s, 1H). |
| 33 | | B | B | 420.5 | 4.94 | (CDCl3, 400 MHz, mixture of diateromers) |
| | | | | | | 6.89(s, 1H), 4.50(m, 1H), 3.50-3.42(m, 2H), |
| | | | | | | 3.02(m, 1H), 2.01-1.84(m, 6H), 1.59-1.40(m, 4H) |
| | | | | | | 1.32(s, 9H), 1.25(m, 2H), 1.15(t, J = 6.8, |
| | | | | | | 7.2 Hz, 3H), 0.89-0.65?(m, 6H). |
| 34 | | B | A | 462.4 | 4.67 | (CDCl3, 400 MHz, mixture of stereoisomers, |
| | | | | | | data for one compound): 6.89 (s, 1H), 6.83 (s, |
| | | | | | | 1H), 4.99 (s, 1H), 4.80 (br. s, 1H), 3.80- |
| | | | | | | 3.70(m, 1H), 3.52-3.40(m, 2H), 3.36(s, 3H), |
| | | | | | | 1.98-1.84(m, 2H), 1.71-1.49(m, 4H), 1.33(s, 9H), |
| | | | | | | 0.98-0.86(m, 2H), 0.77(s, 3H), 0.67(s, 2H) |
| 35 | A | B | B | 474 | 3.73 | (CDCl3, 400 MHz, data for major diastereomer): |
| | | | | | | 6.81(s, 1H), 4.9 (brs, 1H), 4.2(br s, 1H), |
| | | | | | | 3.48(m, 1H), 3.27(s, 3H), 2.07-1.97(m, 2H), |
| | | | | | | 1.79-1.48(m, 9H), 1.32(s, 9H), 1.0-0.65 |
| | | | | | | (series of m, 7H) |
| 36 | A | A | B | 418 | 2.58 | (400 MHz, CDCl3): 6.78 (br. s, 1H), 5.95-5.83 |
| | | | | | | (m, 1H impurity), 5.22 (d, J = 17 Hz, imurity, |
| | | | | | | 1H), 5.13 (d, J = 10.0 Hz, impurity, 1H), 4.49 |
| | | | | | | (s, 1H), 3.93 (s, 1H), 3.07 (s, 1H), 2.03-0.68 |
| | | | | | | (series of m, 26H) |
| 37 | A | B | B | 434.5 | 2.86 | (DMSO, 400 MHz, at 800 C.) 6.86 (s, 1H), |
| | | | | | | 4.54-4.48 (m, 1H), 3.60-3.39(m, 4H), 3.20 (s, |
| | | | | | | 3H), 3.12(s, 3H), 2.04(m, 1H), 1.76-1.73 (m, |
| | | | | | | 1H), 1.58-1.35(m, 5H), 1.29(s, 9H), 0.76 (d, |
| | | | | | | J = 6.8 Hz, 3H), 0.64-0.61(m, 2H) |
| 38 | A | B | B | 486 | 3.96 | (DMSO-d6, 400 MHz, at 80 C.) for major compound): |
| | | | | | | 6.93-6.88 (m, 1H), 4.74-4.72 (m, 1H), 4.38 (b s, 1H), |
| | | | | | | 3.54 (b s, 1H), 3.26-3.07(m, 6H), 2.89-2.81 (m, 1H), |
| | | | | | | 2.08-1.98(b s, 1H), 1.70-1.32 (m, 4H), 1.29-1.17(m, |
| | | | | | | 6H), 0.96- |
| 39 | | B | B | 450.6 | 2.53 | |
| 40 | | A | B | | | (DMSO-d6, 400 MHz): ? 6.82 (s, 1H), 4.49-4.43 |
| | | | | | | (m, 1H), 3.82-3.73 (m, 2H), 3.31-3.23 (m, 2H), |
| | | | | | | 2.86-2.79 (m, 1H), 2.03-1.97 (m, 1H), 1.79- |
| | | | | | | 1.48 (m, 6H), 1.37-1.28 (m, 2H), 1.2 (d, |
| | | | | | | J = 7.2 Hz; 6H), 1.18-1.01 (m, 3H), 0.75 (d, |
| | | | | | | J = 6.0 Hz; 3H), 0.63-0.50 (m, 2 |
| 41 | | A | A | 434.31 | 1.43 | (DMSO-d6, 400 MHz) for major isomer): 6.95 |
| | | | | | | (s, 1H), 4.4 (br s, 1H), 3.2 (s, 3H), 3.05 (s, |
| | | | | | | 2H), 2.9-2.8 (m, 1H), 2.08-1.98 (br s, 1H), |
| | | | | | | 1.70-1.4 (m, 7H), 1.21 (d, J6.8 Hz, 6H), 1.2- |
| | | | | | | 1.14 (m, 2H), 0.9-0.5 (m, 9H) |
| 43 | | A | B | 418.139 | 3.25 | (DMSO-d6, 400 MHz) for major compound |
| | | | | | | from mixture of isomers): 13.6(br)7.02(s, 1H), |
| | | | | | | 4.81-4.78 (m, 1H), 3.23(s, 3H), 3.22-3.20(m, |
| | | | | | | 2H), 3.09(s, 2H), 2.91-2.84 (m, 2H), 1.92- |
| | | | | | | 1.84(m, 2H), 1.70-1.4 (m, 6H), 1.21(d, |
| | | | | | | J = 7.2 Hz, 9H), 0.76(d, J = 7.2 Hz, 3H) |
| 44 | | B | B | 419.1 | 3.37 | 1H NMR (300 MHz, DMSO) d 7.94 (br s, 3H), |
| | | | | | | 7.21 (d, J = 5.5 Hz, 1H), 4.65-4.27 (m, 2H), |
| | | | | | | 3.81 (d, 1H), 3.38 (m, 3H), 2.98 (m, 1H), |
| | | | | | | 2.88 (dd, 1H), 2.72 (s, 2H), 2.24 (m, 2H), 1.83 |
| | | | | | | (m, 2H), 1.76-1.40 (m, 9H), 1.3-1.15 (m, 4H), |
| | | | | | | 1.20 (t, J = 11.9 Hz, 6H), 0.89 (m, 1H), 0.75 |
| | | | | | | (d, 3H), 0.61 (m, 2H). |
| 45 | | A | B | 406.199 | 4.66 | (DMSO-d6, 400 MHz) for major isomer): 6.85 |
| | | | | | | (s, 1H), 4.4 (br s, 1H), 3.2 (s, 3H), 3.05 (s, |
| | | | | | | 2H), 2.9-2.8 (m, 1H), 2.08-1.98 (br s, 1H), |
| | | | | | | 1.70-1.4 (m, 7H), 1.21 (d, J6.8 Hz, 6H), 1.2- |
| | | | | | | 1.14 (m, 2H), 0.9-0.5 (m, 9H) |
| 47 | | A | B | 420.13 | 4.95 | 1H NMR (300 MHz, DMSO) d 7.22 (s, 1H), |
| | | | | | | 4.60 (s, 1H), 3.87-2.80 (m, 12H), 2.14 (t, J = |
| | | | | | | 11.6 Hz, 1H), 1.73-1.00 (m, 16H), 0.77 (d, J = |
| | | | | | | 6.5 Hz, 5H). |
| 48 | | A | B | 461 | 0.8 | (DMSO, 400 MHz) 0.60-0.75 (2H, m), 0.77 |
| | | | | | | (3H, d), 1.20 (6H, d), 1.25-1.70 (6H, m), 2.18 |
| | | | | | | (1H, app t), 2.85 (1H, September), 3.74 (1H, d), |
| | | | | | | 4.55 (1H, d), 7.18 (1H, s), 12.60 (1H, br s), |
| | | | | | | 13.50 (1H, br s). |
| 50 | | A | A | 503.35 | 1.21 | 1H NMR (300 MHz, DMSO) 6.78 (d, J = 8.9 |
| | | | | | | Hz, 1H), 4.85 (d, J = 19.3 Hz, 1H), 4.71 (m, |
| | | | | | | 2H), 4.43 (br s, 1H), 4.11-4.02 (m, 2H), 3.87 |
| | | | | | | (dd, J = 25.7, 14.0 Hz, 1H, 3.39 (d, J = 6.2 Hz, |
| | | | | | | 3H), 3.11 (dd, J = 26.7, 12.9 Hz, 1H), 2.66 (dd, |
| | | | | | | J = 24.0, 11.5 Hz, 1H), 2.05-1.78 (m, 3H), 1.65- |
| | | | | | | 1.5 (m, 5H), 1.49-1.39 (m, 2H), 1.36 (s, 9H), |
| | | | | | | 1.110 (m, 1H), 0.82 (d, J = 6.5 Hz, 3H), 0.76 |
| | | | | | | (m, 2H). |
| 51 | | A | A | 473.34 | 1.2 | 1H NMR (300 MHz, DMSO) d 13.64 (br s, 1H), |
| | | | | | | 7.19 (d, J = 7.2 Hz, 1H), 4.64-4.26 (m, 2H), |
| | | | | | | 3.79 (m, 1H), 3.04 (m, 1H), 1.93 (d, J = |
| | | | | | | 9.4 Hz, 3H), 1.84 (d, J = 11.5 Hz, 2H), 1.70- |
| | | | | | | 1.35 (m, 5H), 1.30 (s, 9H), 1.25-1.04 (m, |
| | | | | | | 4H), 1.04-0.81 (m, 1H), 0.76 (d, J = 6.4 Hz, |
| | | | | | | 3H), 0.59 (dd, J = 26.9, 14.8 Hz, 3H). |
| 52 | | A | B | 446.1 | 4.74 | (CDCl3, 400 MHz): 6.82 (s, 1H), 4.50 (m, 1H), |
| | | | | | | 3.28 (s, 3H), 2.95-2.94 (m, 1H), 2.85-2.82 (m, |
| | | | | | | 1H), 2.09-2.06 (m, 1H), 2.01-1.93 (m, 2H), |
| | | | | | | 1.84-1.81 (m, 1H), 1.65-1.55 (m, 5H), 1.42-1.33 |
| | | | | | | (m, 4H), 1.29 (d, J = 6.4 Hz; 6H), 0.94-0.88 |
| | | | | | | (m, 1H), 0.79 (d |
| 53 | | A | B | 460.199 | 3.12 | (DMSO-d6, 400 MHz): 6.89 (s, 1H), 4.23 (m, |
| | | | | | | 1H), 3.37 (q, J = 7.2 Hz; 2H), 2.99 (m, 1H), |
| | | | | | | 2.87-2.80 (m, 1H), 1.95-1.72 (m, 6H), 1.55- |
| | | | | | | 1.48 (m, 3H), 1.40-1.31 (m, 1H), 1.20 (d, |
| | | | | | | J = 6.8 Hz; 6H), 1.16-1.08 (m, 3H), 1.04 (t, |
| | | | | | | J = 6.8 Hz; 3H), 0.86-0.74 (m, 4H) |
| 54 | | A | B | 457.41 | 1.77 | |
| 55 | | A | B | 459.46 | 0.83 | |
| 56 | | D | B | 434.44 | 3.21 | |
| 57 | | A | A | 417 | 0.59 | 1H NMR (300 MHz, DMSO) d 7.35 (d, J = |
| | | | | | | 29.1 Hz, 1H), 4.79-4.50 (m, 1H), 3.53-2.86 |
| | | | | | | (m, 7H), 2.20-1.96 (m, 1H), 1.94-1.03 (m, |
| | | | | | | 16H), 0.75-0.52 (m, 4H). |
| 58 | | A | A | 417 | 0.59 | 1H NMR (300 MHz, DMSO) d 7.35 (d, J = |
| | | | | | | 29.1 Hz, 1H), 4.79-4.50 (m, 1H), 3.53-2.86 |
| | | | | | | (m, 7H), 2.20-1.96 (m, 1H), 1.94-1.03 (m, |
| | | | | | | 16H), 0.75-0.52 (m, 4H). |
| 59 | | A | A | 459 | 1.15 | 1H NMR (300 MHz, DMSO) d 7.31 (dd, J = |
| | | | | | | 9.9, 6.6 Hz, 1H), 4.95-4.72 (m, 1H), 3.30 (s, |
| | | | | | | 7H), 2.22-1.10 (m, 19H), 0.70-0.52 (m, 4H). |
| 60 | | B | A | 459 | 1.15 | 1H NMR (300 MHz, DMSO) d 7.31 (dd, J = |
| | | | | | | 9.9, 6.6 Hz, 1H), 4.95-4.72 (m, 1H), 3.30 (s, |
| | | | | | | 7H), 2.22-1.10 (m, 19H), 0.70-0.52 (m, 4H). |
| 61 | | B | B | 476.19 | 2.66 | (DMSO-d6, 400 MHz): 6.90 (s, 1H), 4.49 (m, |
| | | | | | | 1H), 4.29 (m, 1H), 3.39-3.16(m, 5H), 2.88- |
| | | | | | | 2.81 (m, 1H), 1.99-1.71 (m, 4H), 1.6-1.40 (m, |
| | | | | | | 2H), 1.37-1.30 (m, 2H), 1.21-1.16 (m, 8H), |
| | | | | | | 1.12-1.00 (m, 1H), 0.75 (d, J = 6.6 Hz; 3H), |
| | | | | | | 0.62-0.49 (m, 2H). |
| 62 | | A | B | | | (DMSO-d6, 400 MHz) 6.98(s, 1H), 4.51(brs, |
| | | | | | | 1H), 4.28-4.25 (m, 1H), 3.41-3.34(m, 4H), |
| | | | | | | 3.02-2.99 (m, 1H), 2.87-2.84 (m, 1H), 1.95- |
| | | | | | | 1.88 (m, 2H), 1.77-1.65 (m, 2H), 1.56-1.35 (m, |
| | | | | | | 4H), 1.23-1.05 (m, 8H), 0.85-0.78 (m, 1H), |
| | | | | | | 0.75 (d, J = 6.6 Hz; 3H), 0. |
| 63 | | A | A | 473 | 1.4 | 1H NMR (300 MHz, DMSO) d 7.28 (s, 1H), |
| | | | | | | 4.92-4.70 (m, 1H), 3.30 (s, 2H), 2.80 (d, J = |
| | | | | | | 8.3 Hz, 7H), 2.35-1.75 (m, 5H), 1.64-1.14 |
| | | | | | | (m, 15H), 0.76 (d, J = 6.5 Hz, 4H). |
| 64 | | A | B | 442.44 | 2.73 | 1H NMR (300 MHz, CDCl3) d 7.03 (s, 1H), |
| | | | | | | 6.90 (s, 1H), 6.77 (s, 1H), 5.53 (d, J = 16.4 |
| | | | | | | Hz, 1H), 4.47 (d, J = 16.1 Hz, 1H), 3.72 (s, |
| | | | | | | 3H), 2.42 (t, J = 11.8 Hz, 1H), 1.93 (d, J = |
| | | | | | | 13.0 Hz, 1H), 1.71-1.13 (m, 15H), 0.96-0.68 |
| | | | | | | (m, 5H). |
| 65 | A | A | B | 459 | 2.19 | 1H NMR (300 MHz, DMSO) d 7.21 (s, 1H), |
| | | | | | | 4.71-4.51 (m, 1H), 3.34 (s, 2H), 2.93 (d, J = |
| | | | | | | 6.3 Hz, 2H), 2.62 (s, 6H), 2.31 (s, 3H), 1.85 |
| | | | | | | (dd, J = 22.9, 11.0 Hz, 1H), 1.67-1.08 (m, |
| | | | | | | 16H), 0.69-0.51 (m, 5H). |
| 66 | | A | A | 441.93 | 2.07 | |
| 67 | | B | A | 442.18 | 1.36 | 1H NMR: 10.97 (s, 1H), 7.34 (s, 1H), 7.29 (s, |
| | | | | | | 1H), 6.75 (s, 1H), 4.61 (dd, J = 32.1, 14.7 Hz, |
| | | | | | | 2H), 3.82 (s, 3H), 2.10 (t, J = 11.5 Hz, 1H), |
| | | | | | | 1.81-1.36 (m, 6H), 1.40-1.20 (m, 10H), 0.89- |
| | | | | | | 0.58 (m, 5H). |
| 68 | | A | A | 495 | 1.53 | 1H NMR (300 MHz, DMSO) d 7.36 (d, J = |
| | | | | | | 12.4 Hz, 1H), 4.96-4.67 (m, 1H), 3.59 (d, J = |
| | | | | | | 6.5 Hz, 2H), 3.22-2.98 (m, 2H), 2.87 (d, J = |
| | | | | | | 18.7, 12.4 Hz, 3H), 2.18-1.08 (m, 19H), 0.88- |
| | | | | | | 0.50 (m, 5H). |
| 69 | | A | A | 495 | 1.53 | 1H NMR (300 MHz, DMSO) d 7.36 (d, J = |
| | | | | | | 12.4 Hz, 1H), 4.96-4.67 (m, 1H), 3.59 (d, J = |
| | | | | | | 6.5 Hz, 2H), 3.22-2.98 (m, 2H), 2.87 (d, J = |
| | | | | | | 18.7, 12.4 Hz, 3H), 2.18-1.08 (m, 19H), 0.88- |
| | | | | | | 0.50 (m, 5H). |
| 70 | | A | A | 459 | 2.24 | 1H NMR (300 MHz, DMSO) d 7.25 (d, J = |
| | | | | | | 25.5 Hz, 1H), 5.04-4.87 (m, 0.65H), 4.68- |
| | | | | | | 4.52 (m, 0.35H), 3.22 (d, J = 7.7 Hz, 1H), 2.93 |
| | | | | | | (d, J = 6.2 Hz, 1H), 2.63 (d, J = 11.1 Hz, 6H), |
| | | | | | | 1.97 (dd, J = 64.8, 9.0 Hz, 6H), 1.67-1.11 (m, |
| | | | | | | 16H), 0.69 (dd, J = 43.4, 9.1 Hz, 5H). |
| 71 | | A | B | 441.99 | 3.45 | 1H NMR (300 MHz, CDCl3) d 7.29 (d, J = 2.3 |
| | | | | | | Hz, 1H), 6.89 (s, 1H), 6.09 (d, J = 2.3 Hz, 1H), |
| | | | | | | 5.56 (d, J = 16.4 Hz, 1H), 4.38 (d, J = 16.3 Hz, |
| | | | | | | 1H), 3.83 (s, 3H), 2.35 (t, J = 11.3 Hz, 1H), |
| | | | | | | 1.88 (d, J = 11.5 Hz, 1H), 1.75-1.46 (m, 4H), |
| | | | | | | 1.45-1.14 (m, 10H), 0.91-0.58 (m, 5H). |
| 72 | | A | B | 475.26 | 4.03 | |
| 73 | | A | B | 456.26 | 1.82 | 1H NMR (300 MHz, CDCl3) d 7.48 (d, J = 3.8 |
| | | | | | | Hz, 0.7H), 7.40 (d, J = 1.4 Hz, 0.3H), 7.35 (s, |
| | | | | | | 0.3H), 6.76 (d, J = 6.6 Hz, 0.7H), 6.24 & 6.15 |
| | | | | | | (d, J = 2.0 & J = 1.9 Hz, 1H), 5.64 (d, J = |
| | | | | | | 6.8 Hz, 1H), 4.89 (s, 1H), 4.70-4.46 (m, 1H), |
| | | | | | | 4.38 (m, 1H), 3.55 (d, J = 8.9 Hz, 1H), 3.26 |
| | | | | | | (dd, J = 13.8, 9.8 Hz, 1H), 2.58 (t, J = 6.6 Hz, |
| | | | | | | 1H), 1.93 (d, J = 45.9 Hz, 1H), 1.50 (m, 7H), |
| | | | | | | 1.32 (2 x s, & m, 10H), 1.28-1.05 (m, 3H), |
| | | | | | | 0.84-0.46 (m, 2H). |
| 74 | | B | B | 438.28 | 2.29 | 1H NMR (300 MHz, CDCl3) d 7.29-7.24 (m, |
| | | | | | | 3H), 7.19 (d, J = 7.7 Hz, 2H), 6.60 (s, 1H), |
| | | | | | | 5.37 (d, J = 14.9 Hz, 1H), 4.30 (d, J = 14.2 Hz, |
| | | | | | | 1H), 2.11 (s, 1H), 1.78-1.46 (m, 7H), 1.32 (s, |
| | | | | | | 9H), 0.83 (d, J = 6.5 Hz, 3H), 0.75 (d, J = |
| | | | | | | 12.1 Hz, 2H). |
| 75 | | A | B | 472.32 | 2.19 | 1H NMR (300 MHz, CDCl3) d 7.05 (s, 0.33H), |
| | | | | | | 6.94 (s, 0.67H), 6.05 (q, J = 7.3 Hz, 0.67H), |
| | | | | | | 5.73-5.58 (q, 0.33H), 2.87-2.65 (2 x q, 2H), |
| | | | | | | 2.07 (m, 1H), 1.80-1.56 (m, 6H), 1.48-1.18 |
| | | | | | | (m & 2 x s, 16H), 0.82 (2 x d, 3H). 0.74 (m, |
| | | | | | | 2H). |
| 76 | | A | A | 432 | 1.24 | 1H NMR (300 MHz, DMSO) d 13.41 (s, 1H), |
| | | | | | | 7.17 (s, 1H), 4.63 (ddd, J = 17.3, 9.7, 7.5 Hz, |
| | | | | | | 1H), 4.34 (s, 1H), 3.14 (s, 2H), 2.10 (dt, J = |
| | | | | | | 17.2, 7.0 Hz, 1H), 2.02-1.13 (m, 21H), 0.72 |
| | | | | | | (t, J = 20.6 Hz, 5H). |
| 77 | | A | A | 442.5 | 1.58 | 1H NMR (300 MHz, CDCl3) d 11.02 (s, 1H), |
| | | | | | | 7.27 (d, J = 2.1 Hz, 1H), 6.87 (s, 1H), 5.81- |
| | | | | | | 5.57 (m, J = 9.2 Hz, 2H), 4.04 (d, J = 15.0 Hz, |
| | | | | | | 1H), 3.91 (s, 3H), 2.30-2.00 (m, 1H), 1.81 (d, |
| | | | | | | J = 11.1 Hz, 1H), 1.76-1.43 (m, 5H), 1.43- |
| | | | | | | 1.16 (m, 10H), 0.93-0.54 (m, 5H). |
| 78 | | A | B | 458.24 | 2.08 | 1H NMR (300 MHz, CDCl3) d 7.09 (s, 1H), |
| | | | | | | 5.44 (d, J = 17.0 Hz, 1H), 4.66 (d, J = 17.0 Hz, |
| | | | | | | 1H), 2.77 (q, J = 7.6 Hz, 2H), 2.19 (dd, J = |
| | | | | | | 15.7, 7.4 Hz, 1H), 1.84-1.43 (m, 8H), 1.35 (s, |
| | | | | | | 9H), 1.33-1.27 (t, 3H), 0.84 (d, J = 6.5 Hz, |
| | | | | | | 3H), 0.75 (m, 1H). |
| 79 | | B | B | 486.27 | 2.43 | 1H NMR (300 MHz, CDCl3) d 7.03 & 6.89 (2 x |
| | | | | | | s, 1H), 6.07 (q, J = 7.3 Hz) & 5.75 (q, J = |
| | | | | | | 7.0 Hz)(2 x q, 1H), 3.21-2.97 (m, 1H), 2.08 (dd, |
| | | | | | | J = 14.8, 7.7 Hz, 1H), 1.79-1.52 (m, 8H), 1.45- |
| | | | | | | 1.24 (m, 18H), 0.82 (d, J = 6.5 Hz, 3H), 0.71 |
| | | | | | | (m, 1H). |
| 80 | | A | B | 456.26 | 1.82 | 1H NMR (300 MHz, CDCl3) d 7.65-7.48 (m, 2H), |
| | | | | | | 6.90 & 6.40 (2 x s, 1H), 6.29 (dd, J = 5.3, |
| | | | | | | 2.2 Hz, 1H), 4.98-4.76 (m, 1H), 4.59-4.40 |
| | | | | | | (m, 1.5H), 4.10 (dd, J = 13.5, 7.6 Hz, |
| | | | | | | 0.5H), 1.96 (q, J = 11.6 Hz, 1H), 1.76-1.39 |
| | | | | | | (m, 6H), 1.34 (2 x s, 9H), 1.24 (d, J = |
| | | | | | | 11.1 Hz, 1H), 1.15 & 1.05 (2 x d, J = 6.9 Hz, 3H), |
| | | | | | | 0.84-0.73 (m, 3H), 0.73-0.51 (m, 2H). |
| 81 | | B | B | 460.01 | | ¹H NMR (400 MHz, DMSO-d₆): δ 7.29 (d, 2H), |
| | | | | | | 7.20 (d, 2H), 6.99 (s, 1H), 4.60 (d, 1H), 4.39- |
| | | | | | | 4.28 (m, 1H), 4.28-4.19 (m, 1H), 3.29-3.17 |
| | | | | | | (m, 1H), 3.15 (d, 2H), 1.97-1.82 (m, 3H), |
| | | | | | | 1.82-1.71 (m, 1H), 1.29-1.20 (m, 9H), 1.01- |
| | | | | | | 0.86 (m, 2H) |
| 82 | | B | B | 494.08 | | |
| 83 | B | B | B | 454.13 | | 1H NMR (400 MHz, DMSO-d6): δ 7.15-7.01 |
| | | | | | | (m, 2H), 6.86 (s, 1H), 6.72 (d, J = 7.7 Hz, 1H), |
| | | | | | | 4.61-4.33 (m, 2H), 3.44-3.21 (m, 2H), 2.18 |
| | | | | | | (d, J = 17.2 Hz, 6H), 2.03-1.96 (d, J = |
| | | | | | | 11.9 Hz, 1H), 1.90-1.75 (m, 3H), 1.45-1.28 (m, |
| | | | | | | 3H), 1.25 (s, 9H), 0.99-0.85 (m, 1H) |
| 84 | | A | B | 474.07 | | |
| 85 | | A | B | 458.11 | | ¹H NMR (400 MHz, DMSO-d₆): δ 13.48 (s, |
| | | | | | | 1H), 7.17 (s, 1H), 7.10 (t, 1H), 6.86 (d, 2H), |
| | | | | | | 4.55 (d, 1H), 4.45-4.32 (m, 1H), 3.31-3.20 |
| | | | | | | (m, 1H), 2.22 (s, 3H), 2.00-1.69 (m, 5H), |
| | | | | | | 1.55-1.39 (m, 2H), 1.28 (s, 9H), 1.07-0.89 |
| | | | | | | (m, 2H) |
| 86 | | A | B | 440.13 | | |
| 87 | | C | A | 461.94 | | ¹H NMR (400 MHz, DMSO-d₆): δ 13.66 (s, 1H), |
| | | | | | | 7.36-7.12 (m, 5H), 4.57 (s, 1H), 4.41 (s, 1H), |
| | | | | | | 3.30-3.22 (m, 1H), 2.09-1.70 (m, 5H), 1.54-1.39 |
| | | | | | | (m, 2H), 1.35-1.18 (m, 9H), 1.03-0.89 (m, 1H) |
| 88 | | B | A | 439.98 | | ¹H NMR (400 MHz, DMSO-d₆): δ 13.49 (s, 1H), |
| | | | | | | 7.24 (s, 1H), 7.13-6.92 (m, 4H), 4.56 (d, 1H), |
| | | | | | | 4.48-4.36 (m, 1H), 3.31-3.22 (m, 1H), 2.23 (s, |
| | | | | | | 3H), 2.06-1.72 (m, 5H), 1.54-1.39 |
| | | | | | | (m, 2H), 1.33-1.31 (m, 1H), 1.26 (s, 9H), |
| | | | | | | 1.04-0.90 (m, 2H) |
| 89 | | A | B | 475.97 | | ¹H NMR (400 MHz, DMSO-d₆): δ 13.56 (s, 1H), |
| | | | | | | 7.25 (s, 1H), 7.03-6.83 (m, 2H), 4.56 (d, 1H), |
| | | | | | | 4.45-4.30 (m, 1H), 3.30-3.17 (m, 1H), 2.19 (s, |
| | | | | | | 3H), 2.05-1.65 (m, 5H), 1.58-1.38 |
| | | | | | | (m, 1H), 1.37-1.15 (m, 10H), 1.07-0.90 (m, 1H) |
| 90 | | B | B | 494.03 | | ¹H NMR (400 MHz, DMSO-d₆): δ 13.71 (s, 1H), |
| | | | | | | 7.54 (d, 1H), 7.35 (dd, 1H), 7.25 (d, 1H), |
| | | | | | | 7.21 (s, 1H), 4.58 (s, 1H), 4.44-4.34 (m, 1H), |
| | | | | | | 3.30-3.23 (m, 1H), 2.05-1.99 (m, 1H), 1.93-1.69 |
| | | | | | | (m, 4H), 1.53-1.41 (m, 2H), 1.27 (s, 9H), 1.02- |
| | | | | | | 0.90 (m, 2H). |
| 91 | B | B | B | 478.04 | | ¹H NMR (400 MHz, DMSO-d₆): δ 7.51 (m, 1H), |
| | | | | | | 7.26 (m, 1H), 7.08 (m, 1H), 6.84 (s, 1H), |
| | | | | | | 4.53 (bs, 1H), 4.34 (m, 1H), 3.25 (m, 1H), |
| | | | | | | 1.95-1.84 (m, 3H), 1.82-1.73 (m, 1H), 1.44-1.12 |
| | | | | | | (m, 12H), 0.99-0.84 (m, 1H) |
| 92 | | C | A | 444.09 | | |
| 93 | B | B | B | 458.11 | | |
| 94 | | B | B | 478.06 | | ¹H NMR (400 MHz, DMSO-d₆): δ 13.59-13.40 |
| | | | | | | (m, 1H), 7.48 (t, 1H), 7.38 (s, 1H), 7.20 |
| | | | | | | (d, 1H), 7.03 (d, 1H), 4.55 (d, 1H), 4.43-4.32 |
| | | | | | | (m, 1H), 3.31-3.21 (m, 1H), 2.02-1.71 (m, 5H), |
| | | | | | | 1.51-1.39 (m, 2H), 1.29 (s, 9H), 1.27-1.17 |
| | | | | | | (m, 1H), 1.08-0.94 (m, 1H) |
| 95 | B | | B | 530.4 | 2.10 | 1H NMR (300 MHz, DMSO) 7.94 (br s, 3H), |
| | | | | | | 7.21 (d, J = 5.5 Hz, 1H), 4.65-4.27 (m, 2H), |
| | | | | | | 3.81 (d, 1H), 3.38 (m, 3H), 2.98 (m, 1H), |
| | | | | | | 2.88 (dd, 1H), 2.72 (s, 2H), 2.24 (m, 2H), 1.83 |
| | | | | | | (m, 2H), 1.76-1.40 (m, 9H), 1.3-1.15 (m, 4H), |
| | | | | | | 1.20 (t, 6H), 0.89 (m, 1H), 0.75 (d, 3H), 0.61 |
| | | | | | | (m, 2H). |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound represented by any one of the following structural formulae or a pharmaceutically acceptable salt thereof:

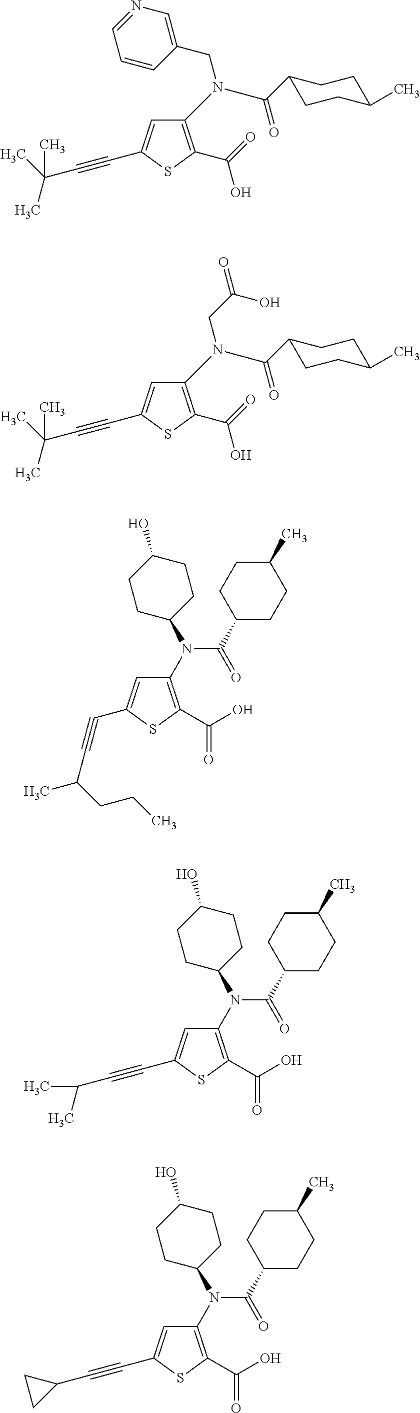

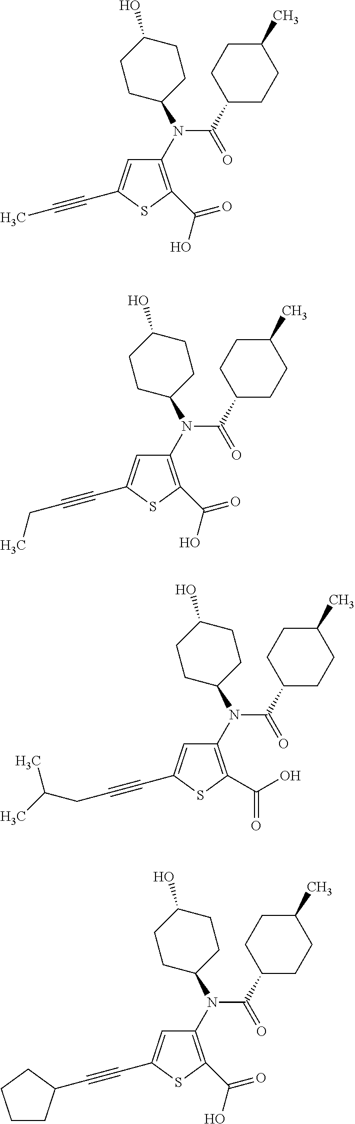

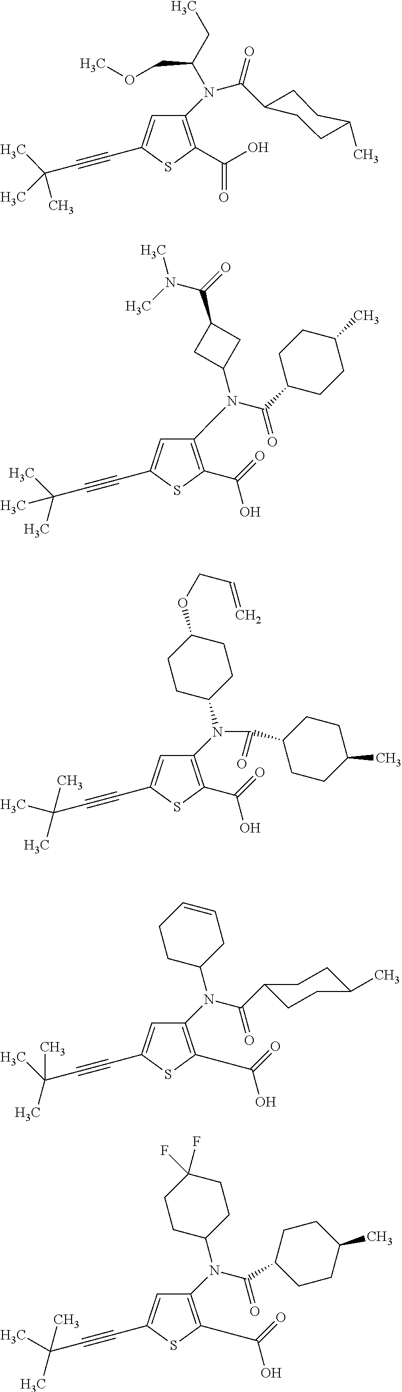

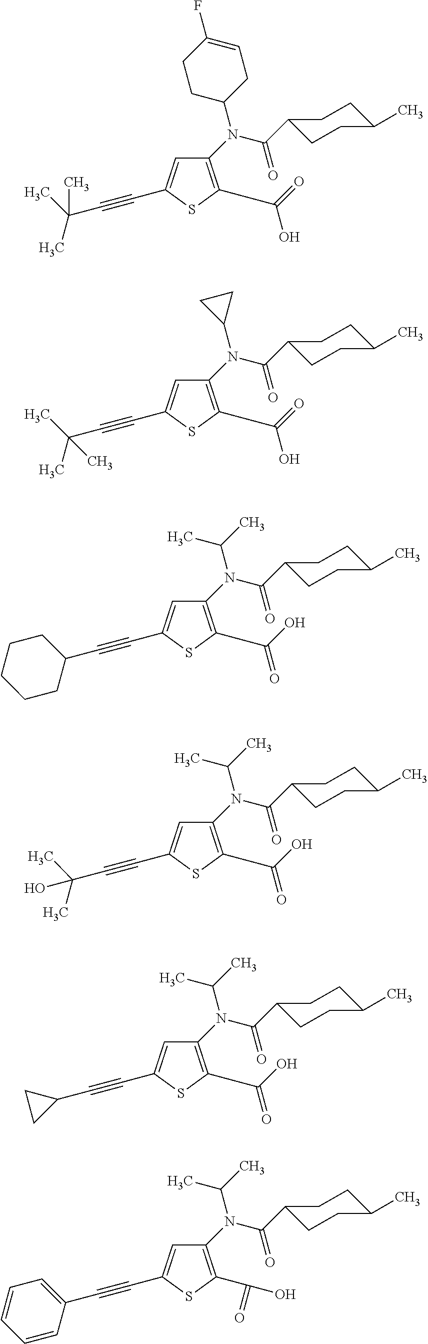

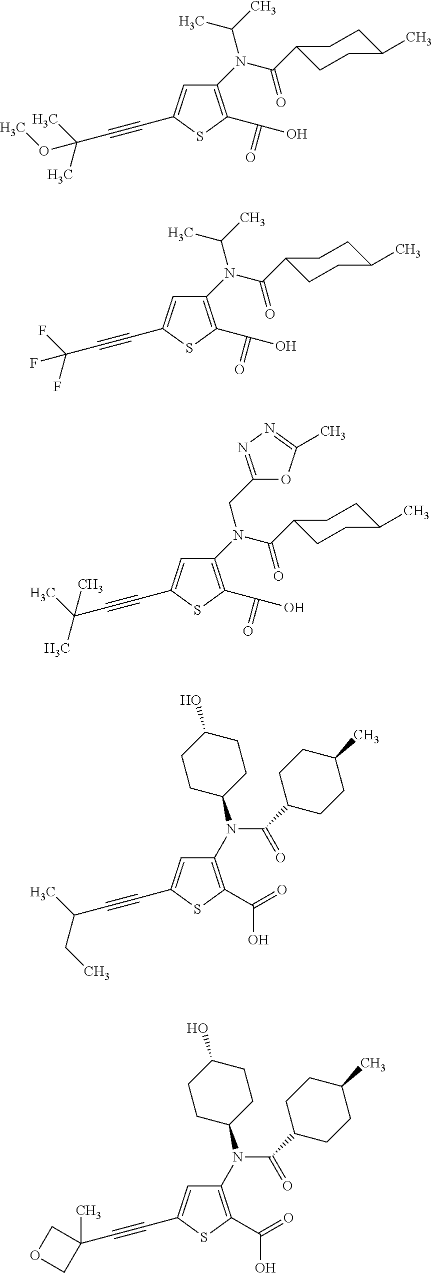

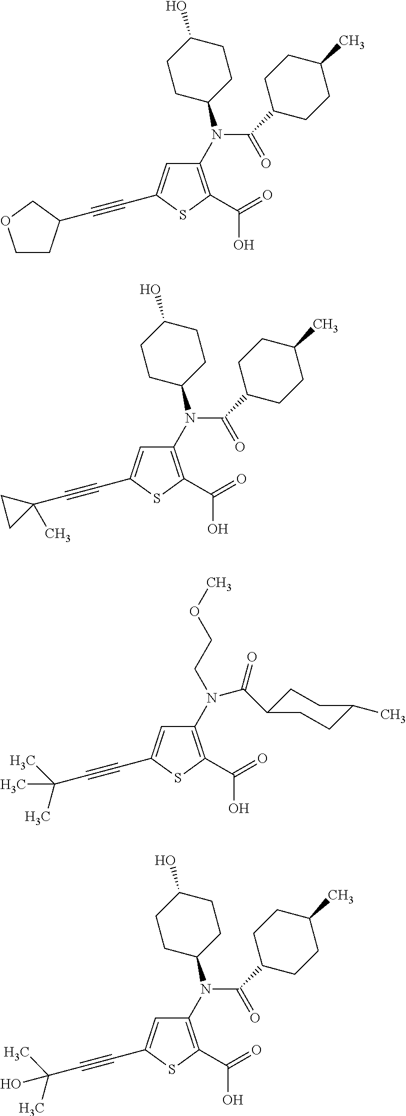

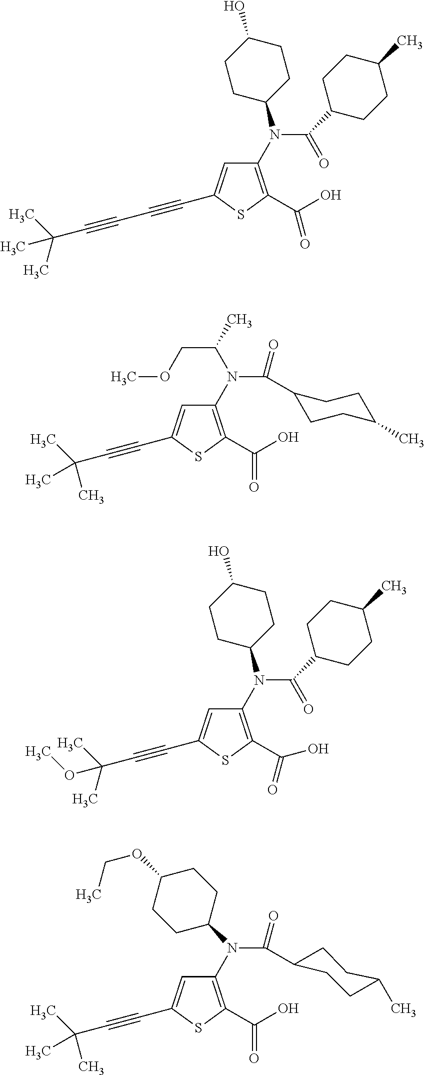

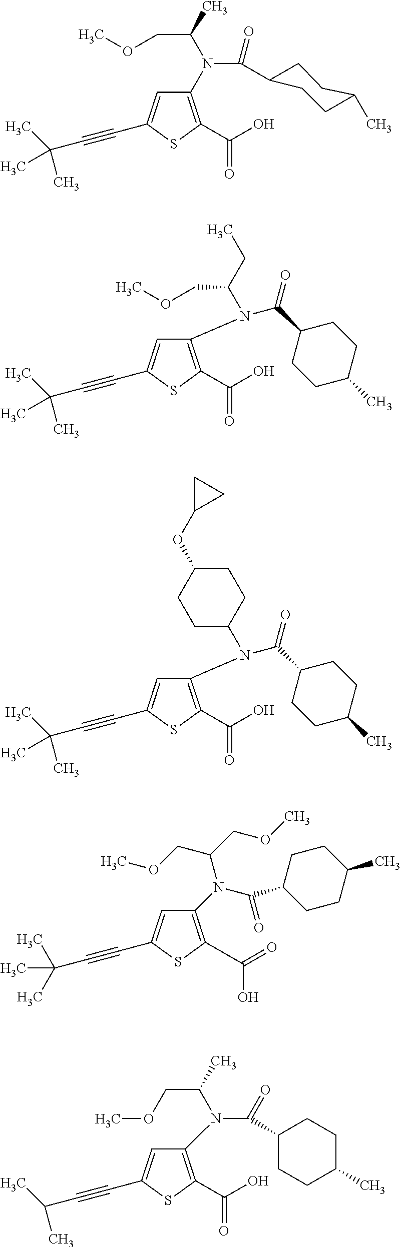

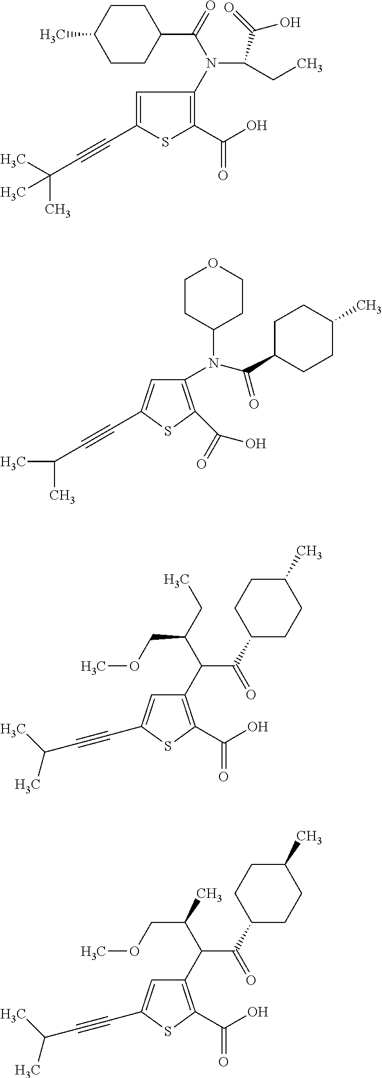

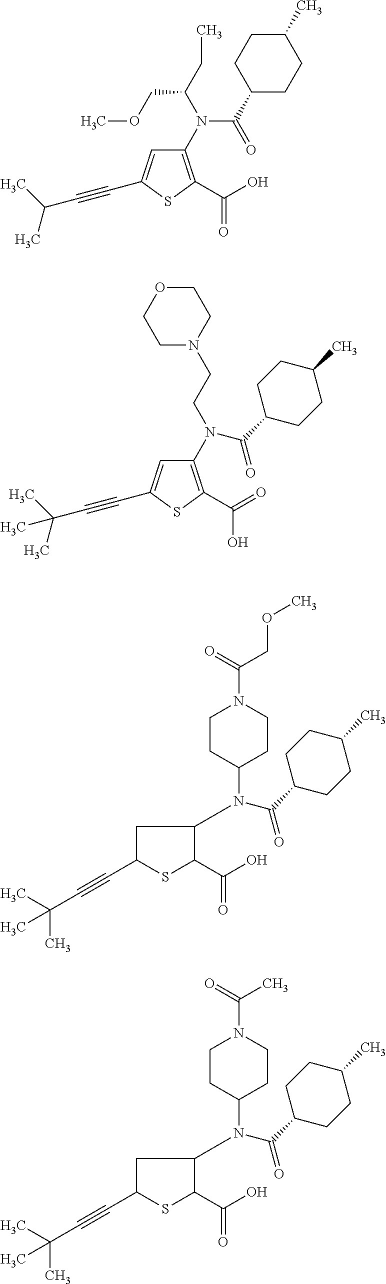

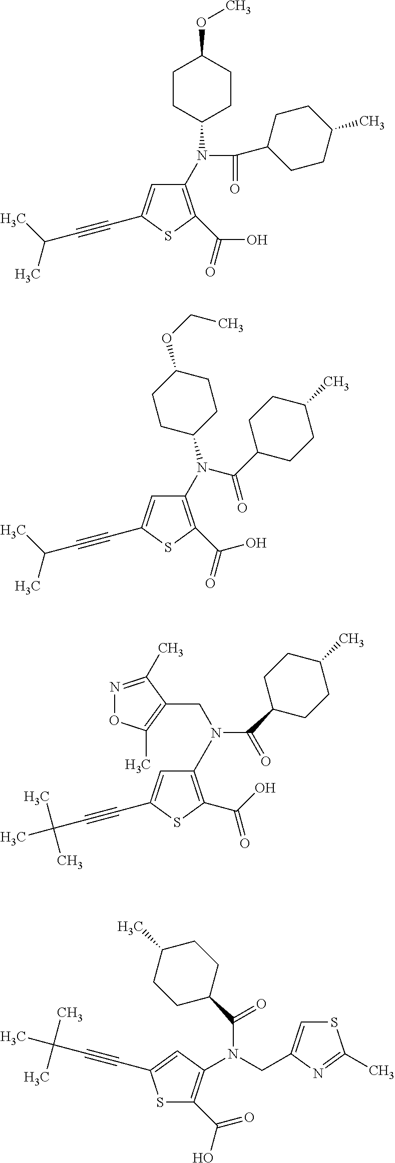

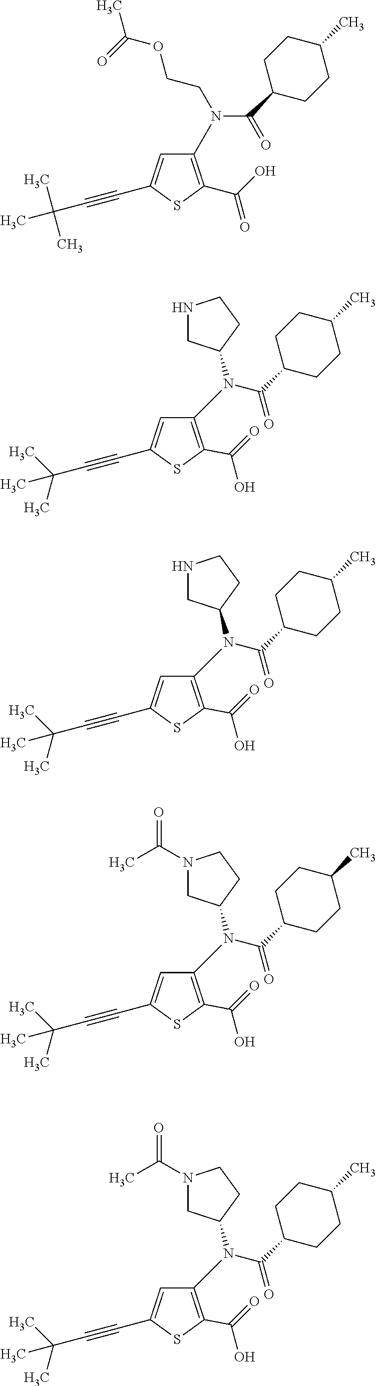

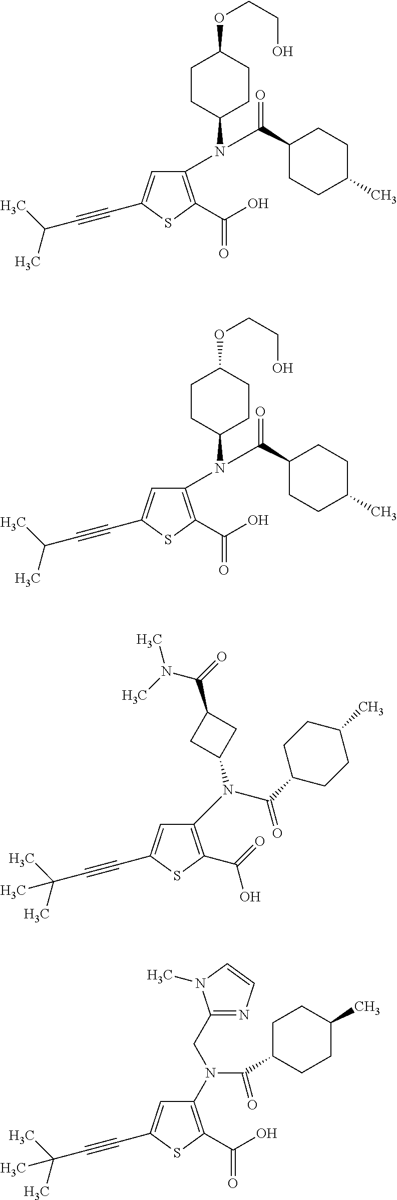

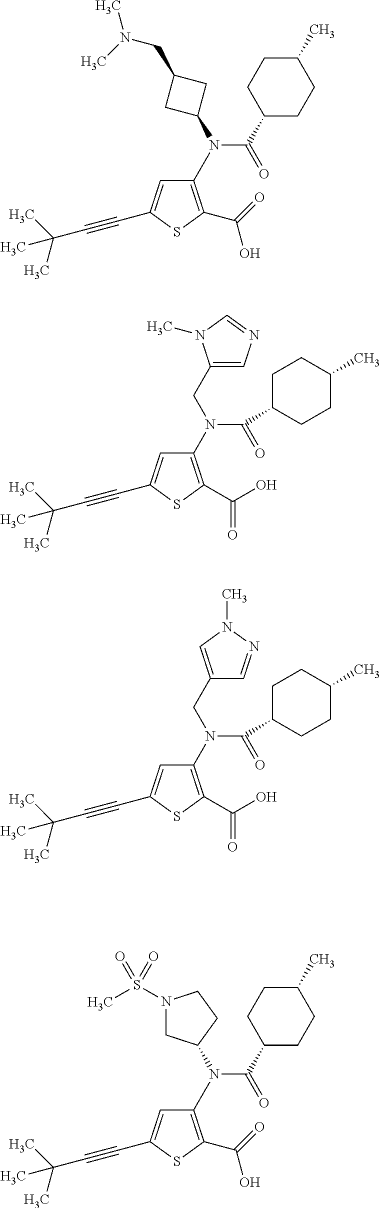

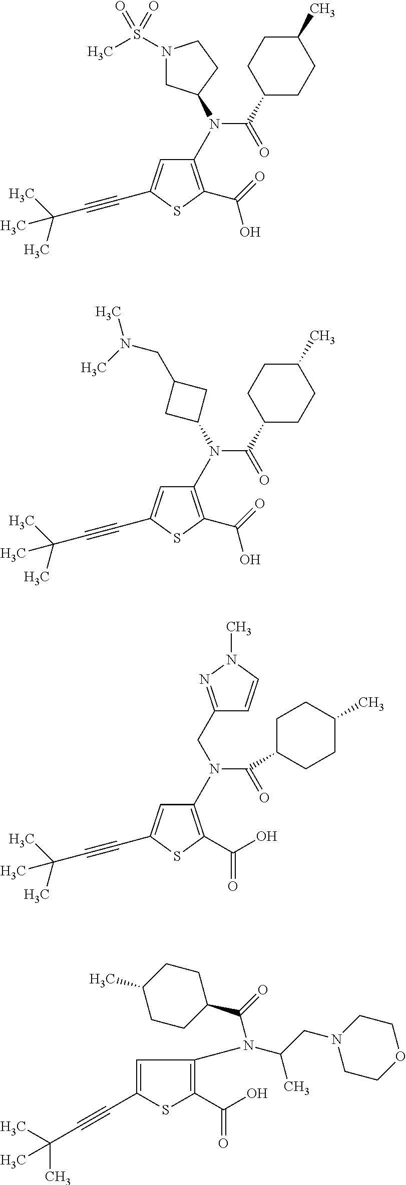

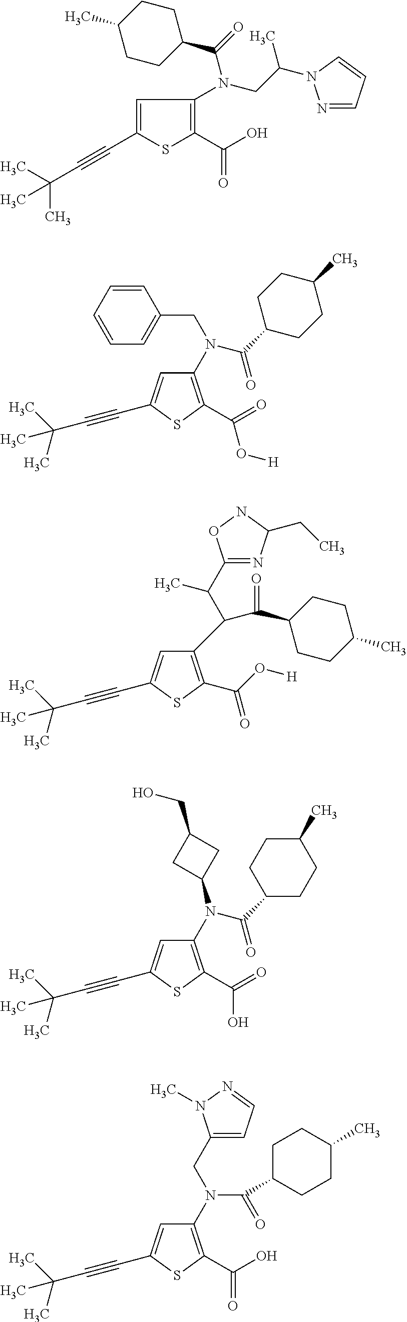

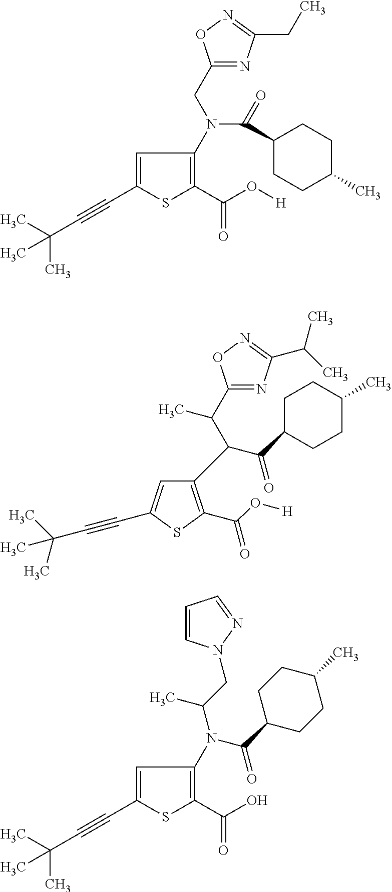

2. The compound of claim 1, selected from the following structural formulae: 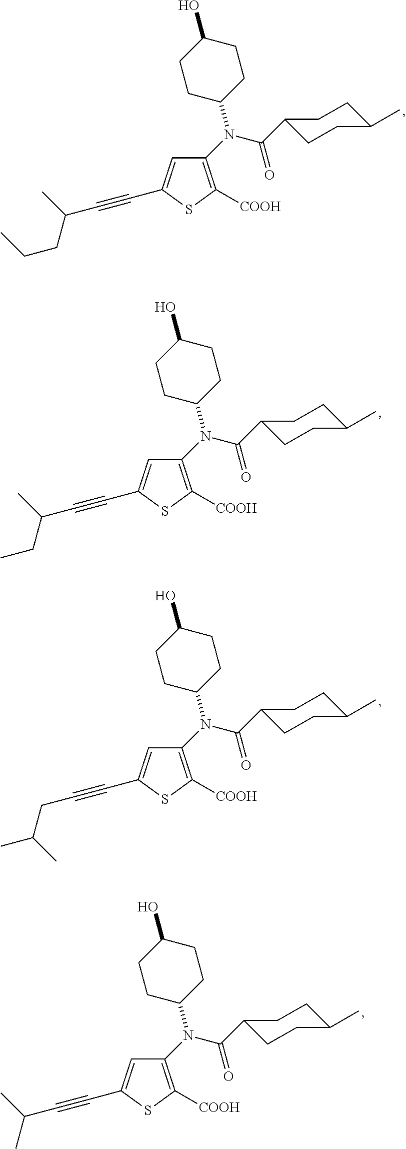 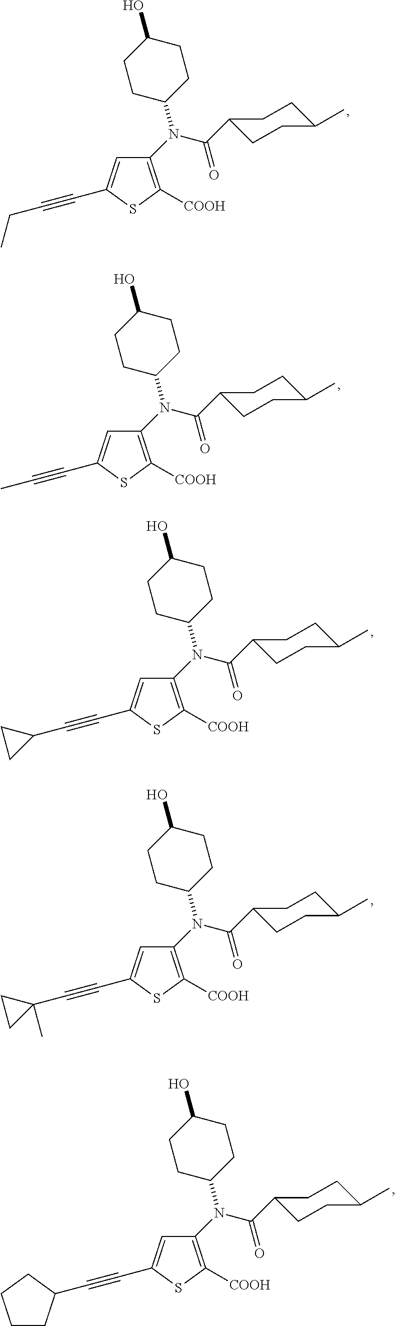 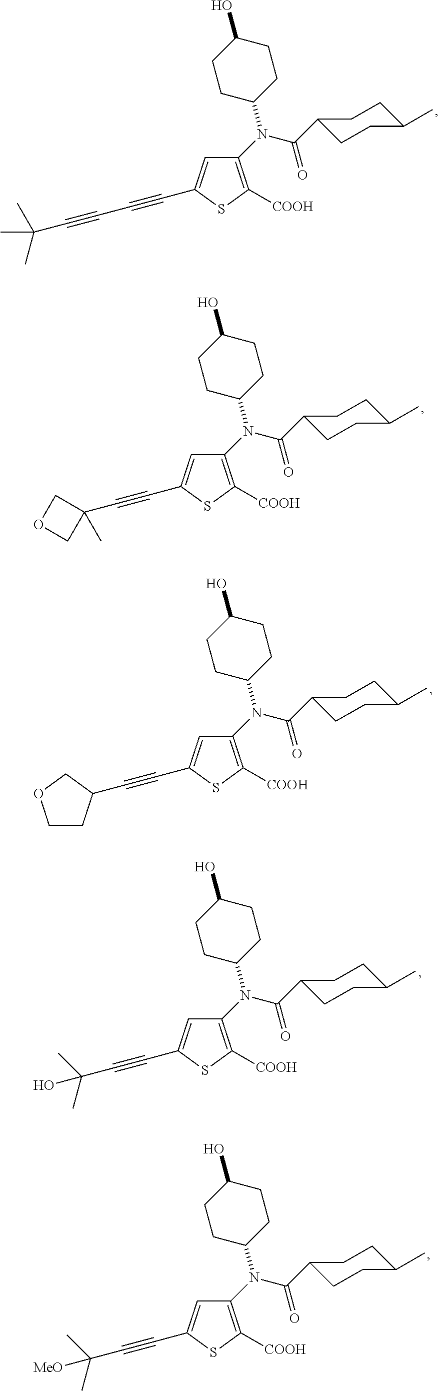 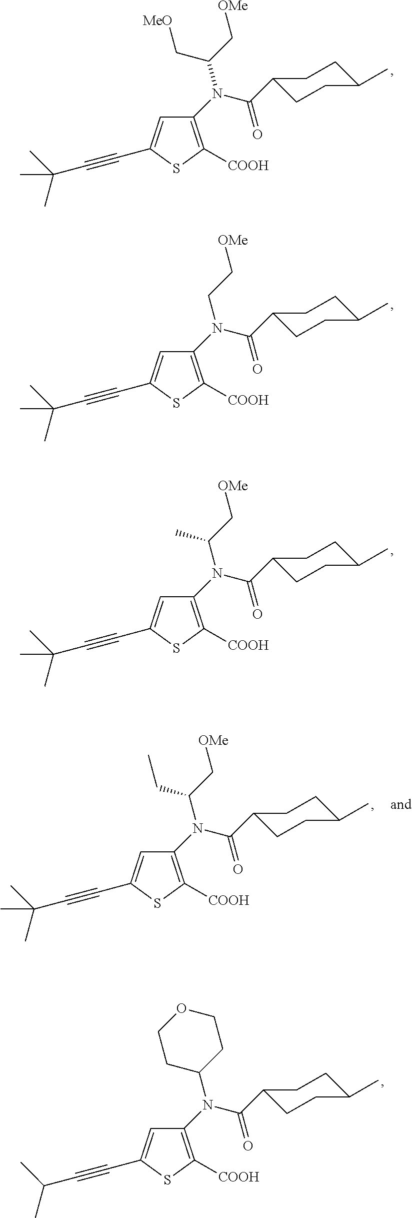
or a pharmaceutically acceptable salt thereof.

3. A compound represented by the following structural formula or a pharmaceutically acceptable salt thereof:

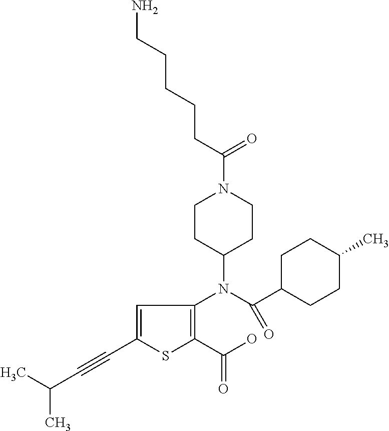

4. A pharmaceutical composition, comprising a compound of claim 1 or claim 2, and a pharmaceutically acceptable carrier or excipient.

5. A pharmaceutical composition, comprising a compound of claim 3 and a pharmaceutically acceptable carrier or excipient.

6. A method of inhibiting or reducing the activity of HCV polymerase in a biological in vitro sample, comprising administering to the sample an effective amount of a compound of any one of claims 1-3.

7. A method of treating a HCV infection in a subject, comprising administering to the subject a therapeutically effective amount of a compound of any one of claims 1-3.

8. A method of inhibiting or reducing the activity of HCV polymerase in a subject, comprising administering to the subject a therapeutically effective amount of a compound of any one of claims 1-3.

9. The method of claim 7 or 8, further comprising co-administering one or more additional therapeutic agents to the subject.

10. The method of claim 9, wherein the additional therapeutic agents include an anti-HCV drug.

11. The method of claim 10, wherein the anti-HCV drug is an HCV protease inhibitor.

12. The method of claim 11, wherein the HCV protease inhibitor is an HCV NS3 inhibitor.

13. The method of claim 11, wherein the HCV protease inhibitor is VX-950.

14. The method of claim 10, wherein the anti-HCV drug is an HCV NS5A inhibitor.

15. The method of any one of claims 9-14, wherein an interferon and/or ribavirin is co-administered.

16. The method of claim 15, wherein the interferon is a pegylated interferon.

17. The method of claim 16, wherein the pegylated interferon is a pegylated interferon-alpha.

18. The method of claim 17, wherein the pegylated interferon is pegylated interferon-alpha 2a or pegylated interferon-alpha 2b.

19. The method of any one of claims 6-18, wherein the HCV is genotype 1.

20. The method of any one of claims 6-18, wherein the HCV is genotype 1a or genotype 1b.
